SEQUENCE LISTING (83 sequences)

SEQ ID NO: 1 - DNA, 10938 bp, Oryza sativa
atggcggcggcggcggccatggcggcgcaccgggccagcttcccgctccggctgcagcagatcctgtccgggagccgcgccgtgtcgccgtcgatcaaggtggagtccgagccgccagcaaaagttaaagcatttattgatcgtgtaatcagtattccactacatgacattgctataccattgtcaggcttccgttgggagttcaataagggaaatttccaccattggaagcctctttttatgcattttgatacatatttcaagacacaaatttcttcgaggaaggatcttcttttatctgatgatatggctgagggtgatcctttgcctaaaaataccatcctgcagattttgagagtaatgcagattgttttggaaaattgccagaacaaaacatcgtttgctggtcttgagcattttaggcttctgctggcatcatcagatcctgagatagttgtggctgctttagagacacttgctgcattggttaaaataaatccttcgaagttgcatatgaacggaaagctcataaattgtggagctataaacagtcatcttctatcattggcacaaggatggggtagcaaggaggaaggtttgggcttatattcttgtgttgtggcaaatgaaagaaaccagcaggagggtttgtgcttattcccagcagacatggagaacaaatacgatggcacgcagcaccgtctcggttcaactcttcattttgaatataatttggcacctgcccaagatcctgaccaatccagtgacaaggctaagccatctaatctgtgtgtgatacatatcccagacttgcaccttcagaaggaggatgacttgagcatattgaagcaatgtgttgataagtttaatgtgccttcagagcacagattttccttgtttacaaggataagatatgcccatgcctttaattcgccacggacatgtaggctatatagccgcataagtcttcttgctttcattgttcttgtgcaatccagcgatgcccatgatgaactcacatctttctttacaaatgagccagagtacataaatgagttaatcagacttgtccgatcagaggaatttgttcctggacccatacgagcgctggctatgcttgcactgggagcacagttagcagcgtatgcatcatctcatgaacgagctcggatacttagtggctcaagtatcatatctgctggtggaaaccgcatggtcttgctcagtgttttgcaaaaagctatatcatcactcagtagccctaatgatacatcatctccattaattgttgatgcccttctgcagttttttctgctccatgtgctatcttcttcgagttctgggaccactgttagaggttcagggatggttcccccgctcttgccccttttgcaagataatgatccttcacacatgcatcttgtctgtctggcagtgaaaactcttcaaaagttgatggagtacagcagccctgctgtttctctatttaaagatttgggtggtgtagaacttttgtctcagaggttgcacgtggaggtgcagcgtgttattggtgttgacagtcataattcaatggttacaagtgatgcattgaaatcagaagaggatcatctctactctcagaagcgattgattaaggcgctgctaaaggcattggggtctgctacatattctcctgcaaatcctgctcgttcacaaagctcaaatgataattctttgcccatctcgctttcccttatatttcagaatgttgacaagtttggtggtgacatttatttctcagcagttactgttatgagtgagataattcacaaggatccaacatgctttccttctttgaaggaacttggtcttccagatgcttttctatcgtcagtgagtgctggggtaataccatcttgtaaagctctcatctgtgtgcctaatggtctgggtgcaatatgccttaataaccaaggacttgaggctgtcagggaaacttcagctctgcgttttcttgttgacacattcaccagcaggaagtacttgataccaatgaatgaaggtgttgtcctattagctaatgcagtggaagagcttctacgtcacgtgcagtccctaagaagcactggggttgacatcattattgaaataattaataaactttcttcacctcgtgaagataagagcaatgaaccagcggccagttctgatgaaagaacagaaatggaaactgacgcggaaggacgtgatttggtaagtgctatggattccagtgaggatggcactaatgatgaacagttttctcatttgagcattttccatgtgatggtattggttcatcggacaatggagaactccgaaacctgccggttatttgtggagaaaggaggcctgcaagcacttttgacactcctgttgcgacctagcattacccaatcatctggaggaatgccgattgctttgcatagcaccatggtattcaagggctttactcagcatcactctactccacttgcacgtgcattttgctcttccttaaaggagcatttaaagaatgccttgcaggaacttgatacagttgcaagctctggtgaagtggcaaagttagaaaaaggagcaattccatctctttttgttgttgagttcttactcttccttgcggcatccaaagataatcgctggatgaatgctctactctcagaatttggagatagcagtagggatgtcctggaagatattggacgagtacaccgagaagtgctttggcaaatttcactttttgaagaaaagaaagttgagcctgaaacaagttctcctttagcaaatgactcccagcaagacgcagctgtgggggatgttgatgatagcagatacacatcctttaggcaatatcttgatcctcttttgaggcgaaggggctctgggtggaatattgaatcacaggtgtctgacctcattaatatctaccgtgatattggccgtgcagctggtgactctcagaggtatcctagtgcagggttgccctcaagttcttctcaagaccagcctcccagttcatctgatgcaagtgctagcacaaaatcagaagaggacaagaaaagatctgagcattcttcctgctgtgacatgatgaggtcactgtcttaccatatcaatcatcttttcatggagcttgggaaagcaatgcttcttacatctcgtcgggagaacagccctgtgaatttatctgcatctattgtatctgttgctagcaatattgcttctattgtgttggagcacctcaattttgaggggcacacaatcagttctgaaagagagactactgtttccacaaaatgccgataccttgggaaggtggttgagttcattgatggtatattgttggacaggccggaatcgtgcaacccaatcatgctgaattcattttattgccgtggtgttattcaggctattttaaccacatttgaagctaccagtgagttgctcttttctatgaacaggcttccgtcatcgcctatggagacagacagtaaaagtgttaaggaagacagggagacagattcgtcatggatatatggtccactctccagctatggtgcaattctggaccatctagtaacatcatcgtttattctttcttcctcaacaagacaattacttgagcagcctatttttagtggaaatatcaggtttccccaagatgcagagaagttcatgaagctgcttcagtcaagagttctgaagactgttcttcccatctggacccatcctcagtttccagaatgtaatgttgagttaattagttcagtcacatctatcatgaggcatgtttactctggggttgaagtgaaaaacactgctatcaacactggtgctcgtttggctggtccaccccctgatgagaatgcaatttctctgattgtagagatgggcttttctcgcgccagagctgaggaagcactcaggcaagttggaacgaacagtgttgaaattgcaactgattggttattctcacacccagaggaaccacaagaggatgacgaacttgctcgagctcttgcaatgtctttaggcaattctgatacgtctgcacaagaggaagatggcaaatcgaatgatcttgaacttgaagaagaaactgttcagctgcctcccatagatgaagtattgtcttcatgtcttaggttgcttcagacaaaggaatcattagctttccctgttcgggacatgcttttgactatgagctcacagaatgatggtcaaaaccgagtaaaggttcttacgtatttgattgatcacctgaaaaattgtctgatgtcatctgatcctttaaagagcactgcattatcagctctttttcatgtccttgctttgattctccatggagatactgctgctcgggaagttgcttcaaaggctggtcttgtcaaggttgctttgaacctgctgtgcagctgggagttggagccgaggcaaggcgagataagtgatgttccaaattgggttccttcatgctttctttctattgataggatgctccagttggacccaaagttgccagatgttactgaactcgatgtccttaaaaaggataattcaaatacacaaacatcagtggtgattgatgatagcaagaaaaaggactcagaagcttcatcgagcacagggttattggacttggaggaccagaagcaacttttgaagatttgctgtaaatgcattcagaagcagttgccttctgctaccatgcatgctattcttcagttatgtgccacgttgactaaacttcatgctgctgctatttgttttcttgagtctggtggtctgcatgcattgctaagtttgcccacaagtagcttgttttctggattcaacagtgtggcttctacaatcattcgtcatattttggaagatccccacactcttcagcaagcaatggaattagagatacgccacagtcttgtcaccgctgcaaatcgtcatgcaaatccaagggttacaccgcgcaattttgtccagaacttggcgtttgttgtatatagagacccagtgatatttatgaaagctgcccaagctgtgtgccagattgagatggttggtgatagaccatatgttgttctgttgaaggatcgtgaaaaagaaaagaacaaggaaaaagagaaggacaagcctgctgataaggataaaacatcaggtgcagccacaaagatgacatcaggggacatggctttaggatctcctgtaagttctcaagggaagcagactgatctgaatacaaagaatgtgaaatctaatcgcaaaccaccacaaagctttgtcactgttattgagtatctgctagatctggttatgtccttcattccacctcctagagcagaagatcgacctgatggtgaatctagtactgcatcatctacagacatggatattgacagctcagcaaaaggcaaaggtaaagctgttgctgtcacacctgaagagtccaagcatgcaattcaagaggctactgcatctctcgctaaaagtgcatttgttctgaagctgctaacagatgttcttctgacttatgcatcatctattcaagttgttcttcgacatgatgctgatttgagcaatgcacgtggtcctaaccggattggtattagcagtggtggggttttcagtcatatactgcagcatttccttccgcattctacaaagcaaaagaaagagaggaaagctgatggagattggaggtacaaattggcaacaagggctaatcaattcttggtggcttcatctattcggtctgcagaaggtagaaaaaggatcttttctgaaatctgcagcatatttgttgacttcacagactcccctgctggttgcaaacccccaatattaaggatgaatgcatatgttgatttgcttaatgatattctgtcagcccgttcgccaactggttcctccttgtcagcagaatctgcagttacttttgttgaagttggtcttgttcagtatttatcaaaaacactgcaagttatagatttggatcatcctgattcagcaaagattgtaactgctattgttaaggcccttgaggttgtcacaaaggaacatgttcattcggcagatttgaatgccaaaggggagaactcatcaaaggttgtgtctgaccagagcaatctagacccgtcttcaaatagattccaagctcttgacacaactcaacccactgagatggttactgatcatagggaagctttcaatgctgttcaaacttcacaaagttcagattcagtggctgatgagatggaccatgaccgtgatctggatggaggatttgctcgtgatggtgaagatgactttatgcacgagattgctgaagatggaactccaaatgagtccacaatggaaatcagatttgaaattccacgaaatagagaggatgatatggctgatgatgacgaggacagtgatgaggacatgtcagccgatgatggtgaggaggttgatgaagatgaagacgaggatgaggatgaagagaacaacaacctggaggaggatgatgcccatcaaatgtctcatcctgacacagatcaggaggaccgtgagatggatgaagaggagtttgacgaggatctgctagaagaagatgatgatgaggatgaggatgaggaaggagtcattcttcgcctcgaagagggtatcaatggaattaatgtgtttgaccatatcgaggtgtttgggggaagcaacaatttgtctggggatacactgcgtgtaatgccgttggacatttttggaacaagacggcaaggtcgtagtacatctatatataaccttcttgggagagcaggcgatcatggtgtttttgaccacccgctcttggaggagccttcttcggtgctacaccttccacagcaaagacaacaagaaaatttagttgagatggccttctctgatcggaatcatgataatagttcttcccgcttggatgcaattttccggagcctgcgaagtggccggagtggacaccgttttaatatgtggctagatgacagtccccaacgcactggatcagctgctcctgcagtacctgaaggcattgaggagctgctggtctctcagttgagacgacccacccctgaacaacctgatgagcagagtacacctgctggtggcgctgaagaaaatgaccaatctaatcagcaacatttgcatcaatcagaaactgaggcaggaggagatgcaccaacagaacaaaatgaaaacaatgataatgcagttactccggcagcaaggtctgagttagatggttctgaaagtgctgatcctgcacctcccagcaatgcacttcaaagagaagtgtctggtgcaagtgagcatgccacggagatgcaatatgaacgtagtgatgctgtagtacgtgatgtggaagcagtcagccaggcaagcagtggtagcggtgctactttaggggaaagccttagaagtttagaggtggagataggaagtgttgaagggcatgatgatggtgatcgccacggagcttcagacaggcttcctttgggtgatttgcaggcagcttcaagatcaaggaggccacctggaagtgttgtgctaggtagcagcagagatatatctctggagagtgtcagcgaggttcctcaaaatcaaaatcaagaatctgatcagaatgctgatgaaggggatcaggagcctaacagagctgctgacactgactcaattgatcctacatttttggaggctcttccagaggatttacgggctgaagttctttcttcacgtcaaaatcaagtgacccagacttctaatgaacaacctcagaatgatggggatattgatcctgaattccttgctgcacttcctcctgatatacgtgaagaagttctagctcaacaacgtgcgcaaaggttgcagcagtcacaggaattagaaggacaaccagttgaaatggatgctgtttcaattatcgcaacattcccttcagaaattcgggaggaggtgcttttaacatctccagatacattactggctacacttacgcctgcactagttgctgaagcaaacatgttaagggagagatttgctcatcggtatcacagtggctccctttttggcatgaactccaggggcaggagaggtgagtcctctcgacgtggtgacataattggttcaggtcttgatagaaatgctggtgattcttctcgacaaccaactagcaagccaattgaaacggaaggatctcctcttgttgacaaggatgctcttaaagctcttattaggctactccgggttgttcagcctctatacaaaggtcaattgcagaggcttctcttgaacctttgtgctcatagggaaagcagaaagtccttggttcaaattctagtggacatgcttatgcttgatctgcagggctcttctaagaaatcaattgatgcaactgagccaccatttaggctatatgggtgccatgcaaatattacgtactcacgccctcaatcgacagatggcgtgcctccattagtttctcgtcgtgttcttgaaactttgacatacttggcaagaaatcatccaaatgtggctaaactcttgctatttcttgagttcccttgccccccaacttgccatgctgaaacatctgatcagaggcgtggcaaggctgttcttatggaaggtgacagtgaacagaacgcttatgcacttgtcctacttttaaccttgttgaatcagccactttatatgaggagcgtagctcatcttgaacagctactaaaccttctcgaagttgttatgctcaatgccgagaatgaaattacacaagctaagctggaagcagcatctgaaaaaccatctggacctgagaatgcaacgcaagatgcccaagagggtgcgaatgctgctggatcatctggatcgaagtccaatgctgaggatagcagcaaactccctcctgttgatggtgaaagtagcctgcaaaaagttctgcagagtcttccccaagcagagcttcgactgctatgttcactgcttgcacatgatgggttgtcagacaatgcgtatctcctggtagcagaagttctgaaaaagattgtagctcttgctccttttttctgttgccatttcataaatgaacttgcacattcaatgcaaaatttgacgctttgtgcaatgaaggagcttcacttgtatgaggattctgaaaaggctcttcttagcacatcatcagccaatggcactgcaattcttagagttgtgcaggctttgagttctcttgtcaccactctgcaagagaaaaaggatccagatcatcctgctgaaaaagatcattctgatgcattgtcccagatttctgaaattaacactgcattggatgcattatggttggagctgagtaattgcataagcaaaatagagagctcttcagaatacgcatcgaatctaagtcctgcttctgcaaatgcagccacattaacaacaggtgtagcacctccattgcctgccggaactcagaacatattaccgtacatagaatcatttttcgtgacatgtgagaagttacgccctgggcaacctgatgctattcaagaagcttcaacatctgacatggaggatgcatcaacttctagtggtgggcagaaatcatctggaagccatgcaaatcttgatgagaagcacaatgcgtttgttaaattctcagagaaacacagaagattgttgaacgcatttatccgccaaaaccctgggctattggagaagtcattctctctgatgttgaaaatccctcgcttgattgaatttgacaacaagcgtgcatatttccggtctaaaattaagcatcagcatgatcatcatcatagccctgttagaatttctgtgcgccgggcatatattttggaggattcatataaccagcttaggatgcgttcaccacaggatttgaagggtagactgactgttcatttccaaggtgaagaaggcattgatgctggtggactaacaagggaatggtatcagctgctatcacgagtgatttttgataagggtgcccttctattcacaactgttggaaatgacttgacatttcaaccaaaccctaactcggtgtatcagactgaacacctctcatatttcaaatttgttgggcgagtggttggtaaagctctatttgatggccaacttttggatgtccattttacaagatctttctacaagcacatactaggtgtcaaggttacataccatgacattgaagctattgatcctgcatactataaaaatttgaaatggatgcttgagaatgacataagcgatgttctggacctctccttcagcatggatgcagatgaagagaagcggatattgtatgagaaggcagaggtgactgattatgagttgattcctggaggccgaaacatcaaggtcaccgaggagaacaagcatgaatatgtgaaccgggttgcagaacatcgtttaaccactgctattaggcctcaaatcacctcttttatggagggatttaatgagctcattcctgaggagctgatatcaatctttaatgacaaagaacttgaactgctaatcagtggactcccagacattgacttggacgatctaaaagcaaatacagaatattctgggtacagcatagcttctccagtcattcagtggttctgggagattgtccaagggttcagcaaggaggacaaagcccggttccttcagtttgttactggcacctcaaaggtacctctggaaggtttcagtgcactccaaggaatatctggaccacaacgattccagatacacaaggcctacggaagcaccaaccatctgccttcagcacatacttgctttaaccaactagaccttcctgagtacacatcgaaagagcagctccaggagagattgctactggctattcatgaggcgaatgaaggtttcggatttggttaa

SEQ ID NO: 2 - protein, 3645 aa, Oryza sativa
MAAAAAMAAHRASFPLRLQQILSGSRAVSPSIKVESEPPAKVKAFIDRVISIPLHDIAIPLSGFRWEFNKGNFHHWKPLFMHFDTYFKTQISSRKDLLLSDDMAEGDPLPKNTILQILRVMQIVLENCQNKTSFAGLEHFRLLLASSDPEIVVAALETLAALVKINPSKLHMNGKLINCGAINSHLLSLAQGWGSKEEGLGLYSCVVANERNQQEGLCLFPADMENKYDGTQHRLGSTLHFEYNLAPAQDPDQSSDKAKPSNLCVIHIPDLHLQKEDDLSILKQCVDKFNVPSEHRFSLFTRIRYAHAFNSPRTCRLYSRISLLAFIVLVQSSDAHDELTSFFTNEPEYINELIRLVRSEEFVPGPIRALAMLALGAQLAAYASSHERARILSGSSIISAGGNRMVLLSVLQKAISSLSSPNDTSSPLIVDALLQFFLLHVLSSSSSGTTVRGSGMVPPLLPLLQDNDPSHMHLVCLAVKTLQKLMEYSSPAVSLFKDLGGVELLSQRLHVEVQRVIGVDSHNSMVTSDALKSEEDHLYSQKRLIKALLKALGSATYSPANPARSQSSNDNSLPISLSLIFQNVDKFGGDIYFSAVTVMSEIIHKDPTCFPSLKELGLPDAFLSSVSAGVIPSCKALICVPNGLGAICLNNQGLEAVRETSALRFLVDTFTSRKYLIPMNEGVVLLANAVEELLRHVQSLRSTGVDIIIEIINKLSSPREDKSNEPAASSDERTEMETDAEGRDLVSAMDSSEDGTNDEQFSHLSIFHVMVLVHRTMENSETCRLFVEKGGLQALLTLLLRPSITQSSGGMPIALHSTMVFKGFTQHHSTPLARAFCSSLKEHLKNALQELDTVASSGEVAKLEKGAIPSLFVVEFLLFLAASKDNRWMNALLSEFGDSSRDVLEDIGRVHREVLWQISLFEEKKVEPETSSPLANDSQQDAAVGDVDDSRYTSFRQYLDPLLRRRGSGWNIESQVSDLINIYRDIGRAAGDSQRYPSAGLPSSSSQDQPPSSSDASASTKSEEDKKRSEHSSCCDMMRSLSYHINHLFMELGKAMLLTSRRENSPVNLSASIVSVASNIASIVLEHLNFEGHTISSERETTVSTKCRYLGKVVEFIDGILLDRPESCNPIMLNSFYCRGVIQAILTTFEATSELLFSMNRLPSSPMETDSKSVKEDRETDSSWIYGPLSSYGAILDHLVTSSFILSSSTRQLLEQPIFSGNIRFPQDAEKFMKLLQSRVLKTVLPIWTHPQFPECNVELISSVTSIMRHVYSGVEVKNTAINTGARLAGPPPDENAISLIVEMGFSRARAEEALRQVGTNSVEIATDWLFSHPEEPQEDDELARALAMSLGNSDTSAQEEDGKSNDLELEEETVQLPPIDEVLSSCLRLLQTKESLAFPVRDMLLTMSSQNDGQNRVKVLTYLIDHLKNCLMSSDPLKSTALSALFHVLALILHGDTAAREVASKAGLVKVALNLLCSWELEPRQGEISDVPNWVPSCFLSIDRMLQLDPKLPDVTELDVLKKDNSNTQTSVVIDDSKKKDSEASSSTGLLDLEDQKQLLKICCKCIQKQLPSATMHAILQLCATLTKLHAAAICFLESGGLHALLSLPTSSLFSGFNSVASTIIRHILEDPHTLQQAMELEIRHSLVTAANRHANPRVTPRNFVQNLAFVVYRDPVIFMKAAQAVCQIEMVGDRPYVVLLKDREKEKNKEKEKDKPADKDKTSGAATKMTSGDMALGSPVSSQGKQTDLNTKNVKSNRKPPQSFVTVIEYLLDLVMSFIPPPRAEDRPDGESSTASSTDMDIDSSAKGKGKAVAVTPEESKHAIQEATASLAKSAFVLKLLTDVLLTYASSIQVVLRHDADLSNARGPNRIGISSGGVFSHILQHFLPHSTKQKKERKADGDWRYKLATRANQFLVASSIRSAEGRKRIFSEICSIFVDFTDSPAGCKPPILRMNAYVDLLNDILSARSPTGSSLSAESAVTFVEVGLVQYLSKTLQVIDLDHPDSAKIVTAIVKALEVVTKEHVHSADLNAKGENSSKVVSDQSNLDPSSNRFQALDTTQPTEMVTDHREAFNAVQTSQSSDSVADEMDHDRDLDGGFARDGEDDFMHEIAEDGTPNESTMEIRFEIPRNREDDMADDDEDSDEDMSADDGEEVDEDEDEDEDEENNNLEEDDAHQMSHPDTDQEDREMDEEEFDEDLLEEDDDEDEDEEGVILRLEEGINGINVFDHIEVFGGSNNLSGDTLRVMPLDIFGTRRQGRSTSIYNLLGRAGDHGVFDHPLLEEPSSVLHLPQQRQQENLVEMAFSDRNHDNSSSRLDAIFRSLRSGRSGHRFNMWLDDSPQRTGSAAPAVPEGIEELLVSQLRRPTPEQPDEQSTPAGGAEENDQSNQQHLHQSETEAGGDAPTEQNENNDNAVTPAARSELDGSESADPAPPSNALQREVSGASEHATEMQYERSDAVVRDVEAVSQASSGSGATLGESLRSLEVEIGSVEGHDDGDRHGASDRLPLGDLQAASRSRRPPGSVVLGSSRDISLESVSEVPQNQNQESDQNADEGDQEPNRAADTDSIDPTFLEALPEDLRAEVLSSRQNQVTQTSNEQPQNDGDIDPEFLAALPPDIREEVLAQQRAQRLQQSQELEGQPVEMDAVSIIATFPSEIREEVLLTSPDTLLATLTPALVAEANMLRERFAHRYHSGSLFGMNSRGRRGESSRRGDIIGSGLDRNAGDSSRQPTSKPIETEGSPLVDKDALKALIRLLRVVQPLYKGQLQRLLLNLCAHRESRKSLVQILVDMLMLDLQGSSKKSIDATEPPFRLYGCHANITYSRPQSTDGVPPLVSRRVLETLTYLARNHPNVAKLLLFLEFPCPPTCHAETSDQRRGKAVLMEGDSEQNAYALVLLLTLLNQPLYMRSVAHLEQLLNLLEVVMLNAENEITQAKLEAASEKPSGPENATQDAQEGANAAGSSGSKSNAEDSSKLPPVDGESSLQKVLQSLPQAELRLLCSLLAHDGLSDNAYLLVAEVLKKIVALAPFFCCHFINELAHSMQNLTLCAMKELHLYEDSEKALLSTSSANGTAILRVVQALSSLVTTLQEKKDPDHPAEKDHSDALSQISEINTALDALWLELSNCISKIESSSEYASNLSPASANAATLTTGVAPPLPAGTQNILPYIESFFVTCEKLRPGQPDAIQEASTSDMEDASTSSGGQKSSGSHANLDEKHNAFVKFSEKHRRLLNAFIRQNPGLLEKSFSLMLKIPRLIEFDNKRAYFRSKIKHQHDHHHSPVRISVRRAYILEDSYNQLRMRSPQDLKGRLTVHFQGEEGIDAGGLTREWYQLLSRVIFDKGALLFTTVGNDLTFQPNPNSVYQTEHLSYFKFVGRVVGKALFDGQLLDVHFTRSFYKHILGVKVTYHDIEAIDPAYYKNLKWMLENDISDVLDLSFSMDADEEKRILYEKAEVTDYELIPGGRNIKVTEENKHEYVNRVAEHRLTTAIRPQITSFMEGFNELIPEELISIFNDKELELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKEQLQERLLLAIHEANEGFGFG

SEQ ID NO: 3 - DNA, 4870 bp, Oryza sativa
acattaactgtcctatatgcgatgtatttattgttatggtgtattaaatcatcagtatatatagtaaaaaacataacaaagagtgcacgactaatttaaaagataaaagaaaaagtagagtaattgggccaccaaaactaatgattttcgctactagatcgaagctctagccttttttttttttttgccataagcctgcttgacatgtatcttttacttgattttagatgatcctcatattcctttatttctaaacttcccaagcaatcaaaagaatagcaaatgttcatctttacacaaatgaaaactaccattttagcttgattgtgttcttggcccattctaggaagctaaaattatgagaagtagccttttggtagctaaattttgagaatctagaatatatctgagggaaggggatgcaggaactgcattctttcatttgaagataaaggcgagaagcaggaagcttctcattccaatccttgagcatgatggcaggattgccaccacccagcatgacatgcaaagtttggcacgagaatactttgctgcagtgatgtgccctgagtgcagtgacacgaagttgctgcaatttcaccatattcagatggcaacaactgatctctccagcctcgacagtcctttcactgaagatgagatttggtcggctatccgtgctttgcctaatgaaaagtcgccagggccggatggttatacaggcttgttttaccaaagatgttgggagataattaaacctgaattgatcagcgctcttgctaaattctgtaccggtaacagtcagaacttggagaaactgaattcggcaattgtcacgctaataccgaagaaggacagtcctaccctcctcaaggattataggccaattagtttgattcatagtttctctaagatagctgcgaagattatggcgcagcggttagcaccgaagctgaatgtcctcattccatcctcccaaactgcttttatcaagggacgctgcatacacgagaactttgtcttcgtcaaaggattggtacaacaatttcacagacaaaggaaggctatgatgttgctgaaattagacatctcgaaagctttcgacactgtctcctggggttttcttatgtcgatgttacagttcagaggctttggtccactttggagaagatggctctcggcggtttttctcactgcagaaacaagaatattgataaatggtgttctgtctgacacaatcaagccggcgagggggttgaggcagggtgacccactgtcgccgctgctctttgttctagtaatggatgccttgcaagctattgtttcccaggcaaggatggcaagactgctctcgcccctcaacgtacgacagaatttgccaccaatttcagtttatgccgacgatgcggttctgtttttccgccctacagctgaagaagctcgagtcatcaagggtatcctggagttgttcggggctgccacaagtctcaaaaccaatttctccaaaagcgcaatcactccaatccaatgtgacgagcagcagtatgtgcaagttgaatccattctctcctgccgagtggaaaagttcccaatcacttatcttggactccctctctccactaggaaaccaacgaaggccgagatccagccgatccttgataggctggcaaagaaggtagccggttggaagccgaaaatgctgtctattgatgggcgactgtgcttgatcaagtcggtcctaatggcgctgccggtgcactacatgacagtcctgcagctaccgcgatgggcgattaaggacatcgagcggaagtgccgtgggtttctttggaaaggacaggaagagatcagcggcgggcattgcctagtctcgtggcgaaaggtttgctcacccatcgagaaagggggacttggtgtcaaagatcttaatttgttcggtcaagctctccggttgaaatggcttgcaaaatccttggagcagaaggatagaccctggaccttagcaactttccgtcctggaagcgatgtggaagagatctttcgatccgttgctgagcacatcattggtgacggggtgaacacacagttttggacagacaattggacagggaaaggttgcttcgcctggaggtggccggtgttgttttcccatgtgagccgtgccaagctgacagtagctgatgccctgattgctaacagatgggttcgccgattacaaggtgccttgtccaatgaagctctgggtgaattcttccaactttgggatgaagttcacgacgtgtcactgcagcagatggctaaaacgatcaaatggaagttgactgttgatggtaatttctcagtggcctcggcgtatgatctatttttcatagcgacagaggactgttcctacggggacacgctgtggcactccagggtgccgtcgcgtgttcgcttcttcatgtggattgcactcaagggccgctgtctcacggcggacaacctggcaaagagaaactggccgcatgacgccatttgctccctatgccaacacgagaacgaagactgccattatttgcttgtgtcctgtgattatacggcggcggtttggcgcaagctgagacgttggtgcaacattaacattgcaatccctgcggaagatggcatgccgcttgcagattggtggatcgcgacaagacggcgttttcagaacacgtataggacggatttcgatagtctgttaatgctaatttgttggcttatctggaaagagcgaaatacaaggatctttcaacacatcgccaagtcggttgaccggctagcggatgacatcaacgaggaaatcgcaatttggagggcagcagggattttctcccaggctagcgagtaatcccgattagaggcgggacctgccccattttttccttttctttccgggcttgagtttgcttgagaccggcgcgacatccttcatgtcgttgtaattaaaactttatttccctcaatcttaataaaattggccggcctacctttggccgtcccggcaaaaaagaatctagaatatatagctacatattctcaaaatcgaatctggactgttttggagagtagccgctagaaacttcctagaacaaaacccttatatttgttctttaagtcacatcatacttgctgatgaaatcactatccattagttactccatccgtcccaaaaatacttaatctaggagaagatgtgactccttctgatacaataaatttggataaagagctatcagatttgttaggatcacacatttatttgtaggttaagttttttttaacggaagtagtacgcataaaggattggcttacccaattgttaaccggcccggcactggaacagaaaggtcttgaacccaaacgggacgccgagaaggcccttccctgacgaaagcaaagggcttaattagctagcaagaaacccaaaccgacccgagcccgtcacgcgccgcgcccgtgacctaccgtgcgctgcgccgcctcctccctcccacctcccttcacaaaagcagcgacccctcctccctccccaagtttcctccccacaccgcaacccttctctctctctctctctcccctctcgacttctctcctctccgccgcctccgagtcccgccgcgccgcgcgcccgtcttccccggcggccgatgtgtctgcctcgtcggcacgaaaccctagaggtaacccgccgcgccgctccccgccgcttcccgccgcgatcgggggccctcccccctagggttttcgggggacttttgagggtggatgatttgggggtgtggggggctttgggggcggtctaacctgtttgtggtttctggtgcaggtgcggtgcagttgaggggtcccgatcggagatggcggcggcggcggccatggcggcgcaccgggccagcttcccgctccggctgcagcagatcctgtccgggagccgcgccgtgtcgccgtcgatcaaggtggagtccgagccggtgagtccctcgcgccgttcccctgtttcctcgccctagggttttgatcgtcggggttgaggggttgtagatgcgaagttgagatggtatgtaggatcgaatcctccctaggtgcttcctctagggttttgatcggctgcctgtgttgatgtggcgtgctgttggggtgaggtagttaggccgtaaggagtttgctccgtttatgatcggtgttgagcatggggaccagtggtgtggtgtgcagggtagttgttactgctttaggccatctcaaatttgggtttccttggtcaggggtagaagagacaccggtttgaagtttctggttatcttgcttgtgctgttattgtactatattgtagtagggatacatgctcgtgttattctgttaccttgtttaagcatgtctatgcccctcaatgcttagttgccgctgcagccgtaatcttttaggcttagccgcttaggtatccccattacatttgtattatcttgttattactacggtgtcccattggacatttattagttcagactttcttgcacttgtaattccttctgcaaaacatacgagtcaatacagaatgccacatctagcaaattactatgttatcattgatgcttaggtgcccatgatcagtacttatggacttgtactggccattttataatgttattttttcattctgttattgctatagctttttaatccttttttacgtatttttatttctgtgcacaactgcacttatgttgaccaatcctgtatcatgttttggataatggcttactacataaatatatgacgttggatagtagcctcaagattgatgcattgatttagttcacttgatattacagctcaagagttgagacat

SEQ ID NO: 4 - DNA, 10440 bp, Triticum aestivum
  misc_feature (69)..(111): n is a, c, g, or t
atggcagcggcggcgatggcagcgcacagggccagcttcccgctgcggctgcagcagatcctgtctggnnnnnnnnnnnnnnnnnnnnnnnnnnnnnnnnnnnnnnnnnnnccagcaaaagttaaagcatttattgatcgtgtaatcaatattccactacatgacattgccataccactatcaggcttccattgggagttcaataagggaaattttcaccattggaagcctctgtttatgcattttgatacgtatttcaagacatacatctcttctagaaaggatcttctcttgtctgatgatatgtctgagagtgaaccgttgacaaaaaataccatcttacaaattttgagagttatgcaaattgtgttggaaaattgccagaacaaaacaacttttgctggtcttgagcattttaagaatctgttgacatcatcagatcctgaggtagttgttgcggctttggaaactcttgcttcagtggtgaaaataaatccttcaaaactacatatgaatgggaaactgatcagctgtggagctataaatagccatcttctatcattggcccaaggatggggtagcaaggaggaaggtctaggcttgtattcctgtgttgtggcaaatgagcgaaatcagctggaggggttgtgcttattcccagcagacatggagaacaaatatgatggcacacagcatcgtcttgggtccactcttcattttgagtataatttggcacctgtccaagattctgaccaagccaatgacaagtcatctaatctctgtgtgatacatatgccagacctgcaccttcggaaggaggatgacttgagcatattgaagcaatgtatcaacaagtttaatgtgcctccagagcaccggtttgccttgtttacaaggataagatatgcacatgcttttaattctccgaggacatgcaggctatatagccgcataagtctcctttctttcattgttctcgtgcaatccagtgatgcccatgatgaactgacatcgttcttcacaaatgagccggagtacataaatgagttaatccggcttgttcgttcagaagatattgttcctgggcccattcgtgcactggctatgcttgcactgggagcacagctagcagcatatgcatcatctcatgagcgagctaggatactgagcggttcaagtatcatctctgctggtggaaaccgcatggtcttgctcagtgttctgcagaaagcaatatcatccctcagtagccctaatgatacttcatctcctttaattgttgatgcccttctgcagttctttttgcttcatgtgctgtcatcttcgagttctgggaccaccgttaggggttcagggatggttccacctcttctgcctctcctgcaagataaggatccttcccacatgcatcttgtctgtttggcagtaaagacccttcagaagctgatggagtacagcagccctgctgtttccctgtttaaagatttgggtggtgtagaacttttgtctcagaggttacatgtggaggtgcagcgtgttattggtgttgctgaaattacttcagtggttgctagtgatacatcgaaatcagaagatgaccatctgtactcacagaagcgactgattaaggctttgctcaaggcattaggttccgctacatattctcctgcgaatcctgctcgctctcaaagttccaatgataactctttgcccatgtcactttcccttatattccagaatgttggaaagtttggtggtgatatatacttctcttcagttactgttatgagtgagataatacataaggatcctacatgctttcctgctttgaaggaacttggtcttcctgatgctttcctgtcatcagtgactgctggggtgataccatcttgtaaagctcttatatgtgtacccaatggcctgggcgcaatctgccttaataaccaaggacttgagtctgtcagggaaacttcagcgctgcgttttcttgtagagacgttcactagtaagaaatatctgataccaatgaatgaaggtgttgttcttttagcaaatgcagtggaagagcttattcgtcatgtgcagtcccttagaagtactggtgttgacatcatcattgaaataatcaacaaactttcttgtcctcgtggtgataaaatcactgaagcagccagcgctgaagaaaaaacagatatggaaacagatgttgaagggcgtgatttagtaagtgccatggattctggcacagatggaactaatgatgagcagttttctcatttgagcattttccatgttatggtattggtgcacaggacaatggaaaattcagaaacttgcaggctgtttgtagaaaagggaggtctacagactctgttgacactcttgctgcgacctaccatcacccaatcttctggtgggatgcctattgctttgcacagcactatggtatttaagggctttactcaacagcattctactccacttgcgcgtgcattctgctcctctttaaaggagcacttgaagaatgccctacaggaacttgatacagtttttaggtcatgtgaagtgactaagatggaaaaaggagccattccatcactttttattgttgaatttctgctcttcctagcggcatcaaaagacaaccgctggatgaacgctctactctcagaatttggtgatgtcagcagggatgtcctggaagatattggaagagttcaccgggaagtgctttggcaaatttcactttttgatgagaagaaaatagagcctgaagctagttctccttcagcaaatgaggcccagcaagttgatgctgctgtgggcgacactgatgataacagatacacctcctttaggcaatatcttgatcctcttttaaggcgacggggctctgggtggaacattgagtctcaggtgtctgaccttatcaatatctatcgtgatatgggccgtgcagctactgactctcacagagttggtgctgatagataccctagtacagggttgccctcaagttcccaggatcagccatccagttcatctgatgcaaatgcgaagtcagaagaggacaagaaaaggtctgagcattcatcctgctgtgacatgatgaggtcactttcttaccatattaaccatctgtttatggagcttgggaaagcaatgctgcttacatctcgccgtgagaacagtcctataaatctacctccatctgttgtgtctgttgctagcaatattgcttctatcgtgttggagcacctcaattttgaggggcatacaatcagtccagagagggagattactgttgctacaaagtgccgataccttggaaaggttgttgagtttattgatgggatattgttggacagaccagaatcatgtaatccaatcatggtgaattctttttattgccgtggtgtaattcaggctatcttaaccacatttgaagctaccagtgagttgctttttgcaatgaacaggccaccctcgtcacctatggagactgatagtaaaactgggatggaagagaaggatactgattgttcatggatttatggccccctttccagctatggtgcagctatggaccatctcgtaacatcatcatttgttctttcttcatcgacaagacaactacttgagcaacctatttttagtggaactgtcaggtttccacaagatgcagagagattcatgaagttgcttcaatctaaagtcttaaagactgttcttcccatatgggcccatccacagtttccagaatgcaatcttgagctaatcagttcagtcacatcgatcatgaggcatgtttactctggggtagaagtaaaaaacaatgttagcaacatggctgctcgtttggctggtccaccccctgacgagaatgcaatttccttgattatagagatgggcttttctcgtgccagagctgaagaagcattaagacaggttggaacaaatagcgttgagatcgcgactgactggttgttctcacacccggaggaaccaccagaggatgatgaacttgctcgagctcttgctatgtctctagggaattctgatacacctgttcaagaggaagatgacagaactaatgatcttgagctcgaggaagtaaatgttcagctccctcccatggatgaagtattatcttcatgtcttaggctgctacaggcaaaggaaacattagcttttcctgtccgggatatgcttgtgactatcagctcacagaatgacggccaaaaccgtgtgaaggtgcttacatatttgattgatcatctgaagcagtgtcttgtggcatctgatcctttaaagaacactgcactatctgcttttttccatgttcttgctttgattctccatggagataccgctgcccgtgaagttgcctcaaaggctggtcttgtcaaggttgttttgaatctgctgtgcagctgggagttggagccaagggaaggtcaaacaaccaaggttcccaattgggttacttcctgtttcctttctgttgatagaatgctccagttggaacccaagttgccagatgttactgagcttgatgttctcaaaaaggataattcacctacacaaacatctgttgtgattgatgacagcaagaagaaggattcagaatcttcctcaagtgtagggttactggacttggaggaccaggatcaacttttgagggtatgctgtaagtgcattcagaagcagttgccttccggtacaatgcatgctattcttcagttgtgtgctacactgacgaaagtccatgtggctgctatcagttttctcgagtctggtggcctacatgcgttgctaagtttgccgacaagtagcttattttctggattcaacagtgtggtttctacaattatccgtcatattttggaggatccccatactcttcagcaagctatggaattggagatacgccatagtcttgtcacggctgctaatcggcatgcaaatcctagggttacaccacgtaattttgttcaaaatttggcgtttgttgtatacagggacccagtgatatttatgaaagctgcccaagctgtgtgccagattgagatggttggagatagaccgtatgttgttctattgaaggaccgtgagaaggaaaagagcaaggaaaaagagaaggataagctggttgataaggataaatcatcaggtgttgccacaaagataacatcaggggacatggttatggcatcccctgtaagtgccaaagggaaacaatctgatttcagtgcaaggaatatgaaatctcatcgcaagccaccacaaacctttgtcactgttattgagcatctattagatctagtcatgtcctttgtcccaccacaaagagctgaggaccaatctgatggttcgtcatccatggacatggatattgactctagttcagcaaaaggtaaagggaaggctgttgctgtcacacatgaagagtccaagcaagcaatccaagatgctactgcatgtctggctaaaaacgcatttgtcctaaagctgctcacagatgtactattaacttatgcttcttcggttcaagttgttcttcgccacgatgctgagttcagcagcacgcgaggtcctactcggaccagtggtggaatatttaatcatatattgcagcatttgcttccacatgctacaaagcaaaagaaagaaaggaagcctgatggtgattggaggtacaaattggcaacaaggggtaatcaatttttagtggcttcatctattcgttcttcagaaggacgaaaaaggatctgttctgaaatctgcagtatatttgttgagttcacagacaattctactggttgcaaacctccaatgttgagaatgaatgcttatgttgatttgctcaatgatattctgtcagctcgctcaccaacgggctcctcactttcagcagaatctgtagttacatttgttgaggttggccttgtccagtgtttaacgaaaacccttcaggttcttgatttggatcattctgattcagcaaagattgtaactggtattgtcaaggctcttgaagtggttactaaggagcacgttcatttagcagattttaatgctaaaggagagaactcatcaaagactgttttggagcagaacaatgtagattcatcatcaaataggttccaggttcttgacacaacttctcaaccaactgcaatggtaactgatcacagggaaactttcaatgctgttcatgcttcaagaagttcagattcagtggcagatgagatggatcatgaccgtgatatagatggaggttttgcccatgatggtgaagatgattttatgcatgagattgctgaagacagaacaggaaatgagtccacaatggatattcggtttgatataccacgtaatagagaggatgatatggctgaagatgaagatgacagtgatgaagatatgtcagccgatgatggtgaggaggttgatgaagatgatgatgatgaggaaaataacaacctggaggaagacgatgcccatcagatatctcatgctgacacggatcaggatgaccgtgagattgatgaagaagaatttgatgaagatctgctagaagaagaagatgatgatgaggaggatgaggagggtgtaatccttcgcctagaggaggggatcaatggaattaatgtgtttgaccatattgaggtttttggggggagcaacaatgtttctggggatacactgcgtgtaatgccattggacatttttggcacaagacggcaaggccgtagtacatctatatacaatctgcttgggagagcaagtgaccaaggtgtacttgaccacccactcttggaggaaccttcgatgttacttccacaacagaggcaaccagaaaatttagttgagatggctttctctgatcggaatcaggaaaatagttcttcccgtttggacgctatattcagaagcttgcgaagtggacggaatggacaccgatttaatatgtggctggatgatggcccccaacgcaatggatctgctgcccctacagtacctgaaggcattgaagaacttctgctctctcagttgagaaggcctatggctgaacatcctgatgagcagagcacacctgctgttgatgctcaagtaaacgatcctcccagtaatttccatgggccagaaactgatgcaagggaaggatcagcagaacaaaatgaaaacaacgaaaacgatgatattcctgcagtaaggtctgaggtggatggctctgcaagtgctggtcctgcagctcctcacagtgatgaacttcaaagagatgcatccaatgcaagtgagcatgtcgcagatatgcaatatgaacgtagtgacacagctgtccgtgatgtggaagcagtaagccaagcaagcagtggtagtggtgctactctaggtgaaagcctcagaagtttagatgtggaaattggaagtgttgaaggacatgatgatggtgaccggcatggcgcttcagacaggacacctctgggtgatgtacaggcagctactcgatcgcggaggccttctggaaatgcagtaccagtcagcagcagggacatatcattggagagtgttagggagatcccccaaaacacagtccaggaatccgatcagaatgccagtgagggggatcaggagcctaacagggctactgggactgactcaatagatcctacatttttggaggctcttccagaggatttacgggctgaagttctttcttcacgtcaaaatcaagtgactcagacttccagtgagcagcctcagcatgatgcggacattgatcctgaattccttgctgcactgccacctgatatacgtgaagaggttctagctcaacaacgtgcccaacggttgcaacaacagtctcaagagcttgaaggacaaccagttgaaatggacgctgtatcaattattgcaaccttcccttcggaaatacgggaggaggtgcttttgacgtctcctgatactctacttgctacattgacacctgcactagttgctgaagccaacatgttgcgtgagagatttgctcatcgatatcacagcggttcactatttggcatgaactccaggaacaggaggggagagtcctctcgtcgtggtgatatcattggttcaggccttgaaagaaatactggtgattcgtcccgtcaaacggccagcaagttaattgaaactgtagggactcctcttgttgacaaggatgctctcaatgctcttatacgattacttcgagttgtccagcctatatacaagggccagctgcagaggcttctcttgaacctttgtgctcaccgggaaagcagaaagtcgctggttcaaattctggtggacatgcttatgcttgatctgcagggctcttctaagaaatcaattgatgcaacagagccgtcatttaggctatatgggtgccatgcaaatatcacatattcacgccctcaatcctcagatggtgtgccaccactggtttcccgccgtgtgctggaaactctgacatacctggcaagaaatcatccaaatgtggctaagctcttactctttcttcggttcccctgcccgcccacctgccatactgaaacacttgaccagaggcatggaaaggctgtccttgtggaagacggggaacaacagagtgcttttgcactcgttctacttttgactctattaaatcagcctctttatatgagaagtgtagctcatcttgaacagttactcaatcttctggaagttgtcatgcttaatgctgagaatgaagtaaatcaagctaagttggaaagttcatctgagagaccgtctggacctgagaatgcaatacaagatgcccaagaggatgccagtgttgctggatcatctggagcgaagcccaatgcagatgatagtggtaaatcatccgcagataatataagtgacctgcaagctgttttgcatagccttccgcaagccgagcttcggttgctatgttcattgcttgcgcatgatgggttatcagacaatgcatatctccttgtagcagaagttctgaaaaagattgtagctttggctcctttcatctgttgccatttcataaatgagctttcacgttcgatgcaaaatttgactgtttgtgccatgaatgagcttcacttgtatgaagattctgaaaaggcaattctgagcacatcatcagccaatggcatggcagttctaagagttgtgcaggccttgagttctcttgtcacatctctgcaagagagaaaagatccagagcttcttgcagagaaggaccattcagattccctgtcccagatttctgatattaacacagcattggatgcattgtggttggagctaagccactgcataagcaaaatagagagctcttcagaatatacatcaaatttgagtccaacttctgcaaatgcaactagagtatcaactggtgtagcacctcccttgcctgctggaacccagaatatattaccgtacatagaatccttttttgtgacatgcgagaagttacgtccaggacaacctgatgctgttcaagagccttcaacatctgatatggaggatgcttcaacatctagtagtgggcaaaaatcatctgcaagccataccagtcttgatgagaagcacactgcttttgttaaattctcggagaagcacagaagattgttaaatgcttttatccgacaaaactctgggttgctagaaaaatcattctctctgatgttgaaggtcccccgcttgattgattttgacaacaaacgtgcatatttccggtctaaaattaagcatcagcatgaccatcatcacagtcctgttagaatttctgtccgacgggcgtacattttggaagactcttataatcagcttaggatgcgctcaccccaggacttgaagggtcgactcacggttcatttccaaggagaagaaggcattgatgccggtggactcacaagagagtggtatcagttactatcacgagtgatttttgacaagggtgcccttctattcacgactgttggaaatgacttgacatttcaaccaaaccctaactctgtgtatcagactgaacacctctcatatttcaaatttgttggacgagtggttggaaaagcgctctttgatgcccaacttttggatgtccatttcactagatctttctacaagcacatacttggtgccaaggttacttaccatgatattgaagccattgatcctgcttactacagaaaccttaagtggatgcttgagaatgacataagtgatgtcctggacctcacatttagcatggatgcagatgaagagaagctgatattatatgagaaagctgaggtaactgattgtgagttgattcctggagggcgtaacattagggtcaccgaggagaacaagcatgaatatgtggatagggtagcagagcatcgtttaaccactgcaattaggcctcag

SEQ ID NO: 5 - protein, 3632 aa, Triticum aestivum
  misc_feature (24)..(37): Xaa can be any naturally occurring amino acid
MAAAAMAAHRASFPLRLQQILSGXXXXXXXXXXXXXXPAKVKAFIDRVINIPLHDIAIPLSGFHWEFNKGNFHHWKPLFMHFDTYFKTYISSRKDLLLSDDMSESEPLTKNTILQILRVMQIVLENCQNKTTFAGLEHFKNLLTSSDPEVVVAALETLASVVKINPSKLHMNGKLISCGAINSHLLSLAQGWGSKEEGLGLYSCVVANERNQLEGLCLFPADMENKYDGTQHRLGSTLHFEYNLAPVQDSDQANDKSSNLCVIHMPDLHLRKEDDLSILKQCINKFNVPPEHRFALFTRIRYAHAFNSPRTCRLYSRISLLSFIVLVQSSDAHDELTSFFTNEPEYINELIRLVRSEDIVPGPIRALAMLALGAQLAAYASSHERARILSGSSIISAGGNRMVLLSVLQKAISSLSSPNDTSSPLIVDALLQFFLLHVLSSSSSGTTVRGSGMVPPLLPLLQDKDPSHMHLVCLAVKTLQKLMEYSSPAVSLFKDLGGVELLSQRLHVEVQRVIGVAEITSVVASDTSKSEDDHLYSQKRLIKALLKALGSATYSPANPARSQSSNDNSLPMSLSLIFQNVGKFGGDIYFSSVTVMSEIIHKDPTCFPALKELGLPDAFLSSVTAGVIPSCKALICVPNGLGAICLNNQGLESVRETSALRFLVETFTSKKYLIPMNEGVVLLANAVEELIRHVQSLRSTGVDIIIEIINKLSCPRGDKITEAASAEEKTDMETDVEGRDLVSAMDSGTDGTNDEQFSHLSIFHVMVLVHRTMENSETCRLFVEKGGLQTLLTLLLRPTITQSSGGMPIALHSTMVFKGFTQQHSTPLARAFCSSLKEHLKNALQELDTVFRSCEVTKMEKGAIPSLFIVEFLLFLAASKDNRWMNALLSEFGDVSRDVLEDIGRVHREVLWQISLFDEKKIEPEASSPSANEAQQVDAAVGDTDDNRYTSFRQYLDPLLRRRGSGWNIESQVSDLINIYRDMGRAATDSHRVGADRYPSTGLPSSSQDQPSSSSDANAKSEEDKKRSEHSSCCDMMRSLSYHINHLFMELGKAMLLTSRRENSPINLPPSVVSVASNIASIVLEHLNFEGHTISPEREITVATKCRYLGKVVEFIDGILLDRPESCNPIMVNSFYCRGVIQAILTTFEATSELLFAMNRPPSSPMETDSKTGMEEKDTDCSWIYGPLSSYGAAMDHLVTSSFVLSSSTRQLLEQPIFSGTVRFPQDAERFMKLLQSKVLKTVLPIWAHPQFPECNLELISSVTSIMRHVYSGVEVKNNVSNMAARLAGPPPDENAISLIIEMGFSRARAEEALRQVGTNSVEIATDWLFSHPEEPPEDDELARALAMSLGNSDTPVQEEDDRTNDLELEEVNVQLPPMDEVLSSCLRLLQAKETLAFPVRDMLVTISSQNDGQNRVKVLTYLIDHLKQCLVASDPLKNTALSAFFHVLALILHGDTAAREVASKAGLVKVVLNLLCSWELEPREGQTTKVPNWVTSCFLSVDRMLQLEPKLPDVTELDVLKKDNSPTQTSVVIDDSKKKDSESSSSVGLLDLEDQDQLLRVCCKCIQKQLPSGTMHAILQLCATLTKVHVAAISFLESGGLHALLSLPTSSLFSGFNSVVSTIIRHILEDPHTLQQAMELEIRHSLVTAANRHANPRVTPRNFVQNLAFVVYRDPVIFMKAAQAVCQIEMVGDRPYVVLLKDREKEKSKEKEKDKLVDKDKSSGVATKITSGDMVMASPVSAKGKQSDFSARNMKSHRKPPQTFVTVIEHLLDLVMSFVPPQRAEDQSDGSSSMDMDIDSSSAKGKGKAVAVTHEESKQAIQDATACLAKNAFVLKLLTDVLLTYASSVQVVLRHDAEFSSTRGPTRTSGGIFNHILQHLLPHATKQKKERKPDGDWRYKLATRGNQFLVASSIRSSEGRKRICSEICSIFVEFTDNSTGCKPPMLRMNAYVDLLNDILSARSPTGSSLSAESVVTFVEVGLVQCLTKTLQVLDLDHSDSAKIVTGIVKALEVVTKEHVHLADFNAKGENSSKTVLEQNNVDSSSNRFQVLDTTSQPTAMVTDHRETFNAVHASRSSDSVADEMDHDRDIDGGFAHDGEDDFMHEIAEDRTGNESTMDIRFDIPRNREDDMAEDEDDSDEDMSADDGEEVDEDDDDEENNNLEEDDAHQISHADTDQDDREIDEEEFDEDLLEEEDDDEEDEEGVILRLEEGINGINVFDHIEVFGGSNNVSGDTLRVMPLDIFGTRRQGRSTSIYNLLGRASDQGVLDHPLLEEPSMLLPQQRQPENLVEMAFSDRNQENSSSRLDAIFRSLRSGRNGHRFNMWLDDGPQRNGSAAPTVPEGIEELLLSQLRRPMAEHPDEQSTPAVDAQVNDPPSNFHGPETDAREGSAEQNENNENDDIPAVRSEVDGSASAGPAAPHSDELQRDASNASEHVADMQYERSDTAVRDVEAVSQASSGSGATLGESLRSLDVEIGSVEGHDDGDRHGASDRTPLGDVQAATRSRRPSGNAVPVSSRDISLESVREIPQNTVQESDQNASEGDQEPNRATGTDSIDPTFLEALPEDLRAEVLSSRQNQVTQTSSEQPQHDADIDPEFLAALPPDIREEVLAQQRAQRLQQQSQELEGQPVEMDAVSIIATFPSEIREEVLLTSPDTLLATLTPALVAEANMLRERFAHRYHSGSLFGMNSRNRRGESSRRGDIIGSGLERNTGDSSRQTASKLIETVGTPLVDKDALNALIRLLRVVQPIYKGQLQRLLLNLCAHRESRKSLVQILVDMLMLDLQGSSKKSIDATEPSFRLYGCHANITYSRPQSSDGVPPLVSRRVLETLTYLARNHPNVAKLLLFLRFPCPPTCHTETLDQRHGKAVLVEDGEQQSAFALVLLLTLLNQPLYMRSVAHLEQLLNLLEVVMLNAENEVNQAKLESSSERPSGPENAIQDAQEDASVAGSSGAKPNADDSGKSSADNISDLQAVLHSLPQAELRLLCSLLAHDGLSDNAYLLVAEVLKKIVALAPFICCHFINELSRSMQNLTVCAMNELHLYEDSEKAILSTSSANGMAVLRVVQALSSLVTSLQERKDPELLAEKDHSDSLSQISDINTALDALWLELSHCISKIESSSEYTSNLSPTSANATRVSTGVAPPLPAGTQNILPYIESFFVTCEKLRPGQPDAVQEPSTSDMEDASTSSSGQKSSASHTSLDEKHTAFVKFSEKHRRLLNAFIRQNSGLLEKSFSLMLKVPRLIDFDNKRAYFRSKIKHQHDHHHSPVRISVRRAYILEDSYNQLRMRSPQDLKGRLTVHFQGEEGIDAGGLTREWYQLLSRVIFDKGALLFTTVGNDLTFQPNPNSVYQTEHLSYFKFVGRVVGKALFDAQLLDVHFTRSFYKHILGAKVTYHDIEAIDPAYYRNLKWMLENDISDVLDLTFSMDADEEKLILYEKAEVTDCELIPGGRNIRVTEENKHEYVDRVAEHRLTTAIRPQINAFMEGFNELIPRELISIFNDKEFELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKDQLQERLLLAIHEANEGFGFG

SEQ ID NO: 6 - DNA, 10887 bp, Triticum aestivum
  misc_feature (105)..(110): n is a, c, g, or t
atggcagcggcggcgatggcagcgcacagggccagcttcccgctgcggctgcagcagatcctgtctggcagccgcgccgtgtcgccggcgatcaaagtcgagtcnnnnnngccagcaaaagttaaagcatttattgatcgtgtaatcaatattccactacatgacattgccataccactatcaggcttccattgggagttcaataagggaaattttcaccattggaagcctctgtttatgcattttgatacgtatttcaagacatacatctcttctagaaaggatcttctcttgtctgatgatatgtctgagagtgaaccgttgacaaaaaataccatcttacaaatcttgagagttatgcaaattgtgttggaaaattgccagaacaaaacaacttttgctggccttgagcattttaagaatctgttggcatcatcagatcctgaggtagttgttgcggctttggaaactcttgcttcagtggtgaaaataaatccttcaaaactacatatgaatgggaaactgatcagctgtggagctataaatagccatcttctatcattggcccaaggatggggtagcaaggaggaaggtctaggcttgtattcctgtgttgtggcaaatgagcgaaaccagctggagggtttgtgcttattcccagcagacatggagaacaaatatgatggcacacagcatcgtcttgggtccactcttcattttgagtataatttggcacctgtccaagattctgaccaagccaatgacaagtcatctaatctctgtgtgatacatatgccagacctgcaccttcggaaggaggatgacttgagcatattgaagcaatgtatcgacaagtttaatgtgcctccagagcaccggtttgccttgtttacaaggataagatatgcacatgcttttaattctccgaggacatgcaggctatatagccgcataagtctcctttctttcattgttctcgtgcaatccagtgatgcccatgatgagctgacatcgttcttcacaaatgagccggagtacataaatgagttaatccggcttgttcggtcagaagatattgttcctgggcccattcgtgcattggctatgcttgcactgggagcacagttagcagcatatgcatcatctcatgagcgagctaggatactgagcggttcaagtatcatctctgctggtggaaaccgcatggtcttgctcagtgttctgcagaaagcaatatcgtccctcagtagccctaatgatacttcatctcctttaattgttgatgcccttctacagttctttttgcttcatgtgctgtcatcttcgagttctgggacgaccgttaggggttcagggatggttccacctctcctgcctctcctgcaagataaggatccttcccacatgcatcttgtctgtttggcagtaaagacccttcagaagctgatggagtacagcagccctgctgtttccctgtttaaagatttgggtggtgtagaacttttgtctcagaggttacatgtggaggtgcagcgtgttattggtgttgctgaaattacttcagtggttgctagtgatacatcgaaatcagaagatgaccatctgtactcacagaagcgactgattaaggctttgctcaaggcattaggttccgctacatattctcctgcgaatcctgctcgctctcaaagttccaatgataactctttgcccatgtcactttcccttatattccagaatgttggaaagtttggtggtgatatatacttctcttcagttactgttatgagtgagataatacataaggatcctacatgctttcctgctttgaaggaacttggtcttcctgatgctttcctgtcatcagtgactgctggggcgataccatcttgtaaagcacttatatgtgtacccaatggcctgggcgcaatctgccttaataaccaaggacttgagtctgtcagggaaacttcagcgctgcgttttcttgtagagacgttcactagtaggaaatatttgataccaatgaatgaaggtgttgttcttttagcaaatgcagtggaagagcttcttcgtcatgtgcagtcccttagaagtactggtgttgacatcatcattgaaataatcaacaaactttcttgtcctcgtggtgataaaatcactgaagcagccagcgctgaagaaaaaacagatatggaaacagatgttgaagggcgtgatttagtaagtgccatggattctggcacagatggaactaatgatgagcagttttctcatttgagcattttccatgttatggtattggtgcacaggacaatggaaaactcagaaacttgcaggctgtttgtagaaaagggaggtctacagactctgttgacactcttgctgcgacctaccatcacccaatcttctggtgggatgcctattgctttgcacagcactatggtatttaagggctttactcaacagcattctactccacttgcgcgtgcattttgctcctctttaaaggagcacttgaagaatgccctacaggaacttgatacagtttttaggtcatgtgaagtgactaagttggaaaaaggagccattccgtcactttttattgttgaatttctgctattcctagcggcatcaaaagacaaccgctggatgaatgctctactctcagaatttggagatgtcagcagggatgtcctggaagatattggaagagttcaccgggaagtgctttggcaaatttcactttttgatgagaagaaaatagagcctgaagccagttctccttcagcaaatgaggcccagcaagttgatgctgctgtgggcgacactggtgataacagatacacctcctttaggcaatatcttgatcctcttttaaggcgacggggctctgggtggaacattgagtctcaggtgtctgaccttatcaatatctatcgtgatatgggccgtgcagctagcgactctcacagagttggtgctgatagataccctagtacagggttgccctcaagttcccaggatcagccatccagttcatctgatgcaaatgcgaagtcagaagaggacaagaaaagatctgagcatttgtcctgctgtgacatgatgaggtcactttcttaccatattaaccatctgtttatggagcttgggaaagcaatgctgcttacatctcgccgtgagaacagtcctataaatctatctccatctgttgtgtctgttgctagcaatattgcttctatcgtgttggagcacctcaattttgaggggcatacaatcagtccagagagggagattactgttgctacaaagtgccgataccttggaaaggttgttgagtttattgatgggatattgttggacagaccagaatcatgtaatccaatcatggtgaattctttttattgccgtggtgtaattcaggctatcttaaccacatttgaagctaccagcgagttgcttttcgcaatgaacaggccaccctcgtcacctatggagactgatagtaaaactgggaaggaagagaaggatgctgattgttcatggatttatggccccctttccagctatggtgcagctatggaccatcttgtaacatcatcatttattctttcttcatcgacaagacaactacttgagcaacctatttttagtggaactgtcaggtttccacaagatgcagagaaattcatgaagttgcttcaatctaaagtcttaaagactgttcttcccatatgggcccatccacagtttccagaatgcaatcttgagctaatcagttcagtcacatcgatcatgaggcatgtttactctggggtagaagtaaaaaacaatgttagcaacattgctgctcgtttggctggtccaccccctgacgagaatgcaatttccctgattatagagatgggcttttctcgtgcaagagctgaagaagcattaagacaggttggaacaaatagcgttgagatcgcgactgattggttgttctcacacccggaggaaccaccagaggatgatgaacttgctcgagctcttgctatgtctctagggaattctgatacacctgttcaagaggaagatgacagaactaatgatcttgagctcgaggaagtaaatgttcagctccctcccatggatgaagtattatcttcatgtcttaggctgctacaggcaaaggaaacattagcttttcctgtccgggatatgcttgtgactatcagctcacagaatgacggccaaaaccgtgtgaaggtgcttacatatttgattgatcatctgaagcaatgtcttgtggcatctgatcctttaaagaacactgcactatctgctttttgccatgttcttgctttgattctccatggagatactgctgctcgtgaagttgcctcaaaggctggtcttgtcaaggtcgttttgagtctgctgtgcagctgggagatggagccaagggaagggcaaacaaccaaggttcccaattgggttacttcctgtttcctttctgttgatagaatgctccagttggaacccaagttgccagatgttactgagcttgatgttctcaaaaaggatagttcacctacacaaacatctgttgtgattgatgacagcaagaagaaggtttcagaatcttcctcaagtgtagggttattggacttggaggaccaggagcaacttttgaggatatgctgtaaatgcattcagaagcagttgccttccggtacaatgcatgctattcttcagttgtgtgctacactgacgaaagtccatgtggctgctatcagttttcttgagtctggtggcctacatgcgttgctaagtttgccgacaagtagcttgttttctggattcaacagtgtggtttctacaattatccgtcatattttggaggatccccatactcttcagcaagctatggaattagagatacgccatagtcttgtcacggctgctaatcggcatgcaaatcctcgggttacaccacgtaattttgttcaaaatttggcgtttgttgtacacagggacccagtgatatttatgaaagctgcccaagctgtgtgccagattgagatggttggagatagaccgtatgttgttctattgaaggaccgtgagaaggaaaagagcaaggaaaaagagaaggataagctggttgataaggataaatcatcaggtgttgccacaaagataacatcaggggacatggttatggcatctcctgtaagtgccaaagggaaacaatctgatttgagtgtacggaatatgaaatctcatcgcaagccaccacaaacctttgtcactgttattgagcatctattagatctagtaatgtcctttgtcccaccacaaagagctgaggaccaatctgatggttcgtcatccatggacatggatattgactctagttcagcaaaaggtaaagggaaggctgttgctgtcacacatgaagagtccaagcaagcaatccaagatgctactgcatgtctggctaaaaacgcatttgtcctaaagctgctcacagatgtactattaacttatgcttcttccgttcaagttgttcttcgccatgatgctgagttgagcagcacgcgaggtcctactcggaccagtggtggaatatttaatcatatattgcagcatttgcttccacatgctacaaagcaaaagaaagaaagaaagcctgatagtgattggaggtacaaattggcaacaaggggtaatcaatttttagtggcttcatctattcgttcttcagaaggacgaaaaaggatctgttctgaaatctgcagtatatttgttgagttcacagacaattctactggttgcaaacctccaatgttgaggatgaatgcttatgttgatttgctcaatgatattctgtcagctcgctcgccaacgggctcctcactttcagcagaatctgtagttacatttgttgaggttggccttgtccagtgtttaacgaaaacccttcaggttcttgatttggatcatcctgattcagcaaagattgtaactggtattgtcaaggctcttgaagtggttaccaaggagcacgttcatttagcagattttaatgctaaaggggagaactcatcaaagactgttttggagcagaacaatgtagattcatcatcaaatagattccaggttcttgacacaacttctcaaccaactgcaatggtaactgatcacagggaaactttcaatgctgttcatgctccaagaagttcagattcagtggcagatgagatggatcatgaccgtgatatagatggaggttttgcccatgatggtgaagatgattttatgcatgagattgctgaagacagaactggaaatgagtccacaatggatattcggtttgatattccacgtaatagagaggatgatatggctgaagatgaagatgacagtgatgaagatatgtcaggagatgatggtgaggaggttgatgaagatgatgatgatgaggaaaataacaacctggaggaagacgatgcccatcagatatctcatgctgacacggatcaggatgaccgtgagattgatgaagaagaatttgatgaagatctgctagaagaagatgatgatgatgaggatgaggagggagtaatccttcgcctagaggaggggatcaatggaattaatgtgtttgaccatattgaggtttttggggggagcaacaatgtttctggggatacactgcgtgtaatgccattggacatttttggcacaagacggcaaggccgtagtacatctatatacaatcttcttgggagagcaagtgaccaaggtgtacttgatcacccactcttggaggaaccttcgatgttacttccacaacagaggcaaccagaaaatttagttgagatggctttctctgatcggaatcatgaaaatagttcttcccgtttggacgctatattcagaagcttgcgaagtggacggaatggacaccgatttaatatgtggctggatgatggcccccaacgcaatggatctgctgcccctacagtacctgaaggcattgaagaacttctgctctctcagttgagaaggcctacggctgaacatcctgatgagcagagcacacctgctgttgatgctcaagtaaacgatcctcccagtaatttccatgggtcagaaactgatgcaagggaaggatcagcagaacaaaatgaaaatgatgatattcctgcagtaaggtctgaggtggatggctctgcaagtgctggtcctgcacctcctcacagtgatgaacttcaaagagatgcatccaatgcaagtgagcatgtcgcagatatgcaatatgaacgtagtgacgcagctgtccgtgatgtggaagcagtaagccaagcaagcagtggtagtggtgctactctaggggaaagcctcagaagtttagatgtggaaattggaagtgttgaaggacatgatgatggtgaccggcatggggcttcagacaggacacctctgggtgatgtacaggcagctactcgatcgcggaggccttctggaaatgcagtactagtcagcagcagggacatatcattggagagtgttagggagatcccccaaaacacagtccaggaatctgatcagaatgccagtgagggggatcaggagcctaacagggctactgggactgactcaatagatcctacatttttggaggctcttccagaggatttacgggctgaagttctttcttcacgtcaaaatcaagtgactcagacttccagtgagcagcctcagcatgatgcggacattgatcctgaattccttgctgcactgccacctgatatacgtgaagaggttctagctcaacaacgtgcccaacggttgcaacaacagtctcaagagcttgaaggacaaccagttgaaatggacgctgtatcaattattgcaaccttcccttcggaaatacgagaggaggtgcttttgacgtctcccgatactctacttgctacattgacacctgcactagttgctgaagccaacatgttgcgtgagagatttgctcatcgatatcacagcggttcactatttggcatgaactccaggaacaggaggggagagtcctctcgtcgtggtgatatcattggttcaggccttgatagaaatactggtgattcgtcccgtcaaacggccagcaagttaattgaaactgtagggactcctcttgttgacaaggatgctctcaatgctcttatacgattacttcgagttgtccagcctatatacaagggccagctgcagaggcttctcttgaacctttgtgctcaccgggaaagcagaaagtcgctggttcaaattctggtggacatgcttatgcttgatctgcagggctcttctaagaaatcaattgatgcaacagagccgtcatttaggctatatgggtgccatgcaaatatcacatattcacgccctcaatcctcagatggtgtgccaccactggtttctcgccgtgtgctggaaactctgacatacctggcaagaaatcatccaaatgtggctaagctcttactctttcttcagttcccctgcccgcccacctgccatactgaaacacttgaccagaggcgtggaaaggctgtccttgtggaagacggggaacaacagagtgcttttgcactcgttctacttttgactctattaaatcagcctctttatatgagaagtgtagctcatcttgaacagttactcaatcttctggaagttgtcatgcttaatgccgagaatgaagtaaatcaagttaagttgcaaagttcatctgagagaccgtctggacctgagaatgcaacacaagatgcccaagaggatgccagtgttcctggatcatctggagcgaagcccaatgccgatgatagtggtaaatcatcgtccgataatataagtgacctgcaagctgttttgcatagccttccgcaagccgagcttcggttgctatgttcattgcttgcgcatgatgggttatcagacaatgcatatctccttgtagcagaagttctgaaaaagattgtagctttggctcctttcatctgttgccatttcataaatgagctttcacgttcgatgcaaaatttgactgtttgtgccatgaatgagcttcacttgtatgaagattctgaaaaggcaattctgagcacatcatcagccaacggcatggcagttctaagagttgtgcaggccttgagttctcttgtcacatctctgcaagagagaaaagatccagagcttcttgcagagaaggaccattcagatgcactgtcccagatttctgatattaacacagcattggatgcgttgtggttggagctaagcaactgcataagcaaaatagagagctcttcagattatacatcaaatttgagtccaacttctgcaaatgcaactagagtatcaactggtgtagcacctcccttgccagctggaacccagaatatattaccgtacatagaatccttttttgtgacatgcgagaagttacgtccaggacaacctgatgctgttcaagagccttcaacatctgatatggaggatgcttcaacatctagtagtgggcaaaaatcatctgcaagccataccagtcttgatgagaagcacactgcttttgttaaattctcggagaaacacagaagattgttaaatgcttttatccgacaaaactctgggttgctagaaaaatctttctctctgatgttgaaggtcccccgcttgattgattttgacaacaaacgtgcatatttccggtctaaaattaagcatcagcatgaccatcatcacagtcctgttagaatttctgtccgacgggcgtacattttggaagactcctacaatcagcttaggatgcgctcaccccaggacttgaagggtagactcactgttcatttccaaggagaagaaggcattgatgccggtggactcacaagagagtggtatcagttactatcacgagttatttttgacaagggtgcccttctattcacgactgttggaaatgacttgacatttcaaccaaaccctaactctgtgtatcagactgaacacctctcatatttcaaatttgttggacgagtggttggaaaagctctctttgatgcccaacttttggatgtccatttcactagatctttctacaagcacatacttggtgccaaggttacttaccatgatattgaagccattgatcctgcttactacagaaaccttaagtggatgcttgagaatgacataagtgatgtcctggacctcacatttagcatggatgcagatgaagagaagctgatattatatgagaaagctgaggtaactgattgtgagttgattcctggagggcgtaacattagggtcactgaggagaacaagcatgaatatgtggatagggtagcagagcatcgtttaaccactgcaattaggcctcagattaatgctttcatggaaggatttaatgagctcattcctcgcgagctaatatccatttttaatgacaaagagtttgaactgctaatcagtggactccctgatattgacttggacgacctaaaggcaaataccgagtattctggctacagcatagcttctccagttattcaatggttctgggaaattgtacaaggtttcagcaaggaggacaaagctcgatttctccagttcgttactggcacttcaaaggtacctttggagggtttcagtgcgctccaaggaatatctgggccacaaaggttccagatacacaaggcatacggcagcaccaaccatctgccctcagctcatacttgctttaaccaactggacttgcctgaatacacatccaaagaccagctgcaagagaggttgctattagctattcatgaggcaaatgaagggtttggatttggttaa

SEQ ID NO: 7 - protein, 3628 aa, Triticum aestivum
  misc_feature (36)..(37): Xaa can be any naturally occurring amino acid
MAAAAMAAHRASFPLRLQQILSGSRAVSPAIKVESXXPAKVKAFIDRVINIPLHDIAIPLSGFHWEFNKGNFHHWKPLFMHFDTYFKTYISSRKDLLLSDDMSESEPLTKNTILQILRVMQIVLENCQNKTTFAGLEHFKNLLASSDPEVVVAALETLASVVKINPSKLHMNGKLISCGAINSHLLSLAQGWGSKEEGLGLYSCVVANERNQLEGLCLFPADMENKYDGTQHRLGSTLHFEYNLAPVQDSDQANDKSSNLCVIHMPDLHLRKEDDLSILKQCIDKFNVPPEHRFALFTRIRYAHAFNSPRTCRLYSRISLLSFIVLVQSSDAHDELTSFFTNEPEYINELIRLVRSEDIVPGPIRALAMLALGAQLAAYASSHERARILSGSSIISAGGNRMVLLSVLQKAISSLSSPNDTSSPLIVDALLQFFLLHVLSSSSSGTTVRGSGMVPPLLPLLQDKDPSHMHLVCLAVKTLQKLMEYSSPAVSLFKDLGGVELLSQRLHVEVQRVIGVAEITSVVASDTSKSEDDHLYSQKRLIKALLKALGSATYSPANPARSQSSNDNSLPMSLSLIFQNVGKFGGDIYFSSVTVMSEIIHKDPTCFPALKELGLPDAFLSSVTAGAIPSCKALICVPNGLGAICLNNQGLESVRETSALRFLVETFTSRKYLIPMNEGVVLLANAVEELLRHVQSLRSTGVDIIIEIINKLSCPRGDKITEAASAEEKTDMETDVEGRDLVSAMDSGTDGTNDEQFSHLSIFHVMVLVHRTMENSETCRLFVEKGGLQTLLTLLLRPTITQSSGGMPIALHSTMVFKGFTQQHSTPLARAFCSSLKEHLKNALQELDTVFRSCEVTKLEKGAIPSLFIVEFLLFLAASKDNRWMNALLSEFGDVSRDVLEDIGRVHREVLWQISLFDEKKIEPEASSPSANEAQQVDAAVGDTGDNRYTSFRQYLDPLLRRRGSGWNIESQVSDLINIYRDMGRAASDSHRVGADRYPSTGLPSSSQDQPSSSSDANAKSEEDKKRSEHLSCCDMMRSLSYHINHLFMELGKAMLLTSRRENSPINLSPSVVSVASNIASIVLEHLNFEGHTISPEREITVATKCRYLGKVVEFIDGILLDRPESCNPIMVNSFYCRGVIQAILTTFEATSELLFAMNRPPSSPMETDSKTGKEEKDADCSWIYGPLSSYGAAMDHLVTSSFILSSSTRQLLEQPIFSGTVRFPQDAEKFMKLLQSKVLKTVLPIWAHPQFPECNLELISSVTSIMRHVYSGVEVKNNVSNIAARLAGPPPDENAISLIIEMGFSRARAEEALRQVGTNSVEIATDWLFSHPEEPPEDDELARALAMSLGNSDTPVQEEDDRTNDLELEEVNVQLPPMDEVLSSCLRLLQAKETLAFPVRDMLVTISSQNDGQNRVKVLTYLIDHLKQCLVASDPLKNTALSAFCHVLALILHGDTAAREVASKAGLVKVVLSLLCSWEMEPREGQTTKVPNWVTSCFLSVDRMLQLEPKLPDVTELDVLKKDSSPTQTSVVIDDSKKKVSESSSSVGLLDLEDQEQLLRICCKCIQKQLPSGTMHAILQLCATLTKVHVAAISFLESGGLHALLSLPTSSLFSGFNSVVSTIIRHILEDPHTLQQAMELEIRHSLVTAANRHANPRVTPRNFVQNLAFVVHRDPVIFMKAAQAVCQIEMVGDRPYVVLLKDREKEKSKEKEKDKLVDKDKSSGVATKITSGDMVMASPVSAKGKQSDLSVRNMKSHRKPPQTFVTVIEHLLDLVMSFVPPQRAEDQSDGSSSMDMDIDSSSAKGKGKAVAVTHEESKQAIQDATACLAKNAFVLKLLTDVLLTYASSVQVVLRHDAELSSTRGPTRTSGGIFNHILQHLLPHATKQKKERKPDSDWRYKLATRGNQFLVASSIRSSEGRKRICSEICSIFVEFTDNSTGCKPPMLRMNAYVDLLNDILSARSPTGSSLSAESVVTFVEVGLVQCLTKTLQVLDLDHPDSAKIVTGIVKALEVVTKEHVHLADFNAKGENSSKTVLEQNNVDSSSNRFQVLDTTSQPTAMVTDHRETFNAVHAPRSSDSVADEMDHDRDIDGGFAHDGEDDFMHEIAEDRTGNESTMDIRFDIPRNREDDMAEDEDDSDEDMSGDDGEEVDEDDDDEENNNLEEDDAHQISHADTDQDDREIDEEEFDEDLLEEDDDDEDEEGVILRLEEGINGINVFDHIEVFGGSNNVSGDTLRVMPLDIFGTRRQGRSTSIYNLLGRASDQGVLDHPLLEEPSMLLPQQRQPENLVEMAFSDRNHENSSSRLDAIFRSLRSGRNGHRFNMWLDDGPQRNGSAAPTVPEGIEELLLSQLRRPTAEHPDEQSTPAVDAQVNDPPSNFHGSETDAREGSAEQNENDDIPAVRSEVDGSASAGPAPPHSDELQRDASNASEHVADMQYERSDAAVRDVEAVSQASSGSGATLGESLRSLDVEIGSVEGHDDGDRHGASDRTPLGDVQAATRSRRPSGNAVLVSSRDISLESVREIPQNTVQESDQNASEGDQEPNRATGTDSIDPTFLEALPEDLRAEVLSSRQNQVTQTSSEQPQHDADIDPEFLAALPPDIREEVLAQQRAQRLQQQSQELEGQPVEMDAVSIIATFPSEIREEVLLTSPDTLLATLTPALVAEANMLRERFAHRYHSGSLFGMNSRNRRGESSRRGDIIGSGLDRNTGDSSRQTASKLIETVGTPLVDKDALNALIRLLRVVQPIYKGQLQRLLLNLCAHRESRKSLVQILVDMLMLDLQGSSKKSIDATEPSFRLYGCHANITYSRPQSSDGVPPLVSRRVLETLTYLARNHPNVAKLLLFLQFPCPPTCHTETLDQRRGKAVLVEDGEQQSAFALVLLLTLLNQPLYMRSVAHLEQLLNLLEVVMLNAENEVNQVKLQSSSERPSGPENATQDAQEDASVPGSSGAKPNADDSGKSSSDNISDLQAVLHSLPQAELRLLCSLLAHDGLSDNAYLLVAEVLKKIVALAPFICCHFINELSRSMQNLTVCAMNELHLYEDSEKAILSTSSANGMAVLRVVQALSSLVTSLQERKDPELLAEKDHSDALSQISDINTALDALWLELSNCISKIESSSDYTSNLSPTSANATRVSTGVAPPLPAGTQNILPYIESFFVTCEKLRPGQPDAVQEPSTSDMEDASTSSSGQKSSASHTSLDEKHTAFVKFSEKHRRLLNAFIRQNSGLLEKSFSLMLKVPRLIDFDNKRAYFRSKIKHQHDHHHSPVRISVRRAYILEDSYNQLRMRSPQDLKGRLTVHFQGEEGIDAGGLTREWYQLLSRVIFDKGALLFTTVGNDLTFQPNPNSVYQTEHLSYFKFVGRVVGKALFDAQLLDVHFTRSFYKHILGAKVTYHDIEAIDPAYYRNLKWMLENDISDVLDLTFSMDADEEKLILYEKAEVTDCELIPGGRNIRVTEENKHEYVDRVAEHRLTTAIRPQINAFMEGFNELIPRELISIFNDKEFELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKDQLQERLLLAIHEANEGFGFG

SEQ ID NO: 8 - DNA, 10896 bp, Triticum aestivum
  misc_feature (77)..(111): n is a, c, g, or t
  misc_feature (10282)..(10320): n is a, c, g, or t
atggcagcggcggcgatggcagcgcacagggccagcttcccgctgcggctgcagcagatcctgtctggcagccgcgnnnnnnnnnnnnnnnnnnnnnnnnnnnnnnnnnnnccagcaaaagttaaagcatttattgatcgtgtaatcaatattccactacatgacattgccataccactatcaggcttccattgggagttcaataagggaaattttcaccattggaagcctctgtttatgcattttgatacgtatttcaagacatacatctcttctagaaaggatcttctcttgtctgatgatatgtctgagagtgaaccgttgacaaaaaataccatcttacaaattttgagagttgtgcaaattgtgttggaaaattgccagaacaaaacaacttttgctggccttgagcattttaagaatctgttggcatcatcagatcctgaggtagttgttgcggctttggaaactcttgcttcagtggtgaaaataaatccttcaaaactacatatgaatgggaaactgatcagctgtggagctataaatagccatcttctatcattggcccaaggatggggtagcaaggaggaaggtctcggcttgtattcctgtgttgtggcaaatgagcgaaatcagctggagggtttgtgcttattcccagcagacatggagaacaaatatgatggcacacagcatcgtcttgggtccactcttcattttgagtataatttggcacctgtccaagattctgaccaagccaatgacaagtcatctaatctctgcgtgatacatatgccagacctgcaccttcggaaggaggatgacttgagcatattgaagcaatgtatcgacaagtttaatgtgcctccagagcaccggtttgccttgtttacaaggataagatatgcacatgcttttaattctccgaggacatgcaggctatatagccgcataagtctcctttctttcattgttctcgtgcaatccagtgatgcccatgatgaactgacatcgttcttcacaaatgagccggagtacataaatgagttaatccggcttgttcggtcagaagatattgttcctgggcccattcgtgcattggctatgcttgcactgggagcacagttagcagcatatgcatcatctcatgagcgagctaggatactgagcggttcaagtatcatctctgctggtggaaaccgcatggtcttgctcagtgttttgcagaaagcaatatcgtccctcagtagccctaatgatacttcatctcctttaattgttgatgcccttctacagttctttttgcttcatgtgctgtcatcttcgagttctgggaccaccgttaggggttcagggatggttccacctctcctgcctctcctgcaagataaggatccttcccacatgcatcttgtctgtttggcagtaaagacccttcagaagctgatggagtacagcagccctgctgtttccctgtttaaagatttgggtggtgtggaacttttgtctcagaggttacatgtggaggtgcagcgtgttattggtgttgctgaaattacttcagtgcttgctagtgatacatcgaaatcagaagatgaccatctgtactcacagaagcgactgattaaggctttgctcaaggcattaggttccgctacatattctcctgcgaatcctgctcgctctcaaagttccaatgataactctttgcccatgtcactttcccttatattccagaatgttggaaagtttggtggtgatatatacttctcttcagttactgttatgagtgagataatacataaggatcctacatgctttcctgctttgaaggaacttggtcttcctgatgctttcctgtcatcagtgactgctggggtgataccatcttgtaaagctcttatatgtgtacccaatggcctgggtgcaatctgccttaataaccaaggacttgagtctgtcagggaaacttcagtgctgcgttttcttgtagagacgttcactagtaggaaatatctgataccaatgaatgaaggtgttgttcttttagcaaatgcagtggaagagcttcttcgtcatgtgcagtcccttagaagtactggtgttgacatcatcattgaaataatcaacaaactttcttgtcctcgtggtgataaaatcactgaagcagccagagctgaagaaaaaacagatatggaaacagatgttgaagggcgtgatttagtaagtgccatggattctggcacagatggaactaatgatgagcagttttctcatttgagcattttccatgttatggtattggtgcacaggacaatggagaattcagaaacttgcaggctgtttgtagaaaagggaggtctacagactctgttgacactcctgctgcgacctaccatcacccaatcttctggtgggatgcctattgctttgcacagcactatggtatttaagggctttactcaacagcattctactccacttgcgcgtgcattctgctcctctttaaaggagcacttgaagaatgccctacaggaacttgatacagtttttaggtcatgtgaagtgactaagctggaaaaaggagccattccatcactttttattgttgaatttctgctcttcctagcggcatcaaaagacaaccgctggatgaacgctctactctcagaatttggagatgtcagcagggatgtcctggaagatattggaagagttcaccgggaagtgctttggcaaatttcactttttgatgagaagaaaatagagcctgaagctagttctccttcagcaaatgaggcccagcaagttgatgctgctgtgggcgacactgatgataacagatacacctcctttaggcaatatcttgatcctcttttaaggcgacggggctctgggtggaacattgagtctcaggtgtctgaccttatcaatatctatcgtgatatgggccgtgcagctactgactctcacagagttggtgctgatagataccctagtacagggttgccctcaagttcccaggatcagccatccagttcatctgatgcaaatgcgaagtcagaagaggacaagaaaaggtctgagcattcgtcctgctgtgacatgatgaggtcactttcttaccatattaaccatctgtttatggagcttgggaaagcaatgctgcttacatctcgccgtgagaacagtcctataaatctatctccatctgttgtgtctgttgctagtaatattgcttctatcgtgttggagcacctcaattttgaggggcatacaatcagtccagagagggagataactgttgctacaaagtgccgataccttggaaaggttgttgagtttattgatgggatattgttggacagaccagaatcatgtaatccaatcatggtgaattctttttattgccgtggtgtaattcaggctatcttaaccacatttgaagctaccagcgagttgcttttcgcaatgaacaggccaccctcgtcacctatggagactgatagtaaaactgggaaggaagagaaggatactgattgttcatggatttatggccccctttccagctatggtgcagctatggaccatcttgtaacatcatcatttattctttcttcatcgacaagacaactacttgagcaacctatttttagtggaactgtcaggtttccgcaagatgcagagagattcatgaagttgcttcaatctaaagtcttgaagactgttcttcccatatgggcccatccacagtttccagaatgcaatcttgagctaatcagttcagtcacatcgatcatgaggcatgtttactctggggtagaagtaaaaaacaatgttagcaacattgctgctcgtttggctggtccaccccctgacgagaatgcaatttccctgattatagagatgggcttttctcgtgccagagctgaagaagcattaagacaggttggaacaaatagcgttgagattgcgactgattggttgttctcacacccggaggaaccaccagaggatgatgaacttgctcgagctcttgctatgtctctagggaattctgatacacctgttcaagaggaagatgacagaactaatgatcttgagctcgaggaagtaaatgttcagctcacttccatggatgaagtattatcttcatgtcttaggctgctacaggcaaaggaaacattagcttttcctgtccgggatatgcttgtgactatcagctcacagaatgacggccaaaaccgtgtgaaggtgcttacatatttgattgatcatctgaagcaatgtcttgtggcatctgatcctttaaagaacactgcactatctgcttttttccatgttcttgctttgattctccatggagataccgctgctcgtgaagttgcctcaaaggctggtcttgtcaaggttgttttgaatctgctgtgcagctgggagttggagccaagggaaggtcaaacaaccaaggttcccaattgggttacttcctgtttcctttctgttgatagaatgctccagttggaacccaagttgccagatgttactgagcttgatgttctcaaaaaggataattcacctacacaaacatctgttgtgattgatgacagcaagaagaaggattcagaatcttcctcaagtgtagggttattggacttggaggaccaggagcaacttttgaggatatgctgtaaatgcattcagaagcagttgccttccggtacaatgcatgctattcttcagttgtgtgctacactgacgaaagtccatgtggctgctatcagttttcttgagtctggtggcctacatgcgttgctaagtttgccgacaagtagcttgttttctggattcaacagtgtggtttctacaattatccgtcatattttggaggatccccatactcttcagcaagctatggaattagagatacgccatagtcttgtcacggctgctaatcggcatgcaaatcctagggttacaccacgtaattttgttcagaatttggcgtttgttgtatacagggacccagtgatatttatgaaagctgcccaagctgtgtgccagattgagatggttggagatagaccgtatgttgttctattgaaggaccgtgagaaggaaaagagcaaggaaaaagagaaggataagctggttgataaggataaatcatcaggtgttgccacaaagataacatcaggggacatggttatggcatctcctgtaagtgccaaagggaaacaatctgatttgagtgcaaggaatatgaaatctcatcgcaagccaccacaaacctttgtcactgttattgagcatctattagatctagtaatgtcctttgtcccaccacaaagagctgaggaccaatctgatggttcgtcatccatggacatggatattgactctagttcagcaaaaggtaaagggaaggccgttgctgtcacacatgaagagtccaagcaagcaatccaagatgctactgcatgtctggctaaaaacgcatttgtcctaaagctgctcacagatgtactattaacttatgcttcttccgttcaagttgttcttcgccatgatgctgagttgagcagcacgcgaggtcctactcggaccagtggtggaatatttaatcatatattgcagcatttgcttccacatgctacaaaacaaaagaaagaaagaaagcctgatggtgattggaggtacaaattggctacaaggggtaatcagtttttagtggcttcatctattcgttcttcagaaggacgaaaaaggatctgttctgaaatctgcagtatatttgttgagttcacagacaatactggttgcaaacctccaatgttgaggatggatgcttatgttgatttgctcaatgatattctgtcagctcgctcaccaacgggctcgtcactttcagcagaatctgtagttacatttgttgaggttggccttgtccagtgtttaacgaaaacccttcaggttcttgatttggatcatcctgattcagcaaagattgtaactggtattgtcaaggctcttgaagtggttactaaggaacacgttcatttagctgattttaatgctaaaggggagaactcatcaaagactgttttggagcagaacaatgtagattcatcatcaaataggttccaggttcttgacacaacttctcaaccaactgcaatggtaactgatcacagggaaactttcaatgctgttcatgcttcaagaagttcagattcagtggcagatgagatggatcatgaccgtgatatagatggaggttttgcccgtgatggtgaagatgattttatgcatgagattgctgaagacagaacaggaaatgagtccacaatggatattcggtttgatatcccacgtaatagagaggatgatatggctgaagatgaagatgacagtgatgaagatatgtcaggagatgatggtgaggaggttgatgaagatgatgatgatgaggaaaataacaacctggaggaagacgatgcccatcagagatctcatgctgacacggatcaggatgaccgtgagattgatgaagaagaatttgatgaagatcttctagaagaagaagatgatgatgatgaggacgaggagggagtaatccttcgcctagaggaggggatcaatggaattaatgtgtttgaccatattgaggtttttggggggagcaacaatgtttctggggatacactgcgtgtaatgccattggacatttttggcacaagacggcaaggccgtagtacatctatatacaatcttcttgggagagcaagtgaccaaggtgtacttgaccacccactcttggaggaaccttcgatgttacttccacaacagaggcaaccagaaaatttagttgagatggctttctctgatcggaatcatgaaaatagttcttcccgtttggacgctatattcagaagcttgcgaagtggacggaatggacaccgatttaatatgtggctggatgatggcccccaacgcaatggatctgctgcccctacagtacctgaaggcattgaagaacttctgctctctcagttgagaaggcctatggctgaacatcctgatgagcagagcacacctgctgttgatgctcaagtaaacgatcctcccagtaattttcatgggccagaaactgatgcaagggaagggtcagcagaacaaaatgaaaataatgaaaacgttgatattcctgcagtaaggtctgaggtggatggctctgcaagtgctggtcctgcacctcctcacagtgatgaacttcaaagagatgcatccaatgcaagtgagcatgtcgcagatatgcaatatgaacgtagtgacacggctgtccgtgatgtggaagcagtaagccaagcaagcagtggtagtggtgctactctaggggaaagcctcagaagtttagatgttgaaattggaagtgttgaaggacatgatgatggtgaccggcatggggcttcagacaggacacctctgggtgatgtacaggcagctactcgatcgcggaggccttctggaaatgcagtaccagtcagcagcagggacatatcattggagagtgttagggagatccccccaaacacagtccaggaatctgatcagaatgccagtgagggggatcaggagcctaacagggctactgggactgactcaatagatcctacatttttggaggctcttccagaggatttgcgggctgaagttctttcttcacgacaaaatcaagtgactcagacttccagtgagcagcctcagcacgatgcggacattgatcctgaattccttgctgcactgccacctgatatacgtgaagaggttctagctcaacaacgtgcccaacggttgcaacaacagtctcaagagcttgaaggacaaccagttgaaatggatgctgtatcaattattgcaaccttcccttcggaaatacgggaggaggtgcttttgacgtctcccgatactctacttgctacattgacacctgcactagttgctgaagccaacatgttgcgtgagagatttgctcatcgatatcacagcggttcactatttggcatgaactccaggaacaggaggggagagtcctctcgtcgtggtgatatcattggttcaggccttgatagaaatactggtgattcgtcccgtcaaacggccagcaagttaattgaaactgtagggactcctcttgttgacaaggatgctctcaatgctcttatacgattacttcgagttgtccagcctatatacaagggccagctgcagaggcttctcttgaacctttgtgctcaccgggaaagcagaaagtcgctggttcaaattctgttggacatgcttatgcttgatctgcagggctcttctaagaaatcaattgatgcaacagagccgtcatttaggctatatgggtgccatgcaaatatcacatattcacgccctcaatcctcagatggtgtgccaccactggtttcccgccgtgtgctggaaactctgacatacctggcaagaaatcatccaaatgtggctaagctcttactctttcttcagttcccctgcccgcccacctgccatactgaaacacttgaccagaggcgtggaaaggctgtccttgtggaagacggggaacaacagagtgcttttgcactcgttctacttttgactctattaaatcagcctctttatatgagaagtgtagctcatcttgaacagttactcaatcttctggaagttgtcatgcttaatgccgagaatgaagtaaatcaagctaagttggaaagttcagctgagagaccgtctggacctgagaatgcaacacaagatgccctagaggatgccagtgttgctggatcatctggagtgaagcccaatgccgatgatagtggtaaatcatccgcagataatataagtgacctgcaagctgttttgcatagccttccgcaagccgagcttcggttgctatgttcattgcttgcgcatgatgggttatcagacaatgcatatctccttgtagcagaagttctgaaaaagattgtagctttggctcctttcatctgttgccatttcataaatgagctttcacgttcgatgcaaaatttgactgtttgtgccatgaacgagcttcacttgtatgaagattctgaaaaggcaattctgagcacatcatcagccaacggcatggcagttctaagagttgtgcaggccttgagttctcttgtcacatctctgcaagagagaaaagatccagagcttcttgcagagaaggaccattcagatgcgctgtcccagatttctgatattaacacagcattggatgcgttgtggttggagctaagcaactgcataagcaaaatagagagctcttcagaatatacatcaaatttgagtccaacttctgcaaatgcaactagagtatcaactggtgtagcacctcccttgccagctggaacccagaatatattaccgtacatagaatccttttttgtgacatgcgagaagttacgtccaggacaacctgatgctgttcaagagccttcaacatctgatatggaggatgcttcaacatctagtagtgggcaaaaatcatctgcaagccataccagtcttgatgagaagcacactgcttttgttaaattctcggagaaacacagaagattgttaaatgcttttatccgacaaaactctgggttgctagaaaaatcattctctctgatgttgaaggtcccccgcttgattgattttgacaacaaacgtgcatatttccggtctaaaattaagcatcagcatgaccatcatcacagtcctgttagaatttctgtccgacgggcgtacattttggaagactcctataatcagcttaggatgcgctcaccccaggacttgaagggtagactcactgttcatttccaaggagaagaaggcattgatgccggtggactcacaagagagtggtatcagttactatcacgagtgatttttgacaagggagcccttctattcacgactgttggaaatgacttgacatttcaaccaaaccctaactctgtgtatcagactgaacacctctcatatttcaaatttgttggacgagtggttggaaaagcgctctttgatgcccaacttttggatgcccatttcactagatctttctacaagcacatacttggtgccaaggttacttaccatgatattgaagccattgatcctgcttactacagaaaccttaagtggatgcttgagaacgacataagtgatgtcctggacctcacatttagcatggatnnnnnnnnnnnnnnnnnnnnnnnnnnnnnnnnnnnnnnngtaactgattgtgagttgattcctggagggcgtaacattagggtcactgaggagaacaagcatgaatatgtggatagggtagcagagcatcgtttaaccactgcaattaggcctcagattaatgctttcatggaaggatttaatgaactcattcctcgcgagctaatatccatttttaacgacaaagagtttgaactgctaatcagtggactccctgatattgacttggacgacctaaaggcaaataccgagtattctggctacagcatagcttctccagttattcaatggttctgggaaattgtacaaggtttcagcaaggaggacaaagctcgatttctccagttcgttactggcacttcaaaggtacctttggagggtttcagtgcgctccaaggaatatctgggccacaaaggttccagatacacaaggcatacggcagcaccaaccatctgccctcagctcatacttgctttaaccaactggacttgcctgaatacacatccaaagaccagctgcaggagaggttgctattggctattcatgaggcaaatgaagggtttggatttggttaa

SEQ ID NO: 9 - protein, 3631 aa, Triticum aestivum
  misc_feature (26)..(37): Xaa can be any naturally occurring amino acid
  misc_feature (3428)..(3440): Xaa can be any naturally occurring amino acid
MAAAAMAAHRASFPLRLQQILSGSRXXXXXXXXXXXXPAKVKAFIDRVINIPLHDIAIPLSGFHWEFNKGNFHHWKPLFMHFDTYFKTYISSRKDLLLSDDMSESEPLTKNTILQILRVVQIVLENCQNKTTFAGLEHFKNLLASSDPEVVVAALETLASVVKINPSKLHMNGKLISCGAINSHLLSLAQGWGSKEEGLGLYSCVVANERNQLEGLCLFPADMENKYDGTQHRLGSTLHFEYNLAPVQDSDQANDKSSNLCVIHMPDLHLRKEDDLSILKQCIDKFNVPPEHRFALFTRIRYAHAFNSPRTCRLYSRISLLSFIVLVQSSDAHDELTSFFTNEPEYINELIRLVRSEDIVPGPIRALAMLALGAQLAAYASSHERARILSGSSIISAGGNRMVLLSVLQKAISSLSSPNDTSSPLIVDALLQFFLLHVLSSSSSGTTVRGSGMVPPLLPLLQDKDPSHMHLVCLAVKTLQKLMEYSSPAVSLFKDLGGVELLSQRLHVEVQRVIGVAEITSVLASDTSKSEDDHLYSQKRLIKALLKALGSATYSPANPARSQSSNDNSLPMSLSLIFQNVGKFGGDIYFSSVTVMSEIIHKDPTCFPALKELGLPDAFLSSVTAGVIPSCKALICVPNGLGAICLNNQGLESVRETSVLRFLVETFTSRKYLIPMNEGVVLLANAVEELLRHVQSLRSTGVDIIIEIINKLSCPRGDKITEAARAEEKTDMETDVEGRDLVSAMDSGTDGTNDEQFSHLSIFHVMVLVHRTMENSETCRLFVEKGGLQTLLTLLLRPTITQSSGGMPIALHSTMVFKGFTQQHSTPLARAFCSSLKEHLKNALQELDTVFRSCEVTKLEKGAIPSLFIVEFLLFLAASKDNRWMNALLSEFGDVSRDVLEDIGRVHREVLWQISLFDEKKIEPEASSPSANEAQQVDAAVGDTDDNRYTSFRQYLDPLLRRRGSGWNIESQVSDLINIYRDMGRAATDSHRVGADRYPSTGLPSSSQDQPSSSSDANAKSEEDKKRSEHSSCCDMMRSLSYHINHLFMELGKAMLLTSRRENSPINLSPSVVSVASNIASIVLEHLNFEGHTISPEREITVATKCRYLGKVVEFIDGILLDRPESCNPIMVNSFYCRGVIQAILTTFEATSELLFAMNRPPSSPMETDSKTGKEEKDTDCSWIYGPLSSYGAAMDHLVTSSFILSSSTRQLLEQPIFSGTVRFPQDAERFMKLLQSKVLKTVLPIWAHPQFPECNLELISSVTSIMRHVYSGVEVKNNVSNIAARLAGPPPDENAISLIIEMGFSRARAEEALRQVGTNSVEIATDWLFSHPEEPPEDDELARALAMSLGNSDTPVQEEDDRTNDLELEEVNVQLTSMDEVLSSCLRLLQAKETLAFPVRDMLVTISSQNDGQNRVKVLTYLIDHLKQCLVASDPLKNTALSAFFHVLALILHGDTAAREVASKAGLVKVVLNLLCSWELEPREGQTTKVPNWVTSCFLSVDRMLQLEPKLPDVTELDVLKKDNSPTQTSVVIDDSKKKDSESSSSVGLLDLEDQEQLLRICCKCIQKQLPSGTMHAILQLCATLTKVHVAAISFLESGGLHALLSLPTSSLFSGFNSVVSTIIRHILEDPHTLQQAMELEIRHSLVTAANRHANPRVTPRNFVQNLAFVVYRDPVIFMKAAQAVCQIEMVGDRPYVVLLKDREKEKSKEKEKDKLVDKDKSSGVATKITSGDMVMASPVSAKGKQSDLSARNMKSHRKPPQTFVTVIEHLLDLVMSFVPPQRAEDQSDGSSSMDMDIDSSSAKGKGKAVAVTHEESKQAIQDATACLAKNAFVLKLLTDVLLTYASSVQVVLRHDAELSSTRGPTRTSGGIFNHILQHLLPHATKQKKERKPDGDWRYKLATRGNQFLVASSIRSSEGRKRICSEICSIFVEFTDNTGCKPPMLRMDAYVDLLNDILSARSPTGSSLSAESVVTFVEVGLVQCLTKTLQVLDLDHPDSAKIVTGIVKALEVVTKEHVHLADFNAKGENSSKTVLEQNNVDSSSNRFQVLDTTSQPTAMVTDHRETFNAVHASRSSDSVADEMDHDRDIDGGFARDGEDDFMHEIAEDRTGNESTMDIRFDIPRNREDDMAEDEDDSDEDMSGDDGEEVDEDDDDEENNNLEEDDAHQRSHADTDQDDREIDEEEFDEDLLEEEDDDDEDEEGVILRLEEGINGINVFDHIEVFGGSNNVSGDTLRVMPLDIFGTRRQGRSTSIYNLLGRASDQGVLDHPLLEEPSMLLPQQRQPENLVEMAFSDRNHENSSSRLDAIFRSLRSGRNGHRFNMWLDDGPQRNGSAAPTVPEGIEELLLSQLRRPMAEHPDEQSTPAVDAQVNDPPSNFHGPETDAREGSAEQNENNENVDIPAVRSEVDGSASAGPAPPHSDELQRDASNASEHVADMQYERSDTAVRDVEAVSQASSGSGATLGESLRSLDVEIGSVEGHDDGDRHGASDRTPLGDVQAATRSRRPSGNAVPVSSRDISLESVREIPPNTVQESDQNASEGDQEPNRATGTDSIDPTFLEALPEDLRAEVLSSRQNQVTQTSSEQPQHDADIDPEFLAALPPDIREEVLAQQRAQRLQQQSQELEGQPVEMDAVSIIATFPSEIREEVLLTSPDTLLATLTPALVAEANMLRERFAHRYHSGSLFGMNSRNRRGESSRRGDIIGSGLDRNTGDSSRQTASKLIETVGTPLVDKDALNALIRLLRVVQPIYKGQLQRLLLNLCAHRESRKSLVQILLDMLMLDLQGSSKKSIDATEPSFRLYGCHANITYSRPQSSDGVPPLVSRRVLETLTYLARNHPNVAKLLLFLQFPCPPTCHTETLDQRRGKAVLVEDGEQQSAFALVLLLTLLNQPLYMRSVAHLEQLLNLLEVVMLNAENEVNQAKLESSAERPSGPENATQDALEDASVAGSSGVKPNADDSGKSSADNISDLQAVLHSLPQAELRLLCSLLAHDGLSDNAYLLVAEVLKKIVALAPFICCHFINELSRSMQNLTVCAMNELHLYEDSEKAILSTSSANGMAVLRVVQALSSLVTSLQERKDPELLAEKDHSDALSQISDINTALDALWLELSNCISKIESSSEYTSNLSPTSANATRVSTGVAPPLPAGTQNILPYIESFFVTCEKLRPGQPDAVQEPSTSDMEDASTSSSGQKSSASHTSLDEKHTAFVKFSEKHRRLLNAFIRQNSGLLEKSFSLMLKVPRLIDFDNKRAYFRSKIKHQHDHHHSPVRISVRRAYILEDSYNQLRMRSPQDLKGRLTVHFQGEEGIDAGGLTREWYQLLSRVIFDKGALLFTTVGNDLTFQPNPNSVYQTEHLSYFKFVGRVVGKALFDAQLLDAHFTRSFYKHILGAKVTYHDIEAIDPAYYRNLKWMLENDISDVLDLTFSMDXXXXXXXXXXXXXVTDCELIPGGRNIRVTEENKHEYVDRVAEHRLTTAIRPQINAFMEGFNELIPRELISIFNDKEFELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKDQLQERLLLAIHEANEGFGFG

SEQ ID NO: 10 - skipped

SEQ ID NO: 11 - DNA, 16630 bp, Zea mays
atttcaacacacacataaaatatagggaatgacttttctcctcttttattatgtattaccaactgggtgttacaaatcctaccccccttaaaaataatctcgtcctcgagatttaagaagaactagagaaaagatggggaaaatctatgtgaagctcttcttctctttcccacgtagcttcatcttctccgtggtgactccattgtactttacacatctttatcaccttatttcttgtaactcgagtcaatgtatcaagaatcttgatcgggtactccatgtaagtcaaatcaccctgaacactgagctcttccattggtaactgttcctcagggacacggagacacttcttgagctgagacacgtggaacacattatgcacatctgatagactagcaggtagctcgagttggtatgccatctccccaactcgcctaaaaatcaagaatggtccaatatagcgcggggacaatttgcccttaactttgaatctcctcattccacgaagtggtgacaccttgaggtacacataatctccttcctcgaattccagtggtcttctattatctacgaagctcttttgtctggtttgagctaccctcaaattctctcgaatgatacggacttgttcttctgcttcttgaatcagttcaggcccaaagaactgtctttctccagtctgatcccaatgtagaggagtcctgcacttcctcccatataaagcttcaaaaggtgacatcttcagactggcctgataactattattatatgagaactcagcatatggtagactcttatcccaacttcctccatgctgaagagcacaagctctcaacatatcttccaatacttgattagtcctttcggtctgtccatccgtctgagggtgataagtcgtactaaaatttagcttagtgttcatattctcatgaaagcttctccagaatcttgaggtaaactgtgaacctcgatcagacacgatcttcttgggtactccatgtaaacacacaatccaagccatatataactccgctagttgagaacctttataagtagtcttgaccggaatgaaatgagccactttagtcaatctatccacaatcacccatatagcatcatatcctttctgggtgcgaggcaatccagtaatgaaatccataccaatctcttctcacttccactcgggtatcttcagtgggtgcaggagtccagctggcctctggtgttcagccttgactctttgacatacatcgcacatagccacatgtacagccacttctctcttcaatccataccaccaatatttctacttcaaatcctggtacatctgggtactaccaggatgaatagaataatccgagtcatgggcctcctttaaaatagtctcacgaaggctttcgatatcaggaacacatatcttgtccttgaaccatatggtgccttgctcatcttccatgaattccggacctcgaccttcagtaatcagatccctaatctcttgtatcttagcatcaccaatctgtccttctgtggatttcttgctccaaggtaggttccacatcaataataactccttcagtgtgagcaactatccccaggttaagtctcctgaaatcctcaacaatctcatcgggtagctgggtaaccacagctgaatgatcatgctcctttcgactcaaggcatctgcaaccaaatttgcctttcccgggtgatagtgaatctccaaatcatagtccttaataagctccaaccaacagtgttgcctaaggttgagatccttctgagtgaatatatacttgaaactcttatgatctgtgtatacttgacacttggtccccataatataatgtctccaaatcttaagcgcgtgcacaacggctgccagttctaagtcatgagtggggtagttcaattcatgtttccataactaacgagacgcataggcaatcacatgtccctcttgcatgagcacacatcctaagccttggccacatgcatcacaataaatatcaaatcccttctgtaggcctggcataaccaacactggtggagacatcaacctcttctttaattgatcaaagctttcttggcacttctcgtcccacttaaattctctgtccttctccagaagcgaggtcataggcttagaaatcttagaaaatccttcagtaaatctctgataatatcctactagtcccaagaaactccgaatatcagtaactgtagtgggtactctccactccattatctccttaaccttagcaggatccactgatattcctccattagaaataatatgaccaagaaatggcacctcgccaatccaaaactcgcacttgctaaacttggcatagagttgattatctcttagcttctgtagcaccaatctcagatgttcctcatgatcgctatcactcttggaatagataagaatatcgtcgatgaatactacgacgaatctgtccaaatactccataaacaccttgttcatcaagttcataaaataagctggtgcattagttagtccaaatgacataacagtgagctcatataatccatatcgagtcgaaaatgttgtcttgggaatatttgatggtttaatcctcatctgacggtaacccgatcggagatcaatcttcgagaatacctttgcacctctcatctgatagaataaatcctcaatacggggtaacggatacttgttcttcaaagtaacatcattaagtgatctataatccacacacatccgttgtgatccatccttcttctgtacaaacagaaccggtgctccccaaggtgaggaactcggacgaatgtacccagcctcttgcaacttcgttaactgtttcttaagttcctttaactcttctacagacatcctgtatggccgtttagaaatagggacagttccaggtaagagatcaataacaaactcaacttccctatctggtggcatccctggtaactcctccggaaagacatccgggaaatctctaaccacacggatgttgtcaccaacaaactcctccggtataaagaacattgcacgtttggatgaggtggttactgcaatattaacttggaatctttctcctttagggctggtgagttctacggtacctctaccacatgctataatggcctttgcctttcttaaccatgacataccaaggatcggatctatactgcttgcctctaatattatagcggtagctccaaattctcttcccataattgttaatgtcaatctacgtgtcatttgatttgcctcaacaggccctttaggtgtaattactatcattggttttctcatatttagcagtggtatttcttttgtgttggcatgtcaagcagacataaatgaatgtgtagcaccagaatcaaataatatggttgcaggaattgcattaatgtaaaacataccaagtacgatgtcagcaccattaggagtagcgtcaacgctgacttgattaaccctggcaatagagaatcccttgccagatcctggagtctgctgcttattctgaattggagtagtagcaatcctctgtgggcaaacatttgcataatgtccaatcttcccacacttgtagcaagtagtgcctgcactctgtcctgagaatttcgtcggggtaccagtgggggtagcctgacgaggcgatggagtcctctgaggtgattgctgaactaggcgctggcctccgccagtgcttactgatgtaccctggggtgactgtagcgtgggcaaacactgctgcttccttgtccctgagatatagctttcctcttcaaacctcctagctgaacgcgcttgtgttcaatagctagagccttatcaagcagcctctgaaatgatggaaatgtatgtgccaccagagcatactgtagaggtccattaagcccttcaataaaatgctcttgcttcctctcatcctcaacaacttcatctggagcatatctggatagctggataaacttgtcgcggtactcgctgactgacatacttccttgcttcagcgataaaaattccttcttcttaatcttcatcaatccagctggtatatgatagttcctgaactgtgtactgaactccacccatgtgatagtgtcagcagcatcgtgggcagcagtatatgaatcccaccaatcagcagcaggacctgtcagacgtccagaggcatataaaaccttctcccggttagtgcattgagcaatgttcaacatcttctctacggacttcagccaatcatccgcgtccagtggatcaggagaacttgcaaacgtcggtggcttatggctcatgaactcccggtgcttatctcggggtggaactggtggtggtggtggaggcaatggtacttgttgctgttgctgttgttgttgttgaaattgctgttgcgcccttctctcctggcgtatctcttttcgctcctggcgtaattcctggcgtatctcttctctttcctggcgcatctcctggcgttcttgctgcatctgagcacgcatatctgccatagtgtctgccatttgttgcatcatcctcatctgagctacaattagtgggtcaactccgtctggtggaggattaggaggattcatctctgctggttgtggctggggttgtctaaatatcctccgagtgtgtcgattgggaggtaaatcaattccaccacccttcctggtattgaccatctgagttagaaatatatggtaagagagaatagtagacaaggcaggtagttacgaatcatgttactttgggagatttattagctaagtagattagtgtgtcacctactaacgctaatacattaccttatggtggtacatgctaagatgcccgtctatactatttcaatcaaccaaagaagcaaatcagacattgatcatcagaacaatcaaccaacaattttaatagagaaaataatttttaatttctttcttttttggtctcatatctcttaaaaaggttataaatgtaggattccaaggtgtgaaatccttcttgtcttagagtagaaaagataagaatggtcagagtagaacagtagaaggtgagacagggtcaagataagtatagaaagaatcagagtaaggtaagtatagaaagaatcagaataaggtaagtaggtaagggtttgtccatttctatctaggtttcgtcctacagtcaacatttcctctgataccacttctgtcacacccgggctttaaaggacaaagccgggtgcatctcatacatgcgccaaagaagacaacatatataacaacagagtgtatagagataaatgtcacaataacatcagagtatttattacatagcggaagtcttacaaaataagtgataaatataaagcgaactaaggatcgtccttggcgcaaagtcaactggggaaacgccacctagatcagatcaaactcctcgctttgtggctcctcctgaaccacctgctcttctcctgtggggggtgtgagacagcaagggtgagctcacacatgatcatcgctcaacaagttgtggggaataatgtggcatgaactcaccaaaggtgggagttcatgtgatgtgtaaggctaatcaatgataggggttagagctgagcattgcttttaagtagttggtcaaaattttattagcagttactaagtgtaagtaaataccaaaccttaagtaatagaacaaaattaataataaacccatgcatatgcaaatgacaaaattgaatttaagttccataaattaatcatcagagagtcctgagctgctcatgaccgtgagctcggctagtataccaattttacgctctgcagaggttgtaccctttacctacaagtcatgttacccatctgccaaggggtcgcgactcccatacacctctacctaggaagcgaggcagggcaacactacgaggcctttacaaagttccactagcttccgaaaacccgctacagtttatgggaagagcacttgcaagaatcccccgtctgaccgccatcgcagctaaatcaacccgagaacctccttgcatgcaatcccctactgcccttgccccttttgggtaaggtagtcttccactagctttcctaattagtcagctaagggcgtcccattaaacccttgtggtggcacgtgtttctcaagttaagctctatgttccaattaacattaatgatcttgacatgaacataaatagaataacaaaagaattggaacatagatataataaatgattatcccaaaaccatgtaaagcaatagcaaactacccaagtgattcaggggtaaacaaggaaatgagataaacaatctagggtgacctattgggtcccatcaaaattaaacctatgcatgattaagtgacattaaagaacattattgggtaaaaagaggtcaagggcacaacttgccttcaatgagctcctgctcagctacttctatctggtgctcaccaggatcctcagtcactgtctcttctactcgccacaatacaaacaagcacagtatatagagaaaattaacatcacaccaaacatgtacacaaaatacacagtaatgatctacacattaaaataaaatcctaggaacaggaatcataattttttttggagttatagattttaagttatgaatttccaaaggttttatgtgcttaaaataggattaagtgtggaattaattttcttactgttgtcatgtcaaaacagagactctaggtgatagagaataaaattacaaaattttagaaattgaaatggatcaatttggattaaaaatggaatttatatgaattttataagttctaccatttattttcatattaaatattcaatttctaattcatttataacatttaaatgagctctagactgggcgtattatttgcagaaaatacaggggtctcggtataatatttcttaagacatagagcgcccctgggtgtactgcgggtttatttcaattaagcagaaggactttttagcaaatgtggcgtagtgaaggcgtacgcaacgctatccaccgttcgatcccaaacgaagcaccatgattagactactagggtaactaggctatatgcattctgggccatcggatcgttaaccagtggtccacattgattgttaacgcggagtgatccgagccgttcaatcgcatatcgacgtcctgagtcgattacgtcgaaccagtattctggatctaatctacaccgtcccgcccaaatccaatggacccgagcacttcgtctacatccagtcaggtcacggcggcgcgagtactccgcgcggcggcgcactccgatgactcacgctatagacgccagggagaccaaacccaatgggcgactggttcaacacgttgagtagaacgaggagaatagaatggttaaggactcactgatgaaagcgttatggtgtggtctgtccacggaaggaggcggacgacagtggcgctccctgctccggcgagcgattactcgacttccggtggtacacaactcaaattgacggcacgattggttccgccctgagtcgtcgaagcttcccaatgctgcggcccaagcactggagtgctcaatcgagatagcctcgacgacggcgctgcaaatttctctcggtgtcaatggtggcgaccacgggtaaaataaatgcagagctctcggcgaactagttcaaaggtgggttgggacggagtaaggcggccgccgagtttatagccaggcgcccaagaccctgcaggcgcggactgacggaggctccacgcaagaagcggtgaaatccgttcacgcggcgcccttttcgcggagacaacgactggcggatgggggacccacacgtcagcgataaaccgagcgagcgtgtgaagctgccgagtgggggccaggtgtcggcacgattttatcagagctgggccgcgcgaccaacgtgaagggagtgggccgaattggttacagaggcccagttagggtttccctttttctttttcttttgcttttgttttctttttattttgaaatccaaattttggatttcaaattttgtgacaaatttgtactcaactatttgagggtatgattcaaacatgccatggtgaaatgaatttatttctctaaggattttatttggtattggccaatatttctctctctcttttctaaattctaacatctcttgtatattctaaattccacttgagactttaatattttcttttcatatgattctatgttgtcacaaaatgcacacaaaatagaataccagcatgatgcataattgaagggtatatctttttattatttgtttgttgtttgaatatggtgttcacatgtgatgataagtaatttcaacacacacataaaatatagggaatgacttttctcctcttttattatgtattaccaactgggtgtttcagatatccatctcccacagccttcctccaaaaacctgctgaaatctagctcttagttggcgcaccagacctgttcggtgcaccactgggctgagtcctatggcagctgctccgagatcttgactcagaccttccttgtcgggctgacactggaccgatcgggtccaccaccggatcgccacatgtcacctgaccgttgaagtagtcgttgcatgccaagtagccgaggtccagtgcaccaccagaccctccactgtagatggttcgatgattttttaacaaaattcccgagactgacgagtatgtacgaatgggggtttagtgcaccacaggactggtctactgcaccaagccccacgaagcctaagtatgggctctctttgtacgcagtctagtgaggaccagacaaatatttttagtgcataatcaattgaactaagtttaggaaactgaaagtcgcctagaggggggtgaataggcaaatctgaaatttataaactttaagcacaactacaagctagggttagcgttagaaataaaaccgagtccgaaagatagggcgaaaacaaatcacaagcaaatgaaagcggatgacacggtgatttgttttaccgaggttcggttcttgcaaacctactccccgttgaggtggtcacaaagaccgggtctctttcaaccctttccctctctccaccgatcactaagaccggcgagcttttctcctcaatcaaatgggtcacttagaccccacaaggaccaccacaacttggtgtctcttgctttgattacaaaggtgttgagaacaagaaatgggaaagaagaaaagcgatccaagcaacaagaactcaaatgcacacaaaaatctctctctcactagccactaaatgtttggagtgaatttggacttggagaggatttgatctcttgtttgtgtcttggagtgaagtctagagctcttgtattgaatgcaatggctgaaaacttggatgccttgaagtggtggtggttgggggtatttatagccccacccaccaaaatggctgttggggaggctgtctgtcgatgggcgcaccggacagtccggtgcgccaccggacactgtctggtgcgccaccggacactgtctggtgcgccagccatgtcacccaaccggtagggttcgaccgttggagctctgacatatggggccaccggacagtccggtggtgcactggacagtcactgttcactgtccggtgcgccttctggcgctgctctgacttctgcgcgaactgtccgtgcactgttcacgtactgttcaccttttacagacgactgttggcgcagtagccgttgcttcgcttggcacaccggacagtccggtgcagcaccggacagtccggtgaattatagcggagtggcatttccaaaaacccgaagctgaacggttcagagttgatctcgctggtgcacaggacactatccggtggcacaccggacagtccggtgcgccatacaagggcagccttcggttgattttgctcctttctttttgaacccaaactttaactttttattggtttgtgttgaacctttggcacctgtagaacatgtaatctagagcaaactagttagtccaattgtttgtgttgggcaattcaaccaccaaaatcatttaggaaaaggtttgaccctatttccctttcaatctccccctttttggtgattgatgccaacacaaaccaaagcaaatatataagtgcagaattgaactagtttgtataaggtaagtgcaaaggttgcttggaattagaccaatttatactttcataagatatgcaaggattgctttttcttattttaacattttggaccacgcttgcaccacttgttttgttttttcaaatgtttttggaaattcgttttcaaagtctttttgcaaatagtcaaaggtatgtgaataagatttcgagaagcattttcaagatttgaaattttctcccctggtttcaaatgattttcctttgactaaacaaaactcccccttaatgaaattctcctcttagtgttcaagagggttttacagattgaaagaagatcaaatattttagataccaatttttgaataactcctcttaaaatatagataccaaatgagatagaatttcttagaggaatactaatttcaaagataccaattgaaaacaactttgtttcgaaaatttttgaaattggcgtggtggtgcggtccttctgctttgggtgtggtggtgcggtccttttgctttgggctaatactctctccccctttggcattaatcaccaaaaatggagtctttagagcccttcttaaattctctccccctttggcaaacaatatatgagtgaaggattataccaatgtggagagcgatgcggagtgacggcgaagggtaaataataccggtagagtggagtggaagccttgttgtcgccgaagactccatttccctttcaatctatgacttagcatgaaatatacttgaaaaccgcattaggcttatcacatgaaagagatatgatcaaaggtttagaaatgagctatgtgtgcaaagtatcaatcaaaattcctagaatcaagaatgtttagctcattcctaagtttggtaaaggttttctcatctagtggtttggtaaagatatcggctaattgttctttggtgctaacataagcaatctcgatatccccccttcgttggtgatccgttaaaaagtgataccgaatgactatgtgcttagtgcggctgtgttcaactggattattcgccatgcggattgcactctcattataacataggagaggaactttggttaatttgtaaccatagtccctaagggtttgcctcatccaaagcaattgcgcacaacaatggcctgccgcaatatactcggcttcgacggttgaaagagctactgagttttgtttctttgaagccaaagacaccagggatcttcccaagaactgacaagtccctaatgtgctctttctatcaattttacacctagcccaatcagcatctgaataacctattaaatcagaggatcccttggggtaccaaagaccaaacttaggtgtataaactaaagatctcaagattcgtttcatggccctaaggtgaacttccttaggatcggcttggaatcttgcacacatgcatatggaaagcataatatccggtcgagatgcacataaatagagtaaagatcctatcatcgactggtataccttttgatctacggatttacctcccgtgtcgaggtcgagatgcctattggttcccatgggtgtcttgatgggcttggcatccatcattccaaacttggtgagtatgtcttgaatgtacttcatttggctgatgaaggtgccctcttggagttgctttacttgaaatcctagaaaatacttcaactcccccatcatagacatctcgaatttttgtaccatgatcctactaaactcttcacaagtagatttgttagtagacccgaatatgatatcatcaacataaatttggcatacaaacaaatcatttgcaatggttttagtaaagagtgtaggatcggcttttccgactttgaagccattagtgataaggaaatctcttaagcattcataccatgctcttcgggcttgcttgagcccataaagggccttagagagtttatacacatggttagggtactcaatatcttcaaagtcgggaggttgctcaacatagacctcttccttgattggtccattgaggaaggcacttttcacgtccatttggtaaagcttaaagccatggtaagtagcataggcaagtaatatacgaattgactcaagcctagctacgggtgcataggtttcaccgaaatccaaaccttcaacttgtgaatatcccttggccacaagccgggctttgttccttgtcaccacaccatgctcatcttgcttgttgcagataatctacttggctcctacaacgttttgattaggacgtggaactaaatgccatacctcattcctcgtgaagttgttgagttcctcttgcatagccagcacccaatctgaatctcttaatgcatcttccaccctgtatggctcaatagaagacacaaaagagtaatgttcacaaaaatgagcgacacgagatcgagtggttacccccttatgaatgtcaccgagaatggagttcaccgggtgatctagttgtatcgcttggtggactcttgggtgtggcggtcttagaccctgatcatcttccttgtcttgatcattggcatctcccccttgatcattgtcctcctcttgaggtggctcattttcttgatgctcattctcatcatcttgagcttgatcctcatcttgagttggtggagatgcttgcatggaagatgacggttgatcttgtgcttgtggaggctcttcgaattccttaggacacacatccccaatggacatgttccttagcgcgacgcatggagcctcttcatcatctacctcattaagatcaacttgctctacttgagagccgttagtctcatcaaacacaatgtcacaagagacttcaactagtccagtgaacttgttaaagaccctatatgcccttgtgtttgagtcataaccaagtaaaaaaccttctacagtcttaggagcaaatttagattttctacctcttttaacaagaatagaacatttgctaccaaagactctaaaatatgaaacattgggctttttactagtgaggagttcataagatgtcttcttgaggattcggtgaaggtagagacagttgatggcgtagcaagcggtgttgattgcttctgcccaaaatcgatctgaagtcttgtactcatcaagcatggtcctcgccatgtcaagtagagttctattcttcctctccactacaccattttgttgtggtgtgtagggagaagagaactcatgtttgatgccctcgtcctcaagaaagccttctatttgagagttcttgaactccgtcccattgtcgcttctaatcttttgtatccttaagccgaactcattttgagcccgtctcaagaatccctttaaggtctcttgggtttgtgatttttcctgcaaaaagatcacccaagtgaagcgagaataatcatccacaattacaagacaatacttactcccaccgatgcttatgtaagcgatcgggctgaatagatccatgtggagtagctcaagtggcctgtcggtcgtcatgatgttcttatgtggatgatgaacaccaacttgcttccctgcttgacatgcgctacaaaccctgtctttctcaaaatgaacatttgttagtcccaaaatgtgttctccctttagaagcttgtgaagattcttcatcccaacatgggctagtcggcgatgccagagccaacccatattagtcttagcaattaagcaagtatcgaattcagctctattgaaatcaactaagtatagctgaccctctaatactcccttaaatgctactaaatcatcacttcttctaaagacagtaacacctatatccgtaaaaagacaattgtagcccattttgcataattgagaaacagaaagcaaattgtaatctaaagaatctacaagaaaaacattggaaatagaatggtcaggagatatagtaattttaccaagtcctttgaccaaccttgatttccatccccgaatgtgatagctctttggggatcttcatttttctcgtaggaggagaacatccttttctcccctatcatgtggtttgtgcacccgctatcaatgatccaacttgagcccccggatgcataaacctacaaaacaaatttaggccttgttcttaggtacccaaacggtcttgggtcctttgacattagagacaagcaccttgggtacccaaacacaagtctttgagcccttgtatttggccccaacatatttggcaactattttgcctgatttgttagttaaaacataagaagcatcaaacgtcttaaatgaaatattaggttcatttgatgcaataggagatttctttttaggcaatttaacatgagtggattgcctagaactagatgcctcactcttatacataaaagcatgatgagagccagagtgagacttcctagaatgaattctcctaattttgtgttcgggataaccagcatgatataaaatttagccctcgttatcctgaaccatgggagctttgcccttaacaaagttagacaatttcttaggagcattaagcttgacattgtctccctgttggaagccaatgccatccttaatgccagggcgtctcccactatagagcatacttctagcaaatttaatttttttattttctaagtcatgctcattaattttagcactaagttgagctatgtgatcattttgttgtttaattaaagctaggttatcatggatagcatcaacattaatgtctctacatctagtacaaatagtaacatgatcaacactagatgtagatggtttgcaagcatttaattcatcaatcttagcatgtaacatttcattttcatttctaagattggaaatcaaaacattgcaaacacttaaatctttagccttagaaattaatttatcattctcaatcttaaggcaagaaattgattcatttaatttgtcaatcttagcaagtaaattagcattatcatttttaagattggcaaaagaatcatcacaaacatttaacttctcaaccttagcaatcaatttagcattctcaaatctaaggttggaaatagtgtcatggcaagtgcttagctcactagtcaatttttcacatttttctacttcttgagcataagcatttttaaccttaacatgcttcttatttttcttaataaggaagtcctcttggctatccaagagttcatccttctcatgaatagcactaatcaattcattcaatttttccttttgttgcatgtttaggttggcaaaaagtgtgagcaaatcatcctcatcatcactagtgctagcctcatcactagatgttgcatatttagtggaagcactagattttaccttcttctttttgtcgtcctttgccatgaggcacttgtggccgatgttggggaagaggaggcctttattgacggcgatgttggcggcgtcctcgtcggaggaggagtcggtggagctctcatcggagtcccattcccgacatacgtgggcatcgccgcccttcttgtagtatctcttcttttccttcctctttcccttcttgtcatcgcccctgtcactatcactagacaatggacatttagcaataaaatgaccgggcttaccacatttgtagcaaactttctcggagcggggcttgtaatccttccccctcctttgcttgaggatttggcggaagctcttgatgatgagcgtcatttcctcattgtcgagcttggaggcgtcgatggggagcctacttgacgtagactcttcgttcttctcctccgtcgctttgaatgcgacgggttgcacctcaggtgtggaggtggcgcctcgctcgatgatttgtttggagcctttgatcatcaactcaaagctcacaaactttcctataacctcctcgggagacattagtttgtatctaggatcaccacgaattaattgtacttgagtaggattaagaaaaacaagtgatcctagaataaccttgaccatttcatggtcatcccattttgtgctcccgaggttgcgcacttggttcaccaaggtcttcagccggttgtacatggcttgtggctcctccccttggttgagcatgaatcgaccgagctccctctcgatcgtttcccgcttggtgatcttggtcacctcatctccttcgtgtgtggtctttagtacgtcccaaatctctttggcacttttcaacccttgcaccttattatactcctctcgacttagagaggcgaggagtatagtagtggcttgggagttgaagtgctggatttgggctacctcatccgagtcatagccttcatcccccacgaatggttcctgcgctccaaactcaacaatgtcccaaatgctagcgtggagtgaggttagatggtgcctcattttatcactccacatacaataatcttcaccgtcaaaaaccggtggtttgcctagtgggacggaaagtaaaggagtgcgtttggaaatgcgaggatagcgtaagggcatcttactaaacttcttgcgctcatggc

SEQ ID NO: 12 - protein, 3640 aa, Zea mays
MAAAAAAMAAHRASFPLRLQQILAGSRAVSPAIKIESEPPANIKAFIDRVVNIPLHDIAIPLSGFCWEFNKGNFHHWRPLFIHFDTYFKTYISSRKDLLLSDDMTEADPMPKNAILKILRVMQIILENCQNRSSFTGLAHLKLLLASSDPEIVVAALETLVALVKINPSKLHMNGKLISCGPINTHLLSLAQGWGSKEEGLGIYSCVVANEGNHQGGLSLFPVDLENKYGGTQHRLGSTLHFEYNLGPAQYPGQTSDKGKSSNLCVIHIPDMHLQKEDDLSILKQCVDKFNVPPEHRFALLTRIRYARAFNSARTCRIYSRISLLSFIVLVQSSDAHDELTYFFTNEPEYINELIRLVRSEDSVPGSIRALAMLALGAQLAAYASSHERARILSGSSIISAGGNRMVLLSVLQKAISSLNSLNDTSSPLIVDALLQFFLLHVLSSSSSGTTVRGSGMVPPLLPLLRDNDPSHMHLVCLAVKTLQKLMEYSSPAVSLFKDLGGVELLSQRLHVEVQRVIGTADGHNSMVTDAVKSDDNHMYSQKRLIKALLKALGSATYSPGNPARSQSSQDNSLPVSLSLIFQNVDKFGGDIYFSAVTVMSEIIHKDPTCFITLKELGVPDAFISSVTAGVIPSCKALICVPNGLGAICLNNQGLEAVRETSALRFLVDTFTSRKYLIPMNEGVVLLANAVEELLRHVQSLRSIGVDIIIEIINKLSSSQEYKNNETATLQEKTDMETDVEGRDLVSAMDSSVDGSNDEQFSHLSIFHVMVLVHRTMENSETCRLFVEKGGLHALLTLLLRPSITQSSGGMPIALHSTMVFKGFTQHHSTPLARAFCSSLKEHLKSALKELDKVSNSFDMTKIEKGAIPSLFVVEFLLFLAASKDNRWMNALLSEFGDASREVLEDVGQVHREVLWKISLFEKNKIVAETSSSSSTSEAQQPDMSASDIGDSRYTSFRQYLDPILRRRGSGWNIESQVSDLINMYRDIGRAASDSQRVGSDRYSSLGLPSSSQDQFSSSSDANASTRSEEDKKKSEHSSCFDMMRSLSYHINHLFLELGKAMLFASRRENSPVNLSPAVISVANNIASIVLEHLNFEGHSVSFERDMTVTTKCRYLGKVVEFVDGMLLDRPESCNSIMVNSFYCRGVIQAILTTFQATSELLFTMSRPPSSPMETDSKTGKDGKEMDSSWIYGPLTSYGAIMDHLVTSSFILSSSTRQLLEQPIFNGSVRFPQDAETFMKLLQSKVLKTVLPIWAHPQFPECNIELISSVMSIMRHVCSGVEVKDTVGNGGARLAGPPPDESAISLIVEMGFSRARAEEALRQVGTNSVEIATDWLFAHPEEPQEEDDELARALAMSLGNSVTPAQEGDSRSNDLELEEATVQPPPIDEMLRSCLQLLQRKEALAFSVRDMLVTISSQNDGQNRVKVLTYLIDNLKQCVVASEPSNDTALSALLHVLALILHGDTAAREVASKAGLVKVALDLLCSWEVQIRESSMIEVPNWVISCFLSVDQMLQLEPKLPDVTELHVLKRDNSNIKTSLVIDDSKRKDSESLPNVGLLDMEDQFQLLKICCKCIGKQLPSASMHAILQLSATLTKVHAAAICFLESGGLNALLSLPTSSLFSGFNNMASTIIRHILEDPHTLQQAMELEIRHSLVTAANRHANPRVTPRNFIQNLAFVVYRDPVIFMKAAQSVCQIEMVGDRPYVVLLKDREKERIKEKDKDKSVDKDKATVAVTKVVSGDTAAGSPANSHGKQSDLNSRNVKSHRKPPQSFVTVIEHLLDLLMSFVPPPRPEDQVDVSGTALSSDMDIDCSSAKGKGKAVSVPPEESKHAIQESTASLAKTAFFLKLLTDVLLTYASSIHVVLRHDAELSNMHGPNRTSARLTSGGIFNHILQHFLPHATRQKKERKNDGDWMYKLATRANQFLVASSIRSAEARKRIFSEICSIFLDFTDSSAGYNAPVPRMNVYVDLLNDILSARSPTGSSLSAESAVIFVEAGLVHSLSTMLQVLDLDHPDSAKIVTAVVKALELVSKEHIHSADNAKGVNSSKIASDSNNVNSSSNRFQALDMTSQPTEMVTDHRETFNAVRTSQISDSVADEMDHDRDMDGGFARDGEDDFMHEMAEDGTGDGSTMEIRIEIPRNREDDMAPAADDTDEDISAEDGEDDEDEDEENNNLEEDDAHRMSHPDTDQEDREMDEEEFDEDLLEEDDEDEDEEGVILRLEEGINGINVLDHVEVFGGSNNLSGDTLRVMPLDIFGTRRQGRSTSIYNLLGRASDHGVLDHPLLEEPSSTTNFSDQGHPENLVEMAFSDRNHESSSSRLDAIFRSLRSGRNGHRFNMWLDDGPQRNGSAAPAVPEGIEELLISHLRRPTPQPDGQRTPVGGAQENDQPNHGSDAEAREVAPAQQNENSESTLNPLDLSECAGPAPPDSDALQRDVSNASELATEMQYERSDAITRDVEAVSQASSGSGATLGESLRSLEVEIGSVEGHDDGDRHGTSGTSERLPLGDIQAAARSRRPSGNAVPVSSRDMSLESVSEVPQNPDQEPDQNASEGNQEPTRAAGADSIDPTFLEALPEDLRAEVLSSRQNQVTQTSNDQPQDDGDIDPEFLAALPPDIREEVLAQQRTQRMQQQSQELEGQPVEMDAVSIIATFPSEIREEVLLTSPDTLLATLTPALVAEANMLRERFAHRYHSSSLFGMNSRNRRGESSRRDIMAAGLDRNTGDPSRSTSKPIETEGAPLVDEDGLKALIRLLRVVQPLYKGQLQKLLVNLCTHRGSRQALVQILVDMLMLDLQGFSKKSIDAPEPPFRLYGCHANIAYSRPQSSDGLPPLVSRRVLETLTNLARSHPNVAKLLLFLEFPCPSRCFPEAHDHRHGKAVLLDDGEEQKTFALVLLLNLLDQPLYMRSVAHLEQLLNLLDVVMHNAENEIKQAKLEASSEKPSAPDNAVQDGKNNSDISVSYGSELNPEDGSKAPAVDNRSNLQAVLRSLPQPELRLLCSLLAHDGLSDSAYLLVGEVLKKIVALAPFFCCHFINELARSMQNLTLRAMKELHLYENSEKALLSSSSANGTAVLRVVQALSSLVNTLQERKDPEQPAEKDHSDAVSQISEINTALDSLWLELSNCISKIESSSEYASNLSPASASAAMLTTGVAPPLPAGTQNLLPYIESFFVTCEKLRPGQPDAVQDASTSDMEDASTSSGGQRSSACQASLDEKQNAFVKFSEKHRRLLNAFIRQNSGLLEKSFSLMLKIPRLIDFDNKRAYFRSKIKHQYDHHHHSPVRISVRRPYILEDSYNQLRMRSPQDLKGRLTVQFQGEEGIDAGGLTREWYQSISRVIVDKSALLFTTVGNDLTFQPNPNSVYQTEHLSYFKFVGRVVGKALFDGQLLDAHFTRSFYKHILGVKVTYHDIEAIDPSYYKNLKWMLENDISDVLDLTFSMDADEEKLILYEKAEVFAVTDCELIPGGRNIRVTEENKHEYVDRVAEHRLTTAIRPQINAFLEGFNELIPRELISIFNDKELELLISGLPDIDLDDLKTNTEYSGYSIASPVVQWFWEIVQGFSKEDKARFLQFVTGTSKVPLEGFSELQGISGPQRFQIHKAYGSTNHLPSAHTCFNQLDLPEYTSKEQLQERLLLAIHEANE

SEQ ID NO: 13 - DNA, 10905 bp, Zea mays
atggcggcggcggcggccgcacatagagccagcttcccgttgcgcctgcagcagatccttgctggaagccgcgctgtctcacctgcgatcaaggtcgagtctgagccgcccgcaaatgtcaaagcatttattgatcaagtaatcaatattccactacatgacattgccataccgctatcaggcttccgttgggagttcaataagggaaatttccaccactggaagcctttatttattcattttgacacatatttcaagacatacatttcttctcggaaggatcttcttttatctgatgatatgactgaggctgaacctatgccaaagaatgccatcttgaaaattttaatagttatgcagattattttggagaactgtcagaatagaagttcttttactggtcttgagcatcttaagcttctcctggcatcgtcagaccctgagatagttgtagcagcattggaaactcttgttgccttggtcaaaataaatccttcaaagttgcatatgaatggaaagctaattagctgcggatccataaacacccatcttctatcattggcccaaggatggggcagcaaggaggaaggcctaggcatatattcttgtgttgtggcaaatgaaggaaaccagcaggggggcctgtccttgttcccagtagacttggagagcaaatatcagcatcgtcttggttcaactcttcattttgagtataatttgggttctgcacagtaccctgaccaaactagtgacaagggcaagtcatctaatttgtgtgtgatacatattccagacatgcatcttcagaaagaggatgacttgagcatactgaagcaatgcgtggacaaattcaatgtgcctccagagcacaggtttgcattgcttacaaggataagatatgcccgcgccttcaactccacacggacttgtagtatctatagccgcataagtcttctttcgttcattgtacttgtgcaatcaagtgatgctcacgatgagctcacatatttctttacaaatgaaccagaatacataaacgagttaatcaggcttgttcggtcagaagattctgtccctgggcccattcgtgcacttgctatgctagcactgggagcacagctggcagcatatgcatcatctcatgagcgtgctaggatattgagtggctcaagtatcatttctgctggtggaaatcgtatggtgttgctcagtgtcttgcagaaagcaatattctcactcaatagcccaaatgatgcctcatctcctctaattgttgatgctcttcttcagttttttctgctccatgtattgtcctcatcaagttcagggactacggttagaggctcaggcatggttcctcctctcttgcccctgctgcgagataatgattcttctcatatgcatcttgtctgcttggcagtgaagactcttcaaaagcttatggagtacagcagccctgctgtttctctgttcaaagacttgggtggtgtagaccttttgtctcggaggttacatgtagaagtgcagcgtgttattggtactgctgatggtcataattcaatggttactgatgcagttaaatcaaaagaggaccacctgtactcccagaagcgattgattaaagctttgcttaaggctttggggtcttctacttattcccctgggattcctgctcgttctcaaagctcccaagataattcattgcctgtgtcactttcccttatatttcagaatgtagaaaagtttgggggcgacatttatttttcagcagttacagttatgagcgaaataatccataaagatcctacatgctttcctgctttgaaagaacttggtcttccagatgcttttctctcatcagtgactgctggggtaataccatcttgtaaagccctgatctgtgttcctaatggcctgggtgcaatatgcctcaataaccaaggacttgaggctgtcagggaaacttcagcactacgatttcttgtatataccttcactagcaggaaatacttgataccactgaatgagggcgttgttcttctagctaatgcagcagaagagcttcttcgtcatgtgcaatccctgagaagtattggtgttgacatcattattgaaataatcaataaactaagttcatctctgaaagatagaaacaatgaaacagccatcttggaagaaaaaacagatatggaaactgatgttgaaggtcgtgatttggtaggtggtatggattccagtgtggagggcagtaatgatgaacagttttctcatttgagcattttccatgtgatggtattggttcaccggacaatggaaaactctgaaacctgccggttatttgtcgaaaagggaggtctaaatgctcttttgacactcttgttgaggcctagcattacactgtcatctggagggatgcctattgctttgcatagcacaatggtgttcaagggatttactcagcatcactctactccacttgcacgtgcattttgctcgtctttaagggagcatttgaagagtgcattaggggaacttgataaagtttctaactcatttgagatgaccaagattgaaaaaggagcgataccttctttgtttgttgttgaatttctgctatttttagctgcctcaaaagacaatcgttggatgaatgctttgctctcagaatttggcgatgctagcagggaggtcctggaagatattgggcgagtccaccgggaagtgctttggaaaatttcgctttttgaagaaaacaagattgacgctgaaattagtttatcttcttccacatctgaagcccagcagcctgacttgtctgccagcgacattggtgatagcagatacacttctttcaggcaatatcttgatccaattttaaggcgaaggggttctgggtggaacatagagtctcaagtgtctgacctcattaatatgtatcgagacattggcagtgcagctagtgactctcagagagttggttctgatagatattctagtctgggtttgccctcaagttcccaggaccagtcttccagttcatctgatgcaaatgtcagcacaagatcagaagaggaaaagaaaaattctgagcattcctcctgctttgacatgatgagatctctctcctaccatatcaaccatctttttatggagcttggtaaagcaatgcttcttacatctcgtcgggagaacagtcctgtaaacctatccccatccgttatatctgttgctaacaacattgcttctattatgttggaacacctcaactttgaaggacattcggtaagttctgagagggagatgactgttaccacaaaatgccaatatcttggaaaggtagctgaatttattgatgggatattattagatcggccagaatcctgtaatccaattatggtaaattcattctattgctgtggtgtcattcaggccatcctgaccacatttcaggccaccagtgagttgctcttcacaatgagcaggccaccatcttcacctatggagacagatagtaaaactggaaaggatggaaaggatatgaattcttcatggatctatggtcccctcatcagctatggcgcaattatggatcatcttgtgacctcgtcttttattctctcttcatcaactagacaattacttgagcagcctatttttaatggatctgtaaggtttcctcaagatgctgagaggttcatgaagttacttcagtctaaggtgttgaagacagttcttcccatctgggctcatcctgagtttccagaatgcaacattgagttgattagttcagtcatgtccatcatgaggcatgtttgctctggggtggaagtgaaaaacactgttggcaatgatggtgctcgtttaactggtccacctcctgatgagagtgcaatttcattgattgtagagatggggttttctcgtgcaagagctgaagaagcattaagacaagttggaaccaacagtgttgaaatagcaactgattggttattctcacacccagaggatgaacttgctcgggcgctcgcaatgtctcttgggaattctgatacacctgcacaagagggaaatggtaggtccaatgaccttgagctggaggaagtaacagtccagctgcctcctatagatgaaatgttacactcatgttttcagctacttcagacaaaggaagcattagctttccctgtcagggatatgcttgttactatcagctcacagaaggatggccaaaaccgagtaaaggtgcttacatatttgattgaaaatctgaaacaatgtgtggtggcatctgagccttcaaatgatactgcactgtctgctcttttacatgttcttgctttgattctccatggagatactgctgctcgtgaagttgcctcaaaggcaggaattgtgaaggttgctttagatctgctttccagctgggagttggaacttagggaaagtggcatgattgaggttcctaattgggtaagttcctgtttcctttctgttgatcagatgctccagttggaaccaaagttgccagatgttacagagctagatgttctgaaaagggataattcaaatataaaaacatcacttgtgattgatgagagcaagaaaaaagattcagaatccttgtcaagtgttggattactagacatggaggaccagtatcaacttttgaagatctgttgcaagtgcatagaaaaacagttgccttcggcttccatgcatgctattcttcagctctctgcaacactgacaaaagttcatgcagctgctatttgtttccttgagtctggtggtctgaatgcattgctatcattgccaacaagtagtttgttttctgggttcaacagtgtggcttctacaataattcgtcatattttggaggaccctcacacccttcagcaagcaatggaattagagatacgtcacagtcttgtcacagctgctaatcgacatacaaatcccagggttaccccacgcaattttgtccagaatttggcatttgtcatatatagggacccagtgatatttatgaaagctgtccagtctgtgtgccagattgagatggttggagatagaccgtatgttgttctgttaaaggatcgtgagaaagaaaggagcaaggagaaggataaggacaaatcagttgataaggataaagcaacaggtgcagtcgctaaggtagtgtccggggacacagctgcaggatctcctgcaaatgctcaggggaaacagtctgatttgaattcaagaaacgtgaaatctcaccgcaagccaccccaaagttttgtcactgttatagagcatctcttagatctggtgatgtcctttgttccgccaccccgtccagaggatcaagctgatgttgtctctggtactgccttatcttctgacatggacattgactgcagttctgcaaaagggaaagggaaagctgtgtctgtcccacctgaagagagcaagcatgctatccaagagtctactgcatcccttgccaaagcttcatttttccttaagttgatgacagatgtgttgttaacttacacatcatccattcaagttgttcttcgtcatgatgctgatctgagcaacatgcacggtccaaatcgaacaaattctggtctaattagtggtggaatctttaaccatatcctgcagcatttccttcctcatgccacaaagcaaaagaaagagagaaaaagtgatggagattggatgtataaattggcaacaagggctaatcagttcttggtggcttcatctattcgttctgcagaggcccgaaaaaaagttttttctgaaatctgcaatatacttcttgatttcaccgactcgtctgcagcttacaaagccccagtagcaaggatgaatgtgtatgttgatttactgaatgatattctgtcagcccgatcaccaactggctcctcattgtcagcagaatctgcagttacatttgttgaagttggtcttgctccttcattattgaaaatgcttcaaaatcttgatttggatcaccctgattctgcaaagatcgttactgccattgttaaggccctggaactcgtcagtaaggaacatgttcattcagcagataatgcaaaaggagagaactcatcaaagattgcttccgacagcaacaatgtgaattcatcacctaataggttccaggctcttgacatgacttctcagcccacagagatgattactgatcacagggaaacatttaatgctgaccaaacttcacaaagttcagattcagtggcagatgagatggatcatgatcgtgatatggatggtggctttgctcgtgatggtgaagatgattttatgcatgagatggctggcgatggaactggtaatgaatctacaatggaaatcagatttgaaatttcacgcaatagagacgatatggctgatgatgatgatgatgatgacaacactgatgaagatatgtctgcagaggatgatgaggaggttaatgaagatgatgaagacgaagatgaggagaataacaatttggaggaagatgatgcccatcagatgtctcatcctgacacagaccaggaagaccgtgaaatggatgaagaagaatttgacgaggatctactagaagatgatgacgatgaggatgaggagggagtcatccttcgcctggaggaggggattaatggcattaatgtgtttgaccatattgaagtatttggtggtagcaacaatttgtctggggatacactgcgtgtgatgcctctggacatttttggtacaagaaggcaaggccgaagtacctccatatacaatcttcttggcagggctagtgatcatggtgttcttgatcacccacttcttgaagagccttcctcgacattgaatttttcacatcaagaacaaccagagaatcttgttgagatggctttctctgatcgaaatcacgaaggtagctcctcccgtttggatgctatattccgcagcttgcgaagtgggcgtaatggacatcgctttaatatgtggctagatgacggtccacaacgcaatggatctgctgcccctgcagtacctgaaggcattgaagaattgctgatctctcatttgagtcggcccactcaacaacctggtgcccagactgttggtggtactcaagaaaatgaccaacccaaacatggctcagctgctgaggcaagggaaggatcgccagctcagcaaaatgagaacagtgaaaatacaacgaatcctgtagatctgtcagaaagtgctggccctgcacctcctgatagtgatgcacttcaaagagttgtgtccaatgcaagtattgagcatgctacggagatgcagtatgaacgcagcgacactataacacgtgatgttgaagcagtgagccaagcaagcagtggcagtggtgctactttaggggagagcctcagaagtttagaggttgaaataggaagtgttgaagggcatgatgatggtgaccggcatggaacttcaggggcctcagaacgattacctttgggtgatattcaagctgctgctcgatcacgcaggccatctggaaatgctgtggctgtaagcagtagagatatgtctctggagagtgttagtgaggttcctcaaaatccagatcaagaacctgatcataatgctagtgaggggaatcaggagcctagaggggttggtgctgacacaattgatcctacatttttggaggctcttcctgaggacttgcgagctgaagttctttcttcacgtcaaaatcaagtgactcagacttctaatgaccaacctcagaatgatggtgatattgatcctgaattccttgctgcactcccccttgatataagagaggaggtgctagctcaacaacgtagtcaaaggatacaacaacaatcacaagaattagaaggacaaccagttgaaatggatgctgtttcaattattgcaacattcccttcagaaattcgggaagaggtccttttgacatctcctgacaccctcctagctacactcacacctgcactagttgctgaggccaacatgttaagggagagatttgctcatcggtatcacagtagttctctttttggtatgaactccaggaacaggagaggtgagtcctcccgacgtgacataatggcagcaggacttgataggaacacaggtgatccctccagatcaactagcaaaccaattgagattgaaggtgctcctcttgttgatgaagatggccttaaagctcttattcggttacttcgagttgttcagcccctatacaagggtcagctgcagaggcttcttgtgaacctttgcacgcatcgggataacagacaggccttggttcaaattctggtggacatgcttatgcttgatctgcaggggttttcaaagaaatcagtagatgcatctgagccaccatttaggttgtatggatgccatgcaaatatcacatattcacgtcctcaatcttcaaatggtgttcctccattggtttcacgtcgtgtgctcgaaacactgacaaacttggcaagaagccatccaaatgttgctaaactcttgctctttcttgagttcccttgcccatcaagatgtcgctctgaagcacatgatcataggcatggcaaggctgttctagaagatggtgaagaacggaaagcctttgcggttgttcttttgttaactctgttgaatcagccactttatatgagaagtgtagctcatcttgaacagttactaaacctccttgaagttgtcatgcataacgctgagaatgaaattaatcaagctaagctagaagcgtcatctgagaagccatctgagaatgcagtgaaagatgtcaaagataacaccagcatttctgattcatatggatcaaagtcgaatcctgaggatggtagcaaagcccttgctgttgacaataaaagtaacttgcgagctgttctgcgaagtcttccccaatcagagcttagattgctatgttcattgcttgcgcatgatggattgtcggacagcgcttatctccttgtaggtgaagtgttgaaaaagattgtggctctggcccctttcttctgttgccatttcataaatgagcttgcacgatcaatgcaaagcttgacattttgtgcaatgaaggagcttcgtttgtacgagaattctgaaaaggctcttcttagttctacatcagccaatggaactgcgattcttagagttgtgcaggctttgagttctcttgtcagtactcttcaagatagaaaggatccagaacagcctgctgagaaggaccattcagatgcagtttcccagatttctgaaattaacacagcattggatgctttgtggttggaattgagcaactgcataagcaaaatagaaagctcttcagagtatgcatcaaatctaactcctgcttctgcaagtgcagctacattaacagcaggagtagcacctccattgcctgcaggaactcaaaacatattaccatatatagagtcattttttgtgacatgtgagaagttgcgtcctgggcaacctgatgctgtacaagaggcttcgacttctgatatggaagatgcttcaacatctagtggtgggcagagatcatattcctgtcaagctagtcttgacgagaagcaaaatgcttttgtgaaattctcagagaagcacagaagattgctgaatgcattcatccatcaaaaccctggattactggagaagtcattctcgctgatgttaaaaattcctcgcctgattgattttgacaacaaacgtgcatatttccggtctaaaatcaagcatcagtatgatcatcatcatcataatcctgttagaatttctgtgcgtcgttcatatattcttgaggactcctacaatcagcttaggatgcgctctcctcaggacttgaagggtagactcactgttcattttcaaggagaagaaggaattgatgctggtgggctgacaagggaatggtaccaatcgctttcacgagtcatttttgacaagagtgcccttctatttacaacagtcgggaatgatttgacgtttcaaccgaatcctaattctgtatatcaaactgagcacctctcatactttaaatttgctggacgagtggttggcaaagctctcttcgatggtcagcttttagatgctcattttactagatctttctacaaacacatacttggtgtcagggttacataccatgacattgaagctatcgatcctgcatactacaagaatttgaaatggatgctcgagaatgacataagtgatgttttggatctcaccttcagcatggatgcggacgaagagaaactgatattgtacgagaaggcagaggtatttgctgtcactgattgtgagttgattccaggagggcgtaacatcagggttactgaggagaataaacatcaatatgtagatcgggtagcagagcatcgactgacgactgcaatcaggcctcaaattaatgccttcctggaagggtttaatgaactcattccacgggagctgatatctatcttcaacgacaaagagcttgaacttctgattagcggtcttccagatattgacttggacgatttgaaagcaaatacagaatattctggctacagcatagcctctccagtcatccagtggttctgggaaatcgttcaggggttcagcaaggaagacaaagcccggttcctccagttcgtcactggcacttcaaaggtaccgttggaaggttttagtgcacttcaagggatctctgggccacaaaggtttcagatacataaggcctacggcagcacgaaccatctgccttcagctcatacttgctttaaccaactagacttgcctgagtacacatccaaagagcagctccaggagcggttactattggcaatccacgaagcaaatgaaggtttcggatttggctaa

SEQ ID NO: 14 - skipped

SEQ ID NO: 15 - protein, 3634 aa, Zea mays
MAAAAAAHRASFPLRLQQILAGSRAVSPAIKVESEPPANVKAFIDQVINIPLHDIAIPLSGFRWEFNKGNFHHWKPLFIHFDTYFKTYISSRKDLLLSDDMTEAEPMPKNAILKILIVMQIILENCQNRSSFTGLEHLKLLLASSDPEIVVAALETLVALVKINPSKLHMNGKLISCGSINTHLLSLAQGWGSKEEGLGIYSCVVANEGNQQGGLSLFPVDLESKYQHRLGSTLHFEYNLGSAQYPDQTSDKGKSSNLCVIHIPDMHLQKEDDLSILKQCVDKFNVPPEHRFALLTRIRYARAFNSTRTCSIYSRISLLSFIVLVQSSDAHDELTYFFTNEPEYINELIRLVRSEDSVPGPIRALAMLALGAQLAAYASSHERARILSGSSIISAGGNRMVLLSVLQKAIFSLNSPNDASSPLIVDALLQFFLLHVLSSSSSGTTVRGSGMVPPLLPLLRDNDSSHMHLVCLAVKTLQKLMEYSSPAVSLFKDLGGVDLLSRRLHVEVQRVIGTADGHNSMVTDAVKSKEDHLYSQKRLIKALLKALGSSTYSPGIPARSQSSQDNSLPVSLSLIFQNVEKFGGDIYFSAVTVMSEIIHKDPTCFPALKELGLPDAFLSSVTAGVIPSCKALICVPNGLGAICLNNQGLEAVRETSALRFLVYTFTSRKYLIPLNEGVVLLANAAEELLRHVQSLRSIGVDIIIEIINKLSSSLKDRNNETAILEEKTDMETDVEGRDLVGGMDSSVEGSNDEQFSHLSIFHVMVLVHRTMENSETCRLFVEKGGLNALLTLLLRPSITLSSGGMPIALHSTMVFKGFTQHHSTPLARAFCSSLREHLKSALGELDKVSNSFEMTKIEKGAIPSLFVVEFLLFLAASKDNRWMNALLSEFGDASREVLEDIGRVHREVLWKISLFEENKIDAEISLSSSTSEAQQPDLSASDIGDSRYTSFRQYLDPILRRRGSGWNIESQVSDLINMYRDIGSAASDSQRVGSDRYSSLGLPSSSQDQSSSSSDANVSTRSEEEKKNSEHSSCFDMMRSLSYHINHLFMELGKAMLLTSRRENSPVNLSPSVISVANNIASIMLEHLNFEGHSVSSEREMTVTTKCQYLGKVAEFIDGILLDRPESCNPIMVNSFYCCGVIQAILTTFQATSELLFTMSRPPSSPMETDSKTGKDGKDMNSSWIYGPLISYGAIMDHLVTSSFILSSSTRQLLEQPIFNGSVRFPQDAERFMKLLQSKVLKTVLPIWAHPEFPECNIELISSVMSIMRHVCSGVEVKNTVGNDGARLTGPPPDESAISLIVEMGFSRARAEEALRQVGTNSVEIATDWLFSHPEDELARALAMSLGNSDTPAQEGNGRSNDLELEEVTVQLPPIDEMLHSCFQLLQTKEALAFPVRDMLVTISSQKDGQNRVKVLTYLIENLKQCVVASEPSNDTALSALLHVLALILHGDTAAREVASKAGIVKVALDLLSSWELELRESGMIEVPNWVSSCFLSVDQMLQLEPKLPDVTELDVLKRDNSNIKTSLVIDESKKKDSESLSSVGLLDMEDQYQLLKICCKCIEKQLPSASMHAILQLSATLTKVHAAAICFLESGGLNALLSLPTSSLFSGFNSVASTIIRHILEDPHTLQQAMELEIRHSLVTAANRHTNPRVTPRNFVQNLAFVIYRDPVIFMKAVQSVCQIEMVGDRPYVVLLKDREKERSKEKDKDKSVDKDKATGAVAKVVSGDTAAGSPANAQGKQSDLNSRNVKSHRKPPQSFVTVIEHLLDLVMSFVPPPRPEDQADVVSGTALSSDMDIDCSSAKGKGKAVSVPPEESKHAIQESTASLAKASFFLKLMTDVLLTYTSSIQVVLRHDADLSNMHGPNRTNSGLISGGIFNHILQHFLPHATKQKKERKSDGDWMYKLATRANQFLVASSIRSAEARKKVFSEICNILLDFTDSSAAYKAPVARMNVYVDLLNDILSARSPTGSSLSAESAVTFVEVGLAPSLLKMLQNLDLDHPDSAKIVTAIVKALELVSKEHVHSADNAKGENSSKIASDSNNVNSSPNRFQALDMTSQPTEMITDHRETFNADQTSQSSDSVADEMDHDRDMDGGFARDGEDDFMHEMAGDGTGNESTMEIRFEISRNRDDMADDDDDDDNTDEDMSAEDDEEVNEDDEDEDEENNNLEEDDAHQMSHPDTDQEDREMDEEEFDEDLLEDDDDEDEEGVILRLEEGINGINVFDHIEVFGGSNNLSGDTLRVMPLDIFGTRRQGRSTSIYNLLGRASDHGVLDHPLLEEPSSTLNFSHQEQPENLVEMAFSDRNHEGSSSRLDAIFRSLRSGRNGHRFNMWLDDGPQRNGSAAPAVPEGIEELLISHLSRPTQQPGAQTVGGTQENDQPKHGSAAEAREGSPAQQNENSENTTNPVDLSESAGPAPPDSDALQRVVSNASIEHATEMQYERSDTITRDVEAVSQASSGSGATLGESLRSLEVEIGSVEGHDDGDRHGTSGASERLPLGDIQAAARSRRPSGNAVAVSSRDMSLESVSEVPQNPDQEPDHNASEGNQEPRGVGADTIDPTFLEALPEDLRAEVLSSRQNQVTQTSNDQPQNDGDIDPEFLAALPLDIREEVLAQQRSQRIQQQSQELEGQPVEMDAVSIIATFPSEIREEVLLTSPDTLLATLTPALVAEANMLRERFAHRYHSSSLFGMNSRNRRGESSRRDIMAAGLDRNTGDPSRSTSKPIEIEGAPLVDEDGLKALIRLLRVVQPLYKGQLQRLLVNLCTHRDNRQALVQILVDMLMLDLQGFSKKSVDASEPPFRLYGCHANITYSRPQSSNGVPPLVSRRVLETLTNLARSHPNVAKLLLFLEFPCPSRCRSEAHDHRHGKAVLEDGEERKAFAVVLLLTLLNQPLYMRSVAHLEQLLNLLEVVMHNAENEINQAKLEASSEKPSENAVKDVKDNTSISDSYGSKSNPEDGSKALAVDNKSNLRAVLRSLPQSELRLLCSLLAHDGLSDSAYLLVGEVLKKIVALAPFFCCHFINELARSMQSLTFCAMKELRLYENSEKALLSSTSANGTAILRVVQALSSLVSTLQDRKDPEQPAEKDHSDAVSQISEINTALDALWLELSNCISKIESSSEYASNLTPASASAATLTAGVAPPLPAGTQNILPYIESFFVTCEKLRPGQPDAVQEASTSDMEDASTSSGGQRSYSCQASLDEKQNAFVKFSEKHRRLLNAFIHQNPGLLEKSFSLMLKIPRLIDFDNKRAYFRSKIKHQYDHHHHNPVRISVRRSYILEDSYNQLRMRSPQDLKGRLTVHFQGEEGIDAGGLTREWYQSLSRVIFDKSALLFTTVGNDLTFQPNPNSVYQTEHLSYFKFAGRVVGKALFDGQLLDAHFTRSFYKHILGVRVTYHDIEAIDPAYYKNLKWMLENDISDVLDLTFSMDADEEKLILYEKAEVFAVTDCELIPGGRNIRVTEENKHQYVDRVAEHRLTTAIRPQINAFLEGFNELIPRELISIFNDKELELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKEQLQERLLLAIHEANEGFGFG

SEQ ID NO: 16 - DNA, 10938 bp, Pennisetum glaucum
atggcggcggcggcgatggcggcgcatcgcgcgagctttccgctgcgcctgcagcagattctggcgggcagccgcgcggtgagcccggcgattaaagtggaaagcgaaccgccggcgaaagtgaaagaatttattgatcgcgtgattaacattccgctgcatgatattgcgattccgctgagcggctttcgctgggaatttaacaaaggcaactttcatcattggaaaccgctgtttatgcattttgatacctattttaaaacctatctgagcagccgcaaagatctgctgctgagcgatgatatggcggaagcggatccgctgccgaaaaacaccattctgaaaattctgcgcgtgatgcagattgtgctggaaaactgccataacaaaagcagctttgcgggcctggaacattttaaactgctgctggcgagcagcgatccggaaattgtggtggcggcgctggaaaccctggcggcgctggtgaaaattaacccgagcaaactgcatatgaacggcaaactgattagctgcggcgcgattaacacccatctgctgagcctggcgcagggctggggcagcaaagaagaaggcctgggcctgtatagctgcgtggtggcgaacgaaggcaaccagcaggaaggcctgagcctgtttccggcggatatggaaaacaaatatgatggcagccagcatcgcctgggcagcaccctgcattttgaatataacctgagcccgacccaggatccggatcagaccagcgataaaagcaaaagcagcaacctgtgcgtgattcatattccggatatgcatctgcagaaagaagatgatctgagcattctgaaacagtgcgtggataaatttaacgtgccgccggaacatcgctttgcgctgctgacccgcattcgctatgcgcgcgcgtttaacagcgcgcgcacctgccgcctgtatagccgcattagcctgctgagctttattgtgctggtgcagagcagcgatgcgcatgatgaactgaccagcttttttaccaacgaaccggaatatattaacgaactgattcgcctggtgcgcagcgaagattttgtgccgggcccgattcgcgcgctggcgatgctggcgctgggcgcgcagctggcggcgtatgcgagcagccatgaacgcgcgcgcattctgagcggcagcagcattattagcgcgggcggcaaccgcatggtgctgctgagcgtgctgcagaaagcgattagcagcctgaacagcccgaacgataccagcgcgccgctgattgtggatgcgctgctgcagttttttctgctgcatgtgctgagcagcagcagcagcggcaccaccgtgcgcggcagcggcatggtgccgccgctgctgccgctgctgcaggataacgatccgagccatatgcatctggtgtgcctggcggtgaaaaccctgcagaaactgatggaatatagcagcccggcggtgagcctgtttaaagatctgggcggcgtggaactgctgagccagcgcctgcatgtggaagtgcagcgcgtgattggcaccgtggatggccataacagcatggtgaccgatgcggtgaaaagcgaagaagatgtgctgtatagccagaaacgcctgattcgcgcgctgctgaaagcgctgggcagcgcgacctatagcccgggcaacccggcgcgcagccagagcagccaggataacagcctgccggtgagcctgagcctgatttttcagaacgtggaaaaatttggcggcgatatttattttagcgcggtgaccgtgatgagcgaaattattcataaagatccgacctgctttccggcgctgaaagaactgggcctgccggatgcgtttctgagcagcgtgaccgcgggcgtgattccgagctgcaaagcgctgatttgcgtgccgaacggcctgggcgcgatttgcctgaacaaccagggcctggaagcggtgcgcgaaaccagcgcgctgcgctttctggtggatacctttaccagccgcaaatatctgatgccgatgaacgaaggcgtggtgctgctggcgaacgcggtggaagaactgctgcgccatgtgcagagcctgcgcagcaccggcgtggatattattattgaaattattaacaaactgtgcagcagccaggaatatcgcagcaacgaaccggcgattagcgaagaagaaaaaaccgatatggaaaccgatgtggaaggccgcgatctggtgagcgcgatggatagcagcgcggaaggcatgcatgatgaacagtttagccatctgagcatttttcatgtgatggtgctggtgcatcgcaccatggaaaacagcgaaacctgccgcctgtttgtggaaaaaggcggcctgcaggcgctgctggcgctgctgctgcgcccgagcattacccagagcagcggcggcatgccgattgcgctgcatagcaccatggtgtttaaaggctttacccagcatcatagcaccccgctggcgcgcgcgttttgcagcagcctgcgcgaacatctgaaaagcgcgctggaagaactggataaagtgagcagcagcgtggaaatgagcaaactggaaaaaggcgcgattccgagcctgtttgtggtggaatttctgctgtttctggcggcgagcaaagataaccgctggatgaacgcgctgctgagcgaatttggcgatgcgagccgcgaagtgctggaagatattggccgcgtgcatcgcgaagtgctgtataaaattagcctgtttgaagaaaacaaaattgatagcgaagcgagcagcagcagcctggcgagcgaagcgcagcagccggatagcagcgcgagcgatattgatgatagccgctataccagctttcgccagtatctggatccgctgctgcgccgccgcggcagcggctggaacattgaaagccaggtgagcgatctgattaacatttatcgcgatattggccgcgcggcgagcgatagccagcgcgtggatagcgatcgctatagcaaccagggcctgccgagcagcagccaggatcagagcagcagcagcagcgatgcgaacgcgagcacccgcagcgaagaagataaaaaaaaaagcgaacatagcagctgctgcgatatgatgcgcagcctgagctatcatattagccatctgtttatggaactgggcaaagcgatgctgctgaccagccgccgcgaaaacagcccggtgaacctgagcccgagcgtgattagcgtggcgggcagcattgcgagcattgtgctggaacatctgaactttgaaggccgcagcgtgagcagcgaaaaagaaattaacgtgaccaccaaatgccgctatctgggcaaagtggtggaatttgtggatggcattctgctggatcgcccggaaagctgcaacccgattatggtgaacagcttttattgccgcggcgtgattcaggcgattctgaccacctttcaggcgaccagcgaactgctgtttaccatgagccgcccgccgagcagcccgatggataccgatagcaaaaccggcaaagatggcaaagaaaccgatagcagctggatttatggcccgctgagcagctatggcgcggtgatggatcatctggtgaccagcagctttattctgagcagcagcacccgccagctgctggaacagccgatttttaacggcagcgtgcgctttccgcaggatgcggaacgctttatgaaactgctgcagagcaaagtgctgaaaaccgtgctgccgatttgggcgcatagccagtttccggaatgcaacattgaactgattagcagcgtgaccagcattatgcgccatgtgtgcaccggcgtggaagtgaaaaacaccgtgggcaacggcagcggccgcctggcgggcccgccgccggatgaaaacgcgattagcctgattgtggaaatgggctttagccgcgcgcgcgcggaagaagcgctgcgccaggtgggcaccaacagcgtggaaattgcgaccgattggctgtttagccatccggaagaaccgcaggaagaagatgatgaactggcgcgcgcgctggcgatgagcctgggcaacagcgataccagcgcgcaggaagaagatagccgcagcaacgatctggaactggaagaagaaaccgtgcagctgccgccgattgatgaaattctgtatagctgcctgcgcctgctgcagaccaaagaagcgctggcgtttccggtgcgcgatatgctggtgaccattagcacccagaacgatggccagaaccgcgaaaaagtgctgacctatctgattgaaaacctgaaacagtgcgtgatggcgagcgaaagcctgaaagataccaccctgagcgcgctgtttcatgtgctggcgctgattctgcatggcgataccgcggcgcgcgaagtggcgagcaaagcgggcctggtgaaagtggcgctggatctgctgtttagctgggaactggaaccgcgcgaaagcgaaatgaccgaagtgccgaactgggtgaccagctgctttctgagcgtggatcgcatgctgcagctggaaccgaaactgccggatgtgaccgaactggatgtgctgaaaaaagataacagcaacgcgaaaaccagcctggtgattgatgatagcaaaaaaaaagatagcgaaagcctgagcagcgtgggcctgctggatctggaagatcagaaacagctgctgaaaatttgctgcaaatgcattgaaaaacagctgccgagcgcgagcatgcatgcgattctgcagctgtgcgcgaccctgaccaaagtgcatgcggcggcgatttgctttctggaaagcggcggcctgaacgcgctgctgagcctgccgaccagcagcttttttagcggctttaacagcgtggcgagcaccattattcgccatattctggaagatccgcataccctgcagcaggcgatggaactggaaattcgccatagcctggtgaccgcggcgaaccgccatgcgaacccgcgcgtgaccccgcgcaactttgtgcagaacctggcgtttgtggtgtatcgcgatccggtgatttttatgaaagcggcgcaggcggtgtgccagattgaaatggtgggcgatcgcccgtatgtggtgctgctgaaagatcgcgaaaaagaacgcagcaaagaaaaagataaagataaaagcgcggataaagataaagcgaccggcgcggtgaccaaagtgaccagcggcgatattgcggcgggcagcccggcgagcgcgcagggcaaacagccggatctgagcgcgcgcaacgtgaaaccgcatcgcaaaccgccgcagagctttgtgaccgtgattgaacatctgctggatctggtgattagctttgtgccgccgccgcgcagcgaagatcaggcggatgtgagcggcaccgcgagcagcagcgatatggatattgattgcagcagcgcgaaaggcaaaggcaaagcggtggcggtggcgccggaagaaagcaaacatgcggcgcaggaagcgaccgcgagcctggcgaaaagcgcgtttgtgctgaaactgctgaccgatgtgctgctgacctatgcgagcagcattcaggtggtgctgcgccatgatgcggatctgagcagcatgcatggcccgaaccgcccgagcgcgggcctggtgagcggcggcatttttaaccatattctgcagcattttctgccgcatgcggtgaaacagaaaaaagatcgcaaaaccgatggcgattggcgctataaactggcgacccgcgcgaaccagtttctggtggcgagcagcattcgcagcgcggaaggccgcaaacgcatttttagcgaaatttgcaacatttttctggattttaccgatagcagcaccgcgtataaagcgccggtgagccgcctgaacgcgtatgtggatctgctgaacgatattctgagcgcgcgcagcccgaccggcagcagcctgagcgcggaaagcgcggtgacctttgtggaagtgggcctggtgcagagcctgagccgcaccctgcaggtgctggatctggatcatccggatagcgcgaaaattgtgagcgcgattgtgaaagcgctggaagtggtgaccaaagaacatgtgcatagcgcggatctgaacgcgaaaggcgataacagcagcaaaattgcgagcgatagcaacaacgtggatctgagcagcaaccgctttcaggcgctggataccaccagccagccgaccgaaatgattaccgatgatcgcgaaacctttaacgcggtgcagaccagccagagcagcgatagcgtggaagatgaaatggatcatgatcgcgatatggatggcggctttgcgcgcgatggcgaagatgattttatgcatgaaatggcggaagatggcaccggcaacgaaagcaccatggaaattcgctttgaaattccgcgcaaccgcgaagatgatatggcggatgatgatgaagataccgatgaagatatgagcgcggatgatggcgaagaagtggatgaagatgatgaagatgaagatgatgatgaagaaaacaacaacctggaagaagatgatgcgcatcagatgagccatccggataccgatcaggatgatcgcgaaatggatgaagaagaatttgatgaagatctgctggaagatgatgatgaagatgaagatgaagaaggcgtgattctgcgcctggaagaaggcattaacggcattaacgtgtttgatcatattgaagtgtttggcggcagcaacaacctgagcggcgataccctgcgcgtgatgccgctggatatttttggcacccgccgccagggccgcagcaccagcatttataacctgctgggccgcgcgagcgatcatggcgtgctggatcatccgctgctggaagaaccgagcagcatgctgaacctgccgcatcagggccagccggaaaacctggtggaaatggcgtttagcgatcgcaaccatgaaagcagcagcagccgcctggatgcgatttttcgcagcctgcgcagcggccgcaacggccatcgctttaacatgtggctggatgatagcccgcagcgcagcggcagcgcggcgccggcggtgccggaaggcattgaagaactgctgattagccatctgcgccgcccgaccccggaacagccggatgatcagcgcaccccggcgggcggcacccaggaaaacgatcagccgaccaacgtgagcgaagcggaagcgcgcgaagaagcgccggcggaacagaacgaaaacaacgaaaacaccgtgaacccggtggatgtgctggaaaacgcgggcccggcgccgccggatagcgatgcgctgcagcgcgatgtgagcaacgcgagcgaacatgcgaccgaaatgcagtatgaacgcagcgatgcggtggtgcgcgatgtggaagcggtgagccaggcgagcagcggcagcggcgcgaccctgggcgaaagcctgcgcagcctggaagtggaaattggcagcgtggaaggccatgatgatggcgatcgccatggcgcgagcggcgcgagcgatcgcctgccgctgggcgatatgcaggcgaccgcgcgcagccgccgcccgagcggcagcgcggtgcaggtgggcggccgcgatattagcctggaaagcgtgagcgaagtgccgcagaacagcaaccaggaaccggatcagaacgcgaacgaaggcaaccaggaaccggcgcgcgcggcggatgcggatagcattgatccgacctttctggaagcgctgccggaagatctgcgcgcggaagtgctgagcagccgccagaaccaggtggcgcagaccagcaacgatcagccgcagaacgatggcgatattgatccggaatttctggcggcgctgccgccggatattcgcgaagaagtgctggcgcagcagcgcgcgcagcgcctgcagcagcagagccaggaactggaaggccagccggtggaaatggatgcggtgagcattattgcgacctttccgagcgaaattcgcgaagaagtgctgctgaccagcccggataccctgctggcgaccctgaccccggcgctggtggcggaagcgaacatgctgcgcgaacgctttgcgcatcgctatcatagcagcagcctgtttggcatgaacagccgcaaccgccgcggcgaaagcagccgccgcgaaattatggcggcgggcctggatcgcaacggcgatccgagccgcagcaccagcaaaccgattgaaaccgaaggcgcgccgctggtggatgaagatgcgctgcgcgcgctgattcgcctgctgcgcgtggtgcagccgctgtataaaggccagctgcagcgcctgctgctgaacctgtgcgcgcatcgcgatagccgcaaaagcctggtgcagattctggtggatatgctgatgctggatctgcagggcagcagcaaaaaaagcattgatgcgaccgaaccgccgtttcgcctgtatggctgccatgcgaacattacctatagccgcccgcagagcagcgatggcgtgccgccgctggtgagccgccgcgtgctggaaaccctgacctatctggcgcgcagccatccgaacgtggcgaaactgctgctgtttctggaatttccgagcccgagccgctgccataccgaagcgctggatcagcgccatggcaaagcggtggtggaagatggcgaagaacagaaagcgtttgcgctggtgctgctgctgaccctgctgaaccagccgctgtatatgcgcagcgtggcgcatctggaacagctgctgaacctgctggaagtggtgatgctgaacgcggaaacccagattaaccaggcgaaactggaagcgagcagcgaaaaaccgagcggcccggaaaacgcggtgcaggatagccaggataacaccaacattagcgaaagcagcggcagcaaaagcaacgcggaagatagcagcaaaaccccggcggtggataacgaaaacattctgcaggcggtgctgcagagcctgccgcagccggaactgcgcctgctgtgcagcctgctggcgcatgatggcctgagcgataacgcgtatctgctggtggcggaagtgctgaaaaaaattgtggcgctggcgccgtttttttgctgccattttattaacgaactggcgcgcagcatgcagaacctgaccctgtgcgcgatgaaagaactgcgcctgtatgaaaacagcgaaaaagcgctgctgagcagcagcagcgcgaacggcaccgcgattctgcgcgtggtgcaggcgctgagcagcctggtgaccaccctgcaggaaaaaaaagatccggaactgccggcggaaaaagatcatagcgatgcggtgagccagattagcgaaattaacaccgcgctggatgcgctgtggctggaactgagcaactgcattagcaaaattgaaagcagcagcgaatatgtgagcaacctgagcccggcggcggcgaacgcgccgaccctggcgaccggcgtggcgccgccgctgccggcgggcacccagaacattctgccgtatattgaaagcttttttgtgacctgcgaaaaactgcgcccgggccagccggatgcggtgcaggaagcgagcaccagcgatatggaagatgcgagcaccagcagcggcggcctgcgcagcagcggcggccaggcgagcctggatgaaaaacagaacgcgtttgtgaaatttagcgaaaaacatcgccgcctgctgaacgcgtttattcgccagaacccgggcctgctggaaaaaagctttagcctgatgctgaaaattccgcgcctgattgattttgataacaaacgcgcgtattttcgcagcaaaattaaacatcagcatgatcatcatcatagcccggtgcgcattagcgtgcgccgcgcgtatattctggaagatagctataaccagctgcgcatgcgcagcccgcaggatctgaaaggccgcctgaccgtgcattttcagggcgaagaaggcattgatgcgggcggcctgacccgcgaatggtatcagagcctgagccgcgtgatttttgataaaggcgcgctgctgtttaccaccgtgggcaacgatctgacctttcagccgaacccgaacagcgtgtatcagaccgaacatctgagctattttaaatttgtgggccgcgtggtgggcaaagcgctgtttgatggccagctgctggatgcgcattttacccgcagcttttataaacatattctgggcgtgaaagtgacctatcatgatattgaagcgattgatccggcgtattataaaaacctgaaatggatgctggaaaacgatattaccgatgtgctggatctgacctttagcatggatgcggatgaagaaaaactgattctgtatgaaaaagcggaagtgaccgatagcgaactgattccgggcggccgcaacattaaagtgaccgaagaaaacaaacatgaatatgtggatcgcgtggtggaacatcgcctgaccaccgcgattcgcccgcagattaacgcgtttctggaaggctttaacgaactgattccgcgcgaactgattagcatttttaacgataaagaactggaactgctgattagcggcctgccggatattgatctggatgatctgaaagcgaacaccgaatatagcggctatagcattgcgagcccggtgattcagtggttttgggaaattgtgcagggctttagcaaagaagataaagcgcgctttctgcagtttgtgaccggcaccagcaaagtgccgctggaaggctttagcgcgctgcagggcattagcggcccgcagcgctttcagattcataaagcgtatggcagcaccaaccatctgccgagcgcgcatacctgctttaaccagctggatctgccggaatataccagcaaagaacagctgcaggaacgcctgctgctggcgattcatgaagcgaacgaaggctttggctttggc

SEQ ID NO: 17 - skipped

SEQ ID NO: 18 - protein, 3646 aa, Pennisetum glaucum
MAAAAMAAHRASFPLRLQQILAGSRAVSPAIKVESEPPAKVKEFIDRVINIPLHDIAIPLSGFRWEFNKGNFHHWKPLFMHFDTYFKTYLSSRKDLLLSDDMAEADPLPKNTILKILRVMQIVLENCHNKSSFAGLEHFKLLLASSDPEIVVAALETLAALVKINPSKLHMNGKLISCGAINTHLLSLAQGWGSKEEGLGLYSCVVANEGNQQEGLSLFPADMENKYDGSQHRLGSTLHFEYNLSPTQDPDQTSDKSKSSNLCVIHIPDMHLQKEDDLSILKQCVDKFNVPPEHRFALLTRIRYARAFNSARTCRLYSRISLLSFIVLVQSSDAHDELTSFFTNEPEYINELIRLVRSEDFVPGPIRALAMLALGAQLAAYASSHERARILSGSSIISAGGNRMVLLSVLQKAISSLNSPNDTSAPLIVDALLQFFLLHVLSSSSSGTTVRGSGMVPPLLPLLQDNDPSHMHLVCLAVKTLQKLMEYSSPAVSLFKDLGGVELLSQRLHVEVQRVIGTVDGHNSMVTDAVKSEEDVLYSQKRLIRALLKALGSATYSPGNPARSQSSQDNSLPVSLSLIFQNVEKFGGDIYFSAVTVMSEIIHKDPTCFPALKELGLPDAFLSSVTAGVIPSCKALICVPNGLGAICLNNQGLEAVRETSALRFLVDTFTSRKYLMPMNEGVVLLANAVEELLRHVQSLRSTGVDIIIEIINKLCSSQEYRSNEPAISEEEKTDMETDVEGRDLVSAMDSSAEGMHDEQFSHLSIFHVMVLVHRTMENSETCRLFVEKGGLQALLALLLRPSITQSSGGMPIALHSTMVFKGFTQHHSTPLARAFCSSLREHLKSALEELDKVSSSVEMSKLEKGAIPSLFVVEFLLFLAASKDNRWMNALLSEFGDASREVLEDIGRVHREVLYKISLFEENKIDSEASSSSLASEAQQPDSSASDIDDSRYTSFRQYLDPLLRRRGSGWNIESQVSDLINIYRDIGRAASDSQRVDSDRYSNQGLPSSSQDQSSSSSDANASTRSEEDKKKSEHSSCCDMMRSLSYHISHLFMELGKAMLLTSRRENSPVNLSPSVISVAGSIASIVLEHLNFEGRSVSSEKEINVTTKCRYLGKVVEFVDGILLDRPESCNPIMVNSFYCRGVIQAILTTFQATSELLFTMSRPPSSPMDTDSKTGKDGKETDSSWIYGPLSSYGAVMDHLVTSSFILSSSTRQLLEQPIFNGSVRFPQDAERFMKLLQSKVLKTVLPIWAHSQFPECNIELISSVTSIMRHVCTGVEVKNTVGNGSGRLAGPPPDENAISLIVEMGFSRARAEEALRQVGTNSVEIATDWLFSHPEEPQEEDDELARALAMSLGNSDTSAQEEDSRSNDLELEEETVQLPPIDEILYSCLRLLQTKEALAFPVRDMLVTISTQNDGQNREKVLTYLIENLKQCVMASESLKDTTLSALFHVLALILHGDTAAREVASKAGLVKVALDLLFSWELEPRESEMTEVPNWVTSCFLSVDRMLQLEPKLPDVTELDVLKKDNSNAKTSLVIDDSKKKDSESLSSVGLLDLEDQKQLLKICCKCIEKQLPSASMHAILQLCATLTKVHAAAICFLESGGLNALLSLPTSSFFSGFNSVASTIIRHILEDPHTLQQAMELEIRHSLVTAANRHANPRVTPRNFVQNLAFVVYRDPVIFMKAAQAVCQIEMVGDRPYVVLLKDREKERSKEKDKDKSADKDKATGAVTKVTSGDIAAGSPASAQGKQPDLSARNVKPHRKPPQSFVTVIEHLLDLVISFVPPPRSEDQADVSGTASSSDMDIDCSSAKGKGKAVAVAPEESKHAAQEATASLAKSAFVLKLLTDVLLTYASSIQVVLRHDADLSSMHGPNRPSAGLVSGGIFNHILQHFLPHAVKQKKDRKTDGDWRYKLATRANQFLVASSIRSAEGRKRIFSEICNIFLDFTDSSTAYKAPVSRLNAYVDLLNDILSARSPTGSSLSAESAVTFVEVGLVQSLSRTLQVLDLDHPDSAKIVSAIVKALEVVTKEHVHSADLNAKGDNSSKIASDSNNVDLSSNRFQALDTTSQPTEMITDDRETFNAVQTSQSSDSVEDEMDHDRDMDGGFARDGEDDFMHEMAEDGTGNESTMEIRFEIPRNREDDMADDDEDTDEDMSADDGEEVDEDDEDEDDDEENNNLEEDDAHQMSHPDTDQDDREMDEEEFDEDLLEDDDEDEDEEGVILRLEEGINGINVFDHIEVFGGSNNLSGDTLRVMPLDIFGTRRQGRSTSIYNLLGRASDHGVLDHPLLEEPSSMLNLPHQGQPENLVEMAFSDRNHESSSSRLDAIFRSLRSGRNGHRFNMWLDDSPQRSGSAAPAVPEGIEELLISHLRRPTPEQPDDQRTPAGGTQENDQPTNVSEAEAREEAPAEQNENNENTVNPVDVLENAGPAPPDSDALQRDVSNASEHATEMQYERSDAVVRDVEAVSQASSGSGATLGESLRSLEVEIGSVEGHDDGDRHGASGASDRLPLGDMQATARSRRPSGSAVQVGGRDISLESVSEVPQNSNQEPDQNANEGNQEPARAADADSIDPTFLEALPEDLRAEVLSSRQNQVAQTSNDQPQNDGDIDPEFLAALPPDIREEVLAQQRAQRLQQQSQELEGQPVEMDAVSIIATFPSEIREEVLLTSPDTLLATLTPALVAEANMLRERFAHRYHSSSLFGMNSRNRRGESSRREIMAAGLDRNGDPSRSTSKPIETEGAPLVDEDALRALIRLLRVVQPLYKGQLQRLLLNLCAHRDSRKSLVQILVDMLMLDLQGSSKKSIDATEPPFRLYGCHANITYSRPQSSDGVPPLVSRRVLETLTYLARSHPNVAKLLLFLEFPSPSRCHTEALDQRHGKAVVEDGEEQKAFALVLLLTLLNQPLYMRSVAHLEQLLNLLEVVMLNAETQINQAKLEASSEKPSGPENAVQDSQDNTNISESSGSKSNAEDSSKTPAVDNENILQAVLQSLPQPELRLLCSLLAHDGLSDNAYLLVAEVLKKIVALAPFFCCHFINELARSMQNLTLCAMKELRLYENSEKALLSSSSANGTAILRVVQALSSLVTTLQEKKDPELPAEKDHSDAVSQISEINTALDALWLELSNCISKIESSSEYVSNLSPAAANAPTLATGVAPPLPAGTQNILPYIESFFVTCEKLRPGQPDAVQEASTSDMEDASTSSGGLRSSGGQASLDEKQNAFVKFSEKHRRLLNAFIRQNPGLLEKSFSLMLKIPRLIDFDNKRAYFRSKIKHQHDHHHSPVRISVRRAYILEDSYNQLRMRSPQDLKGRLTVHFQGEEGIDAGGLTREWYQSLSRVIFDKGALLFTTVGNDLTFQPNPNSVYQTEHLSYFKFVGRVVGKALFDGQLLDAHFTRSFYKHILGVKVTYHDIEAIDPAYYKNLKWMLENDITDVLDLTFSMDADEEKLILYEKAEVTDSELIPGGRNIKVTEENKHEYVDRVVEHRLTTAIRPQINAFLEGFNELIPRELISIFNDKELELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKEQLQERLLLAIHEANEGFGFG

SEQ ID NO: 19 - DNA, 10950 bp, Glycine max
atgacaaccctaagatcaagttggccttcgaggctgcgccaacttctgtccagcgggggcgccattggtccttcagtcaaggtggactccgagccccctcctaagatcaaagccttcattgagaagatcatccagtgtccattacaagatattgccataccactttctggctttcggtgggagtacaataaggggaattttcatcactggagaccgctgttgcttcattttgatacatacttcaagacttatttgtcgtgtagaaatgatctgacgttgttagataatctagaagatgacagccctttaccaaaacatgcaattctgcaaatattgcgagtgatgcaaaaaattttagagaactgtccaaacaagagttcctttgatggcttagagcatttcaagcttttacttgcatcaacagatcctgagattcttgttgctacattggaaactctttctgcacttgtaaaaattaatccctctaagcttcatggaagtccaaagatgatttgctgtggttcggtgaacagctatcttttgtccttagcacaaggctggggaagcaaggaggagggcctaggattgtactcttgtgttatggcaaatgagaaagcccaagatgaagcactgtgcttgtttccttctgaagagattggtcatgaccaatcaaattgccgcataggcactaccctttattttgaattgcatggtcccaatgcccaaagcaaggaacatagtgcagatgcagtttcccctagctcaacagttatacacatgccagatttgcatctgcgcaaagaagatgatttgtcattgatgaagcagtgcactgaagaatttagcattccttctgagctcaggttttccttgctcactagaatcagatatgctcgtgcctttcgttctcctagaatatgcaggctttacagccggatttgcctactttctttcattgttctggtgcagtctggtgatgctcaggaagaacttgtctccttttttgctaatgaaccagaatatacaaatgaattaattagaattgtacgatcagaggaagttatatctggatctatcaggacacttgcaatgcttgctctaggagctcaattagcagcatatacatcatcgcatcatcgggcacggatcagtggatctagtttaacttttgctggtgggaaccgcatgatactcctaaatgtgctccagagggctattttgtcattgaagatttctaatgatccatcatcccttgcctttgttgaagcacttcttcagttctatctgctccatgtggtctcaacctcaacttctggtaataatattagaggttctggcatggtgccaacattcttgccgttgctggaggattttgatcctacacatattcatctagtctgttttgctgtgaaaactcttcagaagcttatggattatagtagctcagctgtatcattgtttaaagaattggggggcattgaacttttggctcagaggttacagaaagaggtacacagagtcattggtttggttggaggaactgataacatgatgcttactggtgaaagcttgggacatagtactgatcaattgtactcccagaagagactcataaaggtctcccttaaggcgcttggttctgcaacatacgcacctgcaaactctaccagatctcaacattctcaagacagttcattacctataactctaagcttgatttttaagaatgtagataagtttggaggtgacatttattattcagctgttactgttatgagtgaaataatccacaaagatcctacctttttttctgctctgcatgaaataggtcttcctgatgcgtttttattgtcagttggatctggaatacttccatcatcaaaggctttgacatgcattccaaatggtcttggggccatttgtcttaatgccaaagggttagaggccgttagagaatcttcatcgctacggttccttgttgacattttcactagcaagaagtatgtcttagccatgaatgaggctattgttcctttggcaaatgctgtggaggaacttctacgccatgtatctacattgagaagcactggtgttgatattatcattgaaatcatccataagatcacatcttttggggatggaaatggtgcaggattttctggaaaagctgagggcaccgccatggaaacagattctgaaaacaaagaaaaagaaggccattgttgcattgtaggcacatcatattcggctgtagaagggataagtgatgagcagtttattcagctatgtgtctttcatttgatggtattagttcataggactatggaaaatgccgagacatgccggttgtttgtggaaaaatcaggaattgaagctttattgaatttgttattacgacccactattgcacaatcctcagatggcatgtctattgctttacatagcacaatggtatttaaagggtttgctcaacatcattcaattcctctggcacatgccttctgttcttctcttagagagcacttaaagaaaactttagtggggtttggtgcagcatcagaacctttgttgctggatccaaggatgacaactgatggtggcatcttttcttcacttttcctggttgagttccttctatttcttgtggcatcgaaagacaatcgttgggtgactgccttgcttacagaatttggaaatgagagtaaggatgttcttgaagacattggatgcgttcaccgtgaagttctgtggcaaatttctctacttgaaaatagaaaacctgagattgaggaagatggtgcttgttcttctgattcacaacaggctgaaggggatgtaagtgaaactgaagagcaaaggttcaattctttcaggcagtatcttgacccattattgagaaggagaacatcaggatggagcattgaatcccagttttttaaccttataaacctgtatcgagatttgggccgttccactggttctcaaaatagattagttggtccgaggtcaagttctagtaatcaggtacagcattctgggtcagatgataattgggggactgctaataagaaggaatctgacaagcagagagcatattatacatcttgttgtgacatggtcagatcactttcatttcacattacccatttgttccaagagttaggaaaagtaatgttgctaccttcacgtcggcgtgatgatgttgtgaatgtaagtcctgcttcaaaatcagtggcttctacatttgcatccattgcttttgatcacatgaattatggtggccgttgtgtaaatctttcgggaacagaagaatccatatcaacaaaatgtcgatattttgggaaagtgattgattttatggataatgttctaatggagaggccagattcatgcaatcctattatgctgaattgcttgtatggacgtggagttattgaaattgtattaactacctttgaagctaccagtcagctgctctttacagttaatcgggcccctgcctcgcccatggatactgatgatgcaaatgcaaagcaagacgacaaggaagatacagataattcatggatttatggttctttagctagttatgggaaattgatggaccatctagtgacctcctcttttatattatcatcattcacaaagcatttgcttgcacagccccttactaatggtgatacacctttcccaagggatgctgagacttttgtgaaggtccttcaatccagagtgttgaagactgtacttcctgtttggactcatcccaagtttgttgactgtagttatgaatttatttctacagttatttctatcattaggcatgtctatacaggtgttgaagtaaaaaatgtgaatggcagtgctggtgctcgcattactgggccgcctcctaatgaaacaactatttcaaccattgtagaaatggggttttccaggtctagagcagaagaagctttgaggcaagttgggtcaaatagtgtggagttggcaatggagtggttgttctctcatccagaggaggcacaagaagatgatgaacttgcccgtgcacttgccatgtcccttggaaactctgaatcagattcaaaggatgcagttgctaatgacaatgccctgcagcttgaggaagagatggtccaacttcctcctgttgatgagttgttatctacttgtacaaaacttttgtcgaaggaaccacttgcttttccagtccgtgacttgcttgtgatgatatgctctcaggatgatggtcaacatagatctaatgtggtctcatttattgtggaacggatcaaagaatgtggtttggttcctagcaatggaaattatgccatgctggctgctctttttcatgttctagctttaattcttaatgaggatgctgtggctagggaagctgcttctacaagtggtttaatcaaaattgcctcagatctactctaccagtgggattctagtcttgatatcaaggagaaacatcaggtaccaaaatgggtgactgctgctttccttgcattagacagattgttgcaagtggatcaaaaattgaattctgaaatcgcagagcagttgaagaaggaagctgtgaatagccagcagacatcaattaccattgatgaagacaggcaaaacaagatgcagtctgcattgggactctctatgaagtatgcagatatacatgaacagaagagacttgttgaggttgcttgtagttgtatgaagaatcaacttccatccgacacaatgcatgctgttctgctactatgttccaatcttacaaggaatcattctgtagctcttacttttttggattctggtggtttaagtctacttctttctttgccaaccagcagtctcttccctgggtttgacaatgttgctgctagtattgttcgtcatgttcttgaagatcctcaaacgctccatcaagcaatggaatctgagataaaacatagtcttgtagttgcatctaaccggcatccaaatggaagggtcaatcctcataatttccttttaaatttagcttctgtgatttctcgggatccagtaatttttatgcaagctgctcaatctgtgtgccaagttgaaatggtaggtgagaggccatacattgtcttgctgaaagatagggataaagacaaagctaaggataaagaaaaggataaggataaaacattggagaaagataaagtacagaacattgatgggaaggttgttttgggaaatactaacacggcacctactggcaatggccatggcaaaattcaagattcaaataccaagagtgccaaaggtcacagaaaacctacccaaagttttattaatgcaatagaacttcttcttgaatctgtatgcacttttgttcctcccttgaagggtgacattgcctcaaatgttcttcctggcaccccagcatcaaccgatatggacattgatgcctccatggttaagggaaaaggaaaagcagttgccactgattctgagggcaatgaaactggtagtcaggatgcttctgcatcacttgcaaagattgtcttcattctaaagcttctgacagagatactattgatgtattcatcatctgttcatgttttacttagacgagatgctgaaatgagcagcattagaggttcttatcaaaagagtcctgcaggtttaagcatgggtgggatattctctcatattcttcataattttcttccatattctcgaaactccaaaaaggacaagaaagctgatggtgattggaggcagaaactagcaaccagggccaaccagtttatggtgggtgcttgtgttcgatctacagaggcaaggaagagggtttttggtgagatttgttgtatcatcaatgaatttgttgattcatgtcatggcattaagcgtccaggaaaagaaattcaggtttttgttgatctactaaatgatgttttggctgctcgtacacccgctggttcatccatttcagctgaggcctctaccacttttattgatgctggtttggttaaatcattcacatgtactctacaagttttggaccttgaccatgctgattcatctgaagttgctacgggtattattaaagctcttgagttggtaaccaaggagcatgtccaattagttgattctagtgcagggaagggtgataattcagcaaagccttctgttctaagtcaacccggaagaacaaataatattggtgacatgtctcagtccatggagacatcacaagccaatcctgattcccttcaagttgaccgtgttgggtcttatgcagtttgctcctatggtgggtctgaagctgttactgatgatatggaacatgatcaagatcttgatgggagctttgctcctgctaatgaggatgattacatgcatgaaaattctgaggatgcaagagatcttgaaaatggaatggaaaatgtgggtctacaatttgaaatccaatctcatggccaagaaaatcttgatgaggatgacgatgaggacgatgatatgtctgaagatgaaggtgaggatgtagatgaagatgaggatgatgatgaggaacacaatgatttggaagaagtccatcatttgccacatcctgacacagatcaagatgagcatgagattgatgatgaagattttgatgatgaagtgatggaggaagaggatgaggatgacgaggaagatgaagatggtgttatactgcaactcgaggaggggattaatggaattaatgtttttgatcatattgaggtttttggcagagataatagttttgcaaatgaagcttttcaagtgatgccggttgaggtttttggatccagacgtcaggggaggacaacatctatttatagtcttttgggaagaactggtgataccgctgtgccttctcgtcacccactcttgcttgaaccttcttcattccccccacctacagggcaatcagatagttcattggagaacaactcattgggtttggataatatatttcgatcgctgaggagtggacgccatggacagcgtttgcacttgtggactgataataaccaacaaagtggtgggacaaacactgttgttgtaccccaaggccttgaggatttgcttgtcactcaattaaggcgaccaatccctgaaaagtcatccaatcagaacattgcagaagcaggttctcatggtaaagttggaacgacccaggcacaagatgcagggggtgcaaggccagaagtccctgttgaaagtaatgctgttctggaagttagtactataactccctcggttgataacagtaacaatgcgggtgtcagaccagctgggactggaccttcacatacaaatgtttcaaacacacactcacaggaagttgagatgcaatttgaacatgctgatggagctgtgagggatgttgaagctgtcagccaggagagtagtggtagtggtgcaacttttggtgaaagccttcggagcttggatgttgagattggaagtgctgatggccatgatgatggtggtgaaaggcaggtttctgctgatagggtggcaggtgattcgcaggcagcacgcacaagaagagcaaatacgcctttgagtcacatttctcctgtggttggaagagatgcgttccttcacagtgtaactgaagtttcagaaaattcaagccgtgatgcagatcaagatggtgcagcagcagagcagcaggtgaacagtgatgcaggatcaggagctattgatcctgcttttctggatgctcttcctgaggagctgcgtgccgaactcctttcagctcagcagggtcaagtggctcagccatcaaatgctgagtctcaaaacactggggatattgatccagagttccttgcagctcttccagctgatattcgagcagaaattctagctcagcagcaagcacagaggctgcatcaatctcaggagctggaaggccaacctgtggaaatggatacagtctcaataattgcaacttttccatcagatttacgagaagaggttctgttgacgtcaccagatactatccttgccaatcttacacctgctcttgttgctgaggcaaatatgttgcgggagaggtttgcacaccgttacagtcgtaccctctttggtatgtatcctagaagtcgtagaggggagacttcaagacgtgaaggtattggttctggtctggatggagcagggggaaccatttcttctcgccgttccaatggagttaaggttgttgaagctgatggagcaccactagttgacacagaagctttgcatgctatgattcggttgttacgcgtagtgcagccactctataaaggccaactccagaggcttctattaaatctttgtgcccatagtgaaacaagaacctctctggtgaaaattctgatggacttgctaatgcttgatgtaaaaaggcctgtcagttattttagtaaagttgagccaccatatagattatatggttgtcagagcaatgtaatgtattcacgtcctcaatcttttgatggagttcccccattgctgtctcgtagaatacttgaaactctcacttatcttgctcgcaatcatctgtatgtggcaaaaattttgcttcagtgttggctaccaaatcctgcaataaaagaaccagatgatgcacggggcaaagccgtgatggttgttgaagatgaagtaaatataggtgaaagtaatgatgggtacatcgccattgcaatgctattgggtctcttgaaccaaccactttatttgaggagcatagcccaccttgagcagctgctaaatttactggatgttatcattgacagtgctggaaacaagtcatctgacaaatccttgatatctactaacccatcatcagctccacaaatttctgccgtggaagccaatgcgaatgcagattctaatattttatcttctgtggatgatgcatctaaagttgatggttcctccaaaccaacgccctctggcataaatgttgaatgtgagtcacatggagtgttgagtaatctttcaaatgcagaactccggctcctgtgctcactgcttgcacaagaaggtttgtcagataatgcatataatcttgttgccgaggtaatgaagaaattggtggccattgctccaacacattgtgagctttttgtcactgagctggcagaagcagttcaaaagttgacttcatctgcaatgaatgagttacgtgtctttagtgaagcaatgaaagctctgcttagtacctcttctactgatggagctgcaattctgagagttttgcaagccttgagttcccttgtcaccttactgacggagaaagagaatgacagaggtactcctgctctatctgaggtttgggaaatcaattcagcattagagcccttgtggcatgagcttagctgttgcataagcaagatagaatcctactcagagtctgcatctgagatttcgacatcttctagtacctttgtgtctaaaccatctggtgtaatgcctccacttcctgctggctctcaaaatatcttaccatacattgaatctttctttgtggtttgtgagaaattgcatcctgctcagccaggtgatagtcatgactcaagtatccctgttatttctgatgttgagtatgccaccacatctgcaactccccagaaagcatctggaactgctgtgaaagtagatgagaaacatatgccttttgtccggttctcagagaagcataggaagctactaaatgcattcttaaggcaaaaccctggtttgcttgagaaatctttctcactcatgctaaaggttccaagatttattgattttgataacaagcgtgcccacttccgatcaaaaattaagcatcagcatgaccatcaccatagcccattgagaatatcagtaagaagggcatatgttctagaagattcttacaaccagcttcgcttgagatcaactcaagatttgaagggaaggttgactgttcacttccaaggggaggagggtattgatgcaggtgggcttacaagggaatggtatcaattattgtccagagttatttttgataaaggagcactgctttttactacagtgggcaatgaatcaacatttcagcctaaccctaactctgtttatcaaacagagcatttatcttatttcaaatttgttggtagagtggttggcaaagcattatttgatggtcaactcttggatgttcattttactcggtcattctacaagcacattcttggagtcaaagttacatatcatgatattgaagccattgatcctcattatttcagaaatttgaaatggatgcttgagaatgacatcagtgatgttctggatcttacttttagcattgatgcagatgaggaaaaattgatcttatatgaacgaacagaggtgactgattatgagttgattcccgggggacggaatatcaaagttactgaggagaacaaacatcaatatgttgatttggttgccgagcatcggctgacaactgccattcgacctcaaataaattctttcttggaagggttcaatgaaatgattcccagggagttgatatcgatattcaatgacaaagagctggaattgttgatcagtggacttcctgatattgacttggatgacttgagagcaaatacagaatattctggatatagtgctgcatcgccagttatccaatggttttgggaggttgttcaaggtttgagcaaagaagacaaagctcgactgttgcaatttgtgacaggcacatccaaggtgcctttggaaggctttagcgctcttcaaggaatttcaggctcccagaagtttcagatacacaaagcatatggaagtcctgatcacttgccttctgctcatacttgcttcaatcaattagatttgccggagtatccatctaaacaccatttagaagagaggttactgctggcaattcacgaagcaagtgagggttttggatttggttga

SEQ ID NO: 20 - DNA, 10950 bp, Glycine max; identical to SEQ ID NO: 19

SEQ ID NO: 21 - DNA, 10956 bp, Glycine max
atgacaagcgtaagatcgagttggccatcaaggctgcgccaacttctttccagcgagggttccattggcccttccgtcaaactcgactctgacccttctcctaagatcaaagccttcattgagaaggtcattcaatgtccattacaagatatagctatacccctctttggctttcggtgggagtataataaggggaattttcatcactggaggccattgtttcttcattttgatacatacttcaagacatatttatcatgtcgaaatgacctgacattgtccgataatctagaagttggcattccattaccaaaacatgcaattctacaaatactacgggtgatgcaaataatcttagagaactgtccaaacaagagttcatttgatggcttagagcacttcaagcttttactagcatcaacagatcctgagattattattgctacattagaaactcttgctgcgcttgtaaaaataaatccttctaagcttcatggaagtgcaaaaatggttggctgtggttcagtaaatagctatctcctgtccctagcacaggggtggggaagcaaggaggagggcatgggtttgtactcttgtattatggcaaatgagaaagcccaggatgaagcactgtgtttgtttccttctgatgcagagaatggtagtgaccactccaattactgcataggttctactctttattttgaattgcgtggacccattgctcaaagcaaggaacaaagtgtagatacagtttcctcaagtttgagagttatacacattccagatatgcatttacacaaagaagatgatttgtcaatgttgaagcaatgcattgagcagtataatgttcctcctgagctccgattttcattgctcacaagaattagatatgctcgtgctttccggtctgcgagaataagcaggctttatagcaggatttgccttcttgctttcactgtgttggtccaatccagtgatgctcatgacgagcttgtgtccttttttgccaacgaaccagagtacacaagcgaattgattagagttgtgcgatctgaagaaacaatatctggatctatcagaacacttgtaatgcttgcattaggagcccagttagcagcatacacatcatctcatgaacgggcacggatactgagtggatctagtatgaacttcactggagggaaccgcatgattctactgaatgtacttcagagggctattttgtcattgaagagttctaatgatccaacttcctttgcttttgttgaggcacttcttcaattctatctgctgcatgtagtgtcaacatcatcttctgggagtaatattagaggttctggcatggtacccacattcttgcctctgctggaggattctgatcttgctcatattcatcttgtttgtttagcagtgaaaacccttcagaagcttatggattatagtagttcagctgtatctttgtttaaagagttggggggtgttgagcatttggctcaaagattacagatagaggttcatagggtcattggttttgctggagagaatgataatgtgatgctcactggtgaaagctcaagacatagtactcatcagctttactctcagaagaggctgataaaagtgtcccttaaggcccttggttctgcaacatatgctcctgcaaactctaccagatctcaacactcccatgacagttcattacctgcaactctagtcatgatttttcagaatgtaaataagttcggaggtgacatttattactcagctgttactgttatgagtgaaataatccacaaagatcctacatgcttctcttctttgcatgaaatgggtcttccaaatgcttttttatcttcagttgcatctggaattcttccttcatcaaaggctctgacatgcattccaaatggcattggggccatttgtcttaatgccaaaggcttagaggttgttcgagagacttcatcactgcagttcctttttaatatctttacaagcaaaaagtatgtcctttccatgaatgaggctattgttccgctagcaaattctgtagaggaacttcttcgacacgtgtctccattgagaagtactggtgttgacatcatcattgaaatcatccataagattgcatcctttggtgatggtattgatacaggatcttcttcaggaaaagctaatgaggatagtgcaatggaaaccaattctgaagacaaaggaaatgaaaaccattgttgcctcgtgggcacagcagagtctgccgctgaagggattaatgatgagcaattcattcagctttgcacttttcatttgatggtattggttcaccggacaatggaaaattctgaaacatgtcggctatttgtagaaaaatcaggaattgaagctttattgaagctgttattacgacctaccattgcacaatcctcggatggcatgtctattgctctgcatagcaccatggtatttaaggggtttgctcaacatcattccgctcctttggcacgtgccttttgttcctctcttaaagagcacttgaatgaagcattaactgggtttgttgcatcttcgggacctttgttgctggatccaaagatgaccacaaataacatcttttcttcacttttcttggttgagtttcttctctttcttgctgcgtcaaaagacaaccgttgggtgactgctttgcttacagaatttggaaatggtagtaaggatgttcttgaaaacattggacgtgtccaccgtgaagttttgtggcaaattgctcttcttgaaaatacgaagcctgatattgaggatgacgtttcttgttctacttctgattcacaacaggcagaagtggatgcaaatgaaactgcagagcaaaggtacaattctatcaggcagtttcttgatccattactcaggaggaggactttaggatggagtgtagaatcacagttttttgatcttattaacctgtatcgagatctgggtcgtgcccctggttcccagcaccgatcaaattctgttggtcctacaaacaggcggttaggatcccctaatccgttgcatccgtctgagtcttcagatgtattgggggatgctagtaagaaagaatgtgacaagcaaagaacatattatacctcttgttgtgacatggccagatcactttcatttcacattatgcatttgttccaagagttaggaaaagtaatgctgcaaccttctcgccgtcgtgatgatgttgcaagtgtaagtcctgcttcaaaatcagtggcttctacttttgcaagcattgctctagatcacatgaattttgggggccatgtagaagaagcatccatatcaacaaaatgtcgttattttggtaaagtcattgattttgtggatggcattctaatggaaaggcctgattcttgcaatcccattttactgaattgcttgtatgggcatggagttattcaatctgtattgaccacatttgaagcaactagtcagttgttatttgcagttaatcggacccctgcatcgccgatggaaattgatgatggaaatgtgaagcaggatgacaaggaagataccgatcatttgtggatatatggttctttagccagttatggtaaatttatggaccatctagtaacctcctctttcatattatcttctttcacaaagcctatacttgcacagccccttagtggtgatacctcatatccccgggatgctgagatatttgtgaaagtcctccaatctatggtgttgaaggctgtgctcccagtttggatgcatccccagtttgttgattgtagtcatggatttatttctaatgttatctctatcatcaggcatgtttattcaggggttgaagtaaaaaatgtaaatggcagcagcagtgctcgtattactgggcctcctcctaatgaaacaacaatttcaaccattgtagagatgggattttccaggtcgagagcagaagaagctttgaggcatgttggatcaaatagtgtggagttggcgatggagtggctgttttcccatccagaggacacacaagaagatgacgaacttgctcgtgcacttgccatgtcccttgggaactctgaatcagacaccaaggatgctgctgcaaatgacagtgtacaactgcttgaggaagaaatggtccatcttcctcctgttgatgagttgttatcaacttgcactaaacttcttcaaaaggaacctcttgcttttcctgtccgtgacttgctcatgatgatatgctctcagaatgatggtcaaaatagatctaatgttctcacttttattgttgaccggatcaaggaatgtggattgatttctggtaacggaaataataccatgcttgctgctctatttcatgttcttgcattgattcttaatgaggatgctgttgcgcgagaagctgcttcaaagagtggtttcataaaaattgcctcagatctactctaccaatgggattctagtcttggtaacagggagaaagaacaggttccaaaatgggtcacagctgcttttcttgcattagacaggctgttgcaagtggatcaaaaattgaattctgaaattgcagagcttttgaagaaggaagctttgaatgttcagcagacatcagttatcattgatgaggataagcaacacaaattgcagtctgcgttgggactttccaccaagtatgcagatatacatgaacagaagagacttgttgagattgcttgtagttgcatgaagaaccaacttccctcagacacaatgcatgctattttgctactatgttccaatcttacaaagaatcactctgttgctcttaccttttttgatgctggtggtttaagtttacttctttctctgccaaccggtagcctctttcctgggtttgacaacgttgctgctggtattgtccgtcatgttattgaagatccacaaactctccagcaagcaatggaatctgagataaaacacagtcttgtagctgcgtctaaccgccatccaaatgggagggtcaatccacaaaattttctgttaagtttagcttctgtaatttcccgggatccaataatatttatgcaagctgctcaatctgcttgccaagttgaaatggtgggtgaaagaccttacattgtcttgctgaaagatcgggataaagagaaatccaaggataaggataagtcactggagaaagataaagcacataatgatggaaaaattggtttgggaagtacagccacagcagcttcagggaatgttcatggaaaacttcatgattcaaactcaaagaatgccaaaagttacaaaaagcctactcaaagttttgttaatgtgatagaacttcttcttgaatctatatgcacatttgttgccccccctttgaaggacaataatgtatcaaatgttgtccctggctccccaacatcaagtgacatggacattgatgtttctacagttagggggaaaggaaaagcagttgccactgtgcctgaggggaatgaaaccagcagtgaggaagcatctgcatcactagcaaagatagtatttattttgaagcttctgatggagatattgttgatgtattcatcgtctgttcatgttctgcttcgacgggatgctgaaatgagcagctctagggacatttatcaaaagaatcatggtagttttggtgcgggagtaatattctaccatattcttcgtaattttcttccttgttctcgaaattccaaaaaagacaagaaagttgatgatgattggaggcagaaactagcaacaagggctaatcagtttatggtagctgcttgtgttcgttcttcagaggcaaggaggcgggtttttactgagattagccatatcattaatgaatttgttgattcatgtaattgtgttaagccaaagccatcaggcaatgaaattctggtttttgttgatctacttaatgatgttttggctgctcggacacctgctggctcaagcatctcagcagaggcctctgtcacttttatggatgctggtctacttaaatcttttacccgtactctccaagttttagacttggaccatgctgactcgtctaaagttgctactggtattatcaaagctcttgaactagtaaccaaggagcatgttcactcagttgaaccgagtgcaggaaagggtgataatcaaactaagccttctgatcctagtcaatccggaagaacagataatattggtcacatgtgtcagtccatggaaacaacatctcaggccaatcacgattcccttcaagttgaccatgttgggtcttacaatgtgattcagtcttatggtgggtctgaagctgttattggtgatatggaacatgatcttgatggggactttgctcctgctaatgaagatgagttcatgcatgaaactggtgaggatgccagaggccatgggaatggaattgaaaatgttgggctacaatttgaaatccaatcccatggacaagaaaatctcgatgatgacgatgatgagggtgatatgtctggagatgagggtgaagatgtagatgaagatgacgaagatgatgaggaacacaatgatttggaagaagatgaagtccatcacttgccacatcctgacactgatcgtgatgatcatgagatggatgatgatgattttgatgaagtgatggagggggaggaggatgaagatgaggatgatgaagatggtgttatactgagacttgaggagggcatcaatggaattaatgtttttgaccatattgaggtttttggaagagacaatagttttccaaatgaatcccttcatgtcatgccagttgaagtttttggatctagacgtccagggcggaccacctctatttacagcctgttgggcagaagtggtgataatgccgccccttcttgccatccacttttagttggtccttcttcctcattccatctatctaatggtcaatcagatagtataacagagaactccacaggcttggataatatctttcgttcattgaggagcggacgtcatgggcaccgcttgaacttgtggagtgataatagccagcaaatcagtgggtcaaatactggcgctgtaccacagggccttgaggagttgcttgtgtctcaattgaggcgacctactgctgagaagtcgtctgataataatatagcagacgctggtcctcataataaagttgaggtcagccagatgcacagttccggaggttcaaagcttgaaatcccagttgaaagcaatgcaattcaggaaggtggtaatgtgactcctgcatcaattgataacactgacatcaatgctgatatcagacctgtaggaaatggaactctgcaagcagatgtatcaaacactcactctcagacagttgagatgcagtttgagaataatgatgcagctgtgcgggatgttgaagctgtgagccaggagagtagtggtagtggggcaacttttggtgaaagccttcggagcctagatgttgagattggaagtgctgatggccatgatgatggtggagaaaggcaggtttctgcggataggatagcaggtgattcacaggctgcacgcacaagaagagcaaccatgtctgttggtcattcttctcctgtaggtgggagagatgcttcccttcatagtgtaactgaagtttcagaaaattcaagccgagatgcagatcaagatggtccagcagctgcggagcaggtgaacagtgatgctggatcaggatcaattgatcctgcctttctggaagctcttcctgaggagctgcgtgctgaagtcctctcatcccagcaaggtcacgtggctcaaccatcaaatgctgagtctcaaaacaatggggatattgatccagaattccttgcagctcttcccccagatattcgagcagaagttctagctcagcagcaagcacaaagactacatcaagctcaggagttggaagggcaacctgttgaaatggacaccgtctcaataattgcaacatttccttctgaattacgagaagaggttctattaacatcctctgatgctatccttgccaaccttacacctgcccttgtcgctgaggcaaatatgttgcgggagaggtttgcacatcgatacagtcgtaccctctttggtatgtatcccagaagtcgtagaggagacacttctaggcgtgatggtattggttctggcctggacggtgcagggggaagtgtcacttcacgcaggtctgctggcgctaaggttattgaagctgatggagcacctctacttgacaccgaagctttgcatgccatgattcggttatttcgcgtagttcagccactatataaaggtcaattgcagaggcttcttttgaatctttgtgcccatagtgaaacccgaatttccctggtgaatattctgatggacttactaatgcttgatgtaagaaagcctgccaattattttagtgccgttgaacctccatacagactatatggttgtcagagcaatgtaatgtattcacgtcctcaatcgtttgatggagttcccccgttactctctcggcgaatacttgaaactctcacctatcttgctcgccatcatccatttgtggcaaaaattttgcttcagtttaggctgcatcctcctgcattaagagaaccagataatgctggtgttgcacgtggcaaagctgtgatggtggttgaagatgaaataaatgctggttacatatccattgctatgcttttgggtctcttgaagcaacccctttatttgaggagcatagctcatcttgagcagttgctaaatttactggatgttatcattgatagtgctggaagcatgcctagttcatctgataaatctcagatatctactgaggcagttgtgggtccacaaatttctgcaatggaggtagatgcgaatattgattcagctacatcttctgctcttgacgcatctcctcaagtcaatgaatcctccaaacccacacctcacagtaataaggaatgtcaggctcagcaagtattgtgtgatctgccgcaggcagaacttcagctcctttgctcattgcttgctcaagaaggtttgtcagataatgcatatggtcttgttgcggaggtaatgaaaaaactagtggccattgctccgattcactgtcagctttttgtcactcatctggcagaagcagttcgaaaattgacttcatctgcaatggatgagttacgcactttcagtgaagcaatgaaagctcttctcagtacaacatcttctgatggcgctgcaattttaagagttttgcaggccttaagttccctggtaatctcattgaccgagaaagagaatgatggattaactcctgccctttctgaagtttggggaattaattcagcattagagcccttgtggcatgagcttagctgttgtataagcaagatagaagcctactctgagtcagtatctgagtctattacctcttctagaacatctgtgtcaaaaccatccagtgtcatgcctccacttccagctggttctcaaaatatcttaccatacatagaatctttttttgtggtctgtgagaagctacatcctgcacagtcaggtgctagtaatgacacaagtgttcctgttatttctgatgtggaagatgctaggacatctggtactcggctgaaaacatctgggcctgctatgaaggtagatgagaaaaatgctgcttttgccaagttttcggagaagcacaggaaactattaaatgcttttatcaggcaaaatcctggcttgcttgaaaagtctctttccctcatgctgaagactccaagatttattgattttgataacaagcgttcccatttccgatcaaaaattaaacatcagcacgaccatcaccacagcccattaagaatatcagtaagaagagcgtatgttctagaagattcatataaccagcttcgcatgagatcaactcaagatttgaagggaaggttgactgttcatttccaaggggaagaaggtatcgatgctggtgggcttacaagggaatggtaccaactgttgtctagagttatttttgacaaaggagcgctacttttcactacagtaggcaatgaatcaacatttcagccaaaccctaactctgtttaccaaacagaacacctatcttatttcaaatttgttggtagagtggttggaaaagctttatttgatggtcagctcttggatgtccattttactcggtcattctacaagcacatcctaggggccaaagttacatatcatgatattgaagccattgatcctgactatttcagaaatttgaaatggatgcttgagaatgatatcagtgatgttctggatcttacttttagcattgatgcagatgaggaaaagttgattttgtatgagcggacagaggtgactgattatgagctaattcctggtggacggaatacgaaagttacggaggagaataagcaccaatatgttgatttggttgctgagcatcggttgaccactgctattcgacctcaaataaatgctttcttggaagggttcaatgaattaattcccagggagttaatatctatattcaatgacaaagagctggaattattgatcagtggacttcctgatattgatttggatgacttgagagcaaatacagaatattctggatatagtggtgcctcaccagttatccaatggttttgggaggctgttcaaggtttcagcaaagaagacaaagctagattgctgcagtttgtgactggcacatccaaggtgcctttggagggttttagcgctcttcaaggaatttcaggtgcccagaggtttcagatacataaggcatatgggagttctgatcacttaccttctgctcatacttgtttcaatcaattagatttgccagagtatccatctaaacaacatttggaagagaggttactgcttgccattcatgaagcaaatgagggattcggatttggttga

SEQ ID NO: 22 - DNA, 11142 bp, Glycine max
atgacaagcgtaagatcgagttggccatcaaggctgcgccaacttctttccagcgagggttccattggcccttccgtcaaactcgactctgacccttctcctaagatcaaagccttcattgagaaggtcattcaatgtccattacaagatatagctatacccctctttggctttcggtgggagtataataaggggaattttcatcactggaggccattgtttcttcattttgatacatacttcaagacatatttatcatgtcgaaatgacctgacattgtccgataatctagaagttggcattccattaccaaaacatgcaattctacaaatactacgggtgatgcaaataatcttagagaactgtccaaacaagagttcatttgatggcttagagcacttcaagcttttactagcatcaacagatcctgagattattattgctacattagaaactcttgctgcgcttgtaaaaataaatccttctaagcttcatggaagtgcaaaaatggttggctgtggttcagtaaatagctatctcctgtccctagcacaggggtggggaagcaaggaggagggcatgggtttgtactcttgtattatggcaaatgagaaagcccaggatgaagcactgtgtttgtttccttctgatgcagagaatggtagtgaccactccaattactgcataggttctactctttattttgaattgcgtggacccattgctcaaagcaaggaacaaagtgtagatacagtttcctcaagtttgagagttatacacattccagatatgcatttacacaaagaagatgatttgtcaatgttgaagcaatgcattgagcagtataatgttcctcctgagctccgattttcattgctcacaagaattagatatgctcgtgctttccggtctgcgagaataagcaggctttatagcaggatttgccttcttgctttcactgtgttggtccaatccagtgatgctcatgacgagcttgtgtccttttttgccaacgaaccagagtacacaagcgaattgattagagttgtgcgatctgaagaaacaatatctggatctatcagaacacttgtaatgcttgcattaggagcccagttagcagcatacacatcatctcatgaacgggcacggatactgagtggatctagtatgaacttcactggagggaaccgcatgattctactgaatgtacttcagagggctattttgtcattgaagagttctaatgatccaacttcctttgcttttgttgaggcacttcttcaattctatctgctgcatgtagtgtcaacatcatcttctgggagtaatattagaggttctggcatggtacccacattcttgcctctgctggaggattctgatcttgctcatattcatcttgtttgtttagcagtgaaaacccttcagaagcttatggattatagtagttcagctgtatctttgtttaaagagttggggggtgttgagcatttggctcaaagattacagatagaggttcatagggtcattggttttgctggagagaatgataatgtgatgctcactggtgaaagctcaagacatagtactcatcagctttactctcagaagaggctgataaaagtgtcccttaaggcccttggttctgcaacatatgctcctgcaaactctaccagatctcaacactcccatgacagttcattacctgcaactctagtcatgatttttcagaatgtaaataagttcggaggtgacatttattactcagctgttactgttatgagtgaaataatccacaaagatcctacatgcttctcttctttgcatgaaatgggtcttccaaatgcttttttatcttcagttgcatctggaattcttccttcatcaaaggctctgacatgcattccaaatggcattggggccatttgtcttaatgccaaaggcttagaggttgttcgagagacttcatcactgcagttcctttttaatatctttacaagcaaaaagtatgtcctttccatgaatgaggctattgttccgctagcaaattctgtagaggaacttcttcgacacgtgtctccattgagaagtactggtgttgacatcatcattgaaatcatccataagattgcatcctttggtgatggtattgatacaggatcttcttcaggaaaagctaatgaggatagtgcaatggaaaccaattctgaagacaaaggaaatgaaaaccattgttgcctcgtgggcacagcagagtctgccgctgaagggattaatgatgagcaattcattcagctttgcacttttcatttgatggtattggttcaccggacaatggaaaattctgaaacatgtcggctatttgtagaaaaatcaggaattgaagctttattgaagctgttattacgacctaccattgcacaatcctcggatggcatgtctattgctctgcatagcaccatggtatttaaggggtttgctcaacatcattccgctcctttggcacgtgccttttgttcctctcttaaagagcacttgaatgaagcattaactgggtttgttgcatcttcgggacctttgttgctggatccaaagatgaccacaaataacatcttttcttcacttttcttggttgagtttcttctctttcttgctgcgtcaaaagacaaccgttgggtgactgctttgcttacagaatttggaaatggtagtaaggatgttcttgaaaacattggacgtgtccaccgtgaagttttgtggcaaattgctcttcttgaaaatacgaagcctgatattgaggatgacgtttcttgttctacttctgattcacaacaggcagaagtggatgcaaatgaaactgcagagcaaaggtacaattctatcaggcagtttcttgatccattactcaggaggaggactttaggatggagtgtagaatcacagttttttgatcttattaacctgtatcgagatctgggtcgtgcccctggttcccagcaccgatcaaattctgttggtcctacaaacaggcggttaggatcccctaatccgttgcatccgtctgagtcttcagatgtattgggggatgctagtaagaaagaatgtgacaagcaaagaacatattatacctcttgttgtgacatggccagatcactttcatttcacattatgcatttgttccaagagttaggaaaagtaatgctgcaaccttctcgccgtcgtgatgatgttgcaagtgtaagtcctgcttcaaaatcagtggcttctacttttgcaagcattgctctagatcacatgaattttgggggccatgtagaagaagcatccatatcaacaaaatgtcgttattttggtaaagtcattgattttgtggatggcattctaatggaaaggcctgattcttgcaatcccattttactgaattgcttgtatgggcatggagttattcaatctgtattgaccacatttgaagcaactagtcagttgttatttgcagttaatcggacccctgcatcgccgatggaaattgatgatggaaatgtgaagcaggatgacaaggaagataccgatcatttgtggatatatggttctttagccagttatggtaaatttatggaccatctagtaacctcctctttcatattatcttctttcacaaagcctatacttgcacagccccttagtggtgatacctcatatccccgggatgctgagatatttgtgaaagtcctccaatctatggtgttgaaggctgtgctcccagtttggatgcatccccagtttgttgattgtagtcatggatttatttctaatgttatctctatcatcaggcatgtttattcaggggttgaagtaaaaaatgtaaatggcagcagcagtgctcgtattactgggcctcctcctaatgaaacaacaatttcaaccattgtagagatgggattttccaggtcgagagcagaagaagctttgaggcatgttggatcaaatagtgtggagttggcgatggagtggctgttttcccatccagaggacacacaagaagatgacgaacttgctcgtgcacttgccatgtcccttgggaactctgaatcagacaccaaggatgctgctgcaaatgacagtgtacaactgcttgaggaagaaatggtccatcttcctcctgttgatgagttgttatcaacttgcactaaacttcttcaaaaggaacctcttgcttttcctgtccgtgacttgctcatgatgatatgctctcagaatgatggtcaaaatagatctaatgttctcacttttattgttgaccggatcaaggaatgtggattgatttctggtaacggaaataataccatgcttgctgctctatttcatgttcttgcattgattcttaatgaggatgctgttgcgcgagaagctgcttcaaagagtggtttcataaaaattgcctcagatctactctaccaatgggattctagtcttggtaacagggagaaagaacaggttccaaaatgggtcacagctgcttttcttgcattagacaggctgttgcaagtggatcaaaaattgaattctgaaattgcagagcttttgaagaaggaagctttgaatgttcagcagacatcagttatcattgatgaggataagcaacacaaattgcagtctgcgttgggactttccaccaagtatgcagatatacatgaacagaagagacttgttgagattgcttgtagttgcatgaagaaccaacttccctcagacacaatgcatgctattttgctactatgttccaatcttacaaagaatcactctgttgctcttaccttttttgatgctggtggtttaagtttacttctttctctgccaaccggtagcctctttcctgggtttgacaacgttgctgctggtattgtccgtcatgttattgaagatccacaaactctccagcaagcaatggaatctgagataaaacacagtcttgtagctgcgtctaaccgccatccaaatgggagggtcaatccacaaaattttctgttaagtttagcttctgtaatttcccgggatccaataatatttatgcaagctgctcaatctgcttgccaagttgaaatggtgggtgaaagaccttacattgtcttgctgaaagatcgggataaagagaaatccaaggataaggataagtcactggagaaagataaagcacataatgatggaaaaattggtttgggaagtacagccacagcagcttcagggaatgttcatggaaaacttcatgattcaaactcaaagaatgccaaaagttacaaaaagcctactcaaagttttgttaatgtgatagaacttcttcttgaatctatatgcacatttgttgccccccctttgaaggacaataatgtatcaaatgttgtccctggctccccaacatcaagtgacatggacattgatgtttctacagttagggggaaaggaaaagcagttgccactgtgcctgaggggaatgaaaccagcagtgaggaagcatctgcatcactagcaaagatagtatttattttgaagcttctgatggagatattgttgatgtattcatcgtctgttcatgttctgcttcgacgggatgctgaaatgagcagctctagggacatttatcaaaagaatcatggtagttttggtgcgggagtaatattctaccatattcttcgtaattttcttccttgttctcgaaattccaaaaaagacaagaaagttgatgatgattggaggcagaaactagcaacaagggctaatcagtttatggtagctgcttgtgttcgttcttcagaggcaaggaggcgggtttttactgagattagccatatcattaatgaatttgttgattcatgtaattgtgttaagccaaagccatcaggcaatgaaattctggtttttgttgatctacttaatgatgttttggctgctcggacacctgctggctcaagcatctcagcagaggcctctgtcacttttatggatgctggtctacttaaatcttttacccgtactctccaagttttagacttggaccatgctgactcgtctaaagttgctactggtattatcaaagctcttgaactagtaaccaaggagcatgttcactcagttgaaccgagtgcaggaaagggtgataatcaaactaagccttctgatcctagtcaatccggaagaacagataatattggtcacatgtgtcagtccatggaaacaacatctcaggccaatcacgattcccttcaagttgaccatgttgggtcttacaatgtgattcagtcttatggtgggtctgaagctgttattggtgatatggaacatgatcttgatggggactttgctcctgctaatgaagatgagttcatgcatgaaactggtgaggatgccagaggccatgggaatggaattgaaaatgttgggctacaatttgaaatccaatcccatggacaagaaaatctcgatgatgacgatgatgagggtgatatgtctggagatgagggtgaagatgtagatgaagatgacgaagatgatgaggaacacaatgatttggaagaagatgaagtccatcacttgccacatcctgacactgatcgtgatgatcatgagatggatgatgatgattttgatgaagtgatggagggggaggaggatgaagatgaggatgatgaagatggtgttatactgagacttgaggagggcatcaatggaattaatgtttttgaccatattgaggtttttggaagagacaatagttttccaaatgaatcccttcatgtcatgccagttgaagtttttggatctagacgtccagggcggaccacctctatttacagcctgttgggcagaagtggtgataatgccgccccttcttgccatccacttttagttggtccttcttcctcattccatctatctaatggtcaatcagatagtataacagagaactccacaggcttggataatatctttcgttcattgaggagcggacgtcatgggcaccgcttgaacttgtggagtgataatagccagcaaatcagtgggtcaaatactggcgctgtaccacagggccttgaggagttgcttgtgtctcaattgaggcgacctactgctgagaagtcgtctgataataatatagcagacgctggtcctcataataaagttgaggtcagccagatgcacagttccggaggttcaaagcttgaaatcccagttgaaagcaatgcaattcaggaaggtggtaatgtgactcctgcatcaattgataacactgacatcaatgctgatatcagacctgtaggaaatggaactctgcaagcagatgtatcaaacactcactctcagacagttgagatgcagtttgagaataatgatgcagctgtgcgggatgttgaagctgtgagccaggagagtagtggtagtggggcaacttttggtgaaagccttcggagcctagatgttgagattggaagtgctgatggccatgatgatggtggagaaaggcaggtttctgcggataggatagcaggtgattcacaggctgcacgcacaagaagagcaaccatgtctgttggtcattcttctcctgtaggtgggagagatgcttcccttcatagtgtaactgaagtttcagaaaattcaagccgagatgcagatcaagatggtccagcagctgcggagcaggtgaacagtgatgctggatcaggatcaattgatcctgcctttctggaagctcttcctgaggagctgcgtgctgaagtcctctcatcccagcaaggtcacgtggctcaaccatcaaatgctgagtctcaaaacaatggggatattgatccagaattccttgcagctcttcccccagatattcgagcagaagttctagctcagcagcaagcacaaagactacatcaagctcaggagttggaagggcaacctgttgaaatggacaccgtctcaataattgcaacatttccttctgaattacgagaagaggttctattaacatcctctgatgctatccttgccaaccttacacctgcccttgtcgctgaggcaaatatgttgcgggagaggtttgcacatcgatacagtcgtaccctctttggtatgtatcccagaagtcgtagaggagacacttctaggcgtgatggtattggttctggcctggacggtgcagggggaagtgtcacttcacgcaggtctgctggcgctaaggttattgaagctgatggagcacctctacttgacaccgaagctttgcatgccatgattcggttatttcgcgtagttcagccactatataaaggtcaattgcagaggcttcttttgaatctttgtgcccatagtgaaacccgaatttccctggtgaatattctgatggacttactaatgcttgatgtaagaaagcctgccaattattttagtgccgttgaacctccatacagactatatggttgtcagagcaatgtaatgtattcacgtcctcaatcgtttgatggagttcccccgttactctctcggcgaatacttgaaactctcacctatcttgctcgccatcatccatttgtggcaaaaattttgcttcagtttaggctgcatcctcctgcattaagagaaccagataatgctggtgttgcacgtggcaaagctgtgatggtggttgaagatgaaataaatgctggttacatatccattgctatgcttttgggtctcttgaagcaacccctttatttgaggagcatagctcatcttgagcagttgctaaatttactggatgttatcattgatagtgctggaagcatgcctagttcatctgataaatctcagatatctactgaggcagttgtgggtccacaaatttctgcaatggaggtagatgcgaatattgattcagctacatcttctgctcttgacgcatctcctcaagtcaatgaatcctccaaacccacacctcacagtaataaggaatgtcaggctcagcaagtattgtgtgatctgccgcaggcagaacttcagctcctttgctcattgcttgctcaagaaggtttgtcagataatgcatatggtcttgttgcggaggtaatgaaaaaactagtggccattgctccgattcactgtcagctttttgtcactcatctggcagaagcagttcgaaaattgacttcatctgcaatggatgagttacgcactttcagtgaagcaatgaaagctcttctcagtacaacatcttctgatggcgctgcaattttaagagttttgcaggccttaagttccctggtaatctcattgaccgagaaagagaatgatggattaactcctgccctttctgaagtttggggaattaattcagcattagagcccttgtggcatgagcttagctgttgtataagcaagatagaagcctactctgagtcagtatctgagtctattacctcttctagaacatctgtgtcaaaaccatccagtgtcatgcctccacttccagctggttctcaaaatatcttaccatacatagaatctttttttgtggtctgtgagaagctacatcctgcacagtcaggtgctagtaatgacacaagtgttcctgttatttctgatgtggaagatgctaggacatctggtactcggctgaaaacatctgggcctgctatgaaggtagatgagaaaaatgctgcttttgccaagttttcggagaagcacaggaaactattaaatgcttttatcaggcaaaatcctggcttgcttgaaaagtctctttccctcatgctgaagactccaagatttattgattttgataacaagcgttcccatttccgatcaaaaattaaacatcagcacgaccatcaccacagcccattaagaatatcagtaagaagagcgtatgttctagaagattcatataaccagcttcgcatgagatcaactcaagatttgaagggaaggttgactgttcatttccaaggggaagaaggtatcgatgctggtgggcttacaagggaatggtaccaactgttgtctagagttatttttgacaaaggagcgctacttttcactacagtaggcaatgaatcaacatttcagccaaaccctaactctgtttaccaaacagaacacctatcttatttcaaatttgttggtagagtggttggaaaagctttatttgatggtcagctcttggatgtccattttactcggtcattctacaagcacatcctaggggccaaagttacatatcatgatattgaagccattgatcctgactatttcagaaatttgaaatggatgcttgagaatgatatcagtgatgttctggatcttacttttagcattgatgcagatgaggaaaagttgattttgtatgagcggacagaggtgactgattatgagctaattcctggtggacggaatacgaaagttacggaggagaataagcaccaatatgttgatttggttgctgagcatcggttgaccactgctattcgacctcaaataaatgctttcttggaagggttcaatgaattaattcccagggagttaatatctatattcaatgacaaagagctggaattattgatcagtggacttcctgatattgatttggatgacttgagagcaaatacagaatattctggatatagtggtgcctcaccagttatccaatggttttgggaggctgttcaaggtttcagcaaagaagacaaagctagattgctgcagtttgtgactggcacatccaaggcttatgatggagataagacacattgggagcctttgcttaacaaatttcaagccaagctctcaaagtggaatcagaaaactttgtctatgggtggtagagttaccttgataaaatctgtcctgagtgcactccctatatatctactatctttcttcaagatcccccaaagaatagtggataagttggtgaccctccaaaggcagtttctgtgggggggaactcaacaccataacagaattccttgggtcaagtgggctgacatctgcaatccgaagattgatgggggattgggaatcaaagacctgtccaatttcaatgcagccttaaggggaagatggatctggggattagcttctaatcacaatcagctttgggccagacttgcagagcagtag

SEQ ID NO: 23 - DNA, 10839 bp, Glycine max
atgacaaccctaagatcgagttggccttcgaggctgcgccaacttctgtcgagcgagggcgccattggtccttccgtcaaggtggacaccgagccccctcctatggtcaaagccttcattgagaagatcatccagtgtccattacaagatattgccataccactttctggctttcggtgggagtacaataaggggaattttcatcactggagactgttgttgcttcattttgatacatacttcaagacttatttgtcgtgtagaaatgatctgacattgttagataatctagaagatgacagcccattaccaaaacatgcaattctgcaaatattgcgagtgttgcaaataattttagagaactgtccaaacaagagttcctttgatggcttagagcatttcaagcttttactagcatcaacagatcctgagattcttattgctacattagaaactctttctgcacttgtaaaaattaatccctctaagcttcatggaagtacaaagatgatttgctgtggttcggtgaacagctatcttttgtccctagcacaaggctggggaagcaaggaggagggcctaggattgtactcttgtgttatggcaaatgagaaagtccaagatgaagcactgtgcttatttccttctgaagagattggtcatgaccaatcaaattgccgcatgggcactaccctttattttgaattgcatggtcccagtgcccaaagcaaggaacatagtgcagatgcagtttcccctggctcaacagttatacatatgccagatttgcatctgcgcaaagaagatgatttgtccttgatgaagcagtgcattgaacaatttagcgttccttctgagctcagattttcattgctcactagaatcagatatgctcgtgcctttcgttctcctagaatatgcaggctttacagcaggatttgcctactttctttcattgttctggtgcagtctggtgatgctcaggaagaactcgtctccttttttgccaatgaaccagaatatacaaatgaattaattagaattgtacgttcagaggaagttatatctggatctatcaggacacttgcaatgcttgctttaggagctcaattagccgcatatacatcatcgcatcatcgggcacgaatactcagtggatctagtttaacttttgctggtgggaaccgcatgatactcctaaatgtgctccagagggctattttgtcattgaagagttctaatgatccatcatcccttgcttttgttgaagcacttcttcagttctatctgctccatgtggtttcaacctcaacttctggtaataatattagaggttctggcatggtgcctacattcttgccgttgctggaggattttgatcctacacatattcatctagtctgttttgctgtgaaaacacttcagaagcttatggattatagtagctcagctgtatcattgtttaaagaattggggggcattgaacttttggctcagagattacagaaagaggtacacagagtcattggtttggttggagaaactgataacattatgcttactggtgaaagcttgagatatagtactgatcaattgtactcccagaagagactcataaaggtctcccttaaggcgcttggttctgcaacatacgcgcctgcaaactctaccagatctcaacattctcaagacagttcattacctgtaactctaagattgatttttcagaatgtagataagtttggaggtgacatttattattcagctgttactgttatgagcgaaataatccacaaagatccaacctgtttttctgctctgcatgaaatgggtcttcctgatgcttttttattgtcagttggatctgaaatacttccatcatcaaaggctttgacatgcattccaaatggtcttggggcaatttgtcttaatgccaaagggttagaggctgttagagaatcttcatcgctacggttccttattgacattttcactagcaagaagtatatcttagccatgaatgaggctattgttcctttggcaaatgctgtggaggaacttctacgtcatgtatctacattgagaagctccagtgttgatattatcattgaaatcatccacaagatcgcatcttttggggatggaaatggtactggattttctggaaaagctgagggcactgccatggaaacagattctgaaaacaaagaaaaagaaggccattgttgcattgtaggcacatcatattcagccatagaagggataagtgatgagcagtttattcagctatgtgtctttcatttaatggtattgattcataggactatggaaaatgccgagacatgccggttgtttgtggaaaaatcaggaattgaagctttattgaatttgttgttgcggccaactattgcacaatcctcagatggcatgtctattgctttacatagcacgatggtatttaaagggtttgctcaacatcattccattcctctggcacatgccttctgttcttctcttagagagcacttgaagaaagctttagcggggcttggtgcagcatcagaacctttgttgctggatccaaggatgacaactgatggtgccatcttttcttcacttttcctggttgagttccttctatttcttgctgcaccaaaagacaatcgttgggtgactgccttgcttacagaatttggaaatggaggtaaggatgttcttgaagacattggacgtgtacaccgtgaagtcctgtggcaaattgctctacttgaaaacagaaagcctgagattgaggaagatggtgcttgtacttctgatttacaacaggccgaaggggatgcaagtgaaactgaagagcaaaggttgaattctttcaggcagtttcttgacccattattgagaagaagaacatcaggatggagcattgaatctcagttttttaaccttataaacctgtatcgagatttgggccgttccactggttctcaacatagatcaaatttagttggtccgaggtcaagttctagtaatcaggtacagcattctgggtcagatgataattctgggactgctgataagaaggaatctgacaagcagagaccatattatacctcttgttgtgacatggtcagatcactttcatttcacattacccatttgttccaagagttgggaaaagtaatgttgctaccttcacgtcgacgtgatgatgttgtgaatgtaagtcctgcttcaaaatcagtggcttctacttttgcatccattgcttttgatcacatgaattatggtggccgttgtgtaaatctttcgggaacagaagaatccatatcaacaaaatgtcgatattttgggaaagtgattgattttatggataatgttctaatggagaggccagattcatgcaatcctattatgctgaattgcttgtatggacgtggagttattgaaactgtattaactacctttgaagctaccagtcagctgctctttacagttaatcgggcccctgcctcgcccatggatactgatgatgcgaatgcaaagcaagatgacaaggaagatacagataattcatggatttatggttctttagctagttatgggaaattgatggaccatctagtgacctcctcttttatattatcatcattcacaaagcatttacttgcacagccccttactaatggtaatacagctttcccaagggatgctgagacttttgtgaaggtccttcaatccagagtgttgaagactgtgcttcctgtttggactcatccccagtttgttgactgtagttatgaatttatttctacagttatttctatcattaggcatgtctatacaggtgttgaagtaaaaaatgtgaacggaagtggtggtgctcgcattactgggccgcctcctaatgaaacaactatttcaaccattgtagagatggggttttccaggtctagagcagaagaagctttgaggcaagttgggtcaaatagtgtggagttggcaatggagtggttgttctctcatccagaggagatacaagaagatgatgaacttgcccgtgcacttgccatgtcccttggaaactcagaatcagatgcaaaggatgcagttgctaatgacaatgccctgcagcttgaagaagagatggtcctactccctcctgttgatgagttgttatctacttgcacaaaacttttgtcgaaggaaccacttgcttttcctgtccgtgacttgcttgtgatgatatgctctcatgatgatggtcaccatagatctaatgtggtctcatttattgtggaacggatcaaagaatgtggtttggttcctagcaatggaaatgttgccacgctggctgctctttttcatgttctagccttaattcttaatgaggatgctgtggctagggaagctgcttctacaagtggtttgatcaaaattgcctcagatctactctaccagtgggattctagtcttgatagcagggagaaacagcaggtaccaaaatgggtgactgctgctttccttgcattagacagattgttgcaagtagatcaaaaattgaattctgaaatcgcagagcagttgaagaaggaagctgtgaatagccagcagacatcgattaccattgatgaagacaggcaaaacaagttgcagtctgcattgggactctctatgaagtatgcagatatacatgaacagaagagacttgttgaggttgcttgtagttgtatgaataatcaacttccatctgacacaatgcatgctattctgctactatgttccaatcttacaaggaatcattctgtagctcttacatttttggatgctggtggtttgaatctacttctttctttgccaaccagcagcctcttccctgggtttgacaatgttgctgctagtattgttcgtcatgttcttgaagatccacaaacgctccagcaagcaatggaatctgagataaaacatagtcttgcagtggcatctaatcggcatccaaatggaagggtcaatcctcataatttccttttaaatttagcttctgttatttatcgggatccagtaatctttatgctagctgctcaatctgtgtgccaagttgaaatggtaggtgagaggccatacattgtcttgctgaaagatagggataaagacaaagctagggagaaagaaaaggataaggataaaacattggagaaagataaagtacagaacagtgatgggaaggttgttttgggaaatacaaacacagcacctactggcaatggccatggcaaaattcaggattcaaataccaagagtgccaaaggtcacagaaaacctaaccaaagttttattaatgtaatagagcttcttcttgaatctatatgcacttttgttcctcccttgaaggatgacattgcctcaaatgttcttcctggaaccccagcatcaactgatatggacattgatgtctccgtggttaagggaaaaggaaaagcagttgccactgtgtctgacggcaacgaaactggtagtcaggttgcttctgcatcacttgcaaagattgtcttcattttaaagcttctgacagagatattattgctgtattcatcatctgttcatgttctacttcgacgagatgctgaaataagctgcattagaggttcttatcaaaagagtcctgcaggtttaagcatgggttggatattttcccatattcttcataattttcttccatattctcgaaactcaaaaaaggacaagaaagctgatggtgattggaggcagaaactagcaaccagggccaaccagtttatagtgggtgcttgtgttcgatctacagaggcaaggaagagggtttttggtgagattagttatatcatcaatgaatttgttgattcatgtcatgacattaagcgtccaggaaatgaaattcaggtttttgttgatctactaaatgatgttttggctgctcgtacacctgctggttcgtacatttcagctgaggcctctaccacttttatagatgctggtttggttaaatcattcacttgcactctacaagttttggaccttgaccatgctggttcatctgaagttgctactggtattattaaagctcttgagttggtaaccaatgagcatgtccattcagttcattctagtgcagggaagggtgataattcaacaaaaccttctgttctaagtcaacctggaagaacaaataatattggtgaactgtctcagtccatggagacatcacaagccaatcctgattcccttcaagttgaccatgttgggtcttatgcagttcactcctatggtgggtctgaagctgttactgatgatatggaacatgatcaagatcttgatgggagctttgttcctgctaatgaggatgattacatgcatgaaaattctgaggatgcaagaaatcttgaaaatggaatggaaaatgtgggtctacaatttgaaatccaacctcatggccaagaaaatcttgatgaggatgacgatgaggatgatgatatgtctggagatgaaggtgaggatgtagatgaagatgatgatgatgaggaggaacacaatgatttggaagaagtccatcatttgccacatcctgacacagatcaagacgagcatgagattgatgatgaagattttgatgatgaagtgatggaggaagacgatgaggatgacgaggaagatgaagatggtgttatactgcgacttgaggagggaattaatggaattaatgtttttgaccatattgaggtttttggcagagataatagttttgcaaatgaagctttacatgtaatgccagttgaggtttttggatccagacgtccggggaggacgacatctatttatagtcttttgggcagaactggtgatgctgctgtgccttctcgtcacccactcttgcttgaaccttcttcattccctccacctacagggcaatcagatagttcaatggagaacaactcagtgggtttggataatatatttcgatcgctgaggagtgggcgccatggacaccgtttgcacttgtggactgataataaccagcaaagtggtgggacaaacactgctgttgtaccacaaggccttgaggagttgcttgtcactcaattaaggcgaccaacccctgaaaagtcatccaatcagaacatagcagaagcaggttctcatggtaaaattggaacaacccaggcacaagatgcagggggtgcaaggccagaagtccccgttgaaagtaatgctattctggaaattagtactataactccctcaattgataacagtaacaatgcggatgtcagaccagcagggactggaccttcacatacaaatgtttcaaacacccaatcacgggcagttgagatgcaatttgaacatactgatggagctgtgagggatattgaagctgtcagccaggagagtagtggtagtggagcaactttcggtgaaagccttcggagcttggaagttgagattggaagtgctgatggccatgatgatggtggtgaaaggctggtttctgctgataggatggcaggtgattcacaggctgcacgcacaagaagagcaaatacacctttgagtcacttttctcctgtggttggaagagatgtgtcccttcatagtgttactgaagtttcagaaaattcaagccgtgatgcagatcaacaaggtcctgcagcagagcagcaggtgaacagtgatgcgggatcaggagctattgatcctgcttttctggatgctcttcctgaggagctacgtgctgaagtcctttcagctcagcagggtcaagtggctcagccatcaaatgttgagtctcaaaacactggggatattgacccagagttcctagcagctcttccagctgatattcgagcagaagttctagctcagcagcaagcacagaggttgcatcagtctcaggagctggaaggtcaacctgtggaaatggatacagtctcaataattgcaacttttccatcagatttacgagaagaggttctcttgacatcaccagatactatccttgccaatcttacacctgctcttgttgctgaggcaaatatgttgcgggagaggtttgcacaccgttatagtcgtaccctctttggtatgtatccaagaagtcgtagaggggagacttcaagacgtgaaggtattggttctggtctggatggagcaggaggaaccatttcttctcgccgctccagtggagttaaggttgttgaagctgatggagcacctttagttgacacagaagctttgcatgctatgattcggttatttcgtgtagtgcagccactctataaaggccaactccagaggcttctattaaatctttgtgcccatagtgaaacaagaacctctctggtgaaaattctcatggacttgctaatgcttgatgtaaaaaggcctgtcagttattttagtaaagttgagccaccatatagattgtatggttgtcagagcaatgtaatgtattcacgtcctcaatcttttgatggagttcccccattgctgtctcgtagaatacttggaattctcacttatcttgctcgcaatcatctgtatgtggcaaaatttttgcttcagtgtaggctgtctcatcctgcaataaaagaaccagatgatccacggggaaaagctgtgatggttgttgaagatgaagtaaatataagtgaaagtaatgatgggtacatcgccattgcaatgctattgggtctgttgaaccaaccactttatttgaggagcatagcccaccttgagcagctgctagatttactggatgttatcattgacagtgctggaaacaagtcatctggcaaatccttgatacctactaacccatcatcagctccacaaatttctgctgcggaagccgatgcgaatgcagattctaacaatttaccttctgcggatgatgcatctaaagttgatggttcctccaaaccgacagtctctggcattaatgttgaatgtgagttacatggagtgttgagtaatcttccaaaagcagaactccggctcctgtgctcactgcttgctcaagaaggtttgtcagataatgcgtataatcttgtagcggaagtaatgaagaaattggtggccattgctccaacacattgtgagctttttgtcactgagctggcagaagcagttcaaaagttgacttcatctgcaatgaatgagttacgtgtctttagtgaagcaatgaaagctctgcttagtacctcttctactgatggagctgcaattttgagagtcttgcaagccttgagttcccttgtcaccttactgacggagaaagagaatgatagaggtactcctgctctttctgaggtttgggaaatcaattcagcattagaacccttgtggcatgagcttagttgttgcataagcaagatagaatcctactcagagtctgcatctgagttttcgacatcttctagtacctttgtgtctaaaccgtctggtgtaatgcctccacttccagctggctctcaaaatatcttaccatacattgaatctttctttgtggtttgtgagaaattgcatcctgctcagccaggtgctagtcacgactcaagtattcctgttatttcggatgttgagtatgccaccacatctgtaactccccagaaagcatctggaactgctgtgaaagtagatgagaaacatatgccttttgtccggttctcagagaagcacaggaagctactaaatgctttcataaggcaaaaccctggtttgcttgaaaaatctttctcactcatgctaaaggttccaagatttattgattttgataacaagcgtgcccacttccgatcaaaaattaagcatcagcatgaccatcaccatagtcccttgagaatatcagtaagaagggcatatgttctagaagattcttacaaccagcttcgcatgagatcaactcaagatttgaagggaaggttgactgtccacttccaaggggaggagggtattgatgcaggtgggcttacaagggaatggtatcaattattgtccagagttatttttgataaaggagcactgctttttactacagtgggcaatgaatcaacatttcagccaaaccctaactctgtttaccaaacagaacatttatcttatttcaaatttgttggtagagtggtcggtaaagcattatttgatggtcaactcttggatgttcattttactcggtcattctacaagcacattcttggagtcaaagttacatatcatgatattgaagccattgatcctgattatttcaaaaatttgaaatggatgcttgagaatgatatcagtgatgttctggatcttacttttagcattgacgcagatgaggaaaaattgatcttatatgaacgaacagaggtgactgattatgagttgattcccgggggacggaatatcaaagttactgaggagaacaagcatcaatatgttgatttggttgccgagcatcggttgacaactgctattcgacctcaaataaattatttcttagaagggttcattgaattgattcccagggagttgatatcgatattcaatgacaaagagctggaattgttgatcagtggacttcctgatattgatttggatgacttgagagcaaatacagaatattctggatatagtgctgcatcgccagttatccaatggttttgggaggttgttcaaggtttgagcaaagaagacaaggctcgactgttgcaatttgtgacaggcacatccaaggaatttcaggctcccagaagtttcagatacacaaagcatatggaagtcctgatcacttgccttctgctcatacttgcttcaatcaattag

SEQ ID NO: 24 - DNA, 10959 bp, Glycine max
atgacaaccctaagatcgagttggccttcgaggctgcgccaacttctgtcgagcgagggcgccattggtccttccgtcaaggtggacaccgagccccctcctatggtcaaagccttcattgagaagatcatccagtgtccattacaagatattgccataccactttctggctttcggtgggagtacaataaggggaattttcatcactggagactgttgttgcttcattttgatacatacttcaagacttatttgtcgtgtagaaatgatctgacattgttagataatctagaagatgacagcccattaccaaaacatgcaattctgcaaatattgcgagtgttgcaaataattttagagaactgtccaaacaagagttcctttgatggcttagagcatttcaagcttttactagcatcaacagatcctgagattcttattgctacattagaaactctttctgcacttgtaaaaattaatccctctaagcttcatggaagtacaaagatgatttgctgtggttcggtgaacagctatcttttgtccctagcacaaggctggggaagcaaggaggagggcctaggattgtactcttgtgttatggcaaatgagaaagtccaagatgaagcactgtgcttatttccttctgaagagattggtcatgaccaatcaaattgccgcatgggcactaccctttattttgaattgcatggtcccagtgcccaaagcaaggaacatagtgcagatgcagtttcccctggctcaacagttatacatatgccagatttgcatctgcgcaaagaagatgatttgtccttgatgaagcagtgcattgaacaatttagcgttccttctgagctcagattttcattgctcactagaatcagatatgctcgtgcctttcgttctcctagaatatgcaggctttacagcaggatttgcctactttctttcattgttctggtgcagtctggtgatgctcaggaagaactcgtctccttttttgccaatgaaccagaatatacaaatgaattaattagaattgtacgttcagaggaagttatatctggatctatcaggacacttgcaatgcttgctttaggagctcaattagccgcatatacatcatcgcatcatcgggcacgaatactcagtggatctagtttaacttttgctggtgggaaccgcatgatactcctaaatgtgctccagagggctattttgtcattgaagagttctaatgatccatcatcccttgcttttgttgaagcacttcttcagttctatctgctccatgtggtttcaacctcaacttctggtaataatattagaggttctggcatggtgcctacattcttgccgttgctggaggattttgatcctacacatattcatctagtctgttttgctgtgaaaacacttcagaagcttatggattatagtagctcagctgtatcattgtttaaagaattggggggcattgaacttttggctcagagattacagaaagaggtacacagagtcattggtttggttggagaaactgataacattatgcttactggtgaaagcttgagatatagtactgatcaattgtactcccagaagagactcataaaggtctcccttaaggcgcttggttctgcaacatacgcgcctgcaaactctaccagatctcaacattctcaagacagttcattacctgtaactctaagattgatttttcagaatgtagataagtttggaggtgacatttattattcagctgttactgttatgagcgaaataatccacaaagatccaacctgtttttctgctctgcatgaaatgggtcttcctgatgcttttttattgtcagttggatctgaaatacttccatcatcaaaggctttgacatgcattccaaatggtcttggggcaatttgtcttaatgccaaagggttagaggctgttagagaatcttcatcgctacggttccttattgacattttcactagcaagaagtatatcttagccatgaatgaggctattgttcctttggcaaatgctgtggaggaacttctacgtcatgtatctacattgagaagctccagtgttgatattatcattgaaatcatccacaagatcgcatcttttggggatggaaatggtactggattttctggaaaagctgagggcactgccatggaaacagattctgaaaacaaagaaaaagaaggccattgttgcattgtaggcacatcatattcagccatagaagggataagtgatgagcagtttattcagctatgtgtctttcatttaatggtattgattcataggactatggaaaatgccgagacatgccggttgtttgtggaaaaatcaggaattgaagctttattgaatttgttgttgcggccaactattgcacaatcctcagatggcatgtctattgctttacatagcacgatggtatttaaagggtttgctcaacatcattccattcctctggcacatgccttctgttcttctcttagagagcacttgaagaaagctttagcggggcttggtgcagcatcagaacctttgttgctggatccaaggatgacaactgatggtgccatcttttcttcacttttcctggttgagttccttctatttcttgctgcaccaaaagacaatcgttgggtgactgccttgcttacagaatttggaaatggaggtaaggatgttcttgaagacattggacgtgtacaccgtgaagtcctgtggcaaattgctctacttgaaaacagaaagcctgagattgaggaagatggtgcttgtacttctgatttacaacaggccgaaggggatgcaagtgaaactgaagagcaaaggttgaattctttcaggcagtttcttgacccattattgagaagaagaacatcaggatggagcattgaatctcagttttttaaccttataaacctgtatcgagatttgggccgttccactggttctcaacatagatcaaatttagttggtccgaggtcaagttctagtaatcaggtacagcattctgggtcagatgataattctgggactgctgataagaaggaatctgacaagcagagaccatattatacctcttgttgtgacatggtcagatcactttcatttcacattacccatttgttccaagagttgggaaaagtaatgttgctaccttcacgtcgacgtgatgatgttgtgaatgtaagtcctgcttcaaaatcagtggcttctacttttgcatccattgcttttgatcacatgaattatggtggccgttgtgtaaatctttcgggaacagaagaatccatatcaacaaaatgtcgatattttgggaaagtgattgattttatggataatgttctaatggagaggccagattcatgcaatcctattatgctgaattgcttgtatggacgtggagttattgaaactgtattaactacctttgaagctaccagtcagctgctctttacagttaatcgggcccctgcctcgcccatggatactgatgatgcgaatgcaaagcaagatgacaaggaagatacagataattcatggatttatggttctttagctagttatgggaaattgatggaccatctagtgacctcctcttttatattatcatcattcacaaagcatttacttgcacagccccttactaatggtaatacagctttcccaagggatgctgagacttttgtgaaggtccttcaatccagagtgttgaagactgtgcttcctgtttggactcatccccagtttgttgactgtagttatgaatttatttctacagttatttctatcattaggcatgtctatacaggtgttgaagtaaaaaatgtgaacggaagtggtggtgctcgcattactgggccgcctcctaatgaaacaactatttcaaccattgtagagatggggttttccaggtctagagcagaagaagctttgaggcaagttgggtcaaatagtgtggagttggcaatggagtggttgttctctcatccagaggagatacaagaagatgatgaacttgcccgtgcacttgccatgtcccttggaaactcagaatcagatgcaaaggatgcagttgctaatgacaatgccctgcagcttgaagaagagatggtcctactccctcctgttgatgagttgttatctacttgcacaaaacttttgtcgaaggaaccacttgcttttcctgtccgtgacttgcttgtgatgatatgctctcatgatgatggtcaccatagatctaatgtggtctcatttattgtggaacggatcaaagaatgtggtttggttcctagcaatggaaatgttgccacgctggctgctctttttcatgttctagccttaattcttaatgaggatgctgtggctagggaagctgcttctacaagtggtttgatcaaaattgcctcagatctactctaccagtgggattctagtcttgatagcagggagaaacagcaggtaccaaaatgggtgactgctgctttccttgcattagacagattgttgcaagtagatcaaaaattgaattctgaaatcgcagagcagttgaagaaggaagctgtgaatagccagcagacatcgattaccattgatgaagacaggcaaaacaagttgcagtctgcattgggactctctatgaagtatgcagatatacatgaacagaagagacttgttgaggttgcttgtagttgtatgaataatcaacttccatctgacacaatgcatgctattctgctactatgttccaatcttacaaggaatcattctgtagctcttacatttttggatgctggtggtttgaatctacttctttctttgccaaccagcagcctcttccctgggtttgacaatgttgctgctagtattgttcgtcatgttcttgaagatccacaaacgctccagcaagcaatggaatctgagataaaacatagtcttgcagtggcatctaatcggcatccaaatggaagggtcaatcctcataatttccttttaaatttagcttctgttatttatcgggatccagtaatctttatgctagctgctcaatctgtgtgccaagttgaaatggtaggtgagaggccatacattgtcttgctgaaagatagggataaagacaaagctagggagaaagaaaaggataaggataaaacattggagaaagataaagtacagaacagtgatgggaaggttgttttgggaaatacaaacacagcacctactggcaatggccatggcaaaattcaggattcaaataccaagagtgccaaaggtcacagaaaacctaaccaaagttttattaatgtaatagagcttcttcttgaatctatatgcacttttgttcctcccttgaaggatgacattgcctcaaatgttcttcctggaaccccagcatcaactgatatggacattgatgtctccgtggttaagggaaaaggaaaagcagttgccactgtgtctgacggcaacgaaactggtagtcaggttgcttctgcatcacttgcaaagattgtcttcattttaaagcttctgacagagatattattgctgtattcatcatctgttcatgttctacttcgacgagatgctgaaataagctgcattagaggttcttatcaaaagagtcctgcaggtttaagcatgggttggatattttcccatattcttcataattttcttccatattctcgaaactcaaaaaaggacaagaaagctgatggtgattggaggcagaaactagcaaccagggccaaccagtttatagtgggtgcttgtgttcgatctacagaggcaaggaagagggtttttggtgagattagttatatcatcaatgaatttgttgattcatgtcatgacattaagcgtccaggaaatgaaattcaggtttttgttgatctactaaatgatgttttggctgctcgtacacctgctggttcgtacatttcagctgaggcctctaccacttttatagatgctggtttggttaaatcattcacttgcactctacaagttttggaccttgaccatgctggttcatctgaagttgctactggtattattaaagctcttgagttggtaaccaatgagcatgtccattcagttcattctagtgcagggaagggtgataattcaacaaaaccttctgttctaagtcaacctggaagaacaaataatattggtgaactgtctcagtccatggagacatcacaagccaatcctgattcccttcaagttgaccatgttgggtcttatgcagttcactcctatggtgggtctgaagctgttactgatgatatggaacatgatcaagatcttgatgggagctttgttcctgctaatgaggatgattacatgcatgaaaattctgaggatgcaagaaatcttgaaaatggaatggaaaatgtgggtctacaatttgaaatccaacctcatggccaagaaaatcttgatgaggatgacgatgaggatgatgatatgtctggagatgaaggtgaggatgtagatgaagatgatgatgatgaggaggaacacaatgatttggaagaagtccatcatttgccacatcctgacacagatcaagacgagcatgagattgatgatgaagattttgatgatgaagtgatggaggaagacgatgaggatgacgaggaagatgaagatggtgttatactgcgacttgaggagggaattaatggaattaatgtttttgaccatattgaggtttttggcagagataatagttttgcaaatgaagctttacatgtaatgccagttgaggtttttggatccagacgtccggggaggacgacatctatttatagtcttttgggcagaactggtgatgctgctgtgccttctcgtcacccactcttgcttgaaccttcttcattccctccacctacagggcaatcagatagttcaatggagaacaactcagtgggtttggataatatatttcgatcgctgaggagtgggcgccatggacaccgtttgcacttgtggactgataataaccagcaaagtggtgggacaaacactgctgttgtaccacaaggccttgaggagttgcttgtcactcaattaaggcgaccaacccctgaaaagtcatccaatcagaacatagcagaagcaggttctcatggtaaaattggaacaacccaggcacaagatgcagggggtgcaaggccagaagtccccgttgaaagtaatgctattctggaaattagtactataactccctcaattgataacagtaacaatgcggatgtcagaccagcagggactggaccttcacatacaaatgtttcaaacacccaatcacgggcagttgagatgcaatttgaacatactgatggagctgtgagggatattgaagctgtcagccaggagagtagtggtagtggagcaactttcggtgaaagccttcggagcttggaagttgagattggaagtgctgatggccatgatgatggtggtgaaaggctggtttctgctgataggatggcaggtgattcacaggctgcacgcacaagaagagcaaatacacctttgagtcacttttctcctgtggttggaagagatgtgtcccttcatagtgttactgaagtttcagaaaattcaagccgtgatgcagatcaacaaggtcctgcagcagagcagcaggtgaacagtgatgcgggatcaggagctattgatcctgcttttctggatgctcttcctgaggagctacgtgctgaagtcctttcagctcagcagggtcaagtggctcagccatcaaatgttgagtctcaaaacactggggatattgacccagagttcctagcagctcttccagctgatattcgagcagaagttctagctcagcagcaagcacagaggttgcatcagtctcaggagctggaaggtcaacctgtggaaatggatacagtctcaataattgcaacttttccatcagatttacgagaagaggttctcttgacatcaccagatactatccttgccaatcttacacctgctcttgttgctgaggcaaatatgttgcgggagaggtttgcacaccgttatagtcgtaccctctttggtatgtatccaagaagtcgtagaggggagacttcaagacgtgaaggtattggttctggtctggatggagcaggaggaaccatttcttctcgccgctccagtggagttaaggttgttgaagctgatggagcacctttagttgacacagaagctttgcatgctatgattcggttatttcgtgtagtgcagccactctataaaggccaactccagaggcttctattaaatctttgtgcccatagtgaaacaagaacctctctggtgaaaattctcatggacttgctaatgcttgatgtaaaaaggcctgtcagttattttagtaaagttgagccaccatatagattgtatggttgtcagagcaatgtaatgtattcacgtcctcaatcttttgatggagttcccccattgctgtctcgtagaatacttggaattctcacttatcttgctcgcaatcatctgtatgtggcaaaatttttgcttcagtgtaggctgtctcatcctgcaataaaagaaccagatgatccacggggaaaagctgtgatggttgttgaagatgaagtaaatataagtgaaagtaatgatgggtacatcgccattgcaatgctattgggtctgttgaaccaaccactttatttgaggagcatagcccaccttgagcagctgctagatttactggatgttatcattgacagtgctggaaacaagtcatctggcaaatccttgatacctactaacccatcatcagctccacaaatttctgctgcggaagccgatgcgaatgcagattctaacaatttaccttctgcggatgatgcatctaaagttgatggttcctccaaaccgacagtctctggcattaatgttgaatgtgagttacatggagtgttgagtaatcttccaaaagcagaactccggctcctgtgctcactgcttgctcaagaaggtttgtcagataatgcgtataatcttgtagcggaagtaatgaagaaattggtggccattgctccaacacattgtgagctttttgtcactgagctggcagaagcagttcaaaagttgacttcatctgcaatgaatgagttacgtgtctttagtgaagcaatgaaagctctgcttagtacctcttctactgatggagctgcaattttgagagtcttgcaagccttgagttcccttgtcaccttactgacggagaaagagaatgatagaggtactcctgctctttctgaggtttgggaaatcaattcagcattagaacccttgtggcatgagcttagttgttgcataagcaagatagaatcctactcagagtctgcatctgagttttcgacatcttctagtacctttgtgtctaaaccgtctggtgtaatgcctccacttccagctggctctcaaaatatcttaccatacattgaatctttctttgtggtttgtgagaaattgcatcctgctcagccaggtgctagtcacgactcaagtattcctgttatttcggatgttgagtatgccaccacatctgtaactccccagaaagcatctggaactgctgtgaaagtagatgagaaacatatgccttttgtccggttctcagagaagcacaggaagctactaaatgctttcataaggcaaaaccctggtttgcttgaaaaatctttctcactcatgctaaaggttccaagatttattgattttgataacaagcgtgcccacttccgatcaaaaattaagcatcagcatgaccatcaccatagtcccttgagaatatcagtaagaagggcatatgttctagaagattcttacaaccagcttcgcatgagatcaactcaagatttgaagggaaggttgactgtccacttccaaggggaggagggtattgatgcaggtgggcttacaagggaatggtatcaattattgtccagagttatttttgataaaggagcactgctttttactacagtgggcaatgaatcaacatttcagccaaaccctaactctgtttaccaaacagaacatttatcttatttcaaatttgttggtagagtggtcggtaaagcattatttgatggtcaactcttggatgttcattttactcggtcattctacaagcacattcttggagtcaaagttacatatcatgatattgaagccattgatcctgattatttcaaaaatttgaaatggatgcttgagaatgatatcagtgatgttctggatcttacttttagcattgacgcagatgaggaaaaattgatcttatatgaacgaacagaggtgactgattatgagttgattcccgggggacggaatatcaaagttactgaggagaacaagcatcaatatgttgatttggttgccgagcatcggttgacaactgctattcgacctcaaataaattatttcttagaagggttcattgaattgattcccagggagttgatatcgatattcaatgacaaagagctggaattgttgatcagtggacttcctgatattgatttggatgacttgagagcaaatacagaatattctggatatagtgctgcatcgccagttatccaatggttttgggaggttgttcaaggtttgagcaaagaagacaaggctcgactgttgcaatttgtgacaggcacatccaaggtgcctttggagggctttagcgctctccaaggaatttcaggctcccagaagtttcagatacacaaagcatatggaagtcctgatcacttgccttctgctcatacttgcttcaatcaattagatttgccggagtatccatctaaacaacatttggaagagaggttactgctggcaattcacgaagcaagtgagggttttggatttggttga

SEQ ID NO: 25 - DNA, 10959 bp, Glycine max; identical to SEQ ID NO: 24

SEQ ID NO: 26 - DNA, 10965 bp, Brassica napus
atgccttcccaagtccagcctcccaagatcaaatcgttcatcaatagcgtcactgctgttcccctcgaccaaattcaagaacccctttcctgttttcactgggatttcgacgacaagggtgacttccatcactgggtggatctcttcaatcatttcgacacatattttgagaagcacattaaagctaggaaggatcttcatgttgagcaacaagactctgaggacgaatctactcctcctctcccaaaggatgctcttcttcagattctccgtgttatccgagttgtgttagataactgcacaaacattcatttttttacttcttatgagcagcatctttctctcctgcttgcatctactgatacagatgtcgttgaagcctgtctgcagacgttggcttcctttttcaagaggcaaaatgatatttacttcataagagatgcttctcttaattcaaaactattttctcttgcccaaggctggggtggcaaagaggaaggtcttggcttgacatcatgtgctactacagaaaacacttgtgatctggtttctcacctccttggttctactcttcattttgagttttatgcttctggtgaatcatcaactgagcttccgggcggtttacaagttatccatctacctgatgtcagcttgcgtgcagagtctgatctggaacttctcaacaaattagtcactgaccataacgttcctcccagtttaaggttcgtgttgttgaccaggttgagatttgcaagggcgttttcatctttgtcgacccggctgcagtacacacgcattcgcttatatgcattcattcttttggttcaagctagtggcgacacccagaaagtggtttctttctttaatggagaacccgagtttgtaaatgagttagttacactgctgagctatgagactactgtcccagagaaaataaggctactgtgcctgctttccttggttgcattatcgcaagatcgaactcggcagacgactgtgttaactgcagtcacgtctcgtggtttactatctggccttatgcagaaagctattgattccgttctttgtaatacttccaagtcgtctctggcttttgcggaagccttgttatcccttgttactgtcttggtctcatcatcgtctggatgttcagccatgcaagaagctggtcttattccaattctagtgcctctcatcaaagataccgatccccagcacttgcatttggtcagtaccgctgtgcatatattagaagccttcatggattacagcaatccagctgctgctttgttcagagatttgggtggcttagatgatactatctttcggttgaaactggaagtctctcgtaccgaagacaatgtcaacgaaaaagtttgcggttcagacagtaatgggagggcttcacatgtccttggtgattctcttaatcggcctgatactgaacagcttccctattctgaggcattaatttcgtattacaggagattgttgttaaaagccttgttatctgcaatctctcttggaacttattctcctggtaatactaacctctatggttccgaggagagcttgctgcctgaatgcttatgcattatcttccggagagcaaaatatttcgggggtggagtattctcgcttgctaccactgtcatgagtgatctcattcataaagatccaacttgttttaatactttagactcgtctggtgtaacttctgcctttcttgatgctatctctgatgaggtcatctgctctgcgcaagccattacatgcatcccgcagtctctggatgctctgtgtctcaacaatagcggtcttcaagctgtcaaggaccgaaatgcactaaggtgttttgttaatatatttacttcttcgtcttatctgcgagctcttactggtgatacacctagtgctttgtcaagtgggcttgatgaactcctaagacaccaatcttcgttgcgcacttatggagttgatatgttcattgaaatcctgaactccatgttgattattggatctgggatggaggcctccacttctgtgtcagcagatgtgcctactgatgctgctaccgctcctatggaaattgacgctgatgagaagagcttggccatttcggatgaggcggaaccatcttctgctgcttctccagcaaatacagagttgtttcttccagattgtgtgtgtaatgttgctcgtctctttgaaatagttcttcagaatgcagaagtatgttctctatttgttgagaagaaaggaattgatgttgtcttgcagctactctctttacccgttatgcctctgtcaacctcctttggtcaaaacttttctgtcgcttttaagaacttctcccctcagcattcggctagtctatctcgtacagtgtgctcctacttacgagaacgtctgaagggaacaaatgagcttttgggtgccattaaaggcactcagcttcttaaactagagtctgcagtccagatgacgattttgagatcccttttctgcctagaaggcatgttgtccctctcaaactttctgttgaaaggaacatcttcagttatcgcggaattaagtgctgctgatgctgatgtactaaaagaacttggtctaacatacaagcaaataatttggcagatggctttgtctagtgagaccaaggaagatgagaaaaagagtgttgatggaggacctgataattcaattttagcctcatctagtactgttgaaagagagagtgaagaggactcaagaaatgcttcagcagttagatacacaaaccatgtatccattagaagaagtacctctcaatctatttggcgtggtggtcgtgatctgtctgttatgcgttccatcgagagtatgcatggtcgtacacgacaagcgatttcccgaacgaggggtgggagaactcgtcgacacctggaggcttttaattttgattctgaaattccacctgatttaccaggtacatcatcttcccatgagctgaaaaagaaaagcactgaagtcctgactgttgaaattttagacaagttgaattgtactctgcgtctttttttcactgcccttgtgaaaggaggattcacctctgcgaatcgtcgcagaattgatggagcaccactgagttccgcatctaagaagacgcttggtaatgccatagctaaagtatttcttgaagctcttaacttcgatgggaatggtgttactgctgagcatgatatatttctgtccgtaaagtgccgataccttggaaaagtggtagatgacatggcttccctgacatttgatactcgaagaagggtctgtttcacagctatgattaatagtttttatgtccatggaacatttaagcaacttctcaccacatttgaagcgacaagccagttgctttggacagtgccgttttctgttactgcatctgatactgagaatgagaagccaggtgaaaggaacatatggtctcgcaagacgtggctggtggatactctgcaaatctattgccgagcactggactattttgttaactctacatttctgttatctccagcctccacttctcaaacgcagcttcttgtccagcaagagcaagcttcaattggtttgtcgatcgaactccatcctgtaccaagggaacctgaaactttcgtgcgaaatctgcagtctcaggttctggatgtcatactacctatatggaaccaccctatgtttcctgattgcaatcctaattttgtggcttcggttacctcccttgttacgcatatatactctggtgttgtggatgctacgcaaaatcaagcccggggtacaaaccaaagagccttgcctctacagcctgacgaaaccattgttggtatgattgttgaaatgggattttcaaggtcaagggcagaatacgcgttacgaagagttggaacaaacagtgttgaaatagctattgagtggttgtttgccaatcctgagcatactgtgcaggaagatgacgagctggcccaagcacttgcactatctcttggcaatgcatccaaaactccaaaacctgtagatgtccctctggaagaagcggatccaaaagaaccatctgttgatgaagttattactgcatcggtgaagttatttgaaagtgatgattctatggctttcccattgatggatttgtttgtaacactttgtagccgaaacaaaggggaagatcggccgaaaattgtgtcgtttcttatacagcaactgaagctagtacaagttgatttctccaaggatactggtgctttgactatgctaccacacattctagcattagttctctcagaggatgacaacacacgagaaattgctgcacaggatggaattgtgaccgtagcaattgatatcttgacgaatttcaagcttaagagtgaatctgaaagtcagattctggctccaaaatgcattagcgctttacttcttatcttgagcatgatgctgcaggctcggacaagaatctcgtctgaatttttggaaggaaatcatggtggatctttggagccgagtgattatccgcaagactcagcagcagcgttaaagaaagtgttatcttcagatgttgctaaagaggagtcgaaaccggatttggaatcagtttttggaaaatctacaggctatctgaccatggaagagggtcaaaaagctctactaatcgcatgtggcctcgtaaagcagtgtgttccagaaatgatcatgcaggctgttcttcagttatgtgcacgtctaactaaaactcatgctttagctatccagtttctggaaaatggagccttatcctcactttttaatcttcccaaaaaatgtttcttccctgggtatgatactgttgcatctgttattgtacgtcatctggttgaagatccacagactctccaaattgctatggaatcagaaatacgacagaccttgagtggaaagagacatgtaggtagggtattacctcagacatttctgacaacaatggcacctgtaatttcgagagatcctgtggttttcatgaaagccgtggcttctacttgtcagctggagtcatcaggagggagggactttgtgattccgtcgaaggaaaaagaaaagccaaaagtttccagcagtgagcagggattgcctctgaatgaaccccttcgaatatccgaaaataagcttcatgatgggtcagggaaatgttcgaaaagccacagacgagtccctgctaatttcatccaagttatcgatcagcttattgatattgtcttaagttttcctagggtgaagaggcaggaagatgatgaaaccaatttaattgcaatggaagttgatgtgccggcaactaaagtgaagggtaagtcaaaagttggtgatccagaggaagcagaatttggatctgaagaattggccagggtaacatttattttgaaattgttgagtgatattgttatcatgtacttgcacggtaccagtgtcatactgaggcgggatacagaaatatctcagcttcggggatccaatctacccgataattcacctggcaatggagggttaatttaccatatcattcaccgattacttcctatatcgctcaaaaattctgttggatctgaagtttggaaagagaagttgtctgaaaaagcttcctggtttctggtcgttttttgcagccgttccagtgagggacgtagaagaataatcagtgagctttcgagtgttttatctgtattggcttccttgggaaagagttcttctagtaaaagtgttctgttacctgataaaagagttcttgcttttgctggcctggtttattcgatattaacaaagaattcatcttccagcaacttacctggttgtggttgctcacctgacgttgcaaagagcatgatagatgggggaattattaagtgtctgaccagcattcttcacgtaattgacctcgaccaccctgatgctccaaagcttgtcactcttattctcaagtctcttgagacactgacgagtgctgcaaatactgctgagcagctaaaatcagcagggtcaaacgagacgaagggcacagattctaatgagagacatgacagtcgtggaacttcaactgaggctgaagttgatgagtcaaaccgaaacaatagcagtctacaacaagtaactgatgccgcagagaatggacaggagcaccctcaaatttcctctcaaagcgaaggtggaaggggttcgagtcaaacccaggctatgcctcaagagatgaggatagaaggcgaggagacaatactgcctgaacctattcagatggatttcatgggagaagaagatgaccaaattgaaatgaattttcatgttgaaaatagggccggagatgatggagatgatgccatgggagacgaagaggatgatgatgaggaaggatttgatgacatcggacccgaactggaggatgatgaggatgcagatttagtggcagacggagctcggagtgttatgtctctttctggaactgatgccgaagaccctgaagatactggcctcggagatgaatataatgatgacatgattgacgaagatgaggatgatatccacgagaatcgtgtaatagaggtgcagtggagggaagctcttgatgggctggatcattttcagattcttgggcgatctggtggtggaaatgaatttattgatgactttgaaggaatgaatatgggcgatctggttactctgcagagacccggctttgatcgtagacgtcaagcagacataaattctttccatcgatctggttcccaagtacatggctttcagcatccgctcttctcgagacctttgcgaactggcaatacggcctcagtttcagcaagtgctggcaggaatgatatatcacagttttacatgtttgatatgccggttataccatttgatcaagtaccaagtaatcctttcagtgatcgcttaggaggtagtggggcacctcctcctttgactgattattctgtggtggatatggattcatcaagaagaggggttggtaatagtcggtggactgatataggtcaccctcaaccaagtagtcaatctgcgtcgattgcccaactgatagaagaacattttatttccaaccttcgtgcttctgcgctagcagatagtgttgtcgaaagggaaactaatagcacggaagtccaagagcagcagcatccatctgttggaagcgaaagcgttttgggggatggtaacgacggtggtcaacaaagtgaagcgcatgaaatgttgaataataatgacaatgttgataacccacctgatgtaacggctggaattttctcccaagctcgagcaaatctagcttcccctgtacttctgcagcctcttcctatgaacagtacaccaaatgagattgacagaatggaagttggggaaggtgatggagtacctattgagcaagcagatgtcgtagctgtggatcttgtctccactgcccagggccaacctgatacgtccagtagtcaaaatgtctctggtatggggacgccaattccagtagatgatcccatttccaattgtcaaccaagtggggatgtacatatgagtagtgatggtgcagagggaaatcaaagtgtggaaccttcactattatcccgtgataacaatgagctctcatccagggaagctacccaagatgcgagtaatgatgagcaacttgctgaaggtagcttggagttggacggtagggcacccgaagcgaattccatcgatcctacatttttagaggcgctccctgaagaattacgggcagaagttcttgcttctcagcaagctcagtccgttcagcccccaacttatgaaccaccttcggtagaagacatagatcctgaatttttggcagcgcttcccccagatatccaaacagaagttcttgctcaacaaagggtacaaaggatggcacatcagtcacaaggacagccaactgacatggataatgcttcaattattgctaccctacctgccgatttacgtgaagaggttctcttaacttcttcagaggcagttttggcagcgttgccttcacctttacttgcagaagcgcagatgctcagagaccgagcaatgagtcactatcaggctcgtagccatagtaatcgaaggaatggtttgggttacaataggctgacggggatgaacaggaacgtcggagtcactattggtcagagggatgtttcatcttttgcagatggcttgaaagtaaaagagatggaaggagaccgtcttgtggatgtcgaggccttgaaatcactaattaggctactacgacttgcacagccgttggggaaaggccttctgcataggcttctcttcaagctgtgtgctcaccgtggtacaagagccaacttggttcaacttctgttggatttgattaggccagagatggaaacatcaccgagcgagttggcaataagtaatcagcaaagactctatggctgtcagtcaaatgttatttatggacgatcccagctgttgaatggtcttcctcctctagtgttccgtcgggtgctagaggttctgacgtatttggctacgaatcattcggctgttgctgacatgttgttctactttgattcgtcacttgtgtcccaattgtcaaagccaaaaccctctgtatgtgaaggcaagggtaaggagactgttactcatgtgacagactcccggaatctggagatacctctcgttgtcttcctaaagctgcttaatcggcctcagcttttgcaaagtacatcccatctagcactggtcattggtttactgcaagaagttgtctacaccgcagcatcccgaattgagggttggtctccgttatcaagtttatctgagaaatcagaagagaaaccggttggtgaagaagcttcaagtgaaacacgaaaagatgcgaagtctgagcaagtggatgaagctgataagcaatctgttgcaagagtaaagaattgtgctgatatatataacatattcttgcagttgccacagtccgatctctgcaatctttgcctacttcttggatatgaagggttatcggataaaatttaccttttagcaggaaaggtgataaaaaagctggctgccgtagatgtggctcatcggaggtttttcgcaaaagaactctcacagttggcaagcgggttgagtgcctcaactgtccgcgagctggcaacactgagcaatacagagaagatgagtcacagtacaggttccatggcaggtgcttcacttctccgtgttctacaggttcttagctcactaacttccactattgatgatggcaatcctggaaccgaaaaggaaacagaacaggaggaacaaaacattatggagagactaaacatggcattagagcccctttggcaggaacttagccagtgtatcagcatgactgaggtgcagctggatcatacttcagccacaacaaccgtgtccagtgtaaaccccggtgatcatgccctaggggtcactgctccgtcccctatttctccgggaactcagaggttcctacctcttattgaggctttctttgttctgtgtgagaaaattcaaactccgtcaatactacatcaggatcaggcgaatgtgacagctggagaagtaaaggagtctgctcttagtttatcatctaagaccagtgtagattctcagaagaaaattgatggctcccttacatttgcaaagtttgcggagaagcataagcgacttttgaattcatttgttaggaaaaacccaagtttactggagaagtccctttcaatgatgctcaaggcaccaaggctgattgattttgacaacaagaaagcttacttcaggtcaaggataaagcaccagcatgatcaacacatttctggtccattgcgtatcagtgtccgccgagcttatatgttggaagattcatacaaccagttacgtatgcgctccctacaggatctgagaggacgtctgaatgtgcagtttcaaggtgaagaaggtgttgatgctggtggtcttacaagagaatggtatcagttagtgtcaagagttatatttgacaaaggagcgttgcttttcactaccgttggaaatgatgccaccttccagccgaatcccaactctgtttaccaaaatgagcatctgtcatacttcaaatttgttggtcgcatggtggcaaaggcgttgtttgatgggcagcttttggatgtttattttacgcgctccttctataaacacatacttggtgtgaaggtaacctatcatgacattgaggcggtggatcctgattactacaagaacttgaagtggctgttagagaatgatgtgagcgacatactcgacctcacatttagtatggacgcagatgaggaaaaacacattctatacgaaaagactgaggtgacggactatgagcttaaacctagaggaagaaacatacgggtaacagaggaaacaaagcatgaatatgttgaccttgtggccggacacatacttaccaatgctattcggcctcaaataaacgccttcctggaaggctttaatgagttaatacctcgtgagctcgtatccatttttaatgataaagagctcgagctcctaatcagcggattgcctgagattgatttcgatgatcttaaagccaataccgagtataccagctacacggctggatcccctgtgattcattggttctgggaggtcgttaaagcttttagcaaggaagacatggctagatttcttcaatttgtcaccggaacatcaaaggttcctttagaaggtttcaaggcactgcaaggtatttctggacctcaaagattacaaatccacaaggcatatggaggtccggagcggctgccatcagctcatacatgttttaaccaactagaccttccagagtatccatctaaggaacaacttgaggaacgtctgctacttgccattcacgaagccagtgaaggtttcgggtttgcttga

SEQ ID NO: 27 - DNA, 20 bp, Oryza sativa
ggcagtcttcgttctcgtgt

SEQ ID NO: 28 - DNA, 20 bp, Oryza sativa
ggcaggtcccgcctctaatc

SEQ ID NO: 29 - DNA, 20 bp, Oryza sativa
gtgccgggccggttaacaat

SEQ ID NO: 30 - DNA, 20 bp, Oryza sativa
ggcgcggcgggttacctcta

SEQ ID NO: 31 - DNA, 20 bp, Oryza sativa
ggagggcccccgatcgcggc

SEQ ID NO: 32 - DNA, 2630 bp, Oryza sativa
ggggatgcaggaactgcattctttcatttgaagataaaggcgagaagcaggaagcttctcattccaatccttgagcatgatggcaggattgccaccacccagcatgacatgcaaagtttggcacgagaatactttgctgcagtgatgtgccctgagtgcagtgacacgaagttgctgcaatttcaccatattcagatggcaacaactgatctctccagcctcgacagtcctttcactgaagatgagatttggtcggctatccgtgctttgcctaatgaaaagtcgccagggccggatggttatacaggcttgttttaccaaagatgttgggagataattaaacctgaattgatcagcgctcttgctaaattctgtaccggtaacagtcagaacttggagaaactgaattcggcaattgtcacgctaataccgaagaaggacagtcctaccctcctcaaggattataggccaattagtttgattcatagtttctctaagatagctgcgaagattatggcgcagcggttagcaccgaagctgaatgtcctcattccatcctcccaaactgcttttatcaagggacgctgcatacacgagaactttgtcttcgtcaaaggattggtacaacaatttcacagacaaaggaaggctatgatgttgctgaaattagacatctcgaaagctttcgacactgtctcctggggttttcttatgtcgatgttacagttcagaggctttggtccactttggagaagatggctctcggcggtttttctcactgcagaaacaagaatattgataaatggtgttctgtctgacacaatcaagccggcgagggggttgaggcagggtgacccactgtcgccgctgctctttgttctagtaatggatgccttgcaagctattgtttcccaggcaaggatggcaagactgctctcgcccctcaacgtacgacagaatttgccaccaatttcagtttatgccgacgatgcggttctgtttttccgccctacagctgaagaagctcgagtcatcaagggtatcctggagttgttcggggctgccacaagtctcaaaaccaatttctccaaaagcgcaatcactccaatccaatgtgacgagcagcagtatgtgcaagttgaatccattctctcctgccgagtggaaaagttcccaatcacttatcttggactccctctctccactaggaaaccaacgaaggccgagatccagccgatccttgataggctggcaaagaaggtagccggttggaagccgaaaatgctgtctattgatgggcgactgtgcttgatcaagtcggtcctaatggcgctgccggtgcactacatgacagtcctgcagctaccgcgatgggcgattaaggacatcgagcggaagtgccgtgggtttctttggaaaggacaggaagagatcagcggcgggcattgcctagtctcgtggcgaaaggtttgctcacccatcgagaaagggggacttggtgtcaaagatcttaatttgttcggtcaagctctccggttgaaatggcttgcaaaatccttggagcagaaggatagaccctggaccttagcaactttccgtcctggaagcgatgtggaagagatctttcgatccgttgctgagcacatcattggtgacggggtgaacacacagttttggacagacaattggacagggaaaggttgcttcgcctggaggtggccggtgttgttttcccatgtgagccgtgccaagctgacagtagctgatgccctgattgctaacagatgggttcgccgattacaaggtgccttgtccaatgaagctctgggtgaattcttccaactttgggatgaagttcacgacgtgtcactgcagcagatggctaaaacgatcaaatggaagttgactgttgatggtaatttctcagtggcctcggcgtatgatctatttttcatagcgacagaggactgttcctacggggacacgctgtggcactccagggtgccgtcgcgtgttcgcttcttcatgtggattgcactcaagggccgctgtctcacggcggacaacctggcaaagagaaactggccgcatgacgccatttgctccctatgccaacacgagaacgaagactgccattatttgcttgtgtcctgtgattatacggcggcggtttggcgcaagctgagacgttggtgcaacattaacattgcaatccctgcggaagatggcatgccgcttgcagattggtggatcgcgacaagacggcgttttcagaacacgtataggacggatttcgatagtctgttaatgctaatttgttggcttatctggaaagagcgaaatacaaggatctttcaacacatcgccaagtcggttgaccggctagcggatgacatcaacgaggaaatcgcaatttggagggcagcagggattttctcccaggctagcgagtaatcccgattagaggcgggacctgccccattttttccttttctttccgggcttgagtttgcttgagaccggcgcgacatccttcatgtcgttgtaattaaaactttatttccctcaatcttaataaaattggccggcctacctttggccgtcccggcaaaaaagaatctagaatatat

SEQ ID NO: 33 - DNA, 23 bp, Triticum aestivum
  misc_feature (21)..(21): n is a, c, g, or t
gtgcttattcccagcagacangg

SEQ ID NO: 34 - DNA, 23 bp, Triticum aestivum
  misc_feature (21)..(21): n is a, c, g, or t
gccagacctgcaccttcggangg

SEQ ID NO: 35 - DNA, 23 bp, Triticum aestivum
  misc_feature (21)..(21): n is a, c, g, or t
gagcgagctaggatactgagngg

SEQ ID NO: 36 - DNA, 23 bp, Triticum aestivum
  misc_feature (21)..(21): n is a, c, g, or t
gtcgcttctgtgagtacagangg

SEQ ID NO: 37 - DNA, 20 bp, Triticum aestivum
gtgcttattcccagcagaca

SEQ ID NO: 38 - DNA, 20 bp, Triticum aestivum
gccagacctgcaccttcgga

SEQ ID NO: 39 - DNA, 20 bp, Triticum aestivum
gagcgagctaggatactgag

SEQ ID NO: 40 - DNA, 20 bp, Triticum aestivum
gtcgcttctgtgagtacaga

SEQ ID NO: 41 - DNA, 23 bp, Zea mays
  misc_feature (21)..(21): n is a, c, g, or t
ggactacggttagaggctcangg

SEQ ID NO: 42 - DNA, 23 bp, Zea mays
  misc_feature (21)..(21): n is a, c, g, or t
gtgcaatccctgagaagtatngg

SEQ ID NO: 43 - DNA, 20 bp, Zea mays
ggactacggttagaggctca

SEQ ID NO: 44 - DNA, 20 bp, Zea mays
gtgcaatccctgagaagtat

SEQ ID NO: 45 - DNA, 23 bp, Pennisetum glaucum
  misc_feature (21)..(21): n is a, c, g, or t
gcctgcagcagatcctggccngg

SEQ ID NO: 46 - DNA, 23 bp, Pennisetum glaucum
  misc_feature (21)..(21): n is a, c, g, or t
gcactggctatgctagcgctngg

SEQ ID NO: 47 - DNA, 20 bp, Pennisetum glaucum
gcctgcagcagatcctggcc

SEQ ID NO: 48 - DNA, 20 bp, Pennisetum glaucum
gcactggctatgctagcgct

SEQ ID NO: 49 - DNA, 20 bp, Glycine max
gcgccaacttctgtccagcg

SEQ ID NO: 50 - DNA, 20 bp, Glycine max
cattggtccttcagtcaagg

SEQ ID NO: 51 - DNA, 20 bp, Glycine max
ctatctcctgtccctagcac

SEQ ID NO: 52 - DNA, 20 bp, Glycine max
gaacgggcacggatactgag

SEQ ID NO: 53 - DNA, 20 bp, Glycine max
cgccaacttctgtcgagcga

SEQ ID NO: 54 - DNA, 20 bp, Glycine max
tcgaaggccaactcgatctt

SEQ ID NO: 55 - DNA, 20 bp, Brassica napus
gggttcttgaatttggtcga

SEQ ID NO: 56 - DNA, 20 bp, Brassica napus
gcaagctgacatcaggtaga

SEQ ID NO: 57 - DNA, 20 bp, Brassica napus
ggttcttgaatttggtcgag

SEQ ID NO: 58 - DNA, 918 bp, Oryza sativa
aaaaataccatcctgcagattttgagagtaatgcagattgttttggaaaattgccagaacaaaacatcgtttgctggtcttgagcattttaggcttctgctggcatcatcagatcctgagatagttgtggctgctttagagacacttgctgcattggttaaaataaatccttcgaagttgcatatgaacggaaagctcataaattgtggagctataaacagtcatcttctatcattggcacaaggatggggtagcaaggaggaaggtttgggcttatattcttgtgttgtggcaaatgaaagaaaccagcaggagggtttgtgcttattcccagcagacatggagaacaaatacgatggcacgcagcaccgtctcggttcaactcttcattttgaatataatttggcacctgcccaagatcctgaccaatccagtgacaaggctaagccatctaatctgtgtgtgatacatatcccagacttgcaccttcagaaggaggatgacttgagcatattgaagcaatgtgttgataagtttaatgtgccttcagagcacagattttccttgtttacaaggataagatatgcccatgcctttaattcgccacggacatgtaggctatatagccgcataagtcttcttgctttcattgttcttgtgcaatccagcgatgcccatgatgaactcacatctttctttacaaatgagccagagtacataaatgagttaatcagacttgtccgatcagaggaatttgttcctggacccatacgagcgctggctatgcttgcactgggagcacagttagcagcgtatgcatcatctcatgaacgagctcggatacttagtggctcaagtatcatatctgctggtggaaaccgcatggtcttgctcagtgttttgcaaaaagctatatca

SEQ ID NO: 59 - DNA, 941 bp, Oryza sativa
gcagtgaaaactcttcaaaagttgatggagtacagcagccctgctgtttctctatttaaagatttgggtggtgtagaacttttgtctcagaggttgcacgtggaggtgcagcgtgttattggtgttgacagtcataattcaatggttacaagtgatgcattgaaatcagaagaggatcatctctactctcagaagcgattgattaaggcgctgctaaaggcattggggtctgctacatattctcctgcaaatcctgctcgttcacaaagctcaaatgataattctttgcccatctcgctttcccttatatttcagaatgttgacaagtttggtggtgacatttatttctcagcagttactgttatgagtgagataattcacaaggatccaacatgctttccttctttgaaggaacttggtcttccagatgcttttctatcgtcagtgagtgctggggtaataccatcttgtaaagctctcatctgtgtgcctaatggtctgggtgcaatatgccttaataaccaaggacttgaggctgtcagggaaacttcagctctgcgttttcttgttgacacattcaccagcaggaagtacttgataccaatgaatgaaggtgttgtcctattagctaatgcagtggaagagcttctacgtcacgtgcagtccctaagaagcactggggttgacatcattattgaaataattaataaactttcttcacctcgtgaagataagagcaatgaaccagcggccagttctgatgaaagaacagaaatggaaactgacgcggaaggacgtgatttggtaagtgctatggattccagtgaggatggcactaatgatgaacagttttctcatttgagcattttccatgtgatggtattggttcatcggacaatggagaactccgaaacctgccggttatttgtggagaaaggagg

SEQ ID NO: 60 - DNA, 114 bp, Oryza sativa
aatgcaatttctctgattgtagagatgggcttttctcgcgccagagctgaggaagcactcaggcaagttggaacgaacagtgttgaaattgcaactgattggttattctcacac

SEQ ID NO: 61 - DNA, 105 bp, Oryza sativa
aacagagctgctgacactgactcaattgatcctacatttttggaggctcttccagaggatttacgggctgaagttctttcttcacgtcaaaatcaagtgacccag

SEQ ID NO: 62 - DNA, 75 bp, Oryza sativa
gaacaacctcagaatgatggggatattgatcctgaattccttgctgcacttcctcctgatatacgtgaagaagtt

SEQ ID NO: 63 - DNA, 261 bp, Oryza sativa
atcagatttgaaattccacgaaatagagaggatgatatggctgatgatgacgaggacagtgatgaggacatgtcagccgatgatggtgaggaggttgatgaagatgaagacgaggatgaggatgaagagaacaacaacctggaggaggatgatgcccatcaaatgtctcatcctgacacagatcaggaggaccgtgagatggatgaagaggagtttgacgaggatctgctagaagaagatgatgatgaggatgaggatgag

SEQ ID NO: 64 - protein, 362 aa, Oryza sativa
RISVRRAYILEDSYNQLRMRSPQDLKGRLTVHFQGEEGIDAGGLTREWYQLLSRVIFDKGALLFTTVGNDLTFQPNPNSVYQTEHLSYFKFVGRVVGKALFDGQLLDVHFTRSFYKHILGVKVTYHDIEAIDPAYYKNLKWMLENDISDVLDLSFSMDADEEKRILYEKAEVTDYELIPGGRNIKVTEENKHEYVNRVAEHRLTTAIRPQITSFMEGFNELIPEELISIFNDKELELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKEQLQERLLLAIHEANEGFGFG

SEQ ID NO: 65 - DNA, 19 bp, Artificial Sequence
  CRISPR construct target sequence for OsUPL2/LARGE2 promoter
tagaatatatctgagggaa

SEQ ID NO: 66 - DNA, 19 bp, Artificial Sequence
  CRISPR construct target sequence for OsUPL2/LARGE2 promoter
gtgaaaggactgtcgaggc

SEQ ID NO: 67 - DNA, 19 bp, Artificial Sequence
  CRISPR construct target sequence for OsUPL2/LARGE2 promoter
atattctcaaaatcgaatc

SEQ ID NO: 68 - DNA, 19 bp, Artificial Sequence
  CRISPR construct target sequence for OsUPL2/LARGE2 promoter
aatcgaatctggactgttt

SEQ ID NO: 69 - DNA, 106 bp, Artificial Sequence
  sgRNA Sequence
gttttagagctagaaatagcaagttaaaataaggctagtccgttatcaacttgaaaaagtggcaccgagtcggtgcttttttttttcgttttgcattgagttttct

SEQ ID NO: 70 - DNA, 19 bp, Artificial Sequence
  CRISPR construct target sequence for OsUPL2/LARGE2 promoter
gcagtcttcgttctcgtgt

SEQ ID NO: 71 - DNA, 19 bp, Artificial Sequence
  CRISPR construct target sequence for OsUPL2/LARGE2 promoter
gcaggtcccgcctctaatc

SEQ ID NO: 72 - DNA, 19 bp, Artificial Sequence
  CRISPR construct target sequence for OsUPL2/LARGE2 promoter
tgccgggccggttaacaat

SEQ ID NO: 73 - DNA, 19 bp, Artificial Sequence
  CRISPR construct target sequence for OsUPL2/LARGE2 promoter
gcgcggcgggttacctcta

SEQ ID NO: 74 - DNA, 19 bp, Artificial Sequence
  CRISPR construct target sequence for OsUPL2/LARGE2 promoter
gagggcccccgatcgcggc

SEQ ID NO: 75 - DNA, 106 bp, Artificial Sequence; identical to SEQ ID NO: 69
  sgRNA

SEQ ID NO: 76 - DNA, 25 bp, Artificial Sequence
  OsU3-FD3 Primer
gacaggcgtcttctactggtgctac

SEQ ID NO: 77 - DNA, 25 bp, Artificial Sequence
  TaU3-RD Primer
ctcacaaattatcagcacgctagtc

SEQ ID NO: 78 - DNA, 25 bp, Artificial Sequence
  RC Primer
gactagcgtgctgataatttgtgag

SEQ ID NO: 79 - DNA, 25 bp, Artificial Sequence
  TaU3-FD Primer
ttagtcccacctcgccagtttacag

SEQ ID NO: 80 - DNA, 25 bp, Artificial Sequence
  TaU3-FD2 Primer
ttgactagcgtgctgataatttgtg

SEQ ID NO: 81 - protein, 362 aa, Oryza sativa
RISVRRAYILEDSYNQLRMRSPQLDKGRLTVHFQGEEGIDAGGLTREWYQLLSRVIFDKGALLFTTVGNDLTFQPNPNSVYQTEHLSYFKFVGRVVGKALFDGQLLDVHFTRSFYKHILGVKVTYHDIEAIDPAYYKNLKWMLENDISDVLDLSFSMDADEEKRILYEKAEVTDYELIPGGRNIKVTEENKHEYVNRVAEHRLTTAIRPQITSFMEGFNELIPEELISIFNDKELELLISGLPDIDLDDLKANTEYSGYSIASPVIQWFWEIVQGFSKEDKARFLQFVTGTSKVPLEGFSALQGISGPQRFQIHKAYGSTNHLPSAHTCFNQLDLPEYTSKEQLQERLLLAIHEANEGFGFG

SEQ ID NO: 82 - protein, 160 aa, Oryza sativa
RISVRRAYILEDSYNQLRMRSPQLDKGRLTVHFQGEEGIDAGGLTREWYQLLSRVIFDKGALLFTTVGNDLTFQPNPNSVYQTEHLSYFKFVGRVVGKALFDGQLLDVHFTRSFYKHILGVKVTYHDIEAIDPAYYKNLKWMLEVNIFFPVQWLTQTLDY

SEQ ID NO: 83 - protein, 156 aa, Oryza sativa
RISVRRAYILEDSYNQLRMRSPQLDKGRLTVHFQGEEGIDAGGLTREWYQLLSRVIFDKGALLFTTVGNDLTFQPNPNSVYQTEHLSYFKFVGRVVGKALFDGQLLDVHFTRSFYKHILGVKVTYHDIEAIDPAYYKNLKWMLERCSGPLLQHGCR

(19) United States
(12) Patent Application Publication
(10) Pub. No.: US 2025/0304988 A1
(43) Pub. Date: Oct. 2, 2025

(54) METHODS OF CONTROLLING GRAIN SIZE

(71) Applicant: INSTITUTE OF GENETICS AND DEVELOPMENTAL BIOLOGY CHINESE ACADEMY OF SCIENCES, Chaoyang District, Beijing (CN)

(72) Inventors: Yunhai LI, Chaoyang District, Beijing (CN); Shanguo YAO, Chaoyang District, Beijing (CN); Luojiang HUANG, Chaoyang District, Beijing (CN); Ruci WANG, Chaoyang District, Beijing (CN); Ran XU, Chaoyang District, Beijing (CN); Kai HUA, Chaoyang District, Beijing (CN)

(21) Appl. No.: 18/259,016

(22) PCT Filed: Dec. 23, 2021
(86) PCT No.: PCT/EP2021/087532

§ 371 Date: Jun. 22, 2023

(30) Foreign Application Priority Data
Dec. 23, 2020 (WO) PCT/CN2020/138605

(51) Int. Cl. C12N 15/82; C12N 9/10; C12Q 1/6895

(52) U.S. Cl. CPC C12N 15/8261; C12N 9/104; C12Q 1/6895; C12Q 2600/13

(57) ABSTRACT

The invention relates to methods of increasing plant yield, and in particular grain or seed number by introducing at least one mutation into at least one UPL2 gene. Also described are genetically altered plants characterised by the above phenotype.

### REFERENCE TO AN ELECTRONIC SEQUENCE LISTING

The contents of the electronic sequence listing (Sequence Listing.txt; Size: 479,285 bytes) was created on Dec. 21, 2023 is herein incorporated by reference in its entirety.

### BACKGROUND

The invention relates to methods of increasing plant yield, and in particular grain or seed number by introducing at least one mutation into a UPL2 gene and/or promoter. Also described are genetically altered plants characterised by the above phenotype.

Grain crops, which include cereals, legumes and oilseed crops, represent a crucial element of the world's food supply. Grain number per plant is a primary determinant of crop yield, and is influenced in large part by the floral architecture of the inflorescences of the plant. Rice for example, is one of the most important cereal crops in the world, and nearly half the world's population feed on rice (Zuo and Li, 2014). Rice grain number is basically determined by inflorescence (panicle) architecture, which refers to the number and length of primary branches and secondary branches, and the number of branches on secondary and higher order branches (Sakamoto and Matsuoka, 2008). Elucidating the genetic and molecular mechanisms of panicle architecture control, and analogous inflorescence structures in other species, is of great importance for high-yield breeding in grain crops. During past decades, several genes involved in the regulation of inflorescence size and grain number have been identified in rice, but the genetic and molecular mechanisms of inflorescence size and grain number control, and the interplay between them, are still not well understood.

In view of the above, there is a need to be able to increase grain number and therefore overall yield, particularly in the important grain crops. The present invention addresses this need.

### SUMMARY

Here we report that LARGE2, which encodes a functional HECT-domain E3 ubiquitin ligase UPL2, regulates panicle (i.e. inflorescence) size and grain number. LARGE2 controls inflorescence size and grain number by influencing meristem activity. LARGE2 associates with APO1 and modulates its stability. Genetic analyses support that LARGE2 acts in a common pathway with APO1 and APO2 to regulate inflorescence size and grain number. These findings reveal a novel mechanism of regulating inflorescence size and grain number control involving the LARGE2-APO1/APO2 regulatory module. We further report that introducing a loss of function mutation into UPL2, increases 1000 grain weight and overall yield.

Accordingly, in a first aspect of the invention, there is provided a genetically altered plant, plant part or plant cell comprising at least one mutation in at least one UPL2 gene and/or UPL2 promoter.

In a further aspect of the invention, there is provided a seed obtained or obtainable from the plant of the invention.

In a further aspect of the invention, there is provided a method of increasing yield in a plant, the method comprising reducing or abolishing the expression of a UPL2 nucleic acid and/or reducing the activity of a UPL2 polypeptide in said plant.

In a further aspect of the invention, there is provided a method of producing a plant with increased yield, the method comprising introducing at least one mutation into a least one nucleic acid sequence encoding a UPL2 gene and/or UPL2 promoter. In one embodiment, the method may comprise introducing at least one mutation into a least one nucleic acid sequence but preferably all copies or homeoalles of a nucleic acid sequence encoding a UPL2 gene and/or UPL2 promoter in a first plant and crossing the first plant with a wild-type or contrpl second plant to produce a F1 hybrid plant that is heterozygous for the mutation.

In a further aspect of the invention, there is provided a plant, plant part, part cell or seed obtained by the method of the invention.

In another aspect of the invention, there is provided a method for identifying and/or selecting a plant that will have an increased yield phenotype, the method comprising detecting in the plant or plant germplasm at least one polymorphism, wherein the polymorphism is a mutation in the UPL2 gene or promoter and selecting said plant.

In a further aspect of the invention, there is provided a nucleic acid construct comprising a nucleic acid sequence encoding a sgRNA, wherein the sgRNA comprises a sequence selected from SEQ ID NO: 27, 28, 29, 30, 31, 33, 34, 35, 36, 41, 42, 45, 46, 49, 50, 51, 52, 53, 54, 65, 66, 67, 68, 70, 71, 72, 73 or 74 or a variant thereof.

In another aspect of the invention, there is provided a genetically altered plant expressing the nucleic acid construct of the invention.

### BRIEF DESCRIPTION OF THE DRAWINGS

The invention is further described in the following non-limiting figures:

FIG. 1. The large2 mutants form large panicles and wide leaves and grains.

(A) Plants of KY131, large2-1, KYJ, large2-2, ZHJ, and large2-3 at the mature stage. (B) panicles of KY131, large2-1, KYJ, large2-2, ZHJ, and large2-3 at the mature stage. (C) Flag leaves of KY131, large2-1, KYJ, large2-2, ZHJ, and large2-3. (D) Mature grains of KY131, large2-1, KYJ, large2-2, ZHJ, and large2-3. (E) panicle length of KY131, large2-1, KYJ, large2-2, ZHJ, and large2-3 (n≥16). (F) Number of primary branches of KY131, large2-1, KYJ, large2-2, ZHJ, and large2-3 panicles (n≥16). (G) Number of secondary branches of KY131, large2-1, KYJ, large2-2, ZHJ, and large2-3 panicles (n≥16). (H) Grain number per panicle of KY131, large2-1, KYJ, large2-2, ZHJ, and large2-3 (n≥ 16). (I) Width of KY131, large2-1, KYJ, large2-2, ZHJ, and large2-3 flag leaves (n=20). (J) Width of KY131, large2-1, KYJ, large2-2, ZHJ, and large2-3 grains (n≥100). Values (E-J) are given as mean±SD. **P<0.01 compared with the corresponding wild-type values by Student's t-test. Bars: (A) 10 cm; (B) 5 cm; (C) 1 cm; (D) 2 mm.

FIG. 2. LARGE2 encodes the HECT ubiquitin ligase OsUPL2.

(A) The gene structure of LARGE2 (LOC_Os12g24080). Black boxes represent exons and lines represent introns. The start codon (ATG) and the stop codon (TAA) are indicated. The mutation sites of nine different alleles are indicated with arrows. (B) The mutation positons and nucleotide changes of the nine large2 mutant alleles. (C) Schematic diagrams of LARGE2 and the nine mutated proteins. The predicted LARGE2 protein contains a DUF908 domain, a DUF913 domain, a UBA domain, a DUF4414 domain, and a HECT domain. (D) Panicles of KY131, LARGE2-RNAi #1, LARGE2-RNAi #2 and LARGE2-RNAi #3. LARGE2-RNAi is KY131 transformed with the LARGE2-RNAi vector. (E-G) Number of primary branches (E), number of secondary branches (F), and grain number per panicle (G) of KY131, LARGE2-RNAi #1, LARGE2-RNAi #2 and LARGE2-RNAi #3 panicles (n≥16). (H) Relative expression levels of LARGE2 in KY131, LARGE2-RNAi #1, LARGE2-RNAi #2 and LARGE2-RNAi #3 panicles. Young panicles (1 mm) were used for qRT-PCR analyses with three biological replicates (n=3). The rice Actin1 was used as the internal control. (I) panicles of KYJ, the large2 mutants in KYJ background, and F1 plants that produced by crossing different mutants. (J) LARGE2 is a functional E3 ubiquitin ligase. The HECT domain of LARGE2 was fused with MBP to test the ubiquitin ligase activity. Ubiquitinated proteins were detected using both anti-His and anti-MBP antibodies. The red arrows indicate ubiquitinated MBP-HECT proteins. Changing the conserved Cys to Ala or Ser abolished the ubiquitin ligase activity. Values (E-H) are given as mean±SD. **P<0.01 compared with KY131 by Student's t-test. Bars: (E) 5 cm; (K) 5 cm.

FIG. 3. LARGE2 regulates the sizes of shoot apical meristems and panicle meristems.

(A-B) Cleared shoot apical meristems (SAMs) of KYJ and large2-2 on 1st day after germination (1 DAG). The length of red lines indicates the SAM length. (C) Average SAM length (SL) of KYJ and large2-2 and cell number (CN) along the SAM lines (1 DAG) (n=12). (D-E) Scanning electron microscope (SEM) images that show the SAM of KYJ and large2-2 at the transition stage from the vegetative to the reproductive phase. The carmine shows the area of rachis meristem (RM). (F) Average rachis meristem (RM) area of KYJ and large2-2 (n=12). (G-H) SEM images that show the primary branch meristems (PBMs) of KYJ and large2-2. The asterisks indicate PBMs. (I) Average PBM number of KYJ and large2-2 (n=12). (J) Relative expression levels of KNOX genes in KYJ and large2-2 panicles. Young panicles (1 mm) were used for qRT-PCR analyses with three biological replicates (n=3). (K) Relative expression levels of genes involved in panicle size regulation in KYJ and large2-2 panicles. Young panicles (1 mm) were used for qRT-PCR analyses with three biological replicates (n=3). Values (C, F, I-K) are given as mean±SD relative to KYJ value set at 100%. **P<0.01 compared with KYJ by Student's t-test. The rice Actin1 was used as the internal control in (J) and (K). Bars: (A-B) 25 μm; (D-E) 100 μm; (G-H) 100 μm.

FIG. 4. Expression pattern of LARGE2.

(A) The expression levels of LARGE2 in roots (R), stems(S), leaves (L), leaf sheaths (LS) and young panicles of 1 cm (YP1) to 20 cm (YP20) of KY131 plants. Samples were used for quantitative real-time RT-PCR analyses with three biological replicates (n=3). Values are given as mean±SD. Different lowercase letters above the columns indicate the significant difference among different groups, one-way ANOVA P-values: P<0.05. The rice Actin1 was used as the internal control. (B) The LARGE2 expression in the SAM of proLARGE2: GUS seedlings. The GUS-stained SAMs were embedded in paraffin, sectioned and observed with a microscope. (C) The LARGE2 expression in the proLARGE2: GUS developing panicle at the primary branch initiation stage. The GUS-stained developing panicles were embedded in paraffin, sectioned and observed with a microscope. The black asterisks indicate primary branch meristems (PBMs). (D) The LARGE2 expression in the proLARGE2: GUS developing panicle at the secondary branch initiation stage. The GUS-stained developing panicles were embedded in paraffin, sectioned and observed with a microscope. The red asterisks indicate secondary branch meristems (SBMs) and the white box indicates a floral meristem. (E) A closer view of the LARGE2 expression in a floral meristem. (F-O) the LARGE2 expression in developing seedlings (F-I), roots (F-I), culm node and internode (J), leaves (G-I, K) and developing young seedlings (L-O) of proLARGE2: GUS plants. The GUS-stained samples were observed with a camera. proLARGE2: GUS is KY131 transformed with the proLARGE2: GUS vector. Bars: (B) 50 μm; (C-D) 200 μm; (E) 50 μm; (F-H) 5 mm; (I) 15 mm; (J-N) 5 mm; (O) 15 mm.

FIG. 5. LARGE2 physically associates with APO1 and APO2.

(A) LARGE2 was divided into five fragments (F1-F5) to analyze its interactions with APO1 and APO2. (B-C) Split luciferase complementation assay showed that the fragment 3 (F3) of LARGE2 interacts with APO1 (B) and APO2 (C). Tobacco leaves expressing different combinations of LARGE2-F3-nLUC and cLUC-APO1/APO2 were tested for LUC activity. LUC activity was observed 48 h after infiltration. (D-E) Co-immunoprecipitation assay showed that the fragment 3 (F3) of LARGE2 associates with APO1 (D) and APO2 (E) in N. benthamiana leaves. The GFP beads were used to immunoprecipitate Myc-LARGE2-F3 proteins. Gel blots were probed with anti-Myc or anti-GFP antibody. IP, immunoprecipitation; IB, immunoblot.

FIG. 6. LARGE2 modulates the stabilities of APO1 and APO2.

(A-B) The proteasome inhibitor MG132 stabilizes APO1. GFP-APO1 was expressed in N. benthamiana leaves for 48 h, and then treated with or without 50 mM MG132 for 24 h. Total protein was extracted and subjected to immunoblot using anti-GFP and anti-Actin antibodies. The GFP-APO1 protein level was quantified relative to the Actin protein level by ImageJ software. Band intensities of triplicate repeats (FIG. 6A) were quantified by the ImageJ software (n=3). Relative levels of GFP-APO1 proteins were shown (B). (C-D) LARGE2 modulates the protein stabilities of APO1 in rice. 35S: GFP-APO1 transgenic lines were crossed with large2-3 to generate 35S: GFP-APO1 (1) and 35S: GFP-APO1; large2-3 (2). Total protein extracts from young panicles (1 cm) of (1) and (2) were subjected to immunoblot analysis using anti-GFP and anti-Actin antibodies. The GFP-APO1 protein level was quantified relative to the Actin protein level by ImageJ software. Band intensities of triplicate repeats (FIG. 6C) were quantified by the ImageJ software (n=3). Relative levels of GFP-APO1 proteins were shown (D). (E) The expression levels of GFP-APO1 in young panicles (1 cm) of 35S: GFP-APO1 (1) and 35S: GFP-APO1;large2-3 (2). Quantitative real-time RT-PCR analyses were performed with three biological replicates (n=3). Values are given as mean±SD. The rice Actin1 was used as the internal control. (F-G) The proteasome inhibitor MG132 stabilizes APO2. GFP-APO2 was expressed in N. benthamiana leaves for 48 h, and then treated with or without 50 mM MG132 for 24 h. Total protein was extracted and subjected to immunoblot using anti-GFP and anti-Actin antibodies. The GFP-APO2 protein level was quantified relative to the Actin protein level by ImageJ software. Band intensities of triplicate repeats (FIG. 6F) were quantified by the ImageJ software (n=3). Relative levels of GFP-APO2 proteins were shown (G). (H-I) LARGE2 modulates the protein stabilities of APO2 in rice. 35S: GFP-APO2 transgenic lines were crossed with large2-3 to generate 35S: GFP-APO2 (3) and 35S: GFP-APO2; large2-3 (4). Total protein extracts from young panicles (1 cm) of (3) and (4) were subjected to immunoblot analysis using anti-GFP and anti-Actin antibodies. The GFP-APO2 protein level was quantified relative to the Actin protein level by ImageJ software. Band intensities of triplicate repeats (FIG. 6H) were quantified by the ImageJ software (n=3). Relative levels of GFP-APO2 proteins were shown (I). (J) The expression levels of GFP-APO2 in young panicles (1 cm) of 35S: GFP-APO2 (3) and 35S: GFP-APO2; large2-3 (4). Quantitative real-time RT-PCR analyses were performed with three biological replicates (n=3). Values are given as mean±SD. The rice Actin1 was used as the internal control.

FIG. 7. The large2 mutants produce large panicles with increased grain number and wide grains.

(A) Panicles of KYJ, large2-4, large2-5, large2-6, large2-7, large2-8, and large2-9. (B) Grains of KYJ, large2-4, large2-5, large2-6, large2-7, large2-8, and large2-9. (C) Panicle length, number of primary branches, number of secondary branches, and grain number per panicle of KYJ, large2-4, large2-5, large2-6, large2-7, large2-8, and large2-9 panicles (n≥16). (D) Grain length (n≥80), grain width (n≥80) and plant height (n≥25) of KYJ, large2-4, large2-5, large2-6, large2-7, large2-8, and large2-9. Values (C-D) are given as the mean±SD relative to the KYJ values set at 100%. **P<0.01 compared with KYJ by Student's t-test. Bars: (A) 10 cm; (B) 2 mm.

FIG. 8. Seven large2 mutants in KYJ background are allelic.

Number of primary panicle branches (NPB), number of secondary panicle branches (NSB), grain number per main panicle (GN) of KYJ and the F1 plants generated by crossing different mutants (n≥16). Values are given as mean±SD relative to the KYJ value set at 100%.

FIG. 9. Silencing of LARGE2 by RNAi results in shortened plant height, wide grains and leaves.

(A) Plants of KY131, LARGE2-RNAi #1, LARGE2-RNAi #2 and LARGE2-RNAi #3. (B) Mature flag leaves of KY131, LARGE2-RNAi #1, LARGE2-RNAi #2 and LARGE2-RNAi #3. (C) Mature grains of KY131, LARGE2-RNAI #1, LARGE2-RNAi #2 and LARGE2-RNAi #3.

(D) Average plant height of KY131, LARGE2-RNAI #1, LARGE2-RNAI #2 and LARGE2-RNAi #3 (n=20). (E) Average grain width of KY131, LARGE2-RNAi #1, LARGE2-RNAI #2 and LARGE2-RNAi #3 (n≥80). (F) Average flag leaf width of KY131, LARGE2-RNAi #1, LARGE2-RNAi #2 and LARGE2-RNAi #3 (n=20). Values (D-F) are given as mean±SD. **P<0.01 compared with their respective parental lines by Student's t-test. Bars: (A) 10 cm; (B) 1 cm; (C) 1 mm.

FIG. 10. The phylogenetic tree of HECT ubiquitin protein ligases in Oryza sativa and Glycine max.

The phylogenetic tree was constructed using the neighbor-joining method of MEGA5.0 program. The full length sequence of HECT ubiquitin protein ligases in Oryza sativa and Glycine max were used to construct the phylogenetic tree. Numbers at the nodes indicate percentage of 1000 bootstrap replicates.

FIG. 11. The phylogenetic tree of HECT ubiquitin protein ligases in Oryza sativa and Brassica napus.

The phylogenetic tree was constructed using the neighbor-joining method of MEGA5.0 program. The full length sequence of HECT ubiquitin protein ligases in Oryza sativa and Brassica napus were used to construct the phylogenetic tree. Numbers at the nodes indicate percentage of 1000 bootstrap replicates.

FIG. 12. The phylogenetic tree of HECT ubiquitin protein ligases in Oryza sativa and Zea mays.

The phylogenetic tree was constructed using the neighbor-joining method of MEGA5.0 program. The full length sequence of HECT ubiquitin protein ligases in Oryza sativa and Zea mays were used to construct the phylogenetic tree. Numbers at the nodes indicate percentage of 1000 bootstrap replicates.

FIG. 13. Identification of large2-9.

(A) RT-PCR analysis of LARGE2 in large2-9 and KYJ. The large2-9 mutation causes two main transcripts. Red arrows show two mutated transcripts, which lead to the two different mutated proteins, LARGE2^{large2-9 #1} and LARGE2^{large2-9 #2}. (B) Alignment of amino acid sequences in the HECT domains of LARGE2, LARGE2^{large2-9 #1} and LARGE2^{large2-9 #2}. Amino acid sequences are used for the alignment using ClustalW method in MEGA5.0 program. The yellow and green boxes indicate the mutated amino acid sequences of LARGE2^{large2-9 #1} and LARGE2^{large2-9 #2}, respectively. The red box indicates the conserved cysteine in the HECT domain.

FIG. 14. Introgression of the large2-9 mutation into the japonica variety Xiushui09 (XS09) increases grain yield.

(A) Plants of XS09 and NIL-large2-9 at the mature stage. (B) Panicles of XS09 and NIL-large2-9. (C-D) Grains of XS09 and NIL-large2-9. (E-G) Number of primary branches (E), number of secondary branches (F), and grain number per panicle (G) of XS09 and NIL-large2-9 panicles. (H-I) Grain width (H) and grain length (I) of XS09 and NIL-large2-9. (J) Tiller number of XS09 and NIL-large2-9. (K) 1000-grain weight of XS09 and NIL-large2-9. (L) Yield per plant of XS09 and NIL-large2-9. (M) Actual yield per plot of XS09 and NIL-large2-9. Values (E-M) are given as mean±SD. **P<0.01 compared with XS09 by Student's t-test. Bars: (A) 10 cm; (B) 5 cm; (C) 2 mm; (D) 5 mm.

FIG. 15. Grains of XS09 and NIL-large2-9.

Grain performances of XS09 and NIL-large2-9. Bar: 2 cm

FIG. 16. Generation of CRISPR constructs of Example 9

FIG. 17. Heterozygous large2 mutant can increase grain yield. (A) Plants of KY131 and KY131/large2-1 at the mature stage. (B) Panicles of KY131 and KY131/large2-1. (C) Grains of KY131 and KY131/large2-1. (D-I) Tiller number (D), panicle length (E), number of primary branches (F), number of secondary branches (G), grain number per panicle (H) and grain yield per plant (I) of KY131 and KY131/large2-1. (J-L) Grain length (J), grain width (K) and 1,000-grain weight (L) of KY131 and KY131/large2-1. KY131/large2-1 is the F1 plant produced by crossing KY131 with large2-1. Values (D-L) are given as mean±SD. **P<0.01 compared with KY131 by Student's t-test. Bars: (A) 10 cm; (B) 5 cm; (C) 2 mm.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombination DNA technology and exclude embodiments that are solely based on generating plants by traditional breeding methods.

In a first aspect of the invention, there is provided a method of increasing yield in a plant, the method comprising reducing or abolishing the expression of at least one nucleic acid encoding a UPL2 polypeptide and/or reducing or abolishing the activity of a UPL2 polypeptide in said plant.

All following embodiments apply to all aspects of the invention.

In one embodiment, the method comprises reducing or abolishing the activity of the UPL2 polypeptide. UPL2 may be referred to as LARGE2 and such terms may be used interchangeably herein. LARGE2 encodes a E3 ubiquitin ligase (UPL2). In one embodiment, the method comprises reducing or abolishing the E3 ubiquitin ligase activity of UPL2. Ubiquitin ligase activity can be measured by any number of techniques in the art.

In another embodiment, the method comprises reducing or abolishing the binding of UPL2 to target proteins, particularly APO (ABERRANT PANICLE ORGANIZATION) 1 and APO2 or homologues thereof.

The term “yield” in general means a measurable produce of economic value, typically related to a specified crop, to an area, and to a period of time. Individual plant parts directly contribute to yield based on their number, size and/or weight. Alternatively, the actual yield is the yield per square meter for a crop and year, which is determined by dividing total production (includes both harvested and appraised production) by planted square meters.

In one embodiment, increased yield comprises an increase in at least one or more of the following yield-related parameters; seed number, seed width, inflorescence size, increased thousand kernel weight (TKW), increased biomass, increased fresh weight Preferably, in the present context, the term “yield” of a plant relates to propagule generation (such as seeds) of that plant. Thus, in a preferred embodiment, the method relates to an increase in seed number, seed yield or total seed yield. According to the invention, seed yield can be measured by assessing one or more of seed number, seed size or a combination of both seed size and seed number. An increase in the TKW can result from an increase in seed size and/or seed weight. Preferably, an increase in seed yield is an increase in at least one of seed number, seed width and TKW. In a further embodiment, seed length is unaffected. Yield is increased relative to a control or wild-type plant.

The skilled person would be able to measure any of the above seed yield parameters using known techniques in the art. The terms “seed” and “grain” as used herein can be used interchangeably.

For example, yield or any one of the above yield-related parameters is increased by at least 10%, 15%, 20%, 25%, 30%, 35%, 40%, 45%, 50%, 55%, 60%, 65%, 70%, 75%, 80%, 85%, 90% or 95% or more compared to a wild-type or control plant. In one embodiment, yield, and in particular, grain number may be increased by between 20 and 95% compared to a wild-type or control plant.

The term “reducing” means a decrease in the levels of UPL2 polypeptide expression and/or activity by up to 10%, 20%, 30%, 40%, 50%, 60%, 70%, 80% or 90% when compared to the level in a wild-type or control plant. Preferably, reducing means a decrease in the level of expression or activity of UPL2 above or around 50%-95%. The term “abolish” expression means that no expression of UPL2 polypeptide is detectable or that no functional UPL2 polypeptide is produced. That is, the UPL2 polypeptide lacks all functional E3 ligase activity or is unable to bind to target proteins, such as APO1 and APO2. Methods for determining the level of endogenous UPL2 expression would be well known to the skilled person. For example, a reduction in the expression and/or content levels of endogenous UPL2 may comprise a measure of protein and/or nucleic acid levels by techniques such as gel electrophoresis or chromatography (e.g. HPLC). By “reducing the activity” means reducing the biological activity of UPL2, for example, reducing the functional E3 ligase activity or reducing the ability to bind to target proteins, such as APO1 and APO2.

Inflorescence size and grain number in particular are important agronomic traits in crops. As shown in FIGS. 2, 7 and 14 we have identified that introducing loss of function mutations in LARGE2, which encodes an E3 ubiquitin ligase, leads to an increase in grain number and yield. In one embodiment, we use RNAi technology to knock-down the expression of LARGE2 or its homologs in crops to increase seed number and yield in these crops.

In another embodiment, the method comprises introducing at least one mutation into the, preferably endogenous, gene encoding UPL2 and/or the UPL2 promoter. Preferably, said mutation is a loss of function or partial loss of function mutation in the UPL2 gene. Alternatively, said mutation in the UPL2 promoter reduces or abolishes UPL2 expression.

By “at least one mutation” means that where the UPL2 gene is present as more than one copy or homeologue (with the same or slightly different sequence) there is at least one mutation in at least one gene. In one embodiment, all genes are mutated such that the plant is homozygous for the mutation. In an alternative embodiment, where the plant is a diploid or polyploid, one or two or half of the copies or homeoalles of the UPL2 gene or promoter are mutated such that the plant is heterozygous for the mutation.

In another embodiment, the sequence of the UPL2 gene comprises or consists of a nucleic acid sequence that encodes a polypeptide as defined in SEQ ID NO: 2 or a functional variant or homologue thereof. In a further embodiment, the sequence of the UPL2 gene comprises or consists of SEQ ID NO: 1 (cDNA), 81 (genomic) or a functional variant or homologue thereof.

By “UPL2 promoter” is meant a region extending for at least 2 kbp upstream of the ATG codon of the UPL2 ORF (open reading frame). In one embodiment, the sequence of the UPL2 promoter comprises or consists of a nucleic acid sequence as defined in SEQ ID NO: 3 or a functional variant or homologue thereof.

Examples of UPL2 homologs are shown in SEQ ID NOs: 4 to 26 and in Table 1 below. Accordingly, in one embodiment, the homolog encodes a polypeptide selected from SEQ ID NOs: 5, 7, 9, 12, 15 and 18. In an alternative embodiment, the homolog comprises or consists of a nucleic acid sequence selected from one of SEQ ID NOS: 4, 6, 8, 10, 11, 13, 14, 16, 17, 19, 20, 21, 22, 23, 24, 25 and 26. In a further or additional embodiment, the sequence of the homologue is selected from one of the sequences in Table 1.

TABLE 1
Examples of homologue sequences:

| | | | SEQ ID NO: of |
|---|---|---|---|
| | | | amino acid |
| | Gene ID | SEQ ID NO of CDS | sequence |
| Maize | GRMZM2G331368 | SEQ ID NO: 10 | SEQ ID NO: 12 |
| Maize | GRMZM2G411536 | SEQ ID NO: 13 | SEQ ID NO: 15 |
| B. napus | Bra038022.1 | SEQ ID NO: 26 | / |
| Soybean | GLYMA_02G216000 | KRH72480 (SEQ ID | / |
| | | NO: 19) or KRH72479 | |
| | | (SEQ ID NO: 20) | |
| Soybean | GLYMA_04G096900 | KRH62267 (SEQ ID | / |
| | | NO: 21) or KRH62268 | |
| | | (SEQ ID NO: 22) | |
| Soybean | GLYMA_14G183000 | >KRH16871 (SEQ ID | / |
| | | NO: 23) or KRH16870 | |
| | | (SEQ ID NO: 24) or | |
| | | KRH16869 (SEQ ID | |
| | | NO: 25) | |
| Wheat - A genome | TraesCS5A02G121600.1 | SEQ ID NO: 4 | SEQ ID NO: 5 |
| Wheat - B genome | TraesCS5B02G112800.1 | SEQ ID NO: 6 | SEQ ID NO: 7 |
| Wheat - D genome | TraesCS5D02G118000.1 | SEQ ID NO: 8 | SEQ ID NO: 9 |
| Millet | Seita.3G302600.1 | SEQ ID NO: 16 | SEQ ID NO: 18 |

The term “functional variant” as used herein with reference to any of the sequences recited herein refers to a variant nucleic acid or amino acid sequence or part of that sequence which retains the biological function of the full non-variant sequence. For example, the variant also has E3 ligase activity. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a UPL2 gene or promoter orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NO: 2 or to the nucleic acid sequences as shown by SEQ ID NOs: 1 or 3. In one embodiment, overall sequence identity is at least 58%. Functional variants of UPL2 homologs as defined above are also within the scope of the invention.

The E3 ubiquitin ligase UPL2 is characterised by a number of conserved domains: DUF908, DUF913, UBA, DUF4414 and HECT domains. In one embodiment, the sequence of these domains is as follows:

DUF908
(SEQ ID NO: 58)
AAAAATACCATCCTGCAGATTTTGAGAGTAATGCAGATTGTTTTGGAAAATTGCCA
GAACAAAACATCGTTTGCTGGTCTTGAGCATTTTAGGCTTCTGCTGGCATCATCAG
ATCCTGAGATAGTTGTGGCTGCTTTAGAGACACTTGCTGCATTGGTTAAAATAAAT
CCTTCGAAGTTGCATATGAACGGAAAGCTCATAAATTGTGGAGCTATAAACAGTCA
TCTTCTATCATTGGCACAAGGATGGGGTAGCAAGGAGGAAGGTTTGGGCTTATAT
TCTTGTGTTGTGGCAAATGAAAGAAACCAGCAGGAGGGTTTGTGCTTATTCCCAG
CAGACATGGAGAACAAATACGATGGCACGCAGCACCGTCTCGGTTCAACTCTTCA
TTTTGAATATAATTTGGCACCTGCCCAAGATCCTGACCAATCCAGTGACAAGGCTA
AGCCATCTAATCTGTGTGTGATACATATCCCAGACTTGCACCTTCAGAAGGAGGAT
GACTTGAGCATATTGAAGCAATGTGTTGATAAGTTTAATGTGCCTTCAGAGCACAG
ATTTTCCTTGTTTACAAGGATAAGATATGCCCATGCCTTTAATTCGCCACGGACAT
GTAGGCTATATAGCCGCATAAGTCTTCTTGCTTTCATTGTTCTTGTGCAATCCAGC
GATGCCCATGATGAACTCACATCTTTCTTTACAAATGAGCCAGAGTACATAAATGA
GTTAATCAGACTTGTCCGATCAGAGGAATTTGTTCCTGGACCCATACGAGCGCTG
GCTATGCTTGCACTGGGAGCACAGTTAGCAGCGTATGCATCATCTCATGAACGAG
CTCGGATACTTAGTGGCTCAAGTATCATATCTGCTGGTGGAAACCGCATGGTCTT
GCTCAGTGTTTTGCAAAAAGCTATATCA
DUF913
(SEQ ID NO: 59)
GCAGTGAAAACTCTTCAAAAGTTGATGGAGTACAGCAGCCCTGCTGTTTCTCTATT
TAAAGATTTGGGTGGTGTAGAACTTTTGTCTCAGAGGTTGCACGTGGAGGTGCAG
CGTGTTATTGGTGTTGACAGTCATAATTCAATGGTTACAAGTGATGCATTGAAATC
AGAAGAGGATCATCTCTACTCTCAGAAGCGATTGATTAAGGCGCTGCTAAAGGCA
TTGGGGTCTGCTACATATTCTCCTGCAAATCCTGCTCGTTCACAAAGCTCAAATGA
TAATTCTTTGCCCATCTCGCTTTCCCTTATATTTCAGAATGTTGACAAGTTTGGTGG
TGACATTTATTTCTCAGCAGTTACTGTTATGAGTGAGATAATTCACAAGGATCCAAC
ATGCTTTCCTTCTTTGAAGGAACTTGGTCTTCCAGATGCTTTTCTATCGTCAGTGA
GTGCTGGGGTAATACCATCTTGTAAAGCTCTCATCTGTGTGCCTAATGGTCTGGG
TGCAATATGCCTTAATAACCAAGGACTTGAGGCTGTCAGGGAAACTTCAGCTCTG
CGTTTTCTTGTTGACACATTCACCAGCAGGAAGTACTTGATACCAATGAATGAAGG
TGTTGTCCTATTAGCTAATGCAGTGGAAGAGCTTCTACGTCACGTGCAGTCCCTAA
GAAGCACTGGGGTTGACATCATTATTGAAATAATTAATAAACTTTCTTCACCTCGTG
AAGATAAGAGCAATGAACCAGCGGCCAGTTCTGATGAAAGAACAGAAATGGAAAC
TGACGCGGAAGGACGTGATTTGGTAAGTGCTATGGATTCCAGTGAGGATGGCACT
AATGATGAACAGTTTTCTCATTTGAGCATTTTCCATGTGATGGTATTGGTTCATCGG
ACAATGGAGAACTCCGAAACCTGCCGGTTATTTGTGGAGAAAGGAGG
UBA
(SEQ ID NO: 60)
AATGCAATTTCTCTGATTGTAGAGATGGGCTTTTCTCGCGCCAGAGCTGAGGAAG
CACTCAGGCAAGTTGGAACGAACAGTGTTGAAATTGCAACTGATTGGTTATTCTCA
CAC
DUF4414
(SEQ ID NO: 61)
AACAGAGCTGCTGACACTGACTCAATTGATCCTACATTTTTGGAGGCTCTTCCAGA
GGATTTACGGGCTGAAGTTCTTTCTTCACGTCAAAATCAAGTGACCCAG
Or
(SEQ ID NO: 62)
GAACAACCTCAGAATGATGGGGATATTGATCCTGAATTCCTTGCTGCACTTCCTCC
TGATATACGTGAAGAAGTT
Glu/Asp-rich domain
(SEQ ID NO: 63)
ATCAGATTTGAAATTCCACGAAATAGAGAGGATGATATGGCTGATGATGACGAGG
ACAGTGATGAGGACATGTCAGCCGATGATGGTGAGGAGGTTGATGAAGATGAAG
ACGAGGATGAGGATGAAGAGAACAACAACCTGGAGGAGGATGATGCCCATCAAA
TGTCTCATCCTGACACAGATCAGGAGGACCGTGAGATGGATGAAGAGGAGTTTGA
CGAGGATCTGCTAGAAGAAGATGATGATGAGGATGAGGATGAG
HECT:
(SEQ ID NO: 64)
RISVRRAYILEDSYNQLRMRSPQDLKGRLTVHFQGEEGIDAGGLTREWYQLLSRVIFD
KGALLFTTVGNDLTFQPNPNSVYQTEHLSYFKFVGRVVGKALFDGQLLDVHFTRSFY
KHILGVKVTYHDIEAIDPAYYKNLKWMLENDISDVLDLSFSMDADEEKRILYEKAEVTD
YELIPGGRNIKVTEENKHEYVNRVAEHRLTTAIRPQITSFMEGFNELIPEELISIFNDKEL
ELLISGLPDIDLDDLKANTEYSGYSIASPVIQWFWEIVQGFSKEDKARFLQFVTGTSKV
PLEGFSALQGISGPQRFQIHKAYGSTNHLPSAHTCFNQLDLPEYTSKEQLQERLLLAIH
EANEGFGFG

Accordingly, in one embodiment, the UPL2 nucleic acid (coding) sequence encodes a UPL2 protein comprising at least one DUF908, DUF913, UBA, DUF4414 or HECT domain as defined in any of SEQ ID Nos 58 to 64, or a variant thereof, wherein the variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to SEQ ID Nos 58 to 64 as defined herein.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue as an E3 ligase can be confirmed using routine methods in the art.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure, such as those described above, can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a variant as used herein can comprise a nucleic acid sequence encoding a UPL2 gene or promoter as defined herein that is capable of hybridising under stringent conditions as defined herein to a nucleic acid sequence as defined in SEQ ID NO: 1, 2 or 3.

In one embodiment, there is provided a method of increasing yield in a plant, as described herein, wherein the method comprises introducing at least one mutation into at least one UPL2 gene and/or promoter as described above, wherein the UPL2 gene comprises or consists of
a. a nucleic acid sequence encoding a polypeptide as defined in one of SEQ ID NO: 2, 5, 7, 9, 12, 15 or 18; or
b. a nucleic acid sequence as defined in one of SEQ ID NO: 1, 4, 6, 8, 10, 11, 13, 14, 16, 17, 19, 20, 21, 22, 23, 24, 25 or 26; or
c. a nucleic acid sequence encoding a polypeptide comprising at least one DUF908, DUF913, UBA, DUF4414 and HECT domain as defined in SEQ ID NO: 58, 59, 60, 61, 62, 63 or 64 or a functional variant thereof;
d. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b) or (c); or
e. a nucleic acid sequence encoding a UPL2 polypeptide as defined herein that is capable of hybridising under stringent conditions as defined herein to the nucleic acid sequence of any of (a) to (d).
   and wherein the UPL2 promoter comprises or consists of
f. a nucleic acid sequence as defined in one of SEQ ID NO: 3
g. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (f); or
h. a nucleic acid sequence capable of hybridising under stringent conditions as defined herein to the nucleic acid sequence of any of (f) to (h).

In a preferred embodiment, the mutation that is introduced into the endogenous UPL2 gene or promoter thereof to completely or partially silence, reduce, or inhibit the biological activity and/or expression levels of the UPL2 gene or protein can be selected from the following mutation types
1. a “missense mutation”, which is a change in the nucleic acid sequence that results in the substitution of an amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more amino acids, due to one or more codons having been added in the coding sequence of the nucleic acid;
4. a “deletion mutation” of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.

In a preferred embodiment, the mutation in the UPL2 gene is a loss of function mutation or partial loss of function mutation. In one example of a loss of function mutation is any mutation that reduces or abolishes UPL2 E3 ligase activity. In another example, the mutation is any mutation that reduces or abolishes the binding of UPL2 to its target proteins. By target protein means any ubiquitin protein substrate. In one embodiment, the target protein is APO1 and/or APO2. Other examples of target proteins may include SPL14/IPA1 (Ideal Plant Architecture 1). In a further example of a loss of function mutation, the mutation is in the coding region of the UPL2 gene. In this manner, the activity of the UPL2 polypeptide can be considered to be reduced or abolished as described herein. A reduction is described above.

In one embodiment, the mutation reduces or abolishes activity of the E3 ubiquitin ligase. As shown in FIG. 2J, an intact HECT domain is required for functional ubiquitin ligase activity. Accordingly, in one embodiment, the mutation results in a non-functional HECT (Homologous to the E6-AP Carboxyl Terminus) domain. The mutation may be in the HECT domain or elsewhere in the UPL2 polypeptide and preferably results in the complete deletion or partial deletion of the HECT domain. In one embodiment, the mutation is a substitution or a deletion of cysteine at position 3612 of SEQ ID NO: 2 or a homologous position in a homologous sequence. More preferably, the mutation is a substitution, and more preferably is a substitution to a serine or alanine. This cysteine is required for ubiquitin-thiolester formation. Mutation of this conserved cysteine abolishes all ubiquitin ligase activity.

In another embodiment, the mutation that reduces or abolishes the binding of UPL2 to its target proteins is a mutation in the Glu/Asp-rich domain, as described herein. Preferably, the mutation is a substitution of one or more amino acids in the Glu/Asp domain. Alternatively, the mutation is the deletion or partial deletion of the Glu/Asp-rich domain. As shown in FIG. 5B deletion of the Glu/Asp-rich domain reduces, preferably abolishes the association of UPL2 with one of its target substrates, APO1.

In another embodiment, the mutation is, as shown in FIG. 2B, selected from one or more of the following:
a G to T substitution at position 7728 of the genomic sequence of OsUPL2 or position 11510 of SEQ ID NO: 81 or a homologous position in a homologous sequence. This mutation may be referred to herein as large2-1;
a G to A substitution at position 13631 of the genomic sequence of OsUPL2 or position 17413 of SEQ ID NO: 81 or a homologous position in a homologous sequence. This mutation may be referred to herein as large2-2;
a deletion of C at position 9785 of the genomic sequence of OsUPL2 or position 13567 of SEQ ID NO: 81 or a homologous position in a homologous sequence. This mutation may be referred to herein as large2-3;
a deletion of AAAG at position 4424 of the genomic sequence of OsUPL2 or position 8205 of SEQ ID NO: 81 or a homologous position in a homologous sequence. This mutation may be referred to herein as large2-4
a G to A substitution at position 8283 position of the genomic sequence of OsUPL2 or position 12065 of SEQ ID NO: 81 SEQ ID NO: 81 or a homologous position in a homologous sequence. This mutation may be referred to herein as large2-5
a deletion of G at position 9399 of the genomic sequence of OsUPL2 or position 13181 of SEQ ID NO: 81 or a homologous position in a homologous sequence. This mutation may be referred to herein as large2-6
a deletion of T at position 11710 of the genomic sequence of OsUPL2 or position 15492 of SEQ ID NO: 81 or a homologous position in a homologous sequence. This mutation may be referred to herein as large2-7;
a deletion of AATGGATGCTTGA at position 12958 of the genomic sequence of OsUPL2 or position 16740 of SEQ ID NO: 81 or a homologous position in a homologous sequence. This mutation may be referred to herein as large2-8; and
a substitution of A to G at position 13081 of the genomic sequence of OsUPL2 or position 16863 of SEQ ID NO: 81 or a homologous position in a homologous sequence. This mutation may be referred to herein as large2-9.

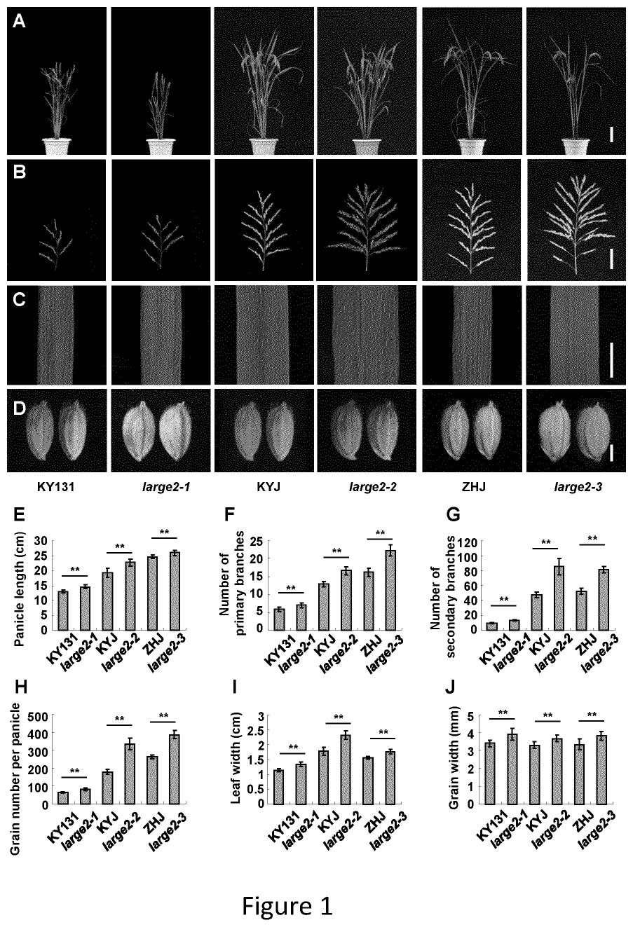

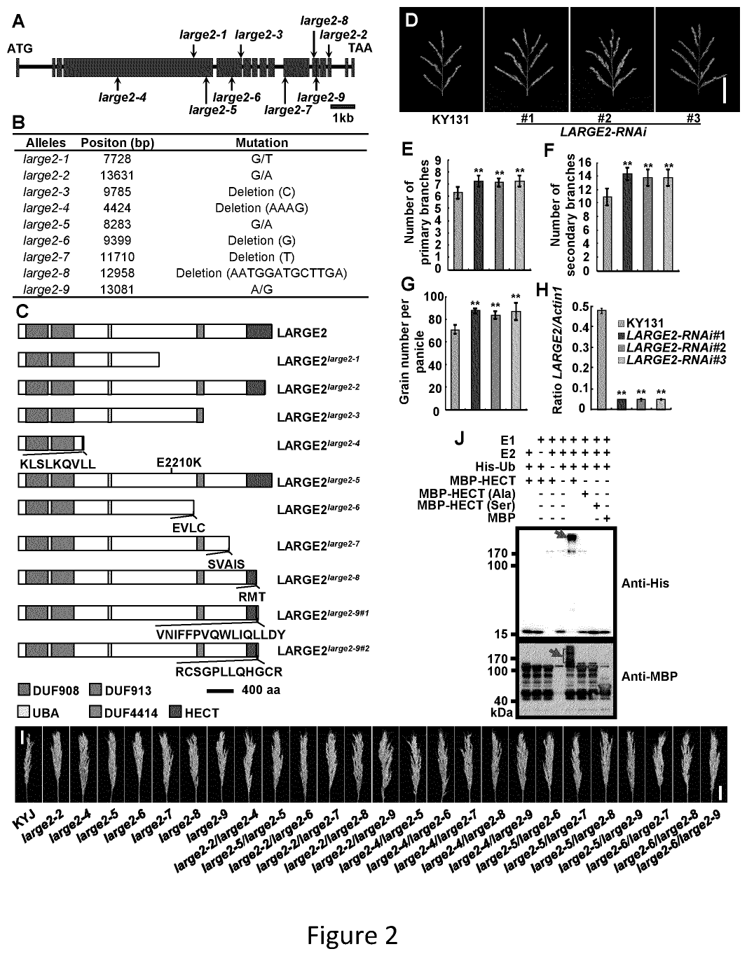

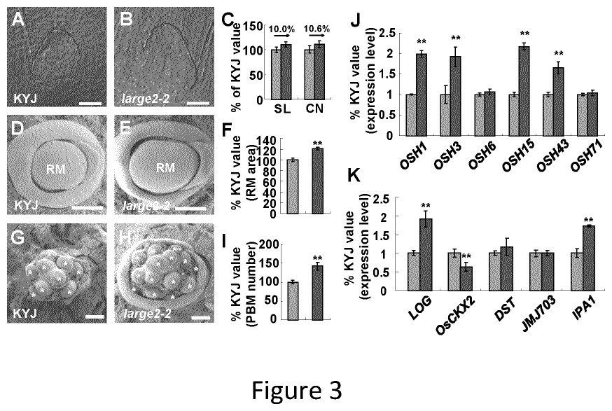

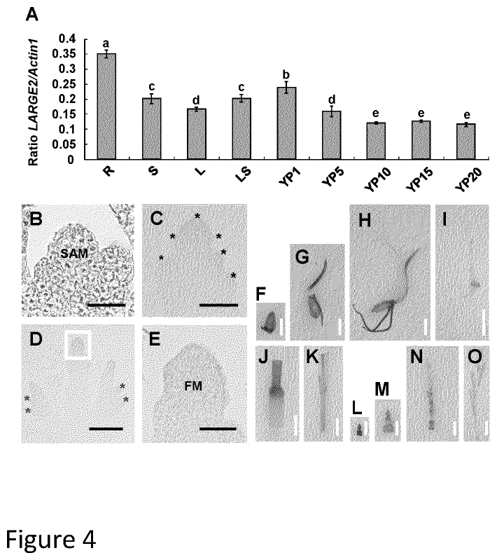

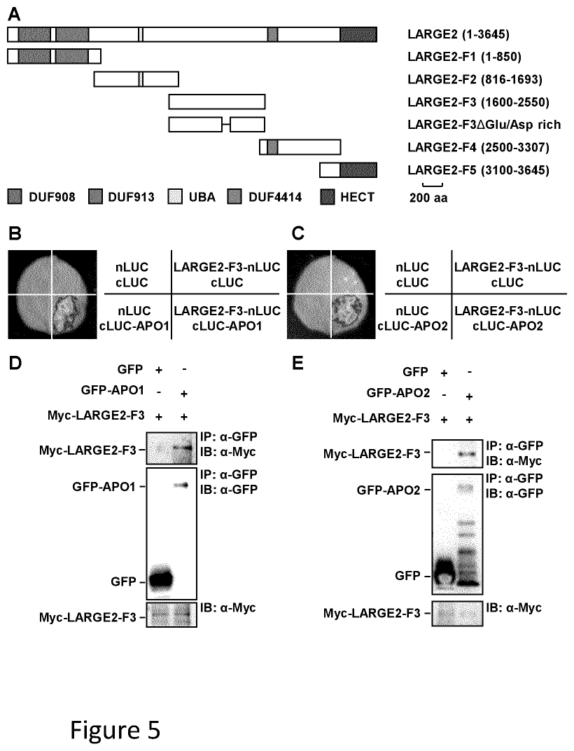

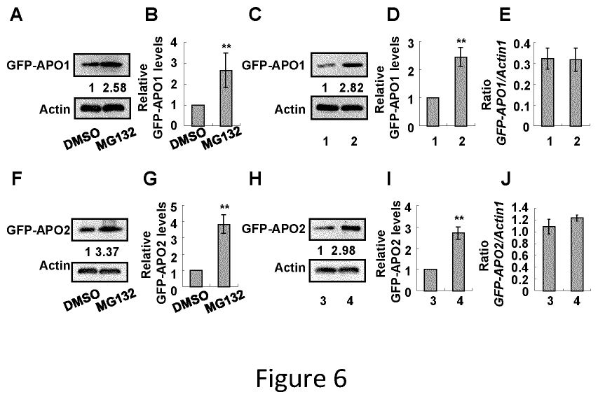

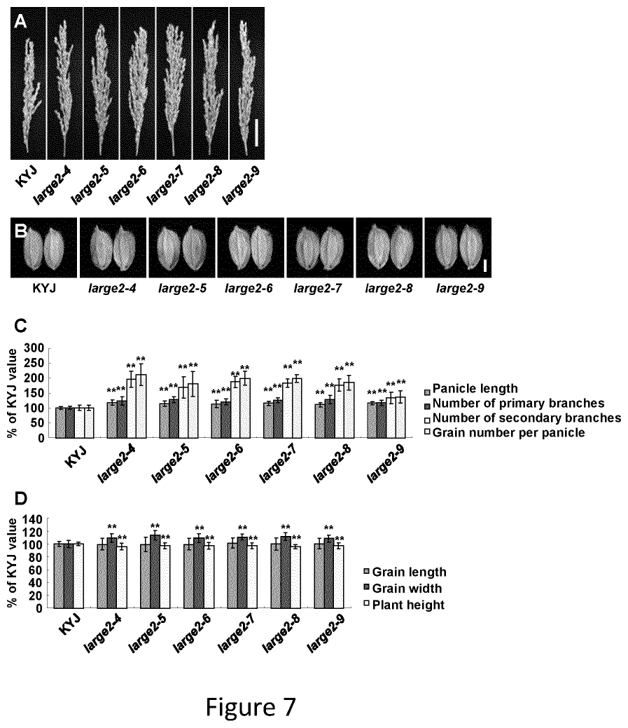

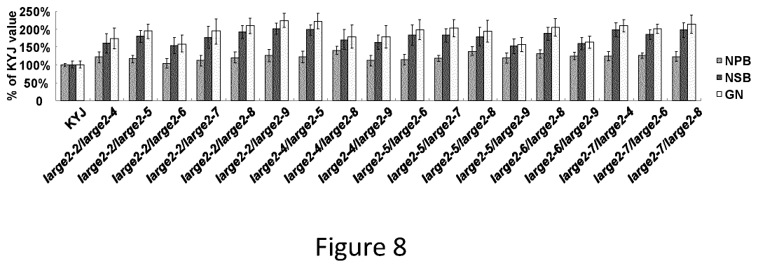

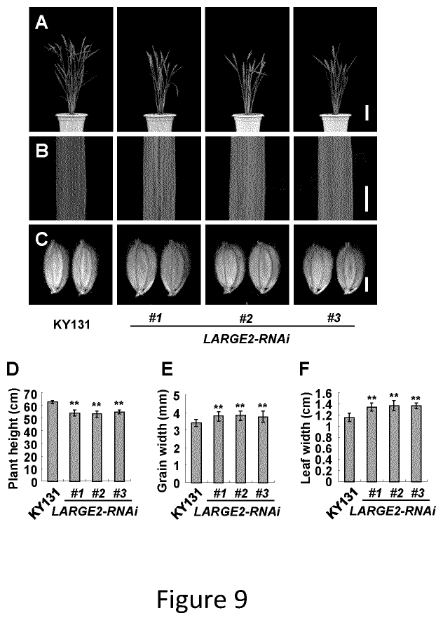

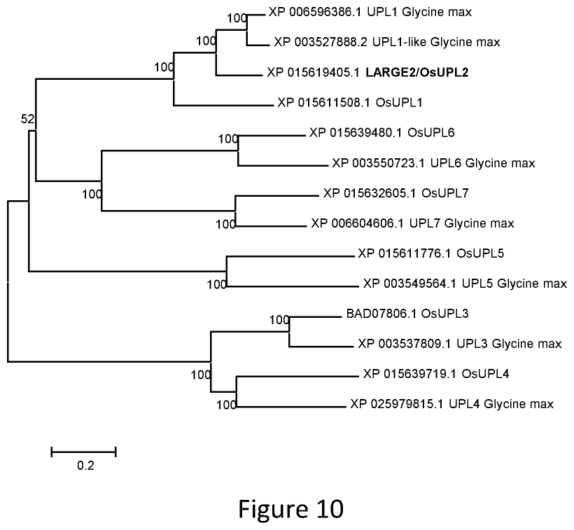

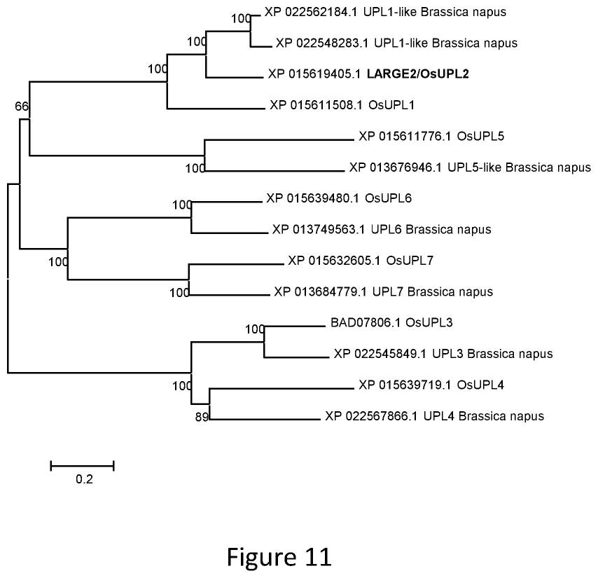

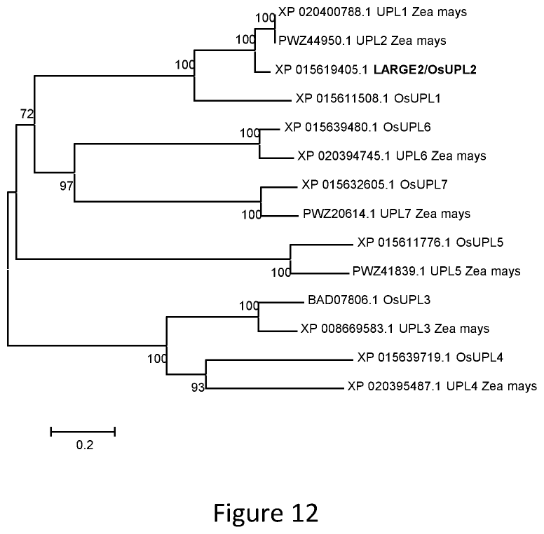

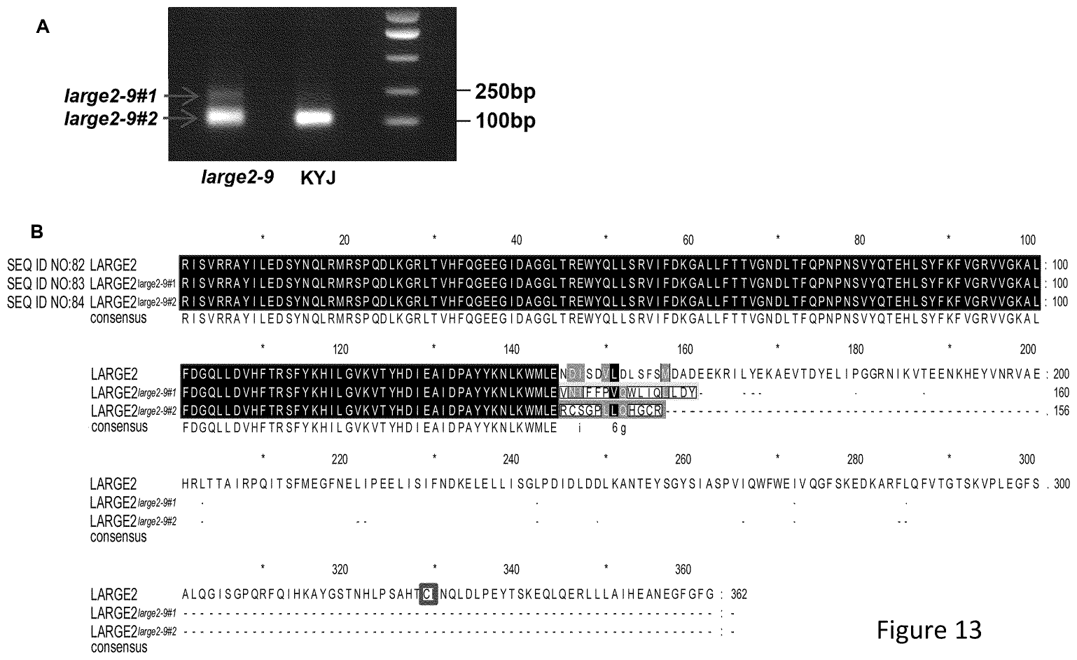

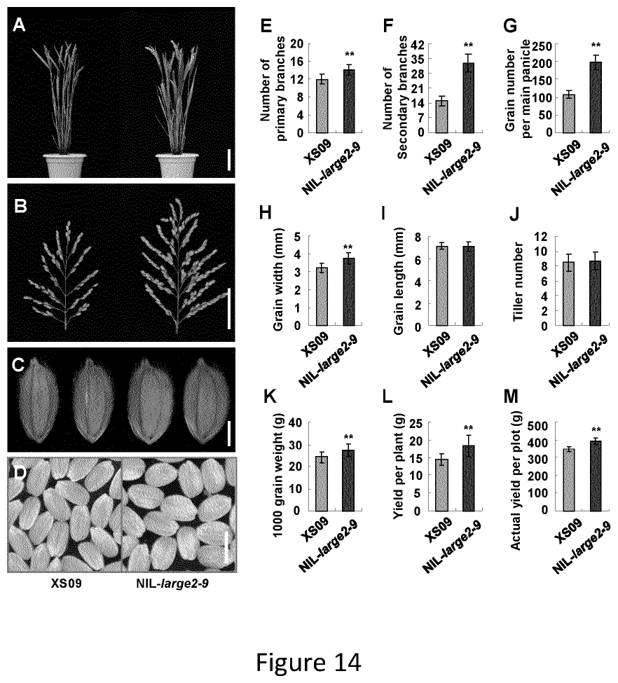

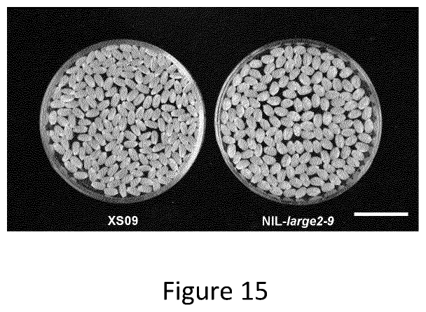

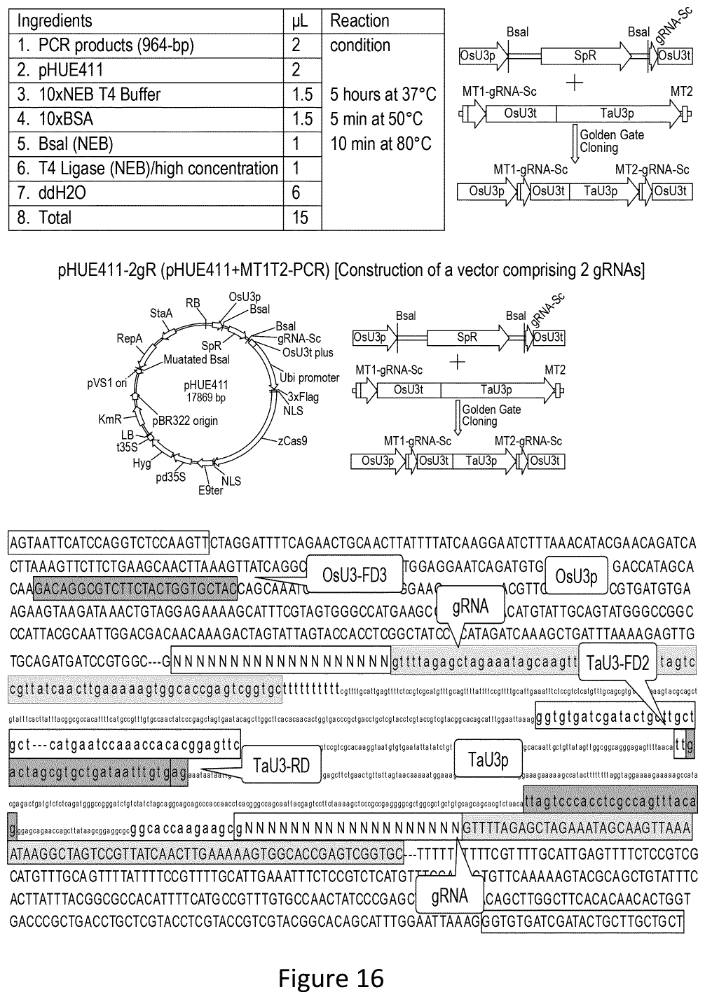

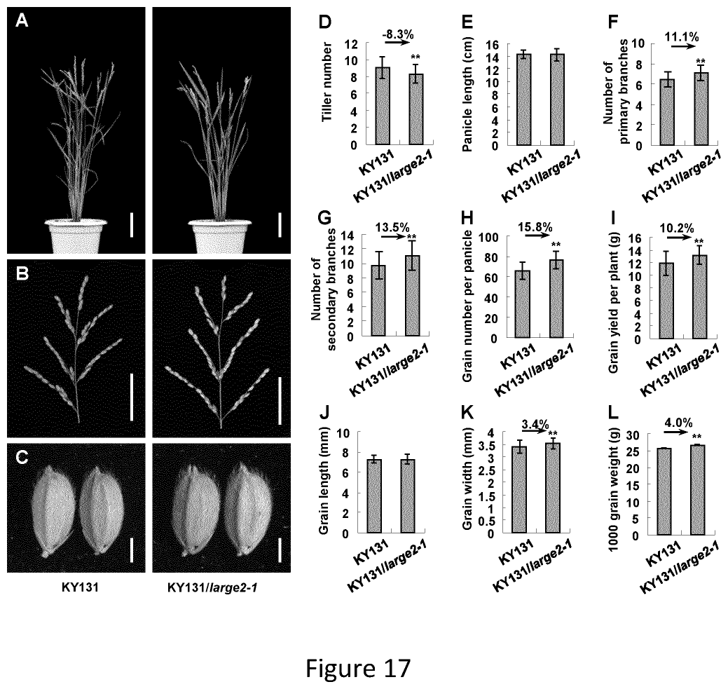

As shown in FIG. 2, the large2-5 mutation results in an amino acid change from Glutamic acid (E) to Lysine (K). Large2-1, 2-2, 2-3, 2-4, 2-6, 2-7 and 2-8 all lead to truncation of the large2 polypeptide and consequently partial or a complete deletion of the HECT domain. The large 2-9 mutation leads to an A to G substitution at the exon-intron boundary and results in two transcripts that, as shown in FIG. 2C, are predicted to encode two different versions of the proteins with truncated HECT domains. As shown in FIGS. 1, 2 and FIGS. 14 and 15, these mutants produced large inflorescences with increased grain numbers and wide grains and increased grain yield. All large-2 mutants are loss of function mutants or partial loss of function mutations.

Where the mutation is complete loss of function, the mutation may be introduced into only one or two (where the plant is a polypolid) copies of the UPL2 gene or promoter; or as described herein, the plant may be crossed with a second plant that is a wild-type or control plant to produce a F1 hybrid heterozygous for the complete loss of function mutation. Alterntaively, where the mutation is a partial loss of function mutation, the mutation may be introduced into all copies of the UPL2 gene and/or promoter.

In a preferred embodimt, the mutation is a substitution of A to G at position 13081 of the genomic sequence of OsUPL2 or position 16863 of SEQ ID NO: 81 or a homologous position in a homologous sequence. In other words, in a preferred embodiment, the mutation is the large2-9 mutation.

In a further embodiment, at least one mutation or structural alteration may be introduced into the UPL2 promoter such that the UPL2 gene is either not expressed (i.e. expression is abolished) or expression is reduced, as defined herein. In any case, the mutation may result in the expression of a UPL2 polypeptide with no, significantly reduced or altered biological activity in vivo. Alternatively, UPL2 may not be expressed at all. In one embodiment, the mutation is the deletion of one or more nucleotides in the UPL2 promoter. In a particular embodiment, the deletion may be the deletion of all or part of SEQ ID NO: 32 from the UPL2 promoter sequence.

In general, the skilled person will understand that at least one mutation as defined above and which leads to the insertion, deletion or substitution of at least one nucleic acid or amino acid compared to the wild-type UPL2 promoter or UPL2 nucleic acid or protein sequence can affect the biological activity of the UPL2 protein.

In one embodiment a mutation may be introduced into the UPL2 promoter and at least one mutation is introduced into the UPL2 gene.

It has been particularly found that plants that are heterozygous for a mutation in UPL2, or equally where the expression or activity of UPL2 is reduced by up to or around 50%, the plants show both a significant increase in grain number, weight and size and also a significant increase in yield. This is shown in FIG. 17.

Accordingly, in one embodiment, the method comprises introducing at least one mutation into a plant such that the plant is heterozygous for a mutation.

In one embodiment, the method may comprise introducing at least one mutation into at least one UPL2 gene and/or promoter, and preferably into all copies or homealleles of the UPL2 gene and/or promoter of a first plant, such that the first plant is homozygous for the mutation, and further crossing the first plant with a second plant (i.e. a wild-type or control plant that does not contain a mutation, such as a loss of function mutation in UPL2) to produce F1 hybrid plants that are heterozygous for the mutation. Also encompassed in the scope of the invention is F1 hybrid seed obtained or obtainable by the cross. This may be particularly useful for rice or maize. Accordingly, in one embodiment, the plant is rice or maize.

In another embodiment, where the plant is a diploid or polyploid, the method comprises introducing a mutation, such as the mutations described above, into one or two homeoalleles in the genome. This may be particularly useful for wheat. Accordingly, in one embodiment, the plant is wheat.

In another embodiment, where RNA silencing is used to reduce the levels of expression of UPL2 the method further comprises the step of selecting plants that show reduced expression of UPL2 by above or around 50%, 55%, 60%, 65% 70%, 75% 80%, 85%, 90% or 95%.

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats).

In a preferred embodiment, the mutation is introduced using CRISPR. The use of this technology in genome editing is well described in the art, for example in U.S. Pat. No. 8,697,359 and references cited herein. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. SgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art, such as such as http://chopchop.cbu.uib.no/it is possible to design sgRNA molecules that target a UPL2 gene or promoter sequence as described herein. In one embodiment, the sgRNA molecules target a sequence selected from SEQ ID No: 27, 28, 29, 30, 31, 33, 34, 35, 36, 41, 42, 45, 46, 49, 50, 51, 52, 53, 54, 65, 66, 67, 68, 70, 71, 72, 73 or 74 or a variant thereof as defined herein. In a further embodiment, the sgRNA molecules comprises a protospacer sequence selected from SEQ ID NO: 27, 28, 29, 30, 31, 37, 38, 39, 40, 43, 44, 47, 48, 49, 50, 51, 52, 53, 54, 65, 66, 67, 68, 70, 71, 72, 73 or 74 or a variant thereof, as defined herein. In a further embodiment, the sgRNA comprises SEQ ID NO: 69 or 75 or a variant thereof.

Cas9 expression plasmids for use in the methods of the invention can be constructed as described in the art.

In one embodiment, the method uses the sgRNA constructs defined in detail below to introduce a targeted mutation into a UPL2 gene and/or promoter.

Alternatively, more conventional mutagenesis methods can be used to introduce at least one mutation into a UPL2 gene or UPL2 promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. In one embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino] acridine dihydrochloride (ICR-170) or formaldehyde. Again, the targeted population can then be screened to identify a UPL2 gene or promoter mutant.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the UPL2 target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the UPL2 nucleic acid sequence may be utilized to amplify the UPL2 nucleic acid sequence within the pooled DNA sample. Preferably, the primer is designed to amplify the regions of the UPL2 gene where useful mutations are most likely to arise, specifically in the areas of the UPL2 gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et. al. 2004.

Rapid high-throughput screening procedures thus allow the analysis of amplification products for identifying a mutation conferring the reduction or inactivation of the f the UPL2 gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene UPL2. Loss of and reduced function mutants with increased grain number compared to a control can thus be identified.

Plants obtained or obtainable by such method which carry a functional mutation in the endogenous UPL2 gene or promoter locus are also within the scope of the invention

In an alternative embodiment, the expression of the UPL2 gene may be reduced at either the level of transcription or translation. For example, expression of a UPL2 nucleic acid, as defined herein, can be reduced or silenced using a number of gene silencing methods known to the skilled person, such as, but not limited to, the use of small interfering nucleic acids (siNA) against UPL2. As shown in FIG. 2D-2H, RNAi against LARGE2 increased the number of primary and secondary branches and grain number.

“Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference. The inhibition of expression and/or activity can be measured by determining the presence and/or amount of UPL2 transcript using techniques well known to the skilled person (such as Northern Blotting, RT-PCR and so on).

Antisense nucleic acid sequences can be designed according to the rules of Watson and Crick base pairing. The antisense nucleic acid sequence may be complementary to the entire UPL2 nucleic acid sequence as defined herein, but may also be an oligonucleotide that is antisense to only a part of the nucleic acid sequence (including the mRNA 5′ and 3′ UTR). The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

In another aspect, the invention extends to a plant obtained or obtainable by a method as described herein.

As shown in FIG. 3, we have found that the large2 regulates the size of the shoot apical meristem and the inflorescence meristem. Accordingly, in a further aspect of the invention, there is provided a method of increasing meristem size and/or activity of a plant, preferably in the grain-width direction, the method comprising introducing at least one mutation, preferably a loss of function mutation into the UPL2 gene as described above. In a preferred embodiment, the method increases the size of apical meristems and inflorescent meristems. An increase in meristem activity may be measured by an increase in the level of expression of meristem activity marker genes, such as but not limited to, LOG, IPA1, SPL14 and KNOX genes, such as OSH1, OSH3, OSH15 and OSH43. Alternatively, an increase in meristem activity may be measured by a decrease in the level of expression of a meristem gene negatively associated with meristem activity such as Gn1a. In one embodiment, meristem size is increased by at least 10%, 15%, 20%, 25%, 30%, 35%, 40%, 45%, 50%, 55%, 60%, 65%, 70%, 75%, 80%, 85%, 90% or 95% or more compared to a control plant.

In another aspect of the invention there is provided a genetically altered plant, part thereof or plant cell characterised in that the plant does not express UPL2 has reduced levels of UPL2 expression, does not express a functional UPL2 protein or expresses a UPL2 with reduced function and/or activity. In a preferred embodiment, the plant expresses a UPL2 polypeptide with reduce or no E3 ligase activity.

For example, the plant is a reduction (knock down) or loss or partial loss of function (knock out) mutant wherein the function of the UPL2 protein is reduced or lost compared to a wild type control plant. To this end, a mutation is introduced into either the UPL2 gene sequence or the corresponding promoter sequence, which disrupts the transcription of the gene. Therefore, preferably said plant comprises at least one mutation in at least one mucelci acid sequence encoding the promoter and/or gene for UPL2. In one embodiment the plant may comprise a mutation in both the promoter and gene for UPL2.

As described in detail above, in a further embodiment, the mutation is any mutation that reduces or abolishes UPL2 E3 ligase activity. Preferably, such a mutation may be in the HECT domain or such mutation leads to a non-functional, truncated or deleted HECT domain. In another embodiment, the mutation is any mutation that reduces or abolishes the binding of UPL2 to its target proteins. Preferably such a mutation is in the Glu/Asp rich domain. By target protein means any ubiquitin protein substrate. In one embodiment, the target protein is APO1 and/or APO2. In a further embodiment, the mutation is in the coding region of the UPL2 gene. In this manner, the activity of the UPL2 polypeptide can be considered to be reduced or abolished as described herein.

In a further aspect of the invention, there is provided a plant, part thereof or plant cell characterised by an increased yield compared to a wild-type or control pant, wherein preferably, the plant, part thereof or plant cell comprises at least one mutation in the UPL2 gene and/or its promoter. Preferably said increase in yield comprises an increase in at least one of seed yield, such as grain number and thousand grain weight.

Preferably, the plant part is a seed. Also provided is progeny plant obtained from the seed as well as seed obtained from that progeny.

The plant may be produced by introducing any one of the above-described mutations into the UPL2 gene and/or promoter sequence by any of the above described methods. Preferably said mutation is introduced into a least one plant cell and a plant regenerated from the at least one mutated plant cell.

As also described above, the plant may be homozygous or heterozygous for the mutation. Where the plant is homozygous for the mutation, the plant may be crossed with a second wild-type or control plant, as described above, to produce a F1 hybrid plant that is heterozygous for the mutation. As shown in FIG. 17, plants that are heterozygous for the mutation also show significant increases in grain size, weight and number as well as produce a significant increase in yield.

Alternatively, the plant or plant cell may comprise a nucleic acid construct expressing an RNAi molecule targeting the UPL2 gene as described herein. In one embodiment, said construct is stably incorporated into the plant genome. These techniques also include gene targeting using vectors that target the gene of interest and which allow integration of a transgene at a specific site. The targeting construct is engineered to recombine with the target gene, which is accomplished by incorporating sequences from the gene itself into the construct. Recombination then occurs in the region of that sequence within the gene, resulting in the insertion of a foreign sequence to disrupt the gene. With its sequence interrupted, the altered gene will be translated into a nonfunctional protein, if it is translated at all.

In another aspect of the invention there is provided a method for producing a genetically altered plant as described herein. In one embodiment, the method comprises introducing at least one mutation into the UPL2 gene and/or UPL2 promoter of preferably at least one plant cell using any mutagenesis technique described herein. Preferably said method further comprising regenerating a plant from the mutated plant cell. In one embodiment, the method may comprise introducing at least one mutation (such as a complete loss of function mutation) into a least one nucleic acid sequence but preferably all copies or homeoalles of a nucleic acid sequence encoding a UPL2 gene and/or UPL2 promoter in a first plant and crossing the first plant with a wild-type or control second plant to produce a F1 hybrid plant that is heterozygous for the mutation.

The method may further comprise selecting one or more mutated plants, preferably for further propagation. Preferably, said selected plants comprise at least one mutation in the UPL2 gene and/or promoter sequence. Preferably said plants are characterised by abolished or a reduced level of UPL2 expression. More preferably, the plants are characterised by a non-functional UPL2 polypeptide. By non-functional is meant, as described above, that the UPL2 polypeptide has reduced or abolished E3 ligase activity and/or is unable to bind its target proteins such as APO1 and APO2.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention there is provided a plant obtained or obtainable by the above-described methods.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an increased yield. Therefore, in this example, increased yield is conferred by the presence of an altered plant genome, for example, a mutated endogenous UPL2 gene or UPL2 promoter sequence. In one embodiment, the endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the various aspects of the invention, methods and uses described herein may be a monocot or a dicot plant. Preferably, the plant is a crop plant. By crop plant is meant any plant which is grown on a commercial scale for human or animal consumption or use. In a preferred embodiment, the plant is a grain crop. In another embodiment the plant is Arabidopsis.

In a most preferred embodiment, the grain crop is a cereal crop (for example, but not limited to rice, wheat, maize, barley, oat, rye, triticale and millet), an oil-seed crop (for example, but not limited to soybean, canola, sunflower, peanut and flax) or a pulse (for example, but not limited to beans, lentils and peas). In one embodiment, the plant may be selected from rice, wheat, maize, soybean, sorghum, oilseed rape and other vegetable brassicas, barley and millet. In one embodiment the plant is rice, preferably the japonica or indica varieties.

We have found that the effect of introducing a loss of function mutation into LARGE2 on yield and grain number is particularly potentiated (i.e. complemented) when combined with a particular plant background. Examples of such backgrounds include those, that when compared with other plant backgrounds, have a higher fertility, better grain filing ability and an increased number of tillers. In one example, where the plant is rice, an example of a particularly useful background is Xiushui09. Other examples would be apparent to the skilled person.

In one particular embodiment, the plant is rice and in particular Xiushui09 and the mutation introduced into the plant is the large2-9 mutation as described above.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed produced from a genetically altered plant as described herein. In an alternative embodiment, the plant part is pollen, a propagule or progeny of the genetically altered plant described herein.

Accordingly, in a further aspect of the invention there is provided pollen, a propagule or progeny of the genetically altered plant as described herein.

A control plant as used herein according to all of the aspects of the invention is a plant which has not been modified according to the methods of the invention. Accordingly, in one embodiment, the control plant does not have reduced expression of a UPL2 nucleic acid and/or reduced activity of a UPL2 polypeptide. In an alternative embodiment, the plant does not contain one or more loss of function mutations in a UPL2 gene or one or more mutations in the UPL2 promoter, as described above. In one embodiment, the control plant is a wild type plant. The control plant is typically of the same plant species, preferably having the same genetic background as the modified plant.

Genome Editing Constructs for Use with the Methods for Targeted Genome Modification Described Herein

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one UPL2 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

By “TAL effector” (transcription activator-like (TAL) effector) or TALE is meant a protein sequence that can bind the genomic DNA target sequence (a sequence within the UPL2 gene or promoter sequence) and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct wherein the nucleic acid construct encodes at least one DNA-binding domain, wherein the DNA-binding domain can bind to a sequence in the UPL2 gene, wherein said sequence is selected from SEQ ID NOs: 33, 34, 35, 36, 41, 42, 45, 46, 49, 50, 51, 52, 53 or 54, or at least one target sequence in the UPL2 promoter sequence, wherein the sequence is selected from SEQ ID NOs: 27, 28, 29, 30, 31, 65, 66, 67, 68, 70, 71, 72, 73 and 74 or a variant thereof. In one embodiment, said construct further comprises a nucleic acid encoding a SSN, such as FokI or a Cas protein.

In one embodiment, the nucleic acid construct encodes at least one protospacer element wherein the sequence of the protospacer element is selected from SEQ ID NOs: 27, 28, 29, 30, 31, 37, 38, 39, 40, 43, 44, 47, 48, 49, 50, 51, 52, 53, 54, 65, 66, 67, 68, 70, 71, 72, 73 or 74 or a variant thereof.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas protein.

In another embodiment, the nucleic acid construct further comprises a tracrRNA sequence. Again, an appropriate tracrRNA sequence would be known to the skilled person as this sequence is defined by the choice of Cas protein.

In a further embodiment, the nucleic acid construct comprises at least one nucleic acid sequence that encodes a sgRNA (or gRNA). Again, as already discussed, sgRNA typically comprises a crRNA sequence, a tracrRNA sequence and preferably a sequence for a linker loop. In a preferred embodiment, the nucleic acid construct comprises at least one nucleic acid sequence that encodes a sgRNA sequence as defined herein in SEQ ID NO: 69 or 75 or variant thereof.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by an endoribonuclease cleavage site.

For example, in one embodiment, at least two sgRNAs are combined as below to introduce a deletion of the below length into the UPL2 promoter sequence.

TABLE 1
Combinations of sgRNAs to introduce a targeted
deletion into the UPL2 promoter sequence

| | | Length of the deletion |
|---|---|---|
| Combinations | Target sites | fragments (base pairs) |
| 1 | proTarget1 (SEQ ID NO: 27) | 344 |
| | proTarget2 (SEQ ID NO: 28) | |
| 2 | proTarget1 (SEQ ID NO: 27) | 807 |
| | proTarget3 (SEQ ID NO: 29) | |
| 3 | proTarget1 (SEQ ID NO: 27) | 1167 |
| | proTarget4 (SEQ ID NO: 30) | |
| 4 | proTarget2 (SEQ ID NO: 28) | 1204 |
| | proTarget3 (SEQ ID NO: 29) | |
| 5 | proTarget2 (SEQ ID NO: 28) | 823 |
| | proTarget4 (SEQ ID NO: 30) | |
| 6 | proTarget3 (SEQ ID NO: 29) | 397 |
| | proTarget5 (SEQ ID NO: 31) | |

Other combinations of target sequences that may be used together in a single construct to introduce a deletion into the UPL2 promoter include: SEQ ID NO: 65 and 67 (referred to herein as MT1T3), SEQ ID: 65 and 68 (referred to herein as MT1T4) and SEQ ID NO: 66 and 67 (referred to herein as MT2T3).

In another embodiment, a nucleic acid construct designed to introduce other mutations into a UPL2 promoter (i.e. other than the above deletion), may comprise the following combinations of sequences in a single construct: SEQ ID NO: 70 and 71 (referred to herein as MT1T3), SEQ ID NO:70 and 72 (referred to herein as MT1T3), SEQ ID NO: 70 and 73 (referred to herein as MT1T4), SEQ ID NO: 70 and 74 (referred to herein as MT1T5) and SEQ ID NO: 72 and 73 (referred to herein as MT3T5).

The term ‘variant’ refers to a nucleotide sequence where the nucleotides are substantially identical to one of the above sequences. The variant may be achieved by modifications such as an insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissue-specific promoter. In one embodiment, suitable plant promoters are selected from, but not limited to U3 and U6.

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is a codon-optimised Cas9 (specific for the plant in question). In another embodiment, the CRISPR enzyme is a protein from the family of Class 2 candidate x proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In an alternative aspect of the invention, the nucleic acid construct comprises at least one nucleic acid sequence that encodes a TAL effector, wherein said effector targets a UPL2 sequence selected from SEQ ID NOs: 27, 28, 29, 30, 31, 33, 34, 35, 36, 41, 42, 45, 46, 49, 50, 51, 52, 53, 54, 65, 66, 67, 68, 70, 71, 72, 73 or 74 or a variant thereof. Methods for designing a TAL effector would be well known to the skilled person, given the target sequence. Examples of suitable methods are given in Sanjana et al., and Cermak T et al, both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is a endonuclease such as FokI. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one sequence selected from SEQ ID NOs: 27, 28, 29, 30, 31, 33, 34, 35, 36, 41, 42, 45, 46, 49, 50, 51, 52, 53, 54, 65, 66, 67, 68, 70, 71, 72, 73 or 74 or a variant thereof.

A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Rahdar et al., 2015, incorporated herein by reference. In this example the crRNA may comprise a phosphorothioate backbone modification, such as 2′-fluoro (2′-F), 2′-O-methyl (2′-O-Me) and S-constrained ethyl (CET) substitutions.

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore is not limited to a single cas function (as would be the case when both cas and sgRNA are encoded on the same nucleic acid construct).

In one embodiment, the nucleic acid construct comprising a cas protein is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a cas protein and co-transfected with at least one nucleic acid construct as defined herein.

Cas9 expression vectors for use in the present invention can be constructed as described in the art. In one example, the expression vector comprises a nucleic acid sequence as defined herein or a functional variant or homolog thereof, wherein said nucleic acid sequence is operably linked to a suitable promoter. Examples of suitable promoters include, but are not limited to Cas9, 35S and Actin.

In an alternative aspect of the present invention, there is provided an isolated plant cell transfected with at least one sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas protein nucleic acid. In other words, the plant is transgene free.

The term “introduction”, “transfection” or “transformation” as referred to anywhere herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation. Transformation of plants is now a routine technique in many species. Any of several transformation methods known to the skilled person may be used to introduce the nucleic acid construct or sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (bioloistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants, can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (B-glucuronidase). Other examples would be readily known to the skilled person. Alternatively, no selection is performed, and the seeds obtained in the above-described manner are planted and grown and UPL2 E3 ligase activity measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the desired mutation (for example, in the HECT domain or the Glu-Asp-rich domain).

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show a reduction in UPL2 E3 ligase activity or an increase in inflorescence size or grain number.

In a further embodiment, the method also comprises the step of screening the genetically modified plant for SSN (preferably CRISPR)-induced mutations in the UPL2 gene or promoter sequence. In one embodiment, the method comprises obtaining a DNA sample from a transformed plant and carrying out DNA amplification to detect a mutation in at least one UPL2 gene or promoter sequence.

In a further embodiment, the methods comprise generating stable T2 plants preferably homozygous for the mutation (that is a mutation in at least one UPL2 gene or promoter sequence).

Plants that have a mutation in at least one UPL2 gene and/or promoter sequence can also be crossed with another plant also containing at least one mutation in at least one UPL2 gene and/or promoter sequence to obtain plants with additional mutations in the UPL2 gene or promoter sequence. The combinations will be apparent to the skilled person. Accordingly, this method can be used to generate a T2 plants with mutations on all or an increased number of homoeologs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the UPL2 gene or promoter sequence. The methods for obtaining the plant of the invention are not exclusively limited to those described in this paragraph; for example, genetic transformation of germ cells from the ear of wheat could be carried out as mentioned, but without having to regenerate a plant afterward.

### Method of Screening Plants for Naturally Occurring Low Levels of UPL2 Expression

In a further aspect of the invention, there is provided a method for screening a population of plants and identifying and/or selecting a plant that will have reduced UPL2 expression or decreased UPL2 E3 ligase activity and/or an increased yield phenotype, preferably an increased seed number or TKW, the method comprising detecting in the plant or plant germplasm at least one polymorphism in the UPL2 gene or promoter. Preferably, said screening comprises determining the presence of at least one polymorphism, wherein said polymorphism is at least one insertion and/or at least one deletion and/or substitution. Preferably said polymorphism leads to a reduced level of UPL2 E3 ligase activity or prevents binding of UPL2 to its target proteins, such as APO1 and/or APO2, compared to a control or wild-type plant.

As a result, the above-described plants will display an increased yield phenotype as described above.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises
a) obtaining a nucleic acid sample from a plant and
b) carrying out nucleic acid amplification of one or more UPL2 gene or promoter alleles using one or more primer pairs.

In a further embodiment, the method may further comprise introgressing the chromosomal region comprising at least one of said UPL2 polymorphisms or the chromosomal region containing the repeat sequence deletion as described above into a second plant or plant germplasm to produce an introgressed plant or plant germplasm. Preferably the expression or activity of UPL2 in said second plant will be reduced or abolished, and more preferably said second plant will display an increase in yield or one of the yield-related parameters as described above.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example 1: Large2 Mutants Produce Large Panicles with Increased Grain Number

To identify new genes for rice panicle size and elucidate the molecular mechanisms underlying panicle size determination, we isolated panicle size mutants by mutagenesis using sodium azide (NaN₃), methanesulfonate (EMS), and cobalt 60, respectively. Nine mutants exhibited similar large-panicle phenotypes, and we named these mutants large2-1 to large2-9 because they had causal mutations in the same gene (see below) (FIG. 1; FIG. 7). The large2-1 mutant was isolated from the NaN₃-treated japonica variety Kongyu131 (KY131). The large2-2 and large2-5 were isolated from the EMS-treated japonica variety Kuanyejing (KYJ). The large2-4, large2-6, large2-7, large2-8, and large2-9 were isolated from the cobalt 60-irradiated KYJ. The large2-3 mutant was isolated from the cobalt 60-irradiated japonica variety Zhonghuajing (ZHJ). All of these nine mutants formed large panicles (FIG. 1B; FIG. 7A). Panicles of these mutants were obviously longer than their respective wild types (FIG. 1E; FIG. 7C). The number of primary panicle branches and the number of secondary panicle branches in large2 mutants were significantly increased, resulting in increased grain number per panicle (FIG. 1F to 1H; FIG. 7C). The grain number of nine large2 mutants (large2-1 to large2-9) was increased by 25.3%, 90.4%, 55.6%, 64.2%, 77.6%, 42.2%, 59.6%, 64.3% and 30.2% compared with that of their respective wild types (FIG. 1H; FIG. 7C). In addition, large2 mutants formed wide leaves and grains (FIG. 1; FIG. 7). Compared with their respective wild types, large2 mutant plants were slightly short, but had thick culms (FIG. 7D). Taken together, these analyses indicate that LARGE2 is involved in the regulation of panicle size, grain number, and grain and organ width in rice.

### Example 2: Cloning of the LARGE2 Gene

The large2-2 and large2-3 mutations were identified using the MutMap approach (Abe et al., 2012; Fang et al., 2016; Huang et al., 2017). We firstly generated F2 populations by crossing large2-2 with KYJ and large2-3 with ZHJ, respectively. For each F2 population, the individuals that showed large-panicle and wide-grain phenotypes were pooled and used for whole-genome resequencing. Meanwhile, the KYJ and ZHJ genomic DNAs were sequenced as controls. We performed sequence analyses and identified candidate causal mutations according to a previous report (Fang et al., 2016). All four SNPs (SNP1-SNP4) were linked to the large-panicle phenotype of large2-2, and three candidate mutations (Indel1, SNP1, and SNP2) were associated with the large-panicle phenotype of large2-3. Interestingly, the SNP2 in large2-2 and the InDel1 in large2-3 happened in the fourteenth exon and fifth exon of the LOC_Os12g24080 gene, respectively (FIG. 2A). Considering that large2-2 and large2-3 showed similar phenotypes, LOC_Os12g24080 could be the causal gene of large2-2 and large2-3.

We also crossed seven mutants (large2-2, large2-4, large2-5, large2-6, large2-7, large2-8 and large2-9) in KYJ background to generate F1 plants with different pairs of these mutations. All the F1 plants produced large panicles with increased primary panicle branches, secondary panicle branches, and grain number per panicle (FIG. 21; FIG. 8), like those observed in large2 mutants. Thus, these results reveal that large2-2, large2-4, large2-5, large2-6, large2-7, large2-8 and large2-9 are allelic, indicating these mutants should have mutations in the same gene (LOC_Os12g24080). To test this sequenced the LOC_Os12g24080 gene in large2-4, large2-5, large2-6, large2-7, large2-8 and large2-9 mutants. As expected, we found that large2-4 contained a 4-bp deletion (AAAG/-) in the fourth exon, large2-5 had a G to A transition in the fourth exon, large2-6 possessed a 1-bp deletion (G/-) in the fifth exon, large2-7 had a 1-bp deletion (T/-) in the tenth exon, large2-8 contained a 13-bp deletion (AATGGATGCTTGA/-) in the eleventh exon, and large2-9 had an A to G change in the exon-intron boundary of intron 11 (FIGS. 2A and 2B). We also sequenced the LOC_Os12g24080 gene in large2-1, which is in the KY131 background, and detected a G to A change in the fourth exon of the LOC_Os12g24080 gene (FIGS. 2A and 2B). Thus, these allelic tests and mutation identifications indicate that LOC_Os12g24080 is the LARGE2 gene.

The genomic sequence of the LOC_Os12g24080 gene is 14.707 kb, and the predicted full-length coding sequence of the LOC_Os12g24080 gene is as long as 10.938 kb. Thus, LOC_Os12g24080 is a very large size gene in rice genome. To further confirm that LOC_Os12g24080 is the LARGE2 gene, we generated LARGE2-RNAi transgenic plants in KY131 background. LARGE2-RNAi transgenic plants showed large panicles with increased primary panicle branch number, secondary panicle branch number, and grain number per panicle compared with KY131 plants (FIG. 2D to 2H). Like large2 mutants, LARGE2-RNAi transgenic plants also produced wide leaves and grains and had the reduced plant height. Taken together, these results reveal that LOC_Os12g24080 is the LARGE2 gene.

### Example 3: LARGE2 Encodes the Functional HECT-Domain E3 Ubiquitin Ligase OsUPL2

LARGE2 encodes the 405-kD E3 ubiquitin ligase OsUPL2, containing the DUF908, DUF913, UBA, DUF4414 and HECT domains (FIG. 2C). Phylogenetic analyses showed that the homologs of LARGE2 are found in plant species and animals (FIGS. 11 and 12), such as Arabidopsis thaliana, Glycine max, Brassica napus, Solanum lycopersicum, Zea mays and Homo sapiens, suggesting that LARGE2 may be an evolutionally conserved protein. In rice, the LARGE2/OsUPL2 family contains seven members (OsUPL1 to OsUPL7). OsUPL1 and LARGE2/OsUPL2 contain more amino acids than other OsUPLs (OsUPL3 to OsUPL7). Rice OsUPL1, OsUPL2/LARGE2 and Arabidopsis AtUPL1/2 are classified into a subgroup, suggesting that they may have conserved functions. However, the role of AtUPL1/2 in panicle development is still unknown so far.

The large2-5 mutation results in an amino acid change from glutamic acid (E) to lysine (K) (FIG. 2C). The other eight large2 mutations lead to different truncated proteins of OsUPL2, which lack partial or whole HECT domain (FIG. 2C). The large2-9 mutation occurs in the exon-intron boundary of intron 11 (FIG. 2A), and results in two main transcripts that are predicted to encode two different versions of proteins lacking the half of the HECT domain (FIG. 2C, FIG. 13). These results indicate that these large2 mutants are loss-of-function alleles.

The HECT domain is required for the activity of HECT-domain E3 ubiquitin ligases in plants and animals (Bates and Vierstra, 1999; Smalle and Vierstra, 2004). As LARGE2/OsUPL2 possesses a HECT domain, we asked if LARGE2 is a functional E3 ubiquitin ligase. To test this, we performed the ubiquitination assay in vitro. The MBP-tagged HECT domain of LARGE2 (MBP-HECT) was expressed in Escherichia coli and then purified for the ubiquitination test. As shown in FIG. 2J, the HECT domain of LARGE2 could be ubiquitinated in the presence of ubiquitin-activating enzyme (E1), ubiquitin-conjugating enzyme (E2) and ubiquitin. For HECT-domain E3 ubiquitin ligases, a conserved cysteine (C) in the HECT domain is required for forming a thioester-linked intermediate with ubiquitin before the modifier is ligated to the substrate (Hershko and Ciechanover, 1998; Callis, 2014). When the conserved cysteine in the HECT domain of LARGE2 was changed to alanine (A) or serine(S), the ubiquitin ligase activity was abolished (FIG. 2J), indicating that an intact HECT domain is required for E3 ubiquitin ligase activity of LARGE2. Thus, these findings indicate that LARGE2 is a functional HECT-domain E3 ubiquitin ligase.

### Example 4: LARGE2 Regulates the Sizes of Shoot Apical Meristems and Panicle Meristems

In the early stage of rice panicle development, the shoot apical meristem (SAM) is converted to the panicle meristem (IM), which turns into two types of meristems, rachis meristem (RM) and branch meristem (BM), according to the developmental stages in rice (Itoh et al., 2005). The sizes of shoot apical meristems and panicle meristems are related to the panicle size in rice (Kurakawa et al., 2007; Huang et al., 2009; Ikeda-Kawakatsu et al., 2012a). Considering large2 alleles had similar panicle and grain number phenotypes, we used large2-2 to investigate the sizes of shoot apical meristems and panicle meristems. We firstly observed SAMs in KYJ and large2-2. As shown in FIGS. 3A and 3B, the SAMs of large2-2 were obviously larger than those of KYJ. We then measured the length of SAMs and counted the number of cells along the SAM length. As shown in FIG. 3C, the SAM length in large2-2 was increased by 10.0% compared to that in KYJ, and cell number was increased by 10.6%, suggesting that LARGE2 regulates meristem size by influencing cell number in the SAMs. After transition to reproductive stage, the RMs of large2-2 were also obviously bigger than those of KYJ (FIG. 3D to 3F). In addition, more primary panicle branch meristems (PBMs) were observed in large2-2 (FIG. 3G to 3I).

In Arabidopsis and rice, several genes involved in the regulation of meristem activity can affect shoot meristem size. Therefore, we asked whether the large sizes of SAMs and RMs and increased number of PBMs in large2-2 could result from the enhanced meristem activity that influences cell number in shoot meristems. To test this, we analyzed the expression of meristem activity marker genes. In rice, knotted1-like homeobox (KNOX) genes, which are recognized as meristem markers, are crucial for establishment and maintenance of the SAM (Tsuda et al., 2011; Tsuda et al., 2014). Mutations in the KNOX gene (OSH1) results in small SAM and reduced grain number (Tsuda et al., 2011). As shown in FIG. 3J, the expression levels of four KNOX genes (OSH1, OSH3, OSH15 and OSH43) were significantly increased in large2-2 compared with those in KYJ. The biosynthesis and signaling of cytokinin are known to regulate the size and activity of reproductive meristems (Werner et al., 2001; Lee et al., 2019). The LONELY GUY (LOG) gene, which encodes a cytokinin-activating enzyme, directly controls meristem activity, and its loss-of-function mutant causes premature termination of shoot meristems and small panicles (Kurakawa et al., 2007). Gn1a, which encodes a cytokinin oxidase/dehydrogenase (OsCKX2), negatively regulates panicle size and grain number in rice (Ashikari et al., 2005). As shown in FIG. 3K, expression of LOG was significantly increased in large2-2 compared with that in KYJ, while expression of OsCKX2 was lower in large2-2 than that in KYJ. Additionally, Ideal Plant Architecture 1 (IPA1)/OsSPL14, Dought and Salt Tolerance (DST) and JMJ703 have been reported to be involved in the regulation of panicle size and grain number (Jiao et al., 2010; Miura et al., 2010; Cui et al., 2013; Li et al., 2013; Liu et al., 2015). The expression level of IPA1/OsSPL14 in large2-2 was significantly increased compared with that in KYJ, while the expression levels of DST and JMJ703 in large2-2 were similar to those in KYJ (FIG. 3K). These results indicate that LARGE2 influences meristem activity, at least in part, by affecting the expression of meristem activity marker genes.

Besides large panicles, the large2 mutants formed wide grains and leaves. The wide grains and leaves could result from increased cell number and/or large cells (Li and Li, 2016). We therefore examined cell size and cell number in the grains and leaves of KYJ and large2-2. Cell width in the transverse direction of the outer surface of large2-2 lemmas was comparable with that of KYJ lemmas. By contrast, cell number in the grain-width direction in large2-2 lemmas was significantly increased compared with that in KYJ lemmas. Similarly, cell number in the transverse direction of large2-2 flag leaves was higher than that of KYJ flag leaves. Thus, these results reveal that LARGE2 controls the width of grains and leaves by restricting cell proliferation.

### Example 5: Expression Pattern of LARGE2

Quantitative real-time reverse-transcriptase PCR (qRT-PCR) analysis was performed to detect the expression pattern of LARGE2. The LARGE2 transcripts were detected in roots, stems, leaves, leaf sheaths and developing panicles (FIG. 4A). The expression of LARGE2 in young panicles was relatively higher than that in old ones (FIG. 4A). In addition, transgenic plants containing the LARGE2 promoter: GUS fusion (proLARGE2: GUS) were generated to analyze the expression pattern of LARGE2. Histological section pictures showed that GUS activity was detected in SAMs (FIG. 4B). In developing panicles, PBMs and floral meristems displayed stronger GUS activity (FIG. 4C to 4E). GUS activity was detected in different tissues, including roots, culms, leaves and developing panicles (FIG. 4F to 4O). Similarly, GUS activity in younger panicles was obviously stronger than that in older panicles (FIG. 4L to 4O). Thus, expression pattern of LARGE2 is consistent with its role in the regulation of meristem activity and cell proliferation.

### Example 6: LARGE2 Associates with APO1 and APO2

APO1 has been reported to regulate panicle development, thereby influencing panicle size and grain number in rice (Ikeda et al., 2007; Ikeda-Kawakatsu et al., 2009). Interestingly, STRONG CULM2 (SCM2), a gain-of-function mutant of APO1, showed large panicles with increased grain number and thick culms (Ookawa et al., 2010), which resembled those observed in large2 mutants. By contrast, loss-of-function mutants apo1 and large2 showed opposite phenotypes in panicle size, grain number, culm thickness and leaf width (FIG. 1; FIG. 7) (Ikeda et al., 2005; Ikeda et al., 2007). Considering that LARGE2 is a functional E3 ubiquitin ligase, we asked whether LARGE2 could physically associate with APO1 to modulate its stability. Split luciferase complementation assay was firstly employed to test the interaction between LARGE2 and APO1. Since LARGE2 is a 405-KD large protein, we divided LARGE2 into five fragments (LARGE2-F1 to LARGE2-F5) to test their interactions with APO1 (FIG. 5A). We co-expressed LARGE2-F1-nLUC, LARGE2-F2-nLUC, LARGE2-F3-nLUC, LARGE2-F4-nLUC, and LARGE2-F5-nLUC (nLUC, N-terminal luciferase) with cLUC-APO1 (cLUC, C-terminal luciferase) in leaves of Nicotiana benthamiana, respectively. As shown in FIG. 5B, co-expression of cLUC-APO1 with LARGE2-F3-nLUC showed luciferase activity, while co-expression of cLUC-APO1 with LARGE2-F1-nLUC, LARGE2-F2-nLUC, LARGE2-F4-nLUC, or LARGE2-F5-nLUC had no luciferase activity, indicating that LARGE2-F3 associates with APO1 in planta. Similarly, a recent study showed that the corresponding region of the human HECT-domain ubiquitin ligase HUWE1, a homolog of LARGE2, contributes to its interaction with another protein (Wang et al., 2014). This corresponding region of human HUWE1 contains a nuclear localization signal (NLS) and a Glu/Asp rich domain (Wang et al., 2014). As both LARGE2-F3 and HUWE1-F3 contain a Glu/Asp rich domain (Wang et al., 2014), we asked whether the Glu/Asp rich domain in LARGE2 is required for the association of LARGE2 with APO1. The split luciferase complementation assay showed that the deletion of the Glu/Asp rich domain abolished the association of LARGE2-F3 with APO1. Thus, these findings indicate that the Glu/Asp rich domain of LARGE2 is required for the association of LARGE2 with APO1.

Previous study has shown that APO2 physically and genetically interacts with APO1 to regulate rice panicle development (Ikeda-Kawakatsu et al., 2012). We sought to investigate if LARGE2 could associate with APO2. As shown in FIG. 5C, co-expression of cLUC-APO2 with LARGE2-F3-nLUC showed luciferase activity, indicating that LARGE2-F3 also associates with APO2 in planta. Meanwhile, the deletion of the Glu/Asp rich domain abolished the association of LARGE2-F3 with APO2, indicating that the Glu/Asp rich domain of LARGE2 is also indispensable for the association of LARGE2 with APO2.

To further verify the association of LARGE2 with APO1 and APO2, we performed co-immunoprecipitation assay. We transiently expressed 35S: Myc-LARGE2-F3 with 35S: GFP-APO1 or 35S: GFP-APO2 in leaves of Nicotiana benthamiana. Total proteins were isolated and incubated with GFP beads. The anti-GFP and anti-Myc antibodies were used to detect immunoprecipitated proteins. As shown in FIGS. 5D and 5E, Myc-LARGE2-F3 was co-immunoprecipitated with GFP-APO1 or GFP-APO2, but not with the negative control (GFP). Taken together, these results indicate that LARGE2 associates with APO1 and APO2 in planta.

### Example 7: LARGE2 Modulates the Stability of APO1 and APO2 in Rice

As LARGE2 is a functional E3 ubiquitin ligase and associates with APO1 and APO2, we sought to test if LARGE2 could modulate the stabilities of APO1 and APO2. GFP-APO1 and GFP-APO2 were expressed in Nicotiana benthamiana leaves respectively, and then treated with proteasome inhibitor MG132. After treatment with MG132, the levels of GFP-APO1 and GFP-APO2 fusion proteins were obviously increased (FIGS. 6A and 6B) and 25F), suggesting that the ubiquitin proteasome affects the stabilities of APO1 and APO2.

We used the rice cell-free system to test whether LARGE2 could influence the degradation of APO1 and APO2. APO1-His and APO2-His fusion proteins were expressed in Escherichia coli and purified with His-MA (magnet) beads. The purified APO1-His and APO2-His fusion proteins were incubated in cell-free extracts from ZHJ and large2-3 seedlings, respectively. The extracts from ZHJ seedlings caused a more rapid degradation of APO1-His and APO2-His than those from large2-3 seedlings.

To further test if LARGE2 influences the stabilities of APO1 and APO2 in rice, we generated 35S: GFP-APO1 and 35S: GFP-APO2 transgenic lines, and crossed them with large2-3 to obtain 35S: GFP-APO1;large2-3 and 35S: GFP-APO2;large2-3 plants respectively. Western blot analyses showed GFP-APO1 proteins in 35S: GFP-APO1; large2-3 young panicles accumulated at a higher level than those in 35S: GFP-APO1 (FIGS. 6C and 6D). By contrast, the transcription levels of GFP-APO1 in 35S: GFP-APO1 and 35S: GFP-APO1;large2-3 were comparable (FIG. 6E). Similarly, GFP-APO2 proteins in 35S: GFP-APO2;large2-3 young panicles accumulated at a higher level than those in 35S: GFP-APO2 (FIG. 6H and 6I). The transcription levels of GFP-APO2 in 35S: GFP-APO2 and 35S: GFP-APO2;large2-3 were similar (FIG. 6J). Thus, these results reveal that LARGE2 modulates the stabilities of APO1 and APO2 in rice.

### DISCUSSION

Panicle/infloresence size and grain number are important agronomic traits (Wang et al., 2018). However, how plants determine their panicle size and grain number remains largely unknown. In this study, we identify the HECT-domain E3 ubiquitin ligase LARGE2/OsUPL2 as a negative regulator of panicle size and grain number in rice. LARGE2 associates with APO1 and APO2, and modulates their stabilities. LARGE2 functions genetically with APO1 and APO2 to regulate panicle size and grain number. Our findings reveal a novel molecular and genetic mechanism of the LARGE2-APO1/APO2 module in controlling panicle size and grain number.

We identified nine large2 alleles in KY131, KYJ and ZHJ varieties, respectively. Although KY131, KYJ and ZHJ varieties showed obvious differences in panicle size and grain number, large2 alleles exhibited dramatic increases in panicle size and grain number compared with their respective wild types, indicating that LARGE2 is a negative regulator of panicle size and grain number. Cellular observations reveal that large2 mutants had large apical meristems (SAMs) and rachis meristems (RMs) and increased primary branch meristems (PBMs). Additionally, the large SAMs in large2 mutants resulted from increased cell number in SAMs. Consistent with this idea, we observed that expressions of several marker genes, which control panicle/panicle development by regulating meristem activity (Kurakawa et al., 2007; Tsuda et al., 2011; Tsuda et al., 2014), were significantly altered in large2-2. For example, mutations in the LOG gene decrease meristem activity and cause small shoot meristems and panicles with reduced grain number (Kurakawa et al., 2007), while mutations in OSH1, a meristem marker crucial for establishment and maintenance of the SAM, result in aberrant SAMs and small panicles (Tsuda et al., 2011). Expressions of LOG and OSH1 were increased in large2 compared with those in the wild type. Thus, it is possible that high meristem activity in large2 mutants causes the increased cell number and large shoot meristems that determine panicle size and grain number. The large2 mutants also showed wide grains and leaves and thick culms, implying that LARGE2 is a regulator of other organ growth. The large2 mutants showed increased cell number in both grain-width and leaf-width directions, indicating that LARGE2 limits cell proliferation. Supporting the roles of LARGE2 in meristematic activity and cell proliferation, higher expression of LARGE2 was detected in younger panicles than that in older ones. Several studies suggested the trade-off between grain number and grain size in rice. For example, loss-of-function mutations in OsMKP1 caused large grains and reduced grain number per panicle, while overexpression of OsMKP1 resulted in small grains and increased grain number per panicle (Guo et al., 2018; Xu et al., 2018a). Interestingly, large2 mutants produced large panicles with increased grain number and wide grains, suggesting the potential utilization of LARGE2 in increasing both grain number and grain size in rice.

LARGE2 encodes a predicted HECT-domain E3 ubiquitin ligase OsUPL2. Our ubiquitination assays demonstrated that the HECT domain is required for the activity of LARGE2 E3 ubiquitin ligase. Homologs of LARGE2/OsUPL2 are found in plant species as well as animals. In Arabidopsis, the AtUPL3 and AtUPL5 have been shown to regulate trichome development and leaf senescence, respectively (Downes et al., 2003; Miao and Zentgraf, 2010; Patra et al., 2013). A recent study has shown that AtUPL3 promotes proteasomal processes and controls plant immunity (Furniss et al., 2018). The oilseed rape HECT-domain E3 ubiquitin ligase BnaUPL3.C03 is associated with seed size and field yields (Miller et al., 2019). In rice, the LARGE2/OsUPL2 family contains seven members (OsUPL1 to OsUPL7), but their functions have not been described previously. In this study, we identified LARGE2 as a negative regulator of panicle size and grain number in rice. Rice OsUPL1 and OsUPL2/LARGE2 share relatively high similarity with Arabidopsis AtUPL1 and AtUPL2, suggesting that they may have conserved functions.

Previous studies showed that APO1 and APO2 influences panicle size and grain number (Ikeda et al., 2007; Ikeda-Kawakatsu et al., 2009). APO1 is an ortholog of Arabidopsis F-box protein UFO (Ikeda-Kawakatsu et al., 2012). In Arabidopsis, UFO interacts with the transcription factor LFY, and functions as a transcriptional cofactor of LFY in the control of floral development (Chae et al., 2008). Interactions between orthologs of LFY and UFO are also observed in several plant species. In petunia, the UFO ortholog DOT interacts with and activates the LFY ortholog ALF by a posttranscriptional mechanism in the control of floral meristem identity establishment (Souer et al., 2008). Likewise, APO1 physically associates with APO2, an ortholog of LFY, and genetically interacts with APO2 to control panicle development in rice (Ikeda-Kawakatsu et al., 2012). Interestingly, apo1 and apo2 mutants had opposite phenotypes to large2 mutants with respect to panicle size, grain number and culm thickness (Ikeda et al., 2005; Ikeda et al., 2007; Ikeda-Kawakatsu et al., 2012). Biochemical analyses revealed that LARGE2 associates with APO1 and APO2 in planta. We also observed that mutations in LARGE2 caused the accumulation of APO1 and APO2 proteins in rice. LARGE2 also influences stabilities of APO1 and APO2 in rice cell-free system. Considering that LARGE2 is a functional E3 ubiquitin ligase, it is plausible that LARGE2 might ubiquitinate APO1 and APO2 and influences their stabilities. Unfortunately, we failed to express and purify the full-length LARGE2 to test if LARGE2 could directly ubiquitinate APO1 in vitro because LARGE2 protein (405-kD) is too large. Consistent with biochemical analyses, our genetic data suggest that LARGE2 acts with APO1 and APO2, at least in part, in a common pathway to control panicle size and grain number. Supporting this, LARGE2, APO1 and APO2 share overlapped expression patterns in apical meristems, rachis meristems, primary branch meristems and floral meristems (Ikeda et al., 2007; Ikeda-Kawakatsu et al., 2012). Therefore, our findings reveal a novel molecular and genetic mechanism of the LARGE2-APO1/APO2 module-mediated control of panicle size and grain number in rice.

### Example 8: Methods

### Plant Materials and Growth Conditions

The large2-1 mutant was isolated from Kongyu131 (KY131) by sodium azide (NaN₃) treatment. The large2-2 and large2-5 mutants were isolated from Kuanyejing (KYJ) by methanesulfonate (EMS) treatment. The large2-3 mutant was isolated from Zhonghuajing (ZHJ) by cobalt 60 irradiation. The large2-4, large2-6, large2-7, large2-8, and large2-9 mutants were isolated from Kuanyejing (KYJ) by cobalt 60 irradiation. Plants were grown in Beijing, Hangzhou (Zhejiang province) and Lingshui (Hainan province) under natural conditions.

### Morphological and Cellular Analyses

Plants were grown in the rice fields. Plants at the mature stage were dug out and put into pots, and then photographed with a Nikon D7000 camera. The main panicles, grains, flag leaves and the third internodes from the mature plants were used for analyses of panicle size, grain width, leaf width and culm thickness, respectively. We used a Scan Marker i560 (Microtek) to scan grains, and measured the grain width with the Rice Test System (WSeen).

Scanning microscopic analyses of rachis meristems, primary branch meristems, grain lemmas and flag leaves were performed according to a previous research (Duan et al., 2014). After fixation in FAA solution (formalin:glacial acetic acid:50% ethanol; 1:1:18) at 4° C. overnight and dehydration in a graded ethanol series, the samples were dried with the critical-point drier (Hitachi HCP-2), and dissected under a microscope (Leica S8APO). We sputter-coated the samples with platinum and observed them with a scanning electron microscope (Hitachi S-3000N). Image J software was used to measure cell size.

Clearing of shoot apical meristems (SAMs) was performed according to a previous research (Ikeda et al., 2005). After fixation in FAA solution (formalin:glacial acetic acid: 50% ethanol; 1:1:18) at 4° C. overnight and dehydration in a graded ethanol series, samples were transferred into BB4-1/2 clearing fluid (Herr, 1982). We observed the cleared samples using the Leica DM2500 microscope with differential interference contrast optics, and photographed the samples using the Spot Flex cooled digital imaging system.

Paraffin sectioning of the third internodes and GUS staining samples was performed according to a previous study (Ikeda et al., 2005). After fixation in FAA solution (formalin:glacial acetic acid:50% ethanol; 1:1:18) at 4° C. overnight and dehydration in a graded ethanol series, samples were transferred to a graded xylene series, embedded in Paraplast Plus (Sigma-Aldrich) and sectioned at 8 μm in thickness with a rotary microtome (Leica). We stained the sections of the third internodes with 0.05% toluidine blue and observed the samples using the Leica DM2500 microscope.

### Identification of the LARGE2 Gene

The large2-2 and large2-3 mutants were crossed with ZHJ and KYJ to generate F2 populations, respectively. The F2 populations were used for cloning the LARGE2 gene. The whole genomes of wild-type and a mixed pool of 50 individual plants with large panicle phenotypes were resequenced using NextSeq 500 (Illumina). MutMap and SNP/INDEL-index analyses were performed according to a previous research (Fang et al., 2016). After whole genome resequencing, the short reads were aligned to the reference genome sequence (Nipponbare), and a certain number of SNPs and INDELs specific for the bulked F2 were obtained. For each SNP/INDEL, we calculated the SNP/INDEL-index, which referred to the ratio between the number of reads for a mutant SNP/INDEL and total number of reads. The SNPs and INDELs with SNP/INDEL-index=1 were selected for further sequence analyses.

### Constructs and Plant Transformation

The primers LARGE2-RNAi-F and LARGE2-RNAi-R were used to amplify the 417-bp sequence of LARGE2 3′UTR, which was cloned into pZH2Bi vector in forward and reverse directions to generate the LARGE2-RNAi vector. The LARGE2-RNAi vector was transformed into the japonica variety KY131 using Agrobacterium GV3101.

The 195-bp fragment of APO1 was amplified using the primers APO1-RNAi-F and APO1-RNAi-R, and then was cloned into pZH2Bi in forward and reverse directions to generate the APO1-RNAi transformation vector. The APO1-RNAi vector was transformed into large2-1 using Agrobacterium GV3101.

The primers GFP-APO1-F and GFP-APO1-R were used to amplify the APO1 CDS, which was then inserted into the pMDC43 to generate the transformation vector 35S: GFP-APO1. The 35S: GFP-APO1 vector was transformed into the japonica variety ZHJ using Agrobacterium GV3101.

The 3,312-bp promoter of LARGE2 was amplified with the primers proLARGE2-GUS-F and proLARGE2-GUS-R, and then was cloned into the pZHEX vector to construct the transformation vector proLARGE2: GUS. The proLARGE2: GUS vector was transformed into the japonica variety KY131 using Agrobacterium GV3101.

### Ubiquitin Ligase Activity Assay

The coding sequence of the HECT domain of LARGE2/OsUPL2 was cloned into the pMAL-2c vector to construct the MBP-HECT vector by using the primers HECT-F/R. The conserved Cysteine was mutated to Alanine and Serine by using the primers HECT (Ala)-F/R and HECT (Ser)-F/R, respectively.

Protein expression and purification was performed according to a previous research (Xia et al., 2013). The MBP-HECT, MBP-HECT (Ala) and MBP-HECT (Ser) vectors were transformed into Escherichia coli BL21 to express MBP-HECT, MBP-HECT (Ala) and MBP-HECT (Ser), respectively. Bacteria lysates for expressing different fusion proteins were induced with 0.8 mM isopropyl-β-D-1-thiogalactopyranoside (IPTG) for 1.5 h. We lysed the bacteria with resuspension buffer (150 mM NaCl, 50 mM HEPES PH 7.4, 1% Triton X-100, 10% glycerol and protease inhibitor cocktail) and sonicated the bacteria.

The lysates were centrifuged at 12,000 rpm for 10 min. The supernatant was incubated with amylose resin (New England Biolabs) at 4° C. with rotation for 1 h. Beads were washed with wash buffer (150 mM NaCl, 50 mM HEPES pH 7.4 and 10% glycerol) for five times, and then added with elution buffer (200 mM NaCl, 20 mM Tris-HCl pH 7.4, 10 mM maltose, 1 mM DTT and 1 mM EDTA) at 4° C. with rotation for 30 min. After centrifugation, the eluted supernatant was the purified MBP fusion protein.

Ubiquitin ligase activity assay was performed according to a previous research (Xia et al., 2013). We incubated 110 ng E1 (Boston Biochem), 170 ng E2 (Boston Biochem), 1 mg His-ubiquitin (Sigma-Aldrich), and 2 mg MBP-HECT or mutated MBP-HECT fusion protein in 20 μL reaction buffer (50 mM Tris-HCl PH 7.4, 20 mM DTT, 5 mM MgCl₂ and 2 mM ATP) at 30° C. for 2 h. SDS-loading buffer (Cwbiotech) was added to stop the reaction, and we put the samples in 98° C. dry bath for 10 min and subjected the samples to the SDS-PAGE analysis. Anti-MBP (Abmart) and anti-His (Abmart) antibodies were used to detect the polyubiquitinated proteins, respectively. The eECL Western Blot Kit (Cwbiotech) was used to detect signals, and Tanon-4500 gel-imaging system was used to analyze the signals according to instructions from the manufacturer.

### Phylogenetic Analysis

The full-length protein sequences of LARGE2/OsUPL2 homologs in different species were used to construct the phylogenetic tree. A neighbor-joining method in MEGA5.0 program was used to construct the phylogenetic tree. The parameters were as follows: complete deletion and bootstrap (1000 replicates).

### Gus Staining

The developing panicles, seedlings and other tissues of proLARGE2: GUS transgenic plants were collected and kept in a GUS staining buffer (750 μg/ml X-gluc, 10 mM EDTA, 3 mM K₃Fe(CN)₆, 100 mM NaPO₄ PH 7 and 0.1% Nonidet-P40) at 37° C. incubator for 6 hours. Then the samples were transferred to 70% ethanol to remove chlorophyll.

### RNA Extraction and Quantitative Real-Time RT-PCR

The plant RNA isolation kit (Tiangen) was used to extract total RNA from different organs. The SuperScript III transcriptase kit (Invitrogen) was used for synthesizing complementary DNA from the RNA sample (5 mg). Taq Master Mix (Cwbiotech) was used for RT-PCR. Quantitative real-time RT-PCR analyses were performed with the Bio-Rad CFX96 real-time PCR detection system using the RealStar Green Fast Mixture (GenStar). The rice Actin1 was used as internal control. The Cycle threshold (Ct) method was used to calculate relative amounts of mRNA.

### Split Luciferase Complementation Assay

The coding sequences of APO1 and LARGE2 fragments were cloned into pCAMBIA-split_cLUC and pCAMBIA-split_nLUC to generate cLUC-APO1 and OsUPL2-Fs-nLUC vectors, respectively. Agrobacterium GV3101 cells containing different combinations of cLUC-APO1 and OsUPL2-Fs-nLUC vector pairs were transformed into N. benthamiana leaves as described previously (Li et al., 2018). We sprayed N. benthamiana leaves with 0.5 mM luciferin and incubated them in NightOWL II LB983 imaging apparatus for 5 min before luminescence detection.

### Co-Immunoprecipitation Assay

The coding sequences of APO1 and LARGE2-F3 were cloned into pMDC43 and pCambia1300-221-Myc to generate GFP-APO1 and Myc-OsUPL2-F3, respectively. Agrobacterium GV3101 cells harboring different combinations of GFP and Myc vector pairs were transformed into N. benthamiana leaves. Co-immunoprecipitation assay was performed as described before (Wang et al., 2016). Total proteins were extracted with the extraction buffer (150 mM NaCl, 50 mM Tris-HCl PH 7.4, 1 mM EDTA, 2% Triton X-100, 20% glycerol, protease inhibitor cocktail and 1 mM PMSF) and incubated with GFP beads (Chromotek) at 4° C. with rotation for 1 h. Beads were washed three times with the wash buffer (150 mM NaCl, 50 mM Tris-HCl PH 7.4, 1 mM EDTA, 20% glycerol, 0.1% Triton X-100 and protease inhibitor cocktail). After adding SDS-loading buffer (Cwbiotech), we put the samples in 98° C. dry bath for 10 min and subjected the samples to the SDS-PAGE analysis. Anti-Myc (Abmart) and anti-GFP (Abmart) antibodies were used to detect the immunoprecipitates, respectively. The eECL Western Blot Kit (Cwbiotech) was used to detect signals, and Tanon-4500 gel-imaging system was used to analyze the signals according to instructions from the manufacturer.

### Protein Stability Analyses

For protein stability assay in rice, total proteins were extracted from young panicles (1 cm) of transgenic plants. For protein stability assay in N. benthamiana leaves, the 35S: GFP-APO1 was transformed into N. benthamiana leaves using Agrobacterium GV3101. After two days, the transformed N. benthamiana leaves were treated with MG132 or DMSO for 24 hours, and then total proteins were extracted. Total protein extraction was performed according to previous studies (Xia et al., 2013; Wang et al., 2016). Total proteins were subjected to SDS-PAGE analysis. We detected the proteins by immunoblot analyses with anti-GFP (Abmart) and anti-Actin (Abmart) antibodies, respectively. The eECL Western Blot Kit (Cwbiotech) was used to detect signals, and Tanon-4500 gel-imaging system was used to analyze the signals according to instructions from the manufacturer. The GFP-APO1 protein level was quantified relative to the Actin protein level by ImageJ software.

### Example 9

In one embodiment, it has been found that compared to Nipponbare (a japonica rice variety that has been sequenced), almost all indica rice varieties have a 2.6-kb deletion in the OsUPL2 promoter region, and almost all japonica varieties have the complete sequence. As indica varieties have larger panicles than japonica varieties, the 2.6-kb sequence in the promoter of OsUPL2 may correlate to panicle size. Without being bound by theory, it is possible that during evolution, the natural variation in the OsUPL2 promoter (i.e. deletion of 2.6 kp sequence) might lead to changes in panicle size between indica and japonica varieties through changing UPL2 expression levels. To test this, we have used CRISPR to obtain different deletions, and in particular to delete the 2.6 kbp sequence in the UPL2 promoter.

An example of suitable CRISPR constructs to target the 2.6-kb in the OsUPL2/LARGE2 promoter are described below. In one example, the target sequence is selected from one of the following:

Target 1 (T1):
(SEQ ID NO: 65)
TAGAATATATCTGAGGGAA
Target 2 (T2):
(SEQ ID NO: 66)
GTGAAAGGACTGTCGAGGC
Target 3 (T3):
(SEQ ID NO: 67)
ATATTCTCAAAATCGAATC
Target 4 (T4):
(SEQ ID NO: 68)
AATCGAATCTGGACTGTTT

In one example, one construct contains to two target sites, one upstream of the 2.6-kb site for deletion and the other downstream. In this example, we constructed three constructs, called MT1T3, MT1T4 and MT2T3.

In one example, the full sgRNA sequence is as follows: (SEQ ID NO: 69)

GTTTTAGAGCTAGAAATAGCAAGTTAAAATAAGGCTAGTCCGTTATCAAC
TTGAAAAAGTGGCACCGAGTCGGTGCTTTTTTTTTTCGTTTTGCATTGAG
TTTTCT

### Part II

CRISPR constructs to obtain different deletions in the OsUPL2/LARGE2 promoter. Examples of CRISPR constructs that may be used to obtain different mutations in the UPL2 promoter are as follows.

In one example, the target sequence may be selected from one of the below target sequences:

Target 1 (T1):
(SEQ ID NO: 70)
GCAGTCTTCGTTCTCGTGT
Target 2 (T2):
(SEQ ID NO: 71)
GCAGGTCCCGCCTCTAATC
Target 3 (T3):
(SEQ ID NO: 72)
TGCCGGGCCGGTTAACAAT
Target 4 (T4):
(SEQ ID NO: 73)
GCGCGGCGGGTTACCTCTA
Target 5 (T5):
(SEQ ID NO: 74)
GAGGGCCCCCGATCGCGGC

One construct contains to two target sites. In one example, we constructed five constructs, MT1T2, MT1T3, MT1T4, MT2T3, MT2T4 and MT3T5.

In one example, the full sgRNA sequence is as follows (SEQ ID NO: 75)

GTTTTAGAGCTAGAAATAGCAAGTTAAAATAAGGCTAGTCCGTTATCAAC
TTGAAAAAGTGGCACCGAGTCGGTGCTTTTTTTTTTCGTTTTGCATTGAG
TTTTCT

Method of CRIPSR constructions (for constructions in both Part I and Part II) An example of a method to produce CRISPR constructs for introducing one or more of mutations into the UPL2 promoter is shown below and in FIG. 16.
1. Input the sequence in http://crispor.tefor.net/and pick up the target sequences from outputs.
2. Design primers for the CRISPR constructions.

MT1T2-F:
AATAATGGTCTCAGGCGNNNNNNNNNNNNNNNNNNN
MT1T2-F0:
GNNNNNNNNNNNNNNNNNNNGTTTTAGAGCTAGAAATAGC
MT1T2-R0:
NNNNNNNNNNNNNNNNNNNCGCTTCTTGGTGCC
MT1T2-R:
ATTATTGGTCTCTAAACNNNNNNNNNNNNNNNNNNN

Replace the 19-nt N with 19-nt target sequence in F/F0. Replace the 19-nt N with 19-nt target sequence (reverse complement) in R/R0.
3. PCR amplification with the four primers in step 2.
Template: pCBC-MT1T2
Primer: MT1T2-F/R 10 μM, MT1T2-F0/R0 0.5 μM
4. Purify the PCR products, and put the following ingredients in the restriction-ligation system. Destination vector: pHUE-411 (Kan). As shown in FIG. 16.
5. Transfer 5 μL of the restriction-ligation system into DH5a. Primers OsU3-FD3 and TaU3-RD are used to identify the bacteria grown in media with kanamycin, and the right PCR products are 831-bp. Primers OsU3-FD3 and TaU3-FD2 are used for sequencing the vectors.

OsU3-FD3:
(SEQ ID NO: 76)
GACAGGCGTCTTCTACTGGTGCTAC
TaU3-RD:
(SEQ ID NO: 77)
CTCACAAATTATCAGCACGCTAGTC
(SEQ ID NO: 78)
[rc: GACTAGCGTGCTGATAATTTGTGAG]
TaU3-FD:
(SEQ ID NO: 79)
TTAGTCCCACCTCGCCAGTTTACAG
TaU3-FD2:
(SEQ ID NO: 80)
TTGACTAGCGTGCTGATAATTTGTG

### Example 10

As shown in FIG. 17, we crossed large2-1 with its wild-type KY131 to get KY131/large2-1. Compared to KY131, although KY131/large2-1 has slightly less tillers, KY131/large2-1 has more primary branches, secondary branches and grain number as well as wider grains, like the phenotypes of large2-1. Additionally, KY131/large2-1 has higher 1,000-grain weight. As a result, KY131/large2-1 has higher grain yield than KY131.

SEQUENCE LISTING

SEQ ID NO: 1: OsUPL2 CDS sequence

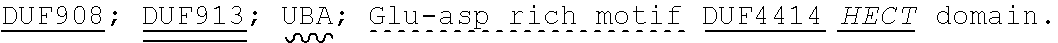

ATGGCGGCGGCGGCGGCCATGGCGGCGCACCGGGCCAGCTTCCCGCTCCGGCT
GCAGCAGATCCTGTCCGGGAGCCGCGCCGTGTCGCCGTCGATCAAGGTGGAGT
CCGAGCCGCCAGCAAAAGTTAAAGCATTTATTGATCGTGTAATCAGTATTCCACTA
CATGACATTGCTATACCATTGTCAGGCTTCCGTTGGGAGTTCAATAAGGGAAATTT
CCACCATTGGAAGCCTCTTTTTATGCATTTTGATACATATTTCAAGACACAAATTTC
TTCGAGGAAGGATCTTCTTTTATCTGATGATATGGCTGAGGGTGATCCTTTGCCTA
AAAATACCATCCTGCAGATTTTGAGAGTAATGCAGATTGTTTTGGAAAATTGCCAG
AACAAAACATCGTTTGCTGGTCTTGAGCATTTTAGGCTTCTGCTGGCATCATCAGA
TCCTGAGATAGTTGTGGCTGCTTTAGAGACACTTGCTGCATTGGTTAAAATAAATC
CTTCGAAGTTGCATATGAACGGAAAGCTCATAAATTGTGGAGCTATAAACAGTCAT
CTTCTATCATTGGCACAAGGATGGGGTAGCAAGGAGGAAGGTTTGGGCTTATATT
CTTGTGTTGTGGCAAATGAAAGAAACCAGCAGGAGGGTTTGTGCTTATTCCCAGC
AGACATGGAGAACAAATACGATGGCACGCAGCACCGTCTCGGTTCAACTCTTCAT
TTTGAATATAATTTGGCACCTGCCCAAGATCCTGACCAATCCAGTGACAAGGCTAA
GCCATCTAATCTGTGTGTGATACATATCCCAGACTTGCACCTTCAGAAGGAGGAT
GACTTGAGCATATTGAAGCAATGTGTTGATAAGTTTAATGTGCCTTCAGAGCACAG
ATTTTCCTTGTTTACAAGGATAAGATATGCCCATGCCTTTAATTCGCCACGGACAT
GTAGGCTATATAGCCGCATAAGTCTTCTTGCTTTCATTGTTCTTGTGCAATCCAGC
GATGCCCATGATGAACTCACATCTTTCTTTACAAATGAGCCAGAGTACATAAATGA
GTTAATCAGACTTGTCCGATCAGAGGAATTTGTTCCTGGACCCATACGAGCGCTG
GCTATGCTTGCACTGGGAGCACAGTTAGCAGCGTATGCATCATCTCATGAACGAG
CTCGGATACTTAGTGGCTCAAGTATCATATCTGCTGGTGGAAACCGCATGGTCTT
GCTCAGTGTTTTGCAAAAAGCTATATCATCACTCAGTAGCCCTAATGATACATCAT
CTCCATTAATTGTTGATGCCCTTCTGCAGTTTTTTCTGCTCCATGTGCTATCTTCTT
CGAGTTCTGGGACCACTGTTAGAGGTTCAGGGATGGTTCCCCCGCTCTTGCCCCT
TTTGCAAGATAATGATCCTTCACACATGCATCTTGTCTGTCTGGCAGTGAAAACTC
TTCAAAAGTTGATGGAGTACAGCAGCCCTGCTGTTTCTCTATTTAAAGATTTGGGT
GGTGTAGAACTTTTGTCTCAGAGGTTGCACGTGGAGGTGCAGCGTGTTATTGGTG
TTGACAGTCATAATTCAATGGTTACAAGTGATGCATTGAAATCAGAAGAGGATCAT
CTCTACTCTCAGAAGCGATTGATTAAGGCGCTGCTAAAGGCATTGGGGTCTGCTA
CATATTCTCCTGCAAATCCTGCTCGTTCACAAAGCTCAAATGATAATTCTTTGCCCA
TCTCGCTTTCCCTTATATTTCAGAATGTTGACAAGTTTGGTGGTGACATTTATTTCT
CAGCAGTTACTGTTATGAGTGAGATAATTCACAAGGATCCAACATGCTTTCCTTCT
TTGAAGGAACTTGGTCTTCCAGATGCTTTTCTATCGTCAGTGAGTGCTGGGGTAAT
ACCATCTTGTAAAGCTCTCATCTGTGTGCCTAATGGTCTGGGTGCAATATGCCTTA
ATAACCAAGGACTTGAGGCTGTCAGGGAAACTTCAGCTCTGCGTTTTCTTGTTGAC
ACATTCACCAGCAGGAAGTACTTGATACCAATGAATGAAGGTGTTGTCCTATTAGC
TAATGCAGTGGAAGAGCTTCTACGTCACGTGCAGTCCCTAAGAAGCACTGGGGTT
GACATCATTATTGAAATAATTAATAAACTTTCTTCACCTCGTGAAGATAAGAGCAAT
GAACCAGCGGCCAGTTCTGATGAAAGAACAGAAATGGAAACTGACGCGGAAGGA
CGTGATTTGGTAAGTGCTATGGATTCCAGTGAGGATGGCACTAATGATGAACAGT
TTTCTCATTTGAGCATTTTCCATGTGATGGTATTGGTTCATCGGACAATGGAGAAC
TCCGAAACCTGCCGGTTATTTGTGGAGAAAGGAGGCCTGCAAGCACTTTTGACAC
TCCTGTTGCGACCTAGCATTACCCAATCATCTGGAGGAATGCCGATTGCTTTGCAT
AGCACCATGGTATTCAAGGGCTTTACTCAGCATCACTCTACTCCACTTGCACGTGC
ATTTTGCTCTTCCTTAAAGGAGCATTTAAAGAATGCCTTGCAGGAACTTGATACAG
TTGCAAGCTCTGGTGAAGTGGCAAAGTTAGAAAAAGGAGCAATTCCATCTCTTTTT
GTTGTTGAGTTCTTACTCTTCCTTGCGGCATCCAAAGATAATCGCTGGATGAATGC
TCTACTCTCAGAATTTGGAGATAGCAGTAGGGATGTCCTGGAAGATATTGGACGA
GTACACCGAGAAGTGCTTTGGCAAATTTCACTTTTTGAAGAAAAGAAAGTTGAGCC
TGAAACAAGTTCTCCTTTAGCAAATGACTCCCAGCAAGACGCAGCTGTGGGGGAT
GTTGATGATAGCAGATACACATCCTTTAGGCAATATCTTGATCCTCTTTTGAGGCG
AAGGGGCTCTGGGTGGAATATTGAATCACAGGTGTCTGACCTCATTAATATCTACC
GTGATATTGGCCGTGCAGCTGGTGACTCTCAGAGGTATCCTAGTGCAGGGTTGCC
CTCAAGTTCTTCTCAAGACCAGCCTCCCAGTTCATCTGATGCAAGTGCTAGCACAA
AATCAGAAGAGGACAAGAAAAGATCTGAGCATTCTTCCTGCTGTGACATGATGAG
GTCACTGTCTTACCATATCAATCATCTTTTCATGGAGCTTGGGAAAGCAATGCTTC
TTACATCTCGTCGGGAGAACAGCCCTGTGAATTTATCTGCATCTATTGTATCTGTT
GCTAGCAATATTGCTTCTATTGTGTTGGAGCACCTCAATTTTGAGGGGCACACAAT
CAGTTCTGAAAGAGAGACTACTGTTTCCACAAAATGCCGATACCTTGGGAAGGTG
GTTGAGTTCATTGATGGTATATTGTTGGACAGGCCGGAATCGTGCAACCCAATCAT
GCTGAATTCATTTTATTGCCGTGGTGTTATTCAGGCTATTTTAACCACATTTGAAGC
TACCAGTGAGTTGCTCTTTTCTATGAACAGGCTTCCGTCATCGCCTATGGAGACAG
ACAGTAAAAGTGTTAAGGAAGACAGGGAGACAGATTCGTCATGGATATATGGTCC
ACTCTCCAGCTATGGTGCAATTCTGGACCATCTAGTAACATCATCGTTTATTCTTTC
TTCCTCAACAAGACAATTACTTGAGCAGCCTATTTTTAGTGGAAATATCAGGTTTCC
CCAAGATGCAGAGAAGTTCATGAAGCTGCTTCAGTCAAGAGTTCTGAAGACTGTT
CTTCCCATCTGGACCCATCCTCAGTTTCCAGAATGTAATGTTGAGTTAATTAGTTC
AGTCACATCTATCATGAGGCATGTTTACTCTGGGGTTGAAGTGAAAAACACTGCTA

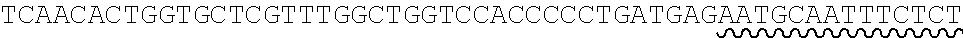

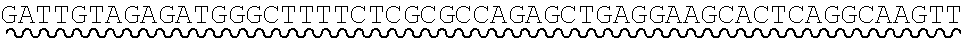

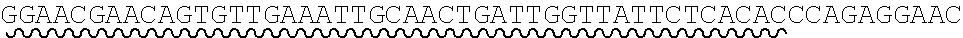

CACAAGAGGATGACGAACTTGCTCGAGCTCTTGCAATGTCTTTAGGCAATTCTGAT
ACGTCTGCACAAGAGGAAGATGGCAAATCGAATGATCTTGAACTTGAAGAAGAAA
CTGTTCAGCTGCCTCCCATAGATGAAGTATTGTCTTCATGTCTTAGGTTGCTTCAG
ACAAAGGAATCATTAGCTTTCCCTGTTCGGGACATGCTTTTGACTATGAGCTCACA
GAATGATGGTCAAAACCGAGTAAAGGTTCTTACGTATTTGATTGATCACCTGAAAA
ATTGTCTGATGTCATCTGATCCTTTAAAGAGCACTGCATTATCAGCTCTTTTTCATG
TCCTTGCTTTGATTCTCCATGGAGATACTGCTGCTCGGGAAGTTGCTTCAAAGGCT
GGTCTTGTCAAGGTTGCTTTGAACCTGCTGTGCAGCTGGGAGTTGGAGCCGAGG
CAAGGCGAGATAAGTGATGTTCCAAATTGGGTTCCTTCATGCTTTCTTTCTATTGA
TAGGATGCTCCAGTTGGACCCAAAGTTGCCAGATGTTACTGAACTCGATGTCCTTA
AAAAGGATAATTCAAATACACAAACATCAGTGGTGATTGATGATAGCAAGAAAAAG
GACTCAGAAGCTTCATCGAGCACAGGGTTATTGGACTTGGAGGACCAGAAGCAAC
TTTTGAAGATTTGCTGTAAATGCATTCAGAAGCAGTTGCCTTCTGCTACCATGCAT
GCTATTCTTCAGTTATGTGCCACGTTGACTAAACTTCATGCTGCTGCTATTTGTTTT
CTTGAGTCTGGTGGTCTGCATGCATTGCTAAGTTTGCCCACAAGTAGCTTGTTTTC
TGGATTCAACAGTGTGGCTTCTACAATCATTCGTCATATTTTGGAAGATCCCCACA
CTCTTCAGCAAGCAATGGAATTAGAGATACGCCACAGTCTTGTCACCGCTGCAAA
TCGTCATGCAAATCCAAGGGTTACACCGCGCAATTTTGTCCAGAACTTGGCGTTT
GTTGTATATAGAGACCCAGTGATATTTATGAAAGCTGCCCAAGCTGTGTGCCAGAT
TGAGATGGTTGGTGATAGACCATATGTTGTTCTGTTGAAGGATCGTGAAAAAGAAA
AGAACAAGGAAAAAGAGAAGGACAAGCCTGCTGATAAGGATAAAACATCAGGTGC
AGCCACAAAGATGACATCAGGGGACATGGCTTTAGGATCTCCTGTAAGTTCTCAA
GGGAAGCAGACTGATCTGAATACAAAGAATGTGAAATCTAATCGCAAACCACCAC
AAAGCTTTGTCACTGTTATTGAGTATCTGCTAGATCTGGTTATGTCCTTCATTCCAC
CTCCTAGAGCAGAAGATCGACCTGATGGTGAATCTAGTACTGCATCATCTACAGA
CATGGATATTGACAGCTCAGCAAAAGGCAAAGGTAAAGCTGTTGCTGTCACACCT
GAAGAGTCCAAGCATGCAATTCAAGAGGCTACTGCATCTCTCGCTAAAAGTGCAT
TTGTTCTGAAGCTGCTAACAGATGTTCTTCTGACTTATGCATCATCTATTCAAGTTG
TTCTTCGACATGATGCTGATTTGAGCAATGCACGTGGTCCTAACCGGATTGGTATT
AGCAGTGGTGGGGTTTTCAGTCATATACTGCAGCATTTCCTTCCGCATTCTACAAA
GCAAAAGAAAGAGAGGAAAGCTGATGGAGATTGGAGGTACAAATTGGCAACAAG
GGCTAATCAATTCTTGGTGGCTTCATCTATTCGGTCTGCAGAAGGTAGAAAAAGGA
TCTTTTCTGAAATCTGCAGCATATTTGTTGACTTCACAGACTCCCCTGCTGGTTGC
AAACCCCCAATATTAAGGATGAATGCATATGTTGATTTGCTTAATGATATTCTGTCA
GCCCGTTCGCCAACTGGTTCCTCCTTGTCAGCAGAATCTGCAGTTACTTTTGTTGA
AGTTGGTCTTGTTCAGTATTTATCAAAAACACTGCAAGTTATAGATTTGGATCATCC
TGATTCAGCAAAGATTGTAACTGCTATTGTTAAGGCCCTTGAGGTTGTCACAAAGG
AACATGTTCATTCGGCAGATTTGAATGCCAAAGGGGAGAACTCATCAAAGGTTGT
GTCTGACCAGAGCAATCTAGACCCGTCTTCAAATAGATTCCAAGCTCTTGACACAA
CTCAACCCACTGAGATGGTTACTGATCATAGGGAAGCTTTCAATGCTGTTCAAACT
TCACAAAGTTCAGATTCAGTGGCTGATGAGATGGACCATGACCGTGATCTGGATG
GAGGATTTGCTCGTGATGGTGAAGATGACTTTATGCACGAGATTGCTGAAGATGG

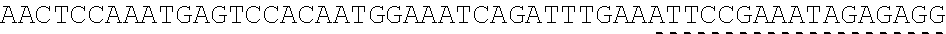

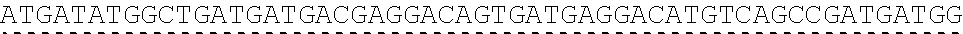

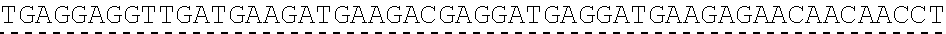

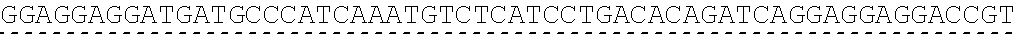

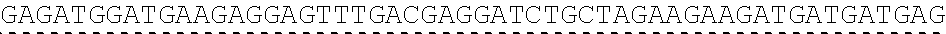

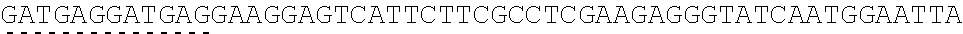

ATGTGTTTGACCATATCGAGGTGTTTGGGGGAAGCAACAATTTGTCTGGGGATAC
ACTGCGTGTAATGCCGTTGGACATTTTTGGAACAAGACGGCAAGGTCGTAGTACA
TCTATATATAACCTTCTTGGGAGAGCAGGCGATCATGGTGTTTTTGACCACCCGCT
CTTGGAGGAGCCTTCTTCGGTGCTACACCTTCCACAGCAAAGACAACAAGAAAAT
TTAGTTGAGATGGCCTTCTCTGATCGGAATCATGATAATAGTTCTTCCCGCTTGGA
TGCAATTTTCCGGAGCCTGCGAAGTGGCCGGAGTGGACACCGTTTTAATATGTGG
CTAGATGACAGTCCCCAACGCACTGGATCAGCTGCTCCTGCAGTACCTGAAGGCA
TTGAGGAGCTGCTGGTCTCTCAGTTGAGACGACCCACCCCTGAACAACCTGATGA
GCAGAGTACACCTGCTGGTGGCGCTGAAGAAAATGACCAATCTAATCAGCAACAT
TTGCATCAATCAGAAACTGAGGCAGGAGGAGATGCACCAACAGAACAAAATGAAA
ACAATGATAATGCAGTTACTCCGGCAGCAAGGTCTGAGTTAGATGGTTCTGAAAG
TGCTGATCCTGCACCTCCCAGCAATGCACTTCAAAGAGAAGTGTCTGGTGCAAGT
GAGCATGCCACGGAGATGCAATATGAACGTAGTGATGCTGTAGTACGTGATGTGG
AAGCAGTCAGCCAGGCAAGCAGTGGTAGCGGTGCTACTTTAGGGGAAAGCCTTA
GAAGTTTAGAGGTGGAGATAGGAAGTGTTGAAGGGCATGATGATGGTGATCGCCA
CGGAGCTTCAGACAGGCTTCCTTTGGGTGATTTGCAGGCAGCTTCAAGATCAAGG
AGGCCACCTGGAAGTGTTGTGCTAGGTAGCAGCAGAGATATATCTCTGGAGAGTG
TCAGCGAGGTTCCTCAAAATCAAAATCAAGAATCTGATCAGAATGCTGATGAAGG
GGATCAGGAGCCTAACAGAGCTGCTGACACTGACTCAATTGATCCTACATTTTTG
GAGGCTCTTCCAGAGGATTTACGGGCTGAAGTTCTTTCTTCACGTCAAAATCAAGT
GACCCAGACTTCTAATGAACAACCTCAGAATGATGGGGATATTGATCCTGAATTCC
TTGCTGCACTTCCTCCTGATATACGTGAAGAAGTTCTAGCTCAACAACGTGCGCAA
AGGTTGCAGCAGTCACAGGAATTAGAAGGACAACCAGTTGAAATGGATGCTGTTT
CAATTATCGCAACATTCCCTTCAGAAATTCGGGAGGAGGTGCTTTTAACATCTCCA
GATACATTACTGGCTACACTTACGCCTGCACTAGTTGCTGAAGCAAACATGTTAAG
GGAGAGATTTGCTCATCGGTATCACAGTGGCTCCCTTTTTGGCATGAACTCCAGG
GGCAGGAGAGGTGAGTCCTCTCGACGTGGTGACATAATTGGTTCAGGTCTTGATA
GAAATGCTGGTGATTCTTCTCGACAACCAACTAGCAAGCCAATTGAAACGGAAGG
ATCTCCTCTTGTTGACAAGGATGCTCTTAAAGCTCTTATTAGGCTACTCCGGGTTG
TTCAGCCTCTATACAAAGGTCAATTGCAGAGGCTTCTCTTGAACCTTTGTGCTCAT
AGGGAAAGCAGAAAGTCCTTGGTTCAAATTCTAGTGGACATGCTTATGCTTGATCT
GCAGGGCTCTTCTAAGAAATCAATTGATGCAACTGAGCCACCATTTAGGCTATATG
GGTGCCATGCAAATATTACGTACTCACGCCCTCAATCGACAGATGGCGTGCCTCC
ATTAGTTTCTCGTCGTGTTCTTGAAACTTTGACATACTTGGCAAGAAATCATCCAAA
TGTGGCTAAACTCTTGCTATTTCTTGAGTTCCCTTGCCCCCCAACTTGCCATGCTG
AAACATCTGATCAGAGGCGTGGCAAGGCTGTTCTTATGGAAGGTGACAGTGAACA
GAACGCTTATGCACTTGTCCTACTTTTAACCTTGTTGAATCAGCCACTTTATATGAG
GAGCGTAGCTCATCTTGAACAGCTACTAAACCTTCTCGAAGTTGTTATGCTCAATG
CCGAGAATGAAATTACACAAGCTAAGCTGGAAGCAGCATCTGAAAAACCATCTGG
ACCTGAGAATGCAACGCAAGATGCCCAAGAGGGTGCGAATGCTGCTGGATCATCT
GGATCGAAGTCCAATGCTGAGGATAGCAGCAAACTCCCTCCTGTTGATGGTGAAA
GTAGCCTGCAAAAAGTTCTGCAGAGTCTTCCCCAAGCAGAGCTTCGACTGCTATG
TTCACTGCTTGCACATGATGGGTTGTCAGACAATGCGTATCTCCTGGTAGCAGAA
GTTCTGAAAAAGATTGTAGCTCTTGCTCCTTTTTTCTGTTGCCATTTCATAAATGAA
CTTGCACATTCAATGCAAAATTTGACGCTTTGTGCAATGAAGGAGCTTCACTTGTA
TGAGGATTCTGAAAAGGCTCTTCTTAGCACATCATCAGCCAATGGCACTGCAATTC
TTAGAGTTGTGCAGGCTTTGAGTTCTCTTGTCACCACTCTGCAAGAGAAAAAGGAT
CCAGATCATCCTGCTGAAAAAGATCATTCTGATGCATTGTCCCAGATTTCTGAAAT
TAACACTGCATTGGATGCATTATGGTTGGAGCTGAGTAATTGCATAAGCAAAATAG
AGAGCTCTTCAGAATACGCATCGAATCTAAGTCCTGCTTCTGCAAATGCAGCCACA
TTAACAACAGGTGTAGCACCTCCATTGCCTGCCGGAACTCAGAACATATTACCGTA
CATAGAATCATTTTTCGTGACATGTGAGAAGTTACGCCCTGGGCAACCTGATGCTA
TTCAAGAAGCTTCAACATCTGACATGGAGGATGCATCAACTTCTAGTGGTGGGCA
GAAATCATCTGGAAGCCATGCAAATCTTGATGAGAAGCACAATGCGTTTGTTAAAT
TCTCAGAGAAACACAGAAGATTGTTGAACGCATTTATCCGCCAAAACCCTGGGCT
ATTGGAGAAGTCATTCTCTCTGATGTTGAAAATCCCTCGCTTGATTGAATTTGACA
ACAAGCGTGCATATTTCCGGTCTAAAATTAAGCATCAGCATGATCATCATCATAGC
CCTGTTAGAATTTCTGTGCGCCGGGCATATATTTTGGAGGATTCATATAACCAGCT
TAGGATGCGTTCACCACAGGATTTGAAGGGTAGACTGACTGTTCATTTCCAAGGT
GAAGAAGGCATTGATGCTGGTGGACTAACAAGGGAATGGTATCAGCTGCTATCAC
GAGTGATTTTTGATAAGGGTGCCCTTCTATTCACAACTGTTGGAAATGACTTGACA
TTTCAACCAAACCCTAACTCGGTGTATCAGACTGAACACCTCTCATATTTCAAATTT
GTTGGGCGAGTGGTTGGTAAAGCTCTATTTGATGGCCAACTTTTGGATGTCCATTT
TACAAGATCTTTCTACAAGCACATACTAGGTGTCAAGGTTACATACCATGACATTG
AAGCTATTGATCCTGCATACTATAAAAATTTGAAATGGATGCTTGAGAATGACATAA
GCGATGTTCTGGACCTCTCCTTCAGCATGGATGCAGATGAAGAGAAGCGGATATT
GTATGAGAAGGCAGAGGTGACTGATTATGAGTTGATTCCTGGAGGCCGAAACATC
AAGGTCACCGAGGAGAACAAGCATGAATATGTGAACCGGGTTGCAGAACATCGTT
TAACCACTGCTATTAGGCCTCAAATCACCTCTTTTATGGAGGGATTTAATGAGCTC
ATTCCTGAGGAGCTGATATCAATCTTTAATGACAAAGAACTTGAACTGCTAATCAG
TGGACTCCCAGACATTGACTTGGACGATCTAAAAGCAAATACAGAATATTCTGGGT
ACAGCATAGCTTCTCCAGTCATTCAGTGGTTCTGGGAGATTGTCCAAGGGTTCAG
CAAGGAGGACAAAGCCCGGTTCCTTCAGTTTGTTACTGGCACCTCAAAGGTACCT
CTGGAAGGTTTCAGTGCACTCCAAGGAATATCTGGACCACAACGATTCCAGATAC
ACAAGGCCTACGGAAGCACCAACCATCTGCCTTCAGCACATACTTGCTTTAACCA
ACTAGACCTTCCTGAGTACACATCGAAAGAGCAGCTCCAGGAGAGATTGCTACTG
GCTATTCATGAGGCGAATGAAGGTTTCGGATTTGGTTAA
SEQ ID NO: 2 Os UPL2 Protein sequence

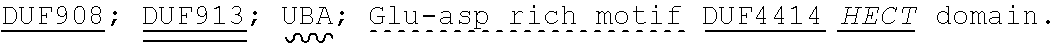

Conserved cysteine residue in the HECT domain is shown by [symbol]
MAAAAAMAAHRASFPLRLQQILSGSRAVSPSIKVESEPPAKVKAFIDRVISIPLHDIAIPL
SGFRWEFNKGNFHHWKPLFMHFDTYFKTQISSRKDLLLSDDMAEGDPLPKNTILQILR
VMQIVLENCQNKTSFAGLEHFRLLLASSDPEIVVAALETLAALVKINPSKLHMNGKLINC
GAINSHLLSLAQGWGSKEEGLGLYSCVVANERNQQEGLCLFPADMENKYDGTQHRL
GSTLHFEYNLAPAQDPDQSSDKAKPSNLCVIHIPDLHLQKEDDLSILKQCVDKFNVPSE
HRFSLFTRIRYAHAFNSPRTCRLYSRISLLAFIVLVQSSDAHDELTSFFTNEPEYINELIR
LVRSEEFVPGPIRALAMLALGAQLAAYASSHERARILSGSSIISAGGNRMVLLSVLQKAI
SSLSSPNDTSSPLIVDALLQFFLLHVLSSSSSGTTVRGSGMVPPLLPLLQDNDPSHMH
LVCLAVKTLQKLMEYSSPAVSLFKDLGGVELLSQRLHVEVQRVIGVDSHNSMVTSDAL
KSEEDHLYSQKRLIKALLKALGSATYSPANPARSQSSNDNSLPISLSLIFQNVDKFGGD
IYFSAVTVMSEIIHKDPTCFPSLKELGLPDAFLSSVSAGVIPSCKALICVPNGLGAICLNN
QGLEAVRETSALRFLVDTFTSRKYLIPMNEGVVLLANAVEELLRHVQSLRSTGVDIIIEII
NKLSSPREDKSNEPAASSDERTEMETDAEGRDLVSAMDSSEDGTNDEQFSHLSIFHV
MVLVHRTMENSETCRLFVEKGGLQALLTLLLRPSITQSSGGMPIALHSTMVFKGFTQH
HSTPLARAFCSSLKEHLKNALQELDTVASSGEVAKLEKGAIPSLFVVEFLLFLAASKDN
RWMNALLSEFGDSSRDVLEDIGRVHREVLWQISLFEEKKVEPETSSPLANDSQQDAA
VGDVDDSRYTSFRQYLDPLLRRRGSGWNIESQVSDLINIYRDIGRAAGDSQRYPSAG
LPSSSSQDQPPSSSDASASTKSEEDKKRSEHSSCCDMMRSLSYHINHLFMELGKAML
LTSRRENSPVNLSASIVSVASNIASIVLEHLNFEGHTISSERETTVSTKCRYLGKVVEFI
DGILLDRPESCNPIMLNSFYCRGVIQAILTTFEATSELLFSMNRLPSSPMETDSKSVKE
DRETDSSWIYGPLSSYGAILDHLVTSSFILSSSTRQLLEQPIFSGNIRFPQDAEKFMKLL
QSRVLKTVLPIWTHPQFPECNVELISSVTSIMRHVYSGVEVKNTAINTGARLAGPPPDE
NAISLIVEMGFSRARAEEALRQVGTNSVEIATDWLFSHPEEPQEDDELARALAMSLGN
SDTSAQEEDGKSNDLELEEETVQLPPIDEVLSSCLRLLQTKESLAFPVRDMLLTMSSQ
NDGQNRVKVLTYLIDHLKNCLMSSDPLKSTALSALFHVLALILHGDTAAREVASKAGLV
KVALNLLCSWELEPRQGEISDVPNWVPSCFLSIDRMLQLDPKLPDVTELDVLKKDNSN
TQTSWIDDSKKKDSEASSSTGLLDLEDQKQLLKICCKCIQKQLPSATMHAILQLCATLT
KLHAAAICFLESGGLHALLSLPTSSLFSGFNSVASTIIRHILEDPHTLQQAMELEIRHSLV
TAANRHANPRVTPRNFVQNLAFVVYRDPVIFMKAAQAVCQIEMVGDRPYVVLLKDRE
KEKNKEKEKDKPADKDKTSGAATKMTSGDMALGSPVSSQGKQTDLNTKNVKSNRKP
PQSFVTVIEYLLDLVMSFIPPPRAEDRPDGESSTASSTDMDIDSSAKGKGKAVAVTPE
ESKHAIQEATASLAKSAFVLKLLTDVLLTYASSIQWVLRHDADLSNARGPNRIGISSGGV
FSHILQHFLPHSTKQKKERKADGDWRYKLATRANQFLVASSIRSAEGRKRIFSEICSIF
VDFTDSPAGCKPPILRMNAYVDLLNDILSARSPTGSSLSAESAVTFVEVGLVQYLSKTL
QVIDLDHPDSAKIVTAIVKALEVVTKEHVHSADLNAKGENSSKVVSDQSNLDPSSNRF
QALDTTQPTEMVTDHREAFNAVQTSQSSDSVADEMDHDRDLDGGFARDGEDDFMH

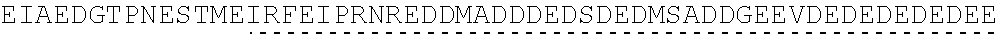

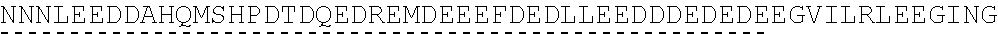

INVFDHIEVFGGSNNLSGDTLRVMPLDIFGTRRQGRSTSIYNLLGRAGDHGVFDHPLL
EEPSSVLHLPQQRQQENLVEMAFSDRNHDNSSSRLDAIFRSLRSGRSGHRFNMWLD
DSPQRTGSAAPAVPEGIEELLVSQLRRPTPEQPDEQSTPAGGAEENDQSNQQHLHQ
SETEAGGDAPTEQNENNDNAVTPAARSELDGSESADPAPPSNALQREVSGASEHAT
EMQYERSDAWVRDVEAVSQASSGSGATLGESLRSLEVEIGSVEGHDDGDRHGASDR
LPLGDLQAASRSRRPPGSVVLGSSRDISLESVSEVPQNQNQESDQNADEGDQEPNR
AADTDSIDPTFLEALPEDLRAEVLSSRQNQVTQTSNEQPQNDGDIDPEFLAALPPDIRE
EVLAQQRAQRLQQSQELEGQPVEMDAVSIIATFPSEIREEVLLTSPDTLLATLTPALVA
EANMLRERFAHRYHSGSLFGMNSRGRRGESSRRGDIIGSGLDRNAGDSSRQPTSKP
IETEGSPLVDKDALKALIRLLRVVQPLYKGQLQRLLLNLCAHRESRKSLVQILVDMLML
DLQGSSKKSIDATEPPFRLYGCHANITYSRPQSTDGVPPLVSRRVLETLTYLARNHPN
VAKLLLFLEFPCPPTCHAETSDQRRGKAVLMEGDSEQNAYALVLLLTLLNQPLYMRSV
AHLEQLLNLLEVVMLNAENEITQAKLEAASEKPSGPENATQDAQEGANAAGSSGSKS
NAEDSSKLPPVDGESSLQKVLQSLPQAELRLLCSLLAHDGLSDNAYLLVAEVLKKIVAL
APFFCCHFINELAHSMQNLTLCAMKELHLYEDSEKALLSTSSANGTAILRVVQALSSLV
TTLQEKKDPDHPAEKDHSDALSQISEINTALDALWLELSNCISKIESSSEYASNLSPASA
NAATLTTGVAPPLPAGTQNILPYIESFFVTCEKLRPGQPDAIQEASTSDMEDASTSSG
GQKSSGSHANLDEKHNAFVKFSEKHRRLLNAFIRQNPGLLEKSFSLMLKIPRLIEFDNK
RAYFRSKIKHQHDHHHSPVRISVRRAYILEDSYNQLRMRSPQDLKGRLTVHFQGEEGI
DAGGLTREWYQLLSRVIFDKGALLFTTVGNDLTFQPNPNSVYQTEHLSYFKFVGRVV
GKALFDGQLLDVHFTRSFYKHILGVKVTYHDIEAIDPAYYKNLKWMLENDISDVLDLSE
SMDADEEKRILYEKAEVTDYELIPGGRNIKVTEENKHEYVNRVAEHRLTTAIRPQITSE
MEGFNELIPEELISIFNDKELELLISGLPDIDLDDLKANTEYSGYSIASPVIQWFWEIVQG
ESKEDKARFLQFVTGTSKVPLEGFSALQGISGPQRFQIHKAYGSTNHLPSAHT [symbol] FNQ
LDLPEYTSKEQLQERLLLAIHEANEGFGFG.
SEQ ID NO: 3 OsUPL2 promoter sequence
acattaactgtcctatatgcgatgtatttattgttatggtgtattaaatcatcagtatatatagtaaaaaacataacaaagagtgcacgacta
atttaaaagataaaagaaaaagtagagtaattgggccaccaaaactaatgattttcgctactagatcgaagctctagccttttttttttttttg
ccataagcctgcttgacatgtatcttttacttgattttagatgatcctcatattcctttatttctaaacttcccaagcaatcaaaagaatagcaa
atgttcatctttacacaaatgaaaactaccattttagcttgattgtgttcttggcccattctaggaagctaaaattatgagaagtagccttttgg
tagctaaattttgagaatctagaatatatctgagggaaggggatgcaggaactgcattctttcatttgaagataaaggcgagaagcag
gaagcttctcattccaatccttgagcatgatggcaggattgccaccacccagcatgacatgcaaagtttggcacgagaatactttgctg
cagtgatgtgccctgagtgcagtgacacgaagttgctgcaatttcaccatattcagatggcaacaactgatctctccagcctcgacagt
cctttcactgaagatgagatttggtcggctatccgtgctttgcctaatgaaaagtcgccagggccggatggttatacaggcttgttttacca
aagatgttgggagataattaaacctgaattgatcagcgctcttgctaaattctgtaccggtaacagtcagaacttggagaaactgaattc
ggcaattgtcacgctaataccgaagaaggacagtcctaccctcctcaaggattataggccaattagtttgattcatagtttctctaagata
gctgcgaagattatggcgcagcggttagcaccgaagctgaatgtcctcattccatcctcccaaactgcttttatcaagggacgctgcat
acacgagaactttgtcttcgtcaaaggattggtacaacaatttcacagacaaaggaaggctatgatgttgctgaaattagacatctcga
aagctttcgacactgtctcctggggttttcttatgtcgatgttacagttcagaggctttggtccactttggagaagatggctctcggcggttttt
ctcactgcagaaacaagaatattgataaatggtgttctgtctgacacaatcaagccggcgagggggttgaggcagggtgacccactg
tcgccgctgctctttgttctagtaatggatgccttgcaagctattgtttcccaggcaaggatggcaagactgctctcgcccctcaacgtacg
acagaatttgccaccaatttcagtttatgccgacgatgcggttctgtttttccgccctacagctgaagaagctcgagtcatcaagggtatc
ctggagttgttcggggctgccacaagtctcaaaaccaatttctccaaaagcgcaatcactccaatccaatgtgacgagcagcagtatg
tgcaagttgaatccattctctcctgccgagtggaaaagttcccaatcacttatcttggactccctctctccactaggaaaccaacgaagg
ccgagatccagccgatccttgataggctggcaaagaaggtagccggttggaagccgaaaatgctgtctattgatgggcgactgtgctt
gatcaagtcggtcctaatggcgctgccggtgcactacatgacagtcctgcagctaccgcgatgggcgattaaggacatcgagcgga
agtgccgtgggtttctttggaaaggacaggaagagatcagcggcgggcattgcctagtctcgtggcgaaaggtttgctcacccatcga
gaaagggggacttggtgtcaaagatcttaatttgttcggtcaagctctccggttgaaatggcttgcaaaatccttggagcagaaggata
gaccctggaccttagcaactttccgtcctggaagcgatgtggaagagatctttcgatccgttgctgagcacatcattggtgacggggtga
acacacagttttggacagacaattggacagggaaaggttgcttcgcctggaggtggccggtgttgttttcccatgtgagccgtgccaag
ctgacagtagctgatgccctgattgctaacagatgggttcgccgattacaaggtgccttgtccaatgaagctctgggtgaattcttccaac
tttgggatgaagttcacgacgtgtcactgcagcagatggctaaaacgatcaaatggaagttgactgttgatggtaatttctcagtggcctc
ggcgtatgatctatttttcatagcgacagaggactgttcctacggggacacgctgtggcactccagggtgccgtcgcgtgttcgcttcttc
atgtggattgcactcaagggccgctgtctcacggcggacaacctggcaaagagaaactggccgcatgacgccatttgctccctatgc
caacacgagaacgaagactgccattatttgcttgtgtcctgtgattatacggcggcggtttggcgcaagctgagacgttggtgcaacatt
aacattgcaatccctgcggaagatggcatgccgcttgcagattggtggatcgcgacaagacggcgttttcagaacacgtataggacg
gatttcgatagtctgttaatgctaatttgttggcttatctggaaagagcgaaatacaaggatotttcaacacatcgccaagtcggttgaccg
gctagcggatgacatcaacgaggaaatcgcaatttggagggcagcagggattttctcccaggctagcgagtaatcccgattagagg
cgggacctgccccattttttccttttctttccgggcttgagtttgcttgagaccggcgcgacatccttcatgtcgttgtaattaaaactttatttcc
ctcaatcttaataaaattggccggcctacctttggccgtcccggcaaaaaagaatctagaatatatagctacatattctcaaaatcgaat
ctggactgttttggagagtagccgctagaaacttcctagaacaaaacccttatatttgttctttaagtcacatcatacttgctgatgaaatca
ctatccattagttactccatccgtcccaaaaatacttaatctaggagaagatgtgactccttctgatacaataaatttggataaagagctat
cagatttgttaggatcacacatttatttgtaggttaagttttttttaacggaagtagtacgcataaaggattggcttacccaattgttaaccgg
cccggcactggaacagaaaggtcttgaacccaaacgggacgccgagaaggcccttccctgacgaaagcaaagggcttaattagc
tagcaagaaacccaaaccgacccgagcccgtcacgcgccgcgcccgtgacctaccgtgcgctgcgccgcctcctccctcccacct
cccttcacaaaagcagcgacccctcctccctccccaagtttcctccccacaccgcaacccttctctctctctctctctcccctctcgacttct
ctcctctccgccgcctccgagtcccgccgcgccgcgcgcccgtcttccccggcggccgatgtgtctgcctcgtcggcacgaaacccta
gaggtaacccgccgcgccgctccccgccgcttcccgccgcgatcgggggccctcccccctagggttttcgggggacttttgagggtg
gatgatttgggggtgtggggggctttgggggcggtctaacctgtttgtggtttctggtgcaggtgcggtgcagttgaggggtcccgatcgg
agATGGCGGCGGCGGCGGCCATGGCGGCGCACCGGGCCAGCTTCCCGCTCCGGCTGCA
GCAGATCCTGTCCGGGAGCCGCGCCGTGTCGCCGTCGATCAAGGTGGAGTCCGAGCCGG
TGAGTCCCTCGCGCCGTTCCCCTGTTTCCTCGCCCTAGGGTTTTGATCGTCGGGGTTGAG
GGGTTGTAGATGCGAAGTTGAGATGGTATGTAGGATCGAATCCTCCCTAGGTGCTTCCTCT
AGGGTTTTGATCGGCTGCCTGTGTTGATGTGGCGTGCTGTTGGGGTGAGGTAGTTAGGCC
GTAAGGAGTTTGCTCCGTTTATGATCGGTGTTGAGCATGGGGACCAGTGGTGTGGTGTGC
AGGGTAGTTGTTACTGCTTTAGGCCATCTCAAATTTGGGTTTCCTTGGTCAGGGGTAGAAG
AGACACCGGTTTGAAGTTTCTGGTTATCTTGCTTGTGCTGTTATTGTACTATATTGTAGTAG
GGATACATGCTCGTGTTATTCTGTTACCTTGTTTAAGCATGTCTATGCCCCTCAATGCTTAG
TTGCCGCTGCAGCCGTAATCTTTTAGGCTTAGCCGCTTAGGTATCCCCATTACATTTGTATT
ATCTTGTTATTACTACGGTGTCCCATTGGACATTTATTAGTTCAGACTTTCTTGCACTTGTAA
TTCCTTCTGCAAAACATACGAGTCAATACAGAATGCCACATCTAGCAAATTACTATGTTATC
ATTGATGCTTAGGTGCCCATGATCAGTACTTATGGACTTGTACTGGCCATTTTATAATGTTA
TTTTTTCATTCTGTTATTGCTATAGCTTTTTAATCCTTTTTTACGTATTTTTATTTCTGTGCACA
ACTGCACTTATGTTGACCAATCCTGTATCATGTTTTGGATAATGGCTTACTACATAAATATAT
GACGTTGGATAGTAGCCTCAAGATTGATGCATTGATTTAGTTCACTTGATATTACAGCTCAA
GAGTTGAGACAT
Homologous sequences
Wheat
SEQ ID NO: 4 UPL2 CDS; A genome
>TraesCS5A02G121600.1(Longest) cds: protein_coding
SEQ ID NO: 5 UPL2 amino acid; A genome; HECT domain underlined.
MAAAAMAAHRASFPLRLQQILSGXXXXXXXXXXXXXXPAKVKAFIDRVINIPLHDIAIPL
SGFHWEFNKGNFHHWKPLFMHFDTYFKTYISSRKDLLLSDDMSESEPLTKNTILQILR
VMQIVLENCQNKTTFAGLEHFKNLLTSSDPEVVVAALETLASVVKINPSKLHMNGKLIS
CGAINSHLLSLAQGWGSKEEGLGLYSCVVANERNQLEGLCLFPADMENKYDGTQHR
LGSTLHFEYNLAPVQDSDQANDKSSNLCVIHMPDLHLRKEDDLSILKQCINKFNVPPE
HRFALFTRIRYAHAFNSPRTCRLYSRISLLSFIVLVQSSDAHDELTSFFTNEPEYINELI
RLVRSEDIVPGPIRALAMLALGAQLAAYASSHERARILSGSSIISAGGNRMVLLSVLQK
AISSLSSPNDTSSPLIVDALLQFFLLHVLSSSSSGTTVRGSGMVPPLLPLLQDKDPSH
MHLVCLAVKTLQKLMEYSSPAVSLFKDLGGVELLSQRLHVEVQRVIGVAEITSWASD
TSKSEDDHLYSQKRLIKALLKALGSATYSPANPARSQSSNDNSLPMSLSLIFQNVGKF
GGDIYFSSVTVMSEIIHKDPTCFPALKELGLPDAFLSSVTAGVIPSCKALICVPNGLGAI
CLNNQGLESVRETSALRFLVETFTSKKYLIPMNEGVVLLANAVEELIRHVQSLRSTGV
DIIIEIINKLSCPRGDKITEAASAEEKTDMETDVEGRDLVSAMDSGTDGTNDEQFSHLSI
FHVMVLVHRTMENSETCRLFVEKGGLQTLLTLLLRPTITQSSGGMPIALHSTMVFKGF
TQQHSTPLARAFCSSLKEHLKNALQELDTVFRSCEVTKMEKGAIPSLFIVEFLLFLAAS
KDNRWMNALLSEFGDVSRDVLEDIGRVHREVLWQISLFDEKKIEPEASSPSANEAQQ
VDAAVGDTDDNRYTSFRQYLDPLLRRRGSGWNIESQVSDLINIYRDMGRAATDSHR
VGADRYPSTGLPSSSQDQPSSSSDANAKSEEDKKRSEHSSCCDMMRSLSYHINHLF
MELGKAMLLTSRRENSPINLPPSVVSVASNIASIVLEHLNFEGHTISPEREITVATKCRY
LGKVVEFIDGILLDRPESCNPIMVNSFYCRGVIQAILTTFEATSELLFAMNRPPSSPME
TDSKTGMEEKDTDCSWIYGPLSSYGAAMDHLVTSSFVLSSSTRQLLEQPIFSGTVRF
PQDAERFMKLLQSKVLKTVLPIWAHPQFPECNLELISSVTSIMRHVYSGVEVKNNVSN
MAARLAGPPPDENAISLIIEMGFSRARAEEALRQVGTNSVEIATDWLFSHPEEPPEDD
ELARALAMSLGNSDTPVQEEDDRTNDLELEEVNVQLPPMDEVLSSCLRLLQAKETLA
FPVRDMLVTISSQNDGQNRVKVLTYLIDHLKQCLVASDPLKNTALSAFFHVLALILHGD
TAAREVASKAGLVKVVLNLLCSWELEPREGQTTKVPNWVTSCFLSVDRMLQLEPKLP
DVTELDVLKKDNSPTQTSWVIDDSKKKDSESSSSVGLLDLEDQDQLLRVCCKCIQKQL
PSGTMHAILQLCATLTKVHVAAISFLESGGLHALLSLPTSSLFSGFNSWVSTIIRHILEDP
HTLQQAMELEIRHSLVTAANRHANPRVTPRNFVQNLAFVVYRDPVIFMKAAQAVCQI
EMVGDRPYWVLLKDREKEKSKEKEKDKLVDKDKSSGVATKITSGDMVMASPVSAKG
KQSDFSARNMKSHRKPPQTFVTVIEHLLDLVMSFVPPQRAEDQSDGSSSMDMDIDS
SSAKGKGKAVAVTHEESKQAIQDATACLAKNAFVLKLLTDVLLTYASSVQVVLRHDAE
FSSTRGPTRTSGGIFNHILQHLLPHATKQKKERKPDGDWRYKLATRGNQFLVASSIR
SSEGRKRICSEICSIFVEFTDNSTGCKPPMLRMNAYVDLLNDILSARSPTGSSLSAESV
VTFVEVGLVQCLTKTLQVLDLDHSDSAKIVTGIVKALEVVTKEHVHLADFNAKGENSS
KTVLEQNNVDSSSNRFQVLDTTSQPTAMVTDHRETFNAVHASRSSDSVADEMDHDR
DIDGGFAHDGEDDFMHEIAEDRTGNESTMDIRFDIPRNREDDMAEDEDDSDEDMSA
DDGEEVDEDDDDEENNNLEEDDAHQISHADTDQDDREIDEEEFDEDLLEEEDDDEE
DEEGVILRLEEGINGINVFDHIEVFGGSNNVSGDTLRVMPLDIFGTRRQGRSTSIYNLL
GRASDQGVLDHPLLEEPSMLLPQQRQPENLVEMAFSDRNQENSSSRLDAIFRSLRS
GRNGHRFNMWLDDGPQRNGSAAPTVPEGIEELLLSQLRRPMAEHPDEQSTPAVDA
QVNDPPSNFHGPETDAREGSAEQNENNENDDIPAVRSEVDGSASAGPAAPHSDEL
QRDASNASEHVADMQYERSDTAVRDVEAVSQASSGSGATLGESLRSLDVEIGSVEG
HDDGDRHGASDRTPLGDVQAATRSRRPSGNAVPVSSRDISLESVREIPQNTVQESD
QNASEGDQEPNRATGTDSIDPTFLEALPEDLRAEVLSSRQNQVTQTSSEQPQHDADI
DPEFLAALPPDIREEVLAQQRAQRLQQQSQELEGQPVEMDAVSIIATFPSEIREEVLLT
SPDTLLATLTPALVAEANMLRERFAHRYHSGSLFGMNSRNRRGESSRRGDIIGSGLE
RNTGDSSRQTASKLIETVGTPLVDKDALNALIRLLRVVQPIYKGQLQRLLLNLCAHRES
RKSLVQILVDMLMLDLQGSSKKSIDATEPSFRLYGCHANITYSRPQSSDGVPPLVSRR
VLETLTYLARNHPNVAKLLLFLRFPCPPTCHTETLDQRHGKAVLVEDGEQQSAFALVL
LLTLLNQPLYMRSVAHLEQLLNLLEVVMLNAENEVNQAKLESSSERPSGPENAIQDA
QEDASVAGSSGAKPNADDSGKSSADNISDLQAVLHSLPQAELRLLCSLLAHDGLSDN
AYLLVAEVLKKIVALAPFICCHFINELSRSMQNLTVCAMNELHLYEDSEKAILSTSSAN
GMAVLRVVQALSSLVTSLQERKDPELLAEKDHSDSLSQISDINTALDALWLELSHCISK
IESSSEYTSNLSPTSANATRVSTGVAPPLPAGTQNILPYIESFFVTCEKLRPGQPDAVQ
EPSTSDMEDASTSSSGQKSSASHTSLDEKHTAFVKFSEKHRRLLNAFIRQNSGLLEK
SFSLMLKVPRLIDFDNKRAYFRSKIKHQHDHHHSPVRISVRRAYILEDSYNQLRMRSP
QDLKGRLTVHFQGEEGIDAGGLTREWYQLLSRVIFDKGALLFTTVGNDLTFQPNPNS
VYQTEHLSYFKFVGRVVGKALFDAQLLDVHFTRSFYKHILGAKVTYHDIEAIDPAYYR
NLKWMLENDISDVLDLTFSMDADEEKLILYEKAEVTDCELIPGGRNIRVTEENKHEYV
DRVAEHRLTTAIRPQINAFMEGFNELIPRELISIFNDKEFELLISGLPDIDLDDLKANTEY
SGYSIASPVIQWFWEIVQGFSKEDKARFLQFVTGTSKVPLEGFSALQGISGPQRFQIH
KAYGSTNHLPSAHTCFNQLDLPEYTSKDQLQERLLLAIHEANEGFGFG
SEQ ID NO: 6 UPL2 CDS; B genome
TraesCS5B02G112800.1
SEQ ID NO: 7 UPL2 amino acid; B genome; HECT domain underlined.
MAAAAMAAHRASFPLRLQQILSGSRAVSPAIKVESXXPAKVKAFIDRVINIPLHDIAIPLS
GFHWEFNKGNFHHWKPLFMHFDTYFKTYISSRKDLLLSDDMSESEPLTKNTILQILRV
MQIVLENCQNKTTFAGLEHFKNLLASSDPEVVVAALETLASVVKINPSKLHMNGKLISC
GAINSHLLSLAQGWGSKEEGLGLYSCVVANERNQLEGLCLFPADMENKYDGTQHRL
GSTLHFEYNLAPVQDSDQANDKSSNLCVIHMPDLHLRKEDDLSILKQCIDKFNVPPEH
RFALFTRIRYAHAFNSPRTCRLYSRISLLSFIVLVQSSDAHDELTSFFTNEPEYINELIRL
VRSEDIVPGPIRALAMLALGAQLAAYASSHERARILSGSSIISAGGNRMVLLSVLQKAIS
SLSSPNDTSSPLIVDALLQFFLLHVLSSSSSGTTVRGSGMVPPLLPLLQDKDPSHMHL
VCLAVKTLQKLMEYSSPAVSLFKDLGGVELLSQRLHVEVQRVIGVAEITSVVASDTSKS
EDDHLYSQKRLIKALLKALGSATYSPANPARSQSSNDNSLPMSLSLIFQNVGKFGGDI
YFSSVTVMSEIIHKDPTCFPALKELGLPDAFLSSVTAGAIPSCKALICVPNGLGAICLNN
QGLESVRETSALRFLVETFTSRKYLIPMNEGVVLLANAVEELLRHVQSLRSTGVDIIIEII
NKLSCPRGDKITEAASAEEKTDMETDVEGRDLVSAMDSGTDGTNDEQFSHLSIFHVM
VLVHRTMENSETCRLFVEKGGLQTLLTLLLRPTITQSSGGMPIALHSTMVFKGFTQQH
STPLARAFCSSLKEHLKNALQELDTVFRSCEVTKLEKGAIPSLFIVEFLLFLAASKDNR
WMNALLSEFGDVSRDVLEDIGRVHREVLWQISLFDEKKIEPEASSPSANEAQQVDAA
VGDTGDNRYTSFRQYLDPLLRRRGSGWNIESQVSDLINIYRDMGRAASDSHRVGAD
RYPSTGLPSSSQDQPSSSSDANAKSEEDKKRSEHLSCCDMMRSLSYHINHLFMELG
KAMLLTSRRENSPINLSPSVVSVASNIASIVLEHLNFEGHTISPEREITVATKCRYLGKV
VEFIDGILLDRPESCNPIMVNSFYCRGVIQAILTTFEATSELLFAMNRPPSSPMETDSKT
GKEEKDADCSWIYGPLSSYGAAMDHLVTSSFILSSSTRQLLEQPIFSGTVRFPQDAEK
FMKLLQSKVLKTVLPIWAHPQFPECNLELISSVTSIMRHVYSGVEVKNNVSNIAARLAG
PPPDENAISLIIEMGFSRARAEEALRQVGTNSVEIATDWLFSHPEEPPEDDELARALAM
SLGNSDTPVQEEDDRTNDLELEEVNVQLPPMDEVLSSCLRLLQAKETLAFPVRDMLV
TISSQNDGQNRVKVLTYLIDHLKQCLVASDPLKNTALSAFCHVLALILHGDTAAREVAS
KAGLVKWVLSLLCSWEMEPREGQTTKVPNWVTSCFLSVDRMLQLEPKLPDVTELDVL
KKDSSPTQTSVVIDDSKKKVSESSSSVGLLDLEDQEQLLRICCKCIQKQLPSGTMHAIL
QLCATLTKVHVAAISFLESGGLHALLSLPTSSLFSGFNSWVSTIIRHILEDPHTLQQAME
LEIRHSLVTAANRHANPRVTPRNFVQNLAFWVHRDPVIFMKAAQAVCQIEMVGDRPYV
VLLKDREKEKSKEKEKDKLVDKDKSSGVATKITSGDMVMASPVSAKGKQSDLSVRNM
KSHRKPPQTFVTVIEHLLDLVMSFVPPQRAEDQSDGSSSMDMDIDSSSAKGKGKAVA
VTHEESKQAIQDATACLAKNAFVLKLLTDVLLTYASSVQVVLRHDAELSSTRGPTRTS
GGIFNHILQHLLPHATKQKKERKPDSDWRYKLATRGNQFLVASSIRSSEGRKRICSEIC
SIFVEFTDNSTGCKPPMLRMNAYVDLLNDILSARSPTGSSLSAESVVTFVEVGLVQCL
TKTLQVLDLDHPDSAKIVTGIVKALEVVTKEHVHLADFNAKGENSSKTVLEQNNVDSS
SNRFQVLDTTSQPTAMVTDHRETFNAVHAPRSSDSVADEMDHDRDIDGGFAHDGED
DFMHEIAEDRTGNESTMDIRFDIPRNREDDMAEDEDDSDEDMSGDDGEEVDEDDDD
EENNNLEEDDAHQISHADTDQDDREIDEEEFDEDLLEEDDDDEDEEGVILRLEEGINGI
NVFDHIEVFGGSNNVSGDTLRVMPLDIFGTRRQGRSTSIYNLLGRASDQGVLDHPLLE
EPSMLLPQQRQPENLVEMAFSDRNHENSSSRLDAIFRSLRSGRNGHRFNMWLDDGP
QRNGSAAPTVPEGIEELLLSQLRRPTAEHPDEQSTPAVDAQVNDPPSNFHGSETDAR
EGSAEQNENDDIPAVRSEVDGSASAGPAPPHSDELQRDASNASEHVADMQYERSDA
AVRDVEAVSQASSGSGATLGESLRSLDVEIGSVEGHDDGDRHGASDRTPLGDVQAA
TRSRRPSGNAVLVSSRDISLESVREIPQNTVQESDQNASEGDQEPNRATGTDSIDPTF
LEALPEDLRAEVLSSRQNQVTQTSSEQPQHDADIDPEFLAALPPDIREEVLAQQRAQR
LQQQSQELEGQPVEMDAVSIIATFPSEIREEVLLTSPDTLLATLTPALVAEANMLRERF
AHRYHSGSLFGMNSRNRRGESSRRGDIIGSGLDRNTGDSSRQTASKLIETVGTPLVD
KDALNALIRLLRWQPIYKGQLQRLLLNLCAHRESRKSLVQILVDMLMLDLQGSSKKSI
DATEPSFRLYGCHANITYSRPQSSDGVPPLVSRRVLETLTYLARNHPNVAKLLLFLQF
PCPPTCHTETLDQRRGKAVLVEDGEQQSAFALVLLLTLLNQPLYMRSVAHLEQLLNLL
EVVMLNAENEVNQVKLQSSSERPSGPENATQDAQEDASVPGSSGAKPNADDSGKS
SSDNISDLQAVLHSLPQAELRLLCSLLAHDGLSDNAYLLVAEVLKKIVALAPFICCHFIN
ELSRSMQNLTVCAMNELHLYEDSEKAILSTSSANGMAVLRVVQALSSLVTSLQERKDP
ELLAEKDHSDALSQISDINTALDALWLELSNCISKIESSSDYTSNLSPTSANATRVSTGV
APPLPAGTQNILPYIESFFVTCEKLRPGQPDAVQEPSTSDMEDASTSSSGQKSSASHT
SLDEKHTAFVKFSEKHRRLLNAFIRQNSGLLEKSFSLMLKVPRLIDFDNKRAYFRSKIK
HQHDHHHSPVRISVRRAYILEDSYNQLRMRSPQDLKGRLTVHFQGEEGIDAGGLTRE
WYQLLSRVIFDKGALLFTTVGNDLTFQPNPNSVYQTEHLSYFKFVGRVVGKALFDAQL
LDVHFTRSFYKHILGAKVTYHDIEAIDPAYYRNLKWMLENDISDVLDLTFSMDADEEKLI
LYEKAEVTDCELIPGGRNIRVTEENKHEYVDRVAEHRLTTAIRPQINAFMEGFNELIPR
ELISIFNDKEFELLISGLPDIDLDDLKANTEYSGYSIASPVIQWFWEIVQGFSKEDKARFL
QFVTGTSKVPLEGFSALQGISGPQRFQIHKAYGSTNHLPSAHTCFNQLDLPEYTSKDQ
LQERLLLAIHEANEGFGFG
SEQ ID NO: 8 UPL2 CDS; D genome
TraesCS5D02G118000:TraesCS5D02G118000.1
SEQ ID NO: 9 UPL2 amino acid; D genome; HECT domain underlined
MAAAAMAAHRASFPLRLQQILSGSRXXXXXXXXXXXXPAKVKAFIDRVINIPLHDIAIPL
SGFHWEFNKGNFHHWKPLFMHFDTYFKTYISSRKDLLLSDDMSESEPLTKNTILQILR
WVQIVLENCQNKTTFAGLEHFKNLLASSDPEVVVAALETLASVVKINPSKLHMNGKLIS
CGAINSHLLSLAQGWGSKEEGLGLYSCVVANERNQLEGLCLFPADMENKYDGTQHR
LGSTLHFEYNLAPVQDSDQANDKSSNLCVIHMPDLHLRKEDDLSILKQCIDKFNVPPE
HRFALFTRIRYAHAFNSPRTCRLYSRISLLSFIVLVQSSDAHDELTSFFTNEPEYINELI
RLVRSEDIVPGPIRALAMLALGAQLAAYASSHERARILSGSSIISAGGNRMVLLSVLQK
AISSLSSPNDTSSPLIVDALLQFFLLHVLSSSSSGTTVRGSGMVPPLLPLLQDKDPSH
MHLVCLAVKTLQKLMEYSSPAVSLFKDLGGVELLSQRLHVEVQRVIGVAEITSVLASD
TSKSEDDHLYSQKRLIKALLKALGSATYSPANPARSQSSNDNSLPMSLSLIFQNVGKE
GGDIYFSSVTVMSEIIHKDPTCFPALKELGLPDAFLSSVTAGVIPSCKALICVPNGLGAI
CLNNQGLESVRETSVLRFLVETFTSRKYLIPMNEGVVLLANAVEELLRHVQSLRSTGV
DIIIEIINKLSCPRGDKITEAARAEEKTDMETDVEGRDLVSAMDSGTDGTNDEQFSHLSI
FHVMVLVHRTMENSETCRLFVEKGGLQTLLTLLLRPTITQSSGGMPIALHSTMVFKGE
TQQHSTPLARAFCSSLKEHLKNALQELDTVFRSCEVTKLEKGAIPSLFIVEFLLFLAAS
KDNRWMNALLSEFGDVSRDVLEDIGRVHREVLWQISLFDEKKIEPEASSPSANEAQQ
VDAAVGDTDDNRYTSFRQYLDPLLRRRGSGWNIESQVSDLINIYRDMGRAATDSHR
VGADRYPSTGLPSSSQDQPSSSSDANAKSEEDKKRSEHSSCCDMMRSLSYHINHLE
MELGKAMLLTSRRENSPINLSPSVVSVASNIASIVLEHLNFEGHTISPEREITVATKCRY
LGKWVEFIDGILLDRPESCNPIMVNSFYCRGVIQAILTTFEATSELLFAMNRPPSSPME
TDSKTGKEEKDTDCSWIYGPLSSYGAAMDHLVTSSFILSSSTRQLLEQPIFSGTVRFP
QDAERFMKLLQSKVLKTVLPIWAHPQFPECNLELISSVTSIMRHVYSGVEVKNNVSNI
AARLAGPPPDENAISLIIEMGFSRARAEEALRQVGTNSVEIATDWLFSHPEEPPEDDE
LARALAMSLGNSDTPVQEEDDRTNDLELEEVNVQLTSMDEVLSSCLRLLQAKETLAF
PVRDMLVTISSQNDGQNRVKVLTYLIDHLKQCLVASDPLKNTALSAFFHVLALILHGDT
AAREVASKAGLVKVVLNLLCSWELEPREGQTTKVPNWVTSCFLSVDRMLQLEPKLP
DVTELDVLKKDNSPTQTSVVIDDSKKKDSESSSSVGLLDLEDQEQLLRICCKCIQKQL
PSGTMHAILQLCATLTKVHVAAISFLESGGLHALLSLPTSSLFSGFNSWVSTIIRHILEDP
HTLQQAMELEIRHSLVTAANRHANPRVTPRNFVQNLAFVVYRDPVIFMKAAQAVCQI
EMVGDRPYVVLLKDREKEKSKEKEKDKLVDKDKSSGVATKITSGDMVMASPVSAKG
KQSDLSARNMKSHRKPPQTFVTVIEHLLDLVMSFVPPQRAEDQSDGSSSMDMDIDS
SSAKGKGKAVAVTHEESKQAIQDATACLAKNAFVLKLLTDVLLTYASSVQWVLRHDAE
LSSTRGPTRTSGGIFNHILQHLLPHATKQKKERKPDGDWRYKLATRGNQFLVASSIRS
SEGRKRICSEICSIFVEFTDNTGCKPPMLRMDAYVDLLNDILSARSPTGSSLSAESVVT
FVEVGLVQCLTKTLQVLDLDHPDSAKIVTGIVKALEVVTKEHVHLADFNAKGENSSKT
VLEQNNVDSSSNRFQVLDTTSQPTAMVTDHRETFNAVHASRSSDSVADEMDHDRDI
DGGFARDGEDDFMHEIAEDRTGNESTMDIRFDIPRNREDDMAEDEDDSDEDMSGD
DGEEVDEDDDDEENNNLEEDDAHQRSHADTDQDDREIDEEEFDEDLLEEEDDDDED
EEGVILRLEEGINGINVFDHIEVFGGSNNVSGDTLRVMPLDIFGTRRQGRSTSIYNLLG
RASDQGVLDHPLLEEPSMLLPQQRQPENLVEMAFSDRNHENSSSRLDAIFRSLRSG
RNGHRFNMWLDDGPQRNGSAAPTVPEGIEELLLSQLRRPMAEHPDEQSTPAVDAQ
VNDPPSNFHGPETDAREGSAEQNENNENVDIPAVRSEVDGSASAGPAPPHSDELQR
DASNASEHVADMQYERSDTAVRDVEAVSQASSGSGATLGESLRSLDVEIGSVEGHD
DGDRHGASDRTPLGDVQAATRSRRPSGNAVPVSSRDISLESVREIPPNTVQESDQN
ASEGDQEPNRATGTDSIDPTFLEALPEDLRAEVLSSRQNQVTQTSSEQPQHDADIDP
EFLAALPPDIREEVLAQQRAQRLQQQSQELEGQPVEMDAVSIIATFPSEIREEVLLTSP
DTLLATLTPALVAEANMLRERFAHRYHSGSLFGMNSRNRRGESSRRGDIIGSGLDRN
TGDSSRQTASKLIETVGTPLVDKDALNALIRLLRVVQPIYKGQLQRLLLNLCAHRESRK
SLVQILLDMLMLDLQGSSKKSIDATEPSFRLYGCHANITYSRPQSSDGVPPLVSRRVL
ETLTYLARNHPNVAKLLLFLQFPCPPTCHTETLDQRRGKAVLVEDGEQQSAFALVLLL
TLLNQPLYMRSVAHLEQLLNLLEVVMLNAENEVNQAKLESSAERPSGPENATQDALE
DASVAGSSGVKPNADDSGKSSADNISDLQAVLHSLPQAELRLLCSLLAHDGLSDNAY
LLVAEVLKKIVALAPFICCHFINELSRSMQNLTVCAMNELHLYEDSEKAILSTSSANGM
AVLRVVQALSSLVTSLQERKDPELLAEKDHSDALSQISDINTALDALWLELSNCISKIES
SSEYTSNLSPTSANATRVSTGVAPPLPAGTQNILPYIESFFVTCEKLRPGQPDAVQEP
STSDMEDASTSSSGQKSSASHTSLDEKHTAFVKFSEKHRRLLNAFIRQNSGLLEKSF
SLMLKVPRLIDFDNKRAYFRSKIKHQHDHHHSPVRISVRRAYILEDSYNQLRMRSPQD
LKGRLTVHFQGEEGIDAGGLTREWYQLLSRVIFDKGALLFTTVGNDLTFQPNPNSVY
QTEHLSYFKFVGRVVGKALFDAQLLDAHFTRSFYKHILGAKVTYHDIEAIDPAYYRNLK
WMLENDISDVLDLTFSMDXXXXXXXXXXXXXVTDCELIPGGRNIRVTEENKHEYVDR
VAEHRLTTAIRPQINAFMEGFNELIPRELISIFNDKEFELLISGLPDIDLDDLKANTEYSG
YSIASPVIQWFWEIVQGFSKEDKARFLQFVTGTSKVPLEGFSALQGISGPQRFQIHKA
YGSTNHLPSAHTCFNQLDLPEYTSKDQLQERLLLAIHEANEGFGFG
Maize
SEQ ID NO: 10 UPL2 CDS sequence
GRMZM2G331368_T02 CDS
SEQ ID NO: 11: UPL2 genome sequence
GRMZM2G331368|10:20707761. . 20724390
SEQ ID NO: 12 UPL2 amino acid sequence
MAAAAAAMAAHRASFPLRLQQILAGSRAVSPAIKIESEPPANIKAFIDRVVNIPLHDIAIP
LSGFCWEFNKGNFHHWRPLFIHFDTYFKTYISSRKDLLLSDDMTEADPMPKNAILKILR
VMQIILENCQNRSSFTGLAHLKLLLASSDPEIVVAALETLVALVKINPSKLHMNGKLISC
GPINTHLLSLAQGWGSKEEGLGIYSCVVANEGNHQGGLSLFPVDLENKYGGTQHRLG
STLHFEYNLGPAQYPGQTSDKGKSSNLCVIHIPDMHLQKEDDLSILKQCVDKFNVPPE
HRFALLTRIRYARAFNSARTCRIYSRISLLSFIVLVQSSDAHDELTYFFTNEPEYINELIRL
VRSEDSVPGSIRALAMLALGAQLAAYASSHERARILSGSSIISAGGNRMVLLSVLQKAI
SSLNSLNDTSSPLIVDALLQFFLLHVLSSSSSGTTVRGSGMVPPLLPLLRDNDPSHMH
LVCLAVKTLQKLMEYSSPAVSLFKDLGGVELLSQRLHVEVQRVIGTADGHNSMVTDA
VKSDDNHMYSQKRLIKALLKALGSATYSPGNPARSQSSQDNSLPVSLSLIFQNVDKFG
GDIYFSAVTVMSEIIHKDPTCFITLKELGVPDAFISSVTAGVIPSCKALICVPNGLGAICLN
NOGLEAVRETSALRFLVDTFTSRKYLIPMNEGVVLLANAVEELLRHVQSLRSIGVDIIIEI
INKLSSSQEYKNNETATLQEKTDMETDVEGRDLVSAMDSSVDGSNDEQFSHLSIFHV
MVLVHRTMENSETCRLFVEKGGLHALLTLLLRPSITQSSGGMPIALHSTMVFKGFTQH
HSTPLARAFCSSLKEHLKSALKELDKVSNSFDMTKIEKGAIPSLFVVEFLLFLAASKDN
RWMNALLSEFGDASREVLEDVGQVHREVLWKISLFEKNKIVAETSSSSSTSEAQQPD
MSASDIGDSRYTSFRQYLDPILRRRGSGWNIESQVSDLINMYRDIGRAASDSQRVGS
DRYSSLGLPSSSQDQFSSSSDANASTRSEEDKKKSEHSSCFDMMRSLSYHINHLFLE
LGKAMLFASRRENSPVNLSPAVISVANNIASIVLEHLNFEGHSVSFERDMTVTTKCRYL
GKVVEFVDGMLLDRPESCNSIMVNSFYCRGVIQAILTTFQATSELLFTMSRPPSSPME
TDSKTGKDGKEMDSSWIYGPLTSYGAIMDHLVTSSFILSSSTRQLLEQPIFNGSVRFP
QDAETFMKLLQSKVLKTVLPIWAHPQFPECNIELISSVMSIMRHVCSGVEVKDTVGNG
GARLAGPPPDESAISLIVEMGFSRARAEEALRQVGTNSVEIATDWLFAHPEEPQEEDD
ELARALAMSLGNSVTPAQEGDSRSNDLELEEATVQPPPIDEMLRSCLQLLQRKEALAF
SVRDMLVTISSQNDGQNRVKVLTYLIDNLKQCVVASEPSNDTALSALLHVLALILHGDT
AAREVASKAGLVKVALDLLCSWEVQIRESSMIEVPNWVISCFLSVDQMLQLEPKLPDV
TELHVLKRDNSNIKTSLVIDDSKRKDSESLPNVGLLDMEDQFQLLKICCKCIGKQLPSA
SMHAILQLSATLTKVHAAAICFLESGGLNALLSLPTSSLFSGFNNMASTIIRHILEDPHTL
QQAMELEIRHSLVTAANRHANPRVTPRNFIQNLAFVVYRDPVIFMKAAQSVCQIEMVG
DRPYVVLLKDREKERIKEKDKDKSVDKDKATVAVTKVVSGDTAAGSPANSHGKQSDL
NSRNVKSHRKPPQSFVTVIEHLLDLLMSFVPPPRPEDQVDVSGTALSSDMDIDCSSAK
GKGKAVSVPPEESKHAIQESTASLAKTAFFLKLLTDVLLTYASSIHVVLRHDAELSNMH
GPNRTSARLTSGGIFNHILQHFLPHATRQKKERKNDGDWMYKLATRANQFLVASSIRS
AEARKRIFSEICSIFLDFTDSSAGYNAPVPRMNVYVDLLNDILSARSPTGSSLSAESAVI
FVEAGLVHSLSTMLQVLDLDHPDSAKIVTAVVKALELVSKEHIHSADNAKGVNSSKIAS
DSNNVNSSSNRFQALDMTSQPTEMVTDHRETFNAVRTSQISDSVADEMDHDRDMD
GGFARDGEDDFMHEMAEDGTGDGSTMEIRIEIPRNREDDMAPAADDTDEDISAEDGE
DDEDEDEENNNLEEDDAHRMSHPDTDQEDREMDEEEFDEDLLEEDDEDEDEEGVIL
RLEEGINGINVLDHVEVFGGSNNLSGDTLRVMPLDIFGTRRQGRSTSIYNLLGRASDH
GVLDHPLLEEPSSTTNFSDQGHPENLVEMAFSDRNHESSSSRLDAIFRSLRSGRNGH
RFNMWLDDGPQRNGSAAPAVPEGIEELLISHLRRPTPQPDGQRTPVGGAQENDQPN
HGSDAEAREVAPAQQNENSESTLNPLDLSECAGPAPPDSDALQRDVSNASELATEM
QYERSDAITRDVEAVSQASSGSGATLGESLRSLEVEIGSVEGHDDGDRHGTSGTSER
LPLGDIQAAARSRRPSGNAVPVSSRDMSLESVSEVPQNPDQEPDQNASEGNQEPTR
AAGADSIDPTFLEALPEDLRAEVLSSRQNQVTQTSNDQPQDDGDIDPEFLAALPPDIR
EEVLAQQRTQRMQQQSQELEGQPVEMDAVSIIATFPSEIREEVLLTSPDTLLATLTPAL
VAEANMLRERFAHRYHSSSLFGMNSRNRRGESSRRDIMAAGLDRNTGDPSRSTSKP
IETEGAPLVDEDGLKALIRLLRVVQPLYKGQLQKLLVNLCTHRGSRQALVQILVDMLML
DLQGFSKKSIDAPEPPFRLYGCHANIAYSRPQSSDGLPPLVSRRVLETLTNLARSHPN
VAKLLLFLEFPCPSRCFPEAHDHRHGKAVLLDDGEEQKTFALVLLLNLLDQPLYMRSV
AHLEQLLNLLDVVMHNAENEIKQAKLEASSEKPSAPDNAVQDGKNNSDISVSYGSELN
PEDGSKAPAVDNRSNLQAVLRSLPQPELRLLCSLLAHDGLSDSAYLLVGEVLKKIVAL
APFFCCHFINELARSMQNLTLRAMKELHLYENSEKALLSSSSANGTAVLRVVQALSSL
VNTLQERKDPEQPAEKDHSDAVSQISEINTALDSLWLELSNCISKIESSSEYASNLSPA
SASAAMLTTGVAPPLPAGTQNLLPYIESFFVTCEKLRPGQPDAVQDASTSDMEDAST
SSGGQRSSACQASLDEKQNAFVKFSEKHRRLLNAFIRQNSGLLEKSFSLMLKIPRLIDF
DNKRAYFRSKIKHQYDHHHHSPVRISVRRPYILEDSYNQLRMRSPQDLKGRLTVQFQ
GEEGIDAGGLTREWYQSISRVIVDKSALLFTTVGNDLTFQPNPNSVYQTEHLSYFKFV
GRVVGKALFDGQLLDAHFTRSFYKHILGVKVTYHDIEAIDPSYYKNLKWMLENDISDVL
DLTFSMDADEEKLILYEKAEVFAVTDCELIPGGRNIRVTEENKHEYVDRVAEHRLTTAI
RPQINAFLEGFNELIPRELISIFNDKELELLISGLPDIDLDDLKTNTEYSGYSIASPVVQW
FWEIVQGFSKEDKARFLQFVTGTSKVPLEGFSELQGISGPQRFQIHKAYGSTNHLPSA
HTCFNQLDLPEYTSKEQLQERLLLAIHEANE
SEQ ID NO: 13 UPL2 CDS sequence
GRMZM2G411536_T03 CDS
SEQ ID NO: 14: UPL2 genome sequence
>GRMZM2G411536|3:111568547. .111585874
SEQ ID NO: 15 UPL2 amino acid sequence
MAAAAAAHRASFPLRLQQILAGSRAVSPAIKVESEPPANVKAFIDQVINIPLHDIAIPLSG
FRWEFNKGNFHHWKPLFIHFDTYFKTYISSRKDLLLSDDMTEAEPMPKNAILKILIVMQI
ILENCQNRSSFTGLEHLKLLLASSDPEIVVAALETLVALVKINPSKLHMNGKLISCGSINT
HLLSLAQGWGSKEEGLGIYSCWVANEGNQQGGLSLFPVDLESKYQHRLGSTLHFEYN
LGSAQYPDQTSDKGKSSNLCVIHIPDMHLQKEDDLSILKQCVDKFNVPPEHRFALLTRI
RYARAFNSTRTCSIYSRISLLSFIVLVQSSDAHDELTYFFTNEPEYINELIRLVRSEDSVP
GPIRALAMLALGAQLAAYASSHERARILSGSSIISAGGNRMVLLSVLQKAIFSLNSPNDA
SSPLIVDALLQFFLLHVLSSSSSGTTVRGSGMVPPLLPLLRDNDSSHMHLVCLAVKTL
QKLMEYSSPAVSLFKDLGGVDLLSRRLHVEVQRVIGTADGHNSMVTDAVKSKEDHLY
SQKRLIKALLKALGSSTYSPGIPARSQSSQDNSLPVSLSLIFQNVEKFGGDIYFSAVTV
MSEIIHKDPTCFPALKELGLPDAFLSSVTAGVIPSCKALICVPNGLGAICLNNQGLEAVR
ETSALRFLVYTFTSRKYLIPLNEGVVLLANAAEELLRHVQSLRSIGVDIIIEIINKLSSSLK
DRNNETAILEEKTDMETDVEGRDLVGGMDSSVEGSNDEQFSHLSIFHVMVLVHRTME
NSETCRLFVEKGGLNALLTLLLRPSITLSSGGMPIALHSTMVFKGFTQHHSTPLARAFC
SSLREHLKSALGELDKVSNSFEMTKIEKGAIPSLFVVEFLLFLAASKDNRWMNALLSEF
GDASREVLEDIGRVHREVLWKISLFEENKIDAEISLSSSTSEAQQPDLSASDIGDSRYT
SFRQYLDPILRRRGSGWNIESQVSDLINMYRDIGSAASDSQRVGSDRYSSLGLPSSS
QDQSSSSSDANVSTRSEEEKKNSEHSSCFDMMRSLSYHINHLFMELGKAMLLTSRRE
NSPVNLSPSVISVANNIASIMLEHLNFEGHSVSSEREMTVTTKCQYLGKVAEFIDGILLD
RPESCNPIMVNSFYCCGVIQAILTTFQATSELLFTMSRPPSSPMETDSKTGKDGKDMN
SSWIYGPLISYGAIMDHLVTSSFILSSSTRQLLEQPIFNGSVRFPQDAERFMKLLQSKVL
KTVLPIWAHPEFPECNIELISSVMSIMRHVCSGVEVKNTVGNDGARLTGPPPDESAISL
IVEMGFSRARAEEALRQVGTNSVEIATDWLFSHPEDELARALAMSLGNSDTPAQEGN
GRSNDLELEEVTVQLPPIDEMLHSCFQLLQTKEALAFPVRDMLVTISSQKDGQNRVKV
LTYLIENLKQCVVASEPSNDTALSALLHVLALILHGDTAAREVASKAGIVKVALDLLSSW
ELELRESGMIEVPNWVSSCFLSVDQMLQLEPKLPDVTELDVLKRDNSNIKTSLVIDESK
KKDSESLSSVGLLDMEDQYQLLKICCKCIEKQLPSASMHAILQLSATLTKVHAAAICFLE
SGGLNALLSLPTSSLFSGFNSVASTIIRHILEDPHTLQQAMELEIRHSLVTAANRHTNPR
VTPRNFVQNLAFVIYRDPVIFMKAVQSVCQIEMVGDRPYVVLLKDREKERSKEKDKDK
SVDKDKATGAVAKVVSGDTAAGSPANAQGKQSDLNSRNVKSHRKPPQSFVTVIEHLL
DLVMSFVPPPRPEDQADVVSGTALSSDMDIDCSSAKGKGKAVSVPPEESKHAIQEST
ASLAKASFFLKLMTDVLLTYTSSIQVVLRHDADLSNMHGPNRTNSGLISGGIFNHILQH
FLPHATKQKKERKSDGDWMYKLATRANQFLVASSIRSAEARKKVFSEICNILLDFTDS
SAAYKAPVARMNVYVDLLNDILSARSPTGSSLSAESAVTFVEVGLAPSLLKMLQNLDL
DHPDSAKIVTAIVKALELVSKEHVHSADNAKGENSSKIASDSNNVNSSPNRFQALDMT
SQPTEMITDHRETFNADQTSQSSDSVADEMDHDRDMDGGFARDGEDDFMHEMAGD
GTGNESTMEIRFEISRNRDDMADDDDDDDNTDEDMSAEDDEEVNEDDEDEDEENNN
LEEDDAHQMSHPDTDQEDREMDEEEFDEDLLEDDDDEDEEGVILRLEEGINGINVFD
HIEVFGGSNNLSGDTLRVMPLDIFGTRRQGRSTSIYNLLGRASDHGVLDHPLLEEPSS
TLNFSHQEQPENLVEMAFSDRNHEGSSSRLDAIFRSLRSGRNGHRFNMWLDDGPQR
NGSAAPAVPEGIEELLISHLSRPTQQPGAQTVGGTQENDQPKHGSAAEAREGSPAQ
QNENSENTTNPVDLSESAGPAPPDSDALQRVVSNASIEHATEMQYERSDTITRDVEA
VSQASSGSGATLGESLRSLEVEIGSVEGHDDGDRHGTSGASERLPLGDIQAAARSRR
PSGNAVAVSSRDMSLESVSEVPQNPDQEPDHNASEGNQEPRGVGADTIDPTFLEAL
PEDLRAEVLSSRQNQVTQTSNDQPQNDGDIDPEFLAALPLDIREEVLAQQRSQRIQQ
QSQELEGQPVEMDAVSIIATFPSEIREEVLLTSPDTLLATLTPALVAEANMLRERFAHR
YHSSSLFGMNSRNRRGESSRRDIMAAGLDRNTGDPSRSTSKPIEIEGAPLVDEDGLK
ALIRLLRVVQPLYKGQLQRLLVNLCTHRDNRQALVQILVDMLMLDLQGFSKKSVDASE
PPFRLYGCHANITYSRPQSSNGVPPLVSRRVLETLTNLARSHPNVAKLLLFLEFPCPS
RCRSEAHDHRHGKAVLEDGEERKAFAVVLLLTLLNQPLYMRSVAHLEQLLNLLEVVM
HNAENEINQAKLEASSEKPSENAVKDVKDNTSISDSYGSKSNPEDGSKALAVDNKSNL
RAVLRSLPQSELRLLCSLLAHDGLSDSAYLLVGEVLKKIVALAPFFCCHFINELARSMQ
SLTFCAMKELRLYENSEKALLSSTSANGTAILRVVQALSSLVSTLQDRKDPEQPAEKD
HSDAVSQISEINTALDALWLELSNCISKIESSSEYASNLTPASASAATLTAGVAPPLPAG
TQNILPYIESFFVTCEKLRPGQPDAVQEASTSDMEDASTSSGGQRSYSCQASLDEKQ
NAFVKFSEKHRRLLNAFIHQNPGLLEKSFSLMLKIPRLIDFDNKRAYFRSKIKHQYDHH
HHNPVRISVRRSYILEDSYNQLRMRSPQDLKGRLTVHFQGEEGIDAGGLTREWYQSL
SRVIFDKSALLFTTVGNDLTFQPNPNSVYQTEHLSYFKFAGRVVGKALFDGQLLDAHF
TRSFYKHILGVRVTYHDIEAIDPAYYKNLKWMLENDISDVLDLTFSMDADEEKLILYEKA
EVFAVTDCELIPGGRNIRVTEENKHQYVDRVAEHRLTTAIRPQINAFLEGFNELIPRELI
SIFNDKELELLISGLPDIDLDDLKANTEYSGYSIASPVIQWFWEIVQGFSKEDKARFLQF
VTGTSKVPLEGFSALQGISGPQRFQIHKAYGSTNHLPSAHTCFNQLDLPEYTSKEQLQ
ERLLLAIHEANEGFGFG
Millet
SEQ ID NO: 16 UPL2 CDS sequence
Seita.3G302600.1
SEQ ID NO: 17 UPL2 genome sequence
>Seita. 3G302600|scaffold_3:34832073. .34846959
SEQ ID NO: 18 UPL2 amino acid sequence
MAAAAMAAHRASFPLRLQQILAGSRAVSPAIKVESEPPAKVKEFIDRVINIPLHDIAIPLS
GFRWEFNKGNFHHWKPLFMHFDTYFKTYLSSRKDLLLSDDMAEADPLPKNTILKILRV
MQIVLENCHNKSSFAGLEHFKLLLASSDPEIVVAALETLAALVKINPSKLHMNGKLISCG
AINTHLLSLAQGWGSKEEGLGLYSCVVANEGNQQEGLSLFPADMENKYDGSQHRLG
STLHFEYNLSPTQDPDQTSDKSKSSNLCVIHIPDMHLQKEDDLSILKQCVDKFNVPPE
HRFALLTRIRYARAFNSARTCRLYSRISLLSFIVLVQSSDAHDELTSFFTNEPEYINELIR
LVRSEDFVPGPIRALAMLALGAQLAAYASSHERARILSGSSIISAGGNRMVLLSVLQKAI
SSLNSPNDTSAPLIVDALLQFFLLHVLSSSSSGTTVRGSGMVPPLLPLLQDNDPSHMH
LVCLAVKTLQKLMEYSSPAVSLFKDLGGVELLSQRLHVEVQRVIGTVDGHNSMVTDA
VKSEEDVLYSQKRLIRALLKALGSATYSPGNPARSQSSQDNSLPVSLSLIFQNVEKFG
GDIYFSAVTVMSEIIHKDPTCFPALKELGLPDAFLSSVTAGVIPSCKALICVPNGLGAICL
NNQGLEAVRETSALRFLVDTFTSRKYLMPMNEGVVLLANAVEELLRHVQSLRSTGVDI
IIEIINKLCSSQEYRSNEPAISEEEKTDMETDVEGRDLVSAMDSSAEGMHDEQFSHLSI
FHVMVLVHRTMENSETCRLFVEKGGLQALLALLLRPSITQSSGGMPIALHSTMVFKGF
TQHHSTPLARAFCSSLREHLKSALEELDKVSSSVEMSKLEKGAIPSLFVVEFLLFLAAS
KDNRWMNALLSEFGDASREVLEDIGRVHREVLYKISLFEENKIDSEASSSSLASEAQQ
PDSSASDIDDSRYTSFRQYLDPLLRRRGSGWNIESQVSDLINIYRDIGRAASDSQRVD
SDRYSNQGLPSSSQDQSSSSSDANASTRSEEDKKKSEHSSCCDMMRSLSYHISHLF
MELGKAMLLTSRRENSPVNLSPSVISVAGSIASIVLEHLNFEGRSVSSEKEINVTTKCR
YLGKVVEFVDGILLDRPESCNPIMVNSFYCRGVIQAILTTFQATSELLFTMSRPPSSPM
DTDSKTGKDGKETDSSWIYGPLSSYGAVMDHLVTSSFILSSSTRQLLEQPIFNGSVRF
PQDAERFMKLLQSKVLKTVLPIWAHSQFPECNIELISSVTSIMRHVCTGVEVKNTVGN
GSGRLAGPPPDENAISLIVEMGFSRARAEEALRQVGTNSVEIATDWLFSHPEEPQEED
DELARALAMSLGNSDTSAQEEDSRSNDLELEEETVQLPPIDEILYSCLRLLQTKEALAF
PVRDMLVTISTQNDGQNREKVLTYLIENLKQCVMASESLKDTTLSALFHVLALILHGDT
AAREVASKAGLVKVALDLLFSWELEPRESEMTEVPNWVTSCFLSVDRMLQLEPKLPD
VTELDVLKKDNSNAKTSLVIDDSKKKDSESLSSVGLLDLEDQKQLLKICCKCIEKQLPS
ASMHAILQLCATLTKVHAAAICFLESGGLNALLSLPTSSFFSGFNSVASTIIRHILEDPHT
LQQAMELEIRHSLVTAANRHANPRVTPRNFVQNLAFVVYRDPVIFMKAAQAVCQIEMV
GDRPYVVLLKDREKERSKEKDKDKSADKDKATGAVTKVTSGDIAAGSPASAQGKQPD
LSARNVKPHRKPPQSFVTVIEHLLDLVISFVPPPRSEDQADVSGTASSSDMDIDCSSA
KGKGKAVAVAPEESKHAAQEATASLAKSAFVLKLLTDVLLTYASSIQVVLRHDADLSS
MHGPNRPSAGLVSGGIFNHILQHFLPHAVKQKKDRKTDGDWRYKLATRANQFLVASS
IRSAEGRKRIFSEICNIFLDFTDSSTAYKAPVSRLNAYVDLLNDILSARSPTGSSLSAES
AVTFVEVGLVQSLSRTLQVLDLDHPDSAKIVSAIVKALEVVTKEHVHSADLNAKGDNSS
KIASDSNNVDLSSNRFQALDTTSQPTEMITDDRETFNAVQTSQSSDSVEDEMDHDRD
MDGGFARDGEDDFMHEMAEDGTGNESTMEIRFEIPRNREDDMADDDEDTDEDMSA
DDGEEVDEDDEDEDDDEENNNLEEDDAHQMSHPDTDQDDREMDEEEFDEDLLEDD
DEDEDEEGVILRLEEGINGINVFDHIEVFGGSNNLSGDTLRVMPLDIFGTRRQGRSTSI
YNLLGRASDHGVLDHPLLEEPSSMLNLPHQGQPENLVEMAFSDRNHESSSSRLDAIF
RSLRSGRNGHRFNMWLDDSPQRSGSAAPAVPEGIEELLISHLRRPTPEQPDDQRTPA
GGTQENDQPTNVSEAEAREEAPAEQNENNENTVNPVDVLENAGPAPPDSDALQRDV
SNASEHATEMQYERSDAVVRDVEAVSQASSGSGATLGESLRSLEVEIGSVEGHDDG
DRHGASGASDRLPLGDMQATARSRRPSGSAVQVGGRDISLESVSEVPQNSNQEPD
QNANEGNQEPARAADADSIDPTFLEALPEDLRAEVLSSRQNQVAQTSNDQPQNDGDI
DPEFLAALPPDIREEVLAQQRAQRLQQQSQELEGQPVEMDAVSIIATFPSEIREEVLLT
SPDTLLATLTPALVAEANMLRERFAHRYHSSSLFGMNSRNRRGESSRREIMAAGLDR
NGDPSRSTSKPIETEGAPLVDEDALRALIRLLRVVQPLYKGQLQRLLLNLCAHRDSRK
SLVQILVDMLMLDLQGSSKKSIDATEPPFRLYGCHANITYSRPQSSDGVPPLVSRRVL
ETLTYLARSHPNVAKLLLFLEFPSPSRCHTEALDQRHGKAVVEDGEEQKAFALVLLLTL
LNQPLYMRSVAHLEQLLNLLEVVMLNAETQINQAKLEASSEKPSGPENAVQDSQDNT
NISESSGSKSNAEDSSKTPAVDNENILQAVLQSLPQPELRLLCSLLAHDGLSDNAYLLV
AEVLKKIVALAPFFCCHFINELARSMQNLTLCAMKELRLYENSEKALLSSSSANGTAILR
WVQALSSLVTTLQEKKDPELPAEKDHSDAVSQISEINTALDALWLELSNCISKIESSSEY
VSNLSPAAANAPTLATGVAPPLPAGTQNILPYIESFFVTCEKLRPGQPDAVQEASTSD
MEDASTSSGGLRSSGGQASLDEKQNAFVKFSEKHRRLLNAFIRQNPGLLEKSFSLML
KIPRLIDFDNKRAYFRSKIKHQHDHHHSPVRISVRRAYILEDSYNQLRMRSPQDLKGRL
TVHFQGEEGIDAGGLTREWYQSLSRVIFDKGALLFTTVGNDLTFQPNPNSVYQTEHLS
YFKFVGRVVGKALFDGQLLDAHFTRSFYKHILGVKVTYHDIEAIDPAYYKNLKWMLEN
DITDVLDLTFSMDADEEKLILYEKAEVTDSELIPGGRNIKVTEENKHEYVDRWVEHRLTT
AIRPQINAFLEGFNELIPRELISIFNDKELELLISGLPDIDLDDLKANTEYSGYSIASPVIQ
WFWEIVQGFSKEDKARFLQFVTGTSKVPLEGFSALQGISGPQRFQIHKAYGSTNHLP
SAHTCFNQLDLPEYTSKEQLQERLLLAIHEANEGFGFG
Soybean
SEQ ID NO: 19 CDS UPL2 KRH72480
ATGACAACCCTAAGATCAAGTTGGCCTTCGAGGCTGCGCCAACTTCTGTCCAGCGGGGGCGCCATTGG
TCCTTCAGTCAAGGTGGACTCCGAGCCCCCTCCTAAGATCAAAGCCTTCATTGAGAAGATCATCCAGTG
TCCATTACAAGATATTGCCATACCACTTTCTGGCTTTCGGTGGGAGTACAATAAGGGGAATTTTCATCAC
TGGAGACCGCTGTTGCTTCATTTTGATACATACTTCAAGACTTATTTGTCGTGTAGAAATGATCTGACGT
TGTTAGATAATCTAGAAGATGACAGCCCTTTACCAAAACATGCAATTCTGCAAATATTGCGAGTGATGC
AAAAAATTTTAGAGAACTGTCCAAACAAGAGTTCCTTTGATGGCTTAGAGCATTTCAAGCTTTTACTTGC
ATCAACAGATCCTGAGATTCTTGTTGCTACATTGGAAACTCTTTCTGCACTTGTAAAAATTAATCCCTCTA
AGCTTCATGGAAGTCCAAAGATGATTTGCTGTGGTTCGGTGAACAGCTATCTTTTGTCCTTAGCACAAG
GCTGGGGAAGCAAGGAGGAGGGCCTAGGATTGTACTCTTGTGTTATGGCAAATGAGAAAGCCCAAGA
TGAAGCACTGTGCTTGTTTCCTTCTGAAGAGATTGGTCATGACCAATCAAATTGCCGCATAGGCACTAC
CCTTTATTTTGAATTGCATGGTCCCAATGCCCAAAGCAAGGAACATAGTGCAGATGCAGTTTCCCCTAG
CTCAACAGTTATACACATGCCAGATTTGCATCTGCGCAAAGAAGATGATTTGTCATTGATGAAGCAGTG
CACTGAAGAATTTAGCATTCCTTCTGAGCTCAGGTTTTCCTTGCTCACTAGAATCAGATATGCTCGTGCC
TTTCGTTCTCCTAGAATATGCAGGCTTTACAGCCGGATTTGCCTACTTTCTTTCATTGTTCTGGTGCAGTC
TGGTGATGCTCAGGAAGAACTTGTCTCCTTTTTTGCTAATGAACCAGAATATACAAATGAATTAATTAGA
ATTGTACGATCAGAGGAAGTTATATCTGGATCTATCAGGACACTTGCAATGCTTGCTCTAGGAGCTCAA
TTAGCAGCATATACATCATCGCATCATCGGGCACGGATCAGTGGATCTAGTTTAACTTTTGCTGGTGGG
AACCGCATGATACTCCTAAATGTGCTCCAGAGGGCTATTTTGTCATTGAAGATTTCTAATGATCCATCAT
CCCTTGCCTTTGTTGAAGCACTTCTTCAGTTCTATCTGCTCCATGTGGTCTCAACCTCAACTTCTGGTAAT
AATATTAGAGGTTCTGGCATGGTGCCAACATTCTTGCCGTTGCTGGAGGATTTTGATCCTACACATATTC
ATCTAGTCTGTTTTGCTGTGAAAACTCTTCAGAAGCTTATGGATTATAGTAGCTCAGCTGTATCATTGTT
TAAAGAATTGGGGGGCATTGAACTTTTGGCTCAGAGGTTACAGAAAGAGGTACACAGAGTCATTGGTT
TGGTTGGAGGAACTGATAACATGATGCTTACTGGTGAAAGCTTGGGACATAGTACTGATCAATTGTACT
CCCAGAAGAGACTCATAAAGGTCTCCCTTAAGGCGCTTGGTTCTGCAACATACGCACCTGCAAACTCTA
CCAGATCTCAACATTCTCAAGACAGTTCATTACCTATAACTCTAAGCTTGATTTTTAAGAATGTAGATAA
GTTTGGAGGTGACATTTATTATTCAGCTGTTACTGTTATGAGTGAAATAATCCACAAAGATCCTACCTTT
TTTTCTGCTCTGCATGAAATAGGTCTTCCTGATGCGTTTTTATTGTCAGTTGGATCTGGAATACTTCCATC
ATCAAAGGCTTTGACATGCATTCCAAATGGTCTTGGGGCCATTTGTCTTAATGCCAAAGGGTTAGAGGC
CGTTAGAGAATCTTCATCGCTACGGTTCCTTGTTGACATTTTCACTAGCAAGAAGTATGTCTTAGCCATG
AATGAGGCTATTGTTCCTTTGGCAAATGCTGTGGAGGAACTTCTACGCCATGTATCTACATTGAGAAGC
ACTGGTGTTGATATTATCATTGAAATCATCCATAAGATCACATCTTTTGGGGATGGAAATGGTGCAGGA
TTTTCTGGAAAAGCTGAGGGCACCGCCATGGAAACAGATTCTGAAAACAAAGAAAAAGAAGGCCATTG
TTGCATTGTAGGCACATCATATTCGGCTGTAGAAGGGATAAGTGATGAGCAGTTTATTCAGCTATGTGT
CTTTCATTTGATGGTATTAGTTCATAGGACTATGGAAAATGCCGAGACATGCCGGTTGTTTGTGGAAAA
ATCAGGAATTGAAGCTTTATTGAATTTGTTATTACGACCCACTATTGCACAATCCTCAGATGGCATGTCT
ATTGCTTTACATAGCACAATGGTATTTAAAGGGTTTGCTCAACATCATTCAATTCCTCTGGCACATGCCTT
CTGTTCTTCTCTTAGAGAGCACTTAAAGAAAACTTTAGTGGGGTTTGGTGCAGCATCAGAACCTTTGTT
GCTGGATCCAAGGATGACAACTGATGGTGGCATCTTTTCTTCACTTTTCCTGGTTGAGTTCCTTCTATTTC
TTGTGGCATCGAAAGACAATCGTTGGGTGACTGCCTTGCTTACAGAATTTGGAAATGAGAGTAAGGAT
GTTCTTGAAGACATTGGATGCGTTCACCGTGAAGTTCTGTGGCAAATTTCTCTACTTGAAAATAGAAAA
CCTGAGATTGAGGAAGATGGTGCTTGTTCTTCTGATTCACAACAGGCTGAAGGGGATGTAAGTGAAAC
TGAAGAGCAAAGGTTCAATTCTTTCAGGCAGTATCTTGACCCATTATTGAGAAGGAGAACATCAGGAT
GGAGCATTGAATCCCAGTTTTTTAACCTTATAAACCTGTATCGAGATTTGGGCCGTTCCACTGGTTCTCA
AAATAGATTAGTTGGTCCGAGGTCAAGTTCTAGTAATCAGGTACAGCATTCTGGGTCAGATGATAATTG
GGGGACTGCTAATAAGAAGGAATCTGACAAGCAGAGAGCATATTATACATCTTGTTGTGACATGGTCA
GATCACTTTCATTTCACATTACCCATTTGTTCCAAGAGTTAGGAAAAGTAATGTTGCTACCTTCACGTCG
GCGTGATGATGTTGTGAATGTAAGTCCTGCTTCAAAATCAGTGGCTTCTACATTTGCATCCATTGCTTTT
GATCACATGAATTATGGTGGCCGTTGTGTAAATCTTTCGGGAACAGAAGAATCCATATCAACAAAATGT
CGATATTTTGGGAAAGTGATTGATTTTATGGATAATGTTCTAATGGAGAGGCCAGATTCATGCAATCCT
ATTATGCTGAATTGCTTGTATGGACGTGGAGTTATTGAAATTGTATTAACTACCTTTGAAGCTACCAGTC
AGCTGCTCTTTACAGTTAATCGGGCCCCTGCCTCGCCCATGGATACTGATGATGCAAATGCAAAGCAAG
ACGACAAGGAAGATACAGATAATTCATGGATTTATGGTTCTTTAGCTAGTTATGGGAAATTGATGGACC
ATCTAGTGACCTCCTCTTTTATATTATCATCATTCACAAAGCATTTGCTTGCACAGCCCCTTACTAATGGT
GATACACCTTTCCCAAGGGATGCTGAGACTTTTGTGAAGGTCCTTCAATCCAGAGTGTTGAAGACTGTA
CTTCCTGTTTGGACTCATCCCAAGTTTGTTGACTGTAGTTATGAATTTATTTCTACAGTTATTTCTATCATT
AGGCATGTCTATACAGGTGTTGAAGTAAAAAATGTGAATGGCAGTGCTGGTGCTCGCATTACTGGGCC
GCCTCCTAATGAAACAACTATTTCAACCATTGTAGAAATGGGGTTTTCCAGGTCTAGAGCAGAAGAAGC
TTTGAGGCAAGTTGGGTCAAATAGTGTGGAGTTGGCAATGGAGTGGTTGTTCTCTCATCCAGAGGAGG
CACAAGAAGATGATGAACTTGCCCGTGCACTTGCCATGTCCCTTGGAAACTCTGAATCAGATTCAAAGG
ATGCAGTTGCTAATGACAATGCCCTGCAGCTTGAGGAAGAGATGGTCCAACTTCCTCCTGTTGATGAGT
TGTTATCTACTTGTACAAAACTTTTGTCGAAGGAACCACTTGCTTTTCCAGTCCGTGACTTGCTTGTGAT
GATATGCTCTCAGGATGATGGTCAACATAGATCTAATGTGGTCTCATTTATTGTGGAACGGATCAAAGA
ATGTGGTTTGGTTCCTAGCAATGGAAATTATGCCATGCTGGCTGCTCTTTTTCATGTTCTAGCTTTAATTC
TTAATGAGGATGCTGTGGCTAGGGAAGCTGCTTCTACAAGTGGTTTAATCAAAATTGCCTCAGATCTAC
TCTACCAGTGGGATTCTAGTCTTGATATCAAGGAGAAACATCAGGTACCAAAATGGGTGACTGCTGCTT
TCCTTGCATTAGACAGATTGTTGCAAGTGGATCAAAAATTGAATTCTGAAATCGCAGAGCAGTTGAAGA
AGGAAGCTGTGAATAGCCAGCAGACATCAATTACCATTGATGAAGACAGGCAAAACAAGATGCAGTCT
GCATTGGGACTCTCTATGAAGTATGCAGATATACATGAACAGAAGAGACTTGTTGAGGTTGCTTGTAGT
TGTATGAAGAATCAACTTCCATCCGACACAATGCATGCTGTTCTGCTACTATGTTCCAATCTTACAAGGA
ATCATTCTGTAGCTCTTACTTTTTTGGATTCTGGTGGTTTAAGTCTACTTCTTTCTTTGCCAACCAGCAGTC
TCTTCCCTGGGTTTGACAATGTTGCTGCTAGTATTGTTCGTCATGTTCTTGAAGATCCTCAAACGCTCCAT
CAAGCAATGGAATCTGAGATAAAACATAGTCTTGTAGTTGCATCTAACCGGCATCCAAATGGAAGGGT
CAATCCTCATAATTTCCTITTAAATTTAGCTTCTGTGATTTCTCGGGATCCAGTAATTTTTATGCAAGCTG
CTCAATCTGTGTGCCAAGTTGAAATGGTAGGTGAGAGGCCATACATTGTCTTGCTGAAAGATAGGGAT
AAAGACAAAGCTAAGGATAAAGAAAAGGATAAGGATAAAACATTGGAGAAAGATAAAGTACAGAACA
TTGATGGGAAGGTTGTTTTGGGAAATACTAACACGGCACCTACTGGCAATGGCCATGGCAAAATTCAA
GATTCAAATACCAAGAGTGCCAAAGGTCACAGAAAACCTACCCAAAGTTTTATTAATGCAATAGAACTT
CTTCTTGAATCTGTATGCACTTTTGTTCCTCCCTTGAAGGGTGACATTGCCTCAAATGTTCTTCCTGGCAC
CCCAGCATCAACCGATATGGACATTGATGCCTCCATGGTTAAGGGAAAAGGAAAAGCAGTTGCCACTG
ATTCTGAGGGCAATGAAACTGGTAGTCAGGATGCTTCTGCATCACTTGCAAAGATTGTCTTCATTCTAA
AGCTTCTGACAGAGATACTATTGATGTATTCATCATCTGTTCATGTTTTACTTAGACGAGATGCTGAAAT
GAGCAGCATTAGAGGTTCTTATCAAAAGAGTCCTGCAGGTTTAAGCATGGGGGGATATTCTCTCATAT
TCTTCATAATTTTCTTCCATATTCTCGAAACTCCAAAAAGGACAAGAAAGCTGATGGTGATTGGAGGCA
GAAACTAGCAACCAGGGCCAACCAGTTTATGGTGGGTGCTTGTGTTCGATCTACAGAGGCAAGGAAGA
GGGTTTTTGGTGAGATTTGTTGTATCATCAATGAATTTGTTGATTCATGTCATGGCATTAAGCGTCCAGG
AAAAGAAATTCAGGTTTTTGTTGATCTACTAAATGATGTTTTGGCTGCTCGTACACCCGCTGGTTCATCC
ATTTCAGCTGAGGCCTCTACCACTTTTATTGATGCTGGTTTGGTTAAATCATTCACATGTACTCTACAAGT
TTTGGACCTTGACCATGCTGATTCATCTGAAGTTGCTACGGGTATTATTAAAGCTCTTGAGTTGGTAACC
AAGGAGCATGTCCAATTAGTTGATTCTAGTGCAGGGAAGGGTGATAATTCAGCAAAGCCTTCTGTTCTA
AGTCAACCCGGAAGAACAAATAATATTGGTGACATGTCTCAGTCCATGGAGACATCACAAGCCAATCCT
GATTCCCTTCAAGTTGACCGTGTTGGGTCTTATGCAGTTTGCTCCTATGGTGGGTCTGAAGCTGTTACTG
ATGATATGGAACATGATCAAGATCTTGATGGGAGCTTTGCTCCTGCTAATGAGGATGATTACATGCATG
AAAATTCTGAGGATGCAAGAGATCTTGAAAATGGAATGGAAAATGTGGGTCTACAATTTGAAATCCAA
TCTCATGGCCAAGAAAATCTTGATGAGGATGACGATGAGGACGATGATATGTCTGAAGATGAAGGTGA
GGATGTAGATGAAGATGAGGATGATGATGAGGAACACAATGATTTGGAAGAAGTCCATCATTTGCCAC
ATCCTGACACAGATCAAGATGAGCATGAGATTGATGATGAAGATTTTGATGATGAAGTGATGGAGGAA
GAGGATGAGGATGACGAGGAAGATGAAGATGGTGTTATACTGCAACTCGAGGAGGGGATTAATGGA
ATTAATGTTTTTGATCATATTGAGGTTTTTGGCAGAGATAATAGTTTTGCAAATGAAGCTTTTCAAGTGA
TGCCGGTTGAGGTTTTTGGATCCAGACGTCAGGGGAGGACAACATCTATTTATAGTCTTTTGGGAAGAA
CTGGTGATACCGCTGTGCCTTCTCGTCACCCACTCTTGCTTGAACCTTCTTCATTCCCCCCACCTACAGGG
CAATCAGATAGTTCATTGGAGAACAACTCATTGGGTTTGGATAATATATTTCGATCGCTGAGGAGTGGA
CGCCATGGACAGCGTTTGCACTTGTGGACTGATAATAACCAACAAAGTGGTGGGACAAACACTGTTGT
TGTACCCCAAGGCCTTGAGGATTTGCTTGTCACTCAATTAAGGCGACCAATCCCTGAAAAGTCATCCAA
TCAGAACATTGCAGAAGCAGGTTCTCATGGTAAAGTTGGAACGACCCAGGCACAAGATGCAGGGGGT
GCAAGGCCAGAAGTCCCTGTTGAAAGTAATGCTGTTCTGGAAGTTAGTACTATAACTCCCTCGGTTGAT
AACAGTAACAATGCGGGTGTCAGACCAGCTGGGACTGGACCTTCACATACAAATGTTTCAAACACACA
CTCACAGGAAGTTGAGATGCAATTTGAACATGCTGATGGAGCTGTGAGGGATGTTGAAGCTGTCAGCC
AGGAGAGTAGTGGTAGTGGTGCAACTTTTGGTGAAAGCCTTCGGAGCTTGGATGTTGAGATTGGAAGT
GCTGATGGCCATGATGATGGTGGTGAAAGGCAGGTTTCTGCTGATAGGGTGGCAGGTGATTCGCAGG
CAGCACGCACAAGAAGAGCAAATACGCCTTTGAGTCACATTTCTCCTGTGGTTGGAAGAGATGCGTTCC
TTCACAGTGTAACTGAAGTTTCAGAAAATTCAAGCCGTGATGCAGATCAAGATGGTGCAGCAGCAGAG
CAGCAGGTGAACAGTGATGCAGGATCAGGAGCTATTGATCCTGCTTTTCTGGATGCTCTTCCTGAGGA
GCTGCGTGCCGAACTCCTTTCAGCTCAGCAGGGTCAAGTGGCTCAGCCATCAAATGCTGAGTCTCAAAA
CACTGGGGATATTGATCCAGAGTTCCTTGCAGCTCTTCCAGCTGATATTCGAGCAGAAATTCTAGCTCA
GCAGCAAGCACAGAGGCTGCATCAATCTCAGGAGCTGGAAGGCCAACCTGTGGAAATGGATACAGTC
TCAATAATTGCAACTTTTCCATCAGATTTACGAGAAGAGGTTCTGTTGACGTCACCAGATACTATCCTTG
CCAATCTTACACCTGCTCTTGTTGCTGAGGCAAATATGTTGCGGGAGAGGTTTGCACACCGTTACAGTC
GTACCCTCTTTGGTATGTATCCTAGAAGTCGTAGAGGGGAGACTTCAAGACGTGAAGGTATTGGTTCTG
GTCTGGATGGAGCAGGGGGAACCATTTCTTCTCGCCGTTCCAATGGAGTTAAGGTTGTTGAAGCTGAT
GGAGCACCACTAGTTGACACAGAAGCTTTGCATGCTATGATTCGGTTGTTACGCGTAGTGCAGCCACTC
TATAAAGGCCAACTCCAGAGGCTTCTATTAAATCTTTGTGCCCATAGTGAAACAAGAACCTCTCTGGTG
AAAATTCTGATGGACTTGCTAATGCTTGATGTAAAAAGGCCTGTCAGTTATTTTAGTAAAGTTGAGCCA
CCATATAGATTATATGGTTGTCAGAGCAATGTAATGTATTCACGTCCTCAATCTTTTGATGGAGTTCCCC
CATTGCTGTCTCGTAGAATACTTGAAACTCTCACTTATCTTGCTCGCAATCATCTGTATGTGGCAAAAATT
TTGCTTCAGTGTTGGCTACCAAATCCTGCAATAAAAGAACCAGATGATGCACGGGGCAAAGCCGTGAT
GGTTGTTGAAGATGAAGTAAATATAGGTGAAAGTAATGATGGGTACATCGCCATTGCAATGCTATTGG
GTCTCTTGAACCAACCACTTTATTTGAGGAGCATAGCCCACCTTGAGCAGCTGCTAAATTTACTGGATGT
TATCATTGACAGTGCTGGAAACAAGTCATCTGACAAATCCTTGATATCTACTAACCCATCATCAGCTCCA
CAAATTTCTGCCGTGGAAGCCAATGCGAATGCAGATTCTAATATTTTATCTTCTGTGGATGATGCATCTA
AAGTTGATGGTTCCTCCAAACCAACGCCCTCTGGCATAAATGTTGAATGTGAGTCACATGGAGTGTTGA
GTAATCTTTCAAATGCAGAACTCCGGCTCCTGTGCTCACTGCTTGCACAAGAAGGTTTGTCAGATAATG
CATATAATCTTGTTGCCGAGGTAATGAAGAAATTGGTGGCCATTGCTCCAACACATTGTGAGCTTTTTGT
CACTGAGCTGGCAGAAGCAGTTCAAAAGTTGACTTCATCTGCAATGAATGAGTTACGTGTCTTTAGTGA
AGCAATGAAAGCTCTGCTTAGTACCTCTTCTACTGATGGAGCTGCAATTCTGAGAGTTTTGCAAGCCTTG
AGTTCCCTTGTCACCTTACTGACGGAGAAAGAGAATGACAGAGGTACTCCTGCTCTATCTGAGGTTTGG
GAAATCAATTCAGCATTAGAGCCCTTGTGGCATGAGCTTAGCTGTTGCATAAGCAAGATAGAATCCTAC
TCAGAGTCTGCATCTGAGATTTCGACATCTTCTAGTACCTTTGTGTCTAAACCATCTGGTGTAATGCCTC
CACTTCCTGCTGGCTCTCAAAATATCTTACCATACATTGAATCTTTCTTTGTGGTTTGTGAGAAATTGCAT
CCTGCTCAGCCAGGTGATAGTCATGACTCAAGTATCCCTGTTATTTCTGATGTTGAGTATGCCACCACAT
CTGCAACTCCCCAGAAAGCATCTGGAACTGCTGTGAAAGTAGATGAGAAACATATGCCTTTTGTCCGGT
TCTCAGAGAAGCATAGGAAGCTACTAAATGCATTCTTAAGGCAAAACCCTGGTTTGCTTGAGAAATCTT
TCTCACTCATGCTAAAGGTTCCAAGATTTATTGATTTTGATAACAAGCGTGCCCACTTCCGATCAAAAAT
TAAGCATCAGCATGACCATCACCATAGCCCATTGAGAATATCAGTAAGAAGGGCATATGTTCTAGAAG
ATTCTTACAACCAGCTTCGCTTGAGATCAACTCAAGATTTGAAGGGAAGGTTGACTGTTCACTTCCAAG
GGGAGGAGGGTATTGATGCAGGTGGGCTTACAAGGGAATGGTATCAATTATTGTCCAGAGTTATTTTT
GATAAAGGAGCACTGCTTTTTACTACAGTGGGCAATGAATCAACATTTCAGCCTAACCCTAACTCTGTTT
ATCAAACAGAGCATTTATCTTATTTCAAATTTGTTGGTAGAGTGGTTGGCAAAGCATTATTTGATGGTCA
ACTCTTGGATGTTCATTTTACTCGGTCATTCTACAAGCACATTCTTGGAGTCAAAGTTACATATCATGATA
TTGAAGCCATTGATCCTCATTATTTCAGAAATTTGAAATGGATGCTTGAGAATGACATCAGTGATGTTCT
GGATCTTACTTTTAGCATTGATGCAGATGAGGAAAAATTGATCTTATATGAACGAACAGAGGTGACTGA
TTATGAGTTGATTCCCGGGGGACGGAATATCAAAGTTACTGAGGAGAACAAACATCAATATGTTGATTT
GGTTGCCGAGCATCGGCTGACAACTGCCATTCGACCTCAAATAAATTCTTTCTTGGAAGGGTTCAATGA
AATGATTCCCAGGGAGTTGATATCGATATTCAATGACAAAGAGCTGGAATTGTTGATCAGTGGACTTCC
TGATATTGACTTGGATGACTTGAGAGCAAATACAGAATATTCTGGATATAGTGCTGCATCGCCAGTTAT
CCAATGGTTTTGGGAGGTTGTTCAAGGTTTGAGCAAAGAAGACAAAGCTCGACTGTTGCAATTTGTGA
CAGGCACATCCAAGGTGCCTTTGGAAGGCTTTAGCGCTCTTCAAGGAATTTCAGGCTCCCAGAAGTTTC
AGATACACAAAGCATATGGAAGTCCTGATCACTTGCCTTCTGCTCATACTTGCTTCAATCAATTAGATTT
GCCGGAGTATCCATCTAAACACCATTTAGAAGAGAGGTTACTGCTGGCAATTCACGAAGCAAGTGAGG
GTTTTGGATTTGGTTGA
SEQ ID NO: 20 CDS UPL2
>KRH72479 cds:protein_coding
ATGACAACCCTAAGATCAAGTTGGCCTTCGAGGCTGCGCCAACTTCTGTCCAGCGGGGGCGCCATTGG
TCCTTCAGTCAAGGTGGACTCCGAGCCCCCTCCTAAGATCAAAGCCTTCATTGAGAAGATCATCCAGTG
TCCATTACAAGATATTGCCATACCACTTTCTGGCTTTCGGTGGGAGTACAATAAGGGGAATTTTCATCAC
TGGAGACCGCTGTTGCTTCATTTTGATACATACTTCAAGACTTATTTGTCGTGTAGAAATGATCTGACGT
TGTTAGATAATCTAGAAGATGACAGCCCTTTACCAAAACATGCAATTCTGCAAATATTGCGAGTGATGC
AAAAAATTTTAGAGAACTGTCCAAACAAGAGTTCCTTTGATGGCTTAGAGCATTTCAAGCTTTTACTTGC
ATCAACAGATCCTGAGATTCTTGTTGCTACATTGGAAACTCTTTCTGCACTTGTAAAAATTAATCCCTCTA
AGCTTCATGGAAGTCCAAAGATGATTTGCTGTGGTTCGGTGAACAGCTATCTTTTGTCCTTAGCACAAG
GCTGGGGAAGCAAGGAGGAGGGCCTAGGATTGTACTCTTGTGTTATGGCAAATGAGAAAGCCCAAGA
TGAAGCACTGTGCTTGTTTCCTTCTGAAGAGATTGGTCATGACCAATCAAATTGCCGCATAGGCACTAC
CCTTTATTTTGAATTGCATGGTCCCAATGCCCAAAGCAAGGAACATAGTGCAGATGCAGTTTCCCCTAG
CTCAACAGTTATACACATGCCAGATTTGCATCTGCGCAAAGAAGATGATTTGTCATTGATGAAGCAGTG
CACTGAAGAATTTAGCATTCCTTCTGAGCTCAGGTTTTCCTTGCTCACTAGAATCAGATATGCTCGTGCC
TTTCGTTCTCCTAGAATATGCAGGCTTTACAGCCGGATTTGCCTACTTTCTTTCATTGTTCTGGTGCAGTC
TGGTGATGCTCAGGAAGAACTTGTCTCCTTTTTTGCTAATGAACCAGAATATACAAATGAATTAATTAGA
ATTGTACGATCAGAGGAAGTTATATCTGGATCTATCAGGACACTTGCAATGCTTGCTCTAGGAGCTCAA
TTAGCAGCATATACATCATCGCATCATCGGGCACGGATCAGTGGATCTAGTTTAACTTTTGCTGGTGGG
AACCGCATGATACTCCTAAATGTGCTCCAGAGGGCTATTTTGTCATTGAAGATTTCTAATGATCCATCAT
CCCTTGCCTTTGTTGAAGCACTTCTTCAGTTCTATCTGCTCCATGTGGTCTCAACCTCAACTTCTGGTAAT
AATATTAGAGGTTCTGGCATGGTGCCAACATTCTTGCCGTTGCTGGAGGATTTTGATCCTACACATATTC
ATCTAGTCTGTTTTGCTGTGAAAACTCTTCAGAAGCTTATGGATTATAGTAGCTCAGCTGTATCATTGTT
TAAAGAATTGGGGGGCATTGAACTTTTGGCTCAGAGGTTACAGAAAGAGGTACACAGAGTCATTGGTT
TGGTTGGAGGAACTGATAACATGATGCTTACTGGTGAAAGCTTGGGACATAGTACTGATCAATTGTACT
CCCAGAAGAGACTCATAAAGGTCTCCCTTAAGGCGCTTGGTTCTGCAACATACGCACCTGCAAACTCTA
CCAGATCTCAACATTCTCAAGACAGTTCATTACCTATAACTCTAAGCTTGATTTTTAAGAATGTAGATAA
GTTTGGAGGTGACATTTATTATTCAGCTGTTACTGTTATGAGTGAAATAATCCACAAAGATCCTACCTTT
TTTTCTGCTCTGCATGAAATAGGTCTTCCTGATGCGTTTTTATTGTCAGTTGGATCTGGAATACTTCCATC
ATCAAAGGCTTTGACATGCATTCCAAATGGTCTTGGGGCCATTTGTCTTAATGCCAAAGGGTTAGAGGC
CGTTAGAGAATCTTCATCGCTACGGTTCCTTGTTGACATTTTCACTAGCAAGAAGTATGTCTTAGCCATG
AATGAGGCTATTGTTCCTTTGGCAAATGCTGTGGAGGAACTTCTACGCCATGTATCTACATTGAGAAGC
ACTGGTGTTGATATTATCATTGAAATCATCCATAAGATCACATCTTTTGGGGATGGAAATGGTGCAGGA
TTTTCTGGAAAAGCTGAGGGCACCGCCATGGAAACAGATTCTGAAAACAAAGAAAAAGAAGGCCATTG
TTGCATTGTAGGCACATCATATTCGGCTGTAGAAGGGATAAGTGATGAGCAGTTTATTCAGCTATGTGT
CTTTCATTTGATGGTATTAGTTCATAGGACTATGGAAAATGCCGAGACATGCCGGTTGTTTGTGGAAAA
ATCAGGAATTGAAGCTTTATTGAATTTGTTATTACGACCCACTATTGCACAATCCTCAGATGGCATGTCT
ATTGCTTTACATAGCACAATGGTATTTAAAGGGTTTGCTCAACATCATTCAATTCCTCTGGCACATGCCTT
CTGTTCTTCTCTTAGAGAGCACTTAAAGAAAACTTTAGTGGGGTTTGGTGCAGCATCAGAACCTTTGTT
GCTGGATCCAAGGATGACAACTGATGGTGGCATCTTTTCTTCACTTTTCCTGGTTGAGTTCCTTCTATTTC
TTGTGGCATCGAAAGACAATCGTTGGGTGACTGCCTTGCTTACAGAATTTGGAAATGAGAGTAAGGAT
GTTCTTGAAGACATTGGATGCGTTCACCGTGAAGTTCTGTGGCAAATTTCTCTACTTGAAAATAGAAAA
CCTGAGATTGAGGAAGATGGTGCTTGTTCTTCTGATTCACAACAGGCTGAAGGGGATGTAAGTGAAAC
TGAAGAGCAAAGGTTCAATTCTTTCAGGCAGTATCTTGACCCATTATTGAGAAGGAGAACATCAGGAT
GGAGCATTGAATCCCAGTTTTTTAACCTTATAAACCTGTATCGAGATTTGGGCCGTTCCACTGGTTCTCA
AAATAGATTAGTTGGTCCGAGGTCAAGTTCTAGTAATCAGGTACAGCATTCTGGGTCAGATGATAATTG
GGGGACTGCTAATAAGAAGGAATCTGACAAGCAGAGAGCATATTATACATCTTGTTGTGACATGGTCA
GATCACTTTCATTTCACATTACCCATTTGTTCCAAGAGTTAGGAAAAGTAATGTTGCTACCTTCACGTCG
GCGTGATGATGTTGTGAATGTAAGTCCTGCTTCAAAATCAGTGGCTTCTACATTTGCATCCATTGCTTTT
GATCACATGAATTATGGTGGCCGTTGTGTAAATCTTTCGGGAACAGAAGAATCCATATCAACAAAATGT
CGATATTTTGGGAAAGTGATTGATTTTATGGATAATGTTCTAATGGAGAGGCCAGATTCATGCAATCCT
ATTATGCTGAATTGCTTGTATGGACGTGGAGTTATTGAAATTGTATTAACTACCTTTGAAGCTACCAGTC
AGCTGCTCTTTACAGTTAATCGGGCCCCTGCCTCGCCCATGGATACTGATGATGCAAATGCAAAGCAAG
ACGACAAGGAAGATACAGATAATTCATGGATTTATGGTTCTTTAGCTAGTTATGGGAAATTGATGGACC
ATCTAGTGACCTCCTCTTTTATATTATCATCATTCACAAAGCATTTGCTTGCACAGCCCCTTACTAATGGT
GATACACCTTTCCCAAGGGATGCTGAGACTTTTGTGAAGGTCCTTCAATCCAGAGTGTTGAAGACTGTA
CTTCCTGTTTGGACTCATCCCAAGTTTGTTGACTGTAGTTATGAATTTATTTCTACAGTTATTTCTATCATT
AGGCATGTCTATACAGGTGTTGAAGTAAAAAATGTGAATGGCAGTGCTGGTGCTCGCATTACTGGGCC
GCCTCCTAATGAAACAACTATTTCAACCATTGTAGAAATGGGGTTTTCCAGGTCTAGAGCAGAAGAAGC
TTTGAGGCAAGTTGGGTCAAATAGTGTGGAGTTGGCAATGGAGTGGTTGTTCTCTCATCCAGAGGAGG
CACAAGAAGATGATGAACTTGCCCGTGCACTTGCCATGTCCCTTGGAAACTCTGAATCAGATTCAAAGG
ATGCAGTTGCTAATGACAATGCCCTGCAGCTTGAGGAAGAGATGGTCCAACTTCCTCCTGTTGATGAGT
TGTTATCTACTTGTACAAAACTTTTGTCGAAGGAACCACTTGCTTTTCCAGTCCGTGACTTGCTTGTGAT
GATATGCTCTCAGGATGATGGTCAACATAGATCTAATGTGGTCTCATTTATTGTGGAACGGATCAAAGA
ATGTGGTTTGGTTCCTAGCAATGGAAATTATGCCATGCTGGCTGCTCTTTTTCATGTTCTAGCTTTAATTC
TTAATGAGGATGCTGTGGCTAGGGAAGCTGCTTCTACAAGTGGTTTAATCAAAATTGCCTCAGATCTAC
TCTACCAGTGGGATTCTAGTCTTGATATCAAGGAGAAACATCAGGTACCAAAATGGGTGACTGCTGCTT
TCCTTGCATTAGACAGATTGTTGCAAGTGGATCAAAAATTGAATTCTGAAATCGCAGAGCAGTTGAAGA
AGGAAGCTGTGAATAGCCAGCAGACATCAATTACCATTGATGAAGACAGGCAAAACAAGATGCAGTCT
GCATTGGGACTCTCTATGAAGTATGCAGATATACATGAACAGAAGAGACTTGTTGAGGTTGCTTGTAGT
TGTATGAAGAATCAACTTCCATCCGACACAATGCATGCTGTTCTGCTACTATGTTCCAATCTTACAAGGA
ATCATTCTGTAGCTCTTACTTTTTTGGATTCTGGTGGTTTAAGTCTACTTCTTTCTTTGCCAACCAGCAGTC
TCTTCCCTGGGTTTGACAATGTTGCTGCTAGTATTGTTCGTCATGTTCTTGAAGATCCTCAAACGCTCCAT
CAAGCAATGGAATCTGAGATAAAACATAGTCTTGTAGTTGCATCTAACCGGCATCCAAATGGAAGGGT
CAATCCTCATAATTTCCTTTTAAATTTAGCTTCTGTGATTTCTCGGGATCCAGTAATTTTTATGCAAGCTG
CTCAATCTGTGTGCCAAGTTGAAATGGTAGGTGAGAGGCCATACATTGTCTTGCTGAAAGATAGGGAT
AAAGACAAAGCTAAGGATAAAGAAAAGGATAAGGATAAAACATTGGAGAAAGATAAAGTACAGAACA
TTGATGGGAAGGTTGTTTTGGGAAATACTAACACGGCACCTACTGGCAATGGCCATGGCAAAATTCAA
GATTCAAATACCAAGAGTGCCAAAGGTCACAGAAAACCTACCCAAAGTTTTATTAATGCAATAGAACTT
CTTCTTGAATCTGTATGCACTTTTGTTCCTCCCTTGAAGGGTGACATTGCCTCAAATGTTCTTCCTGGCAC
CCCAGCATCAACCGATATGGACATTGATGCCTCCATGGTTAAGGGAAAAGGAAAAGCAGTTGCCACTG
ATTCTGAGGGCAATGAAACTGGTAGTCAGGATGCTTCTGCATCACTTGCAAAGATTGTCTTCATTCTAA
AGCTTCTGACAGAGATACTATTGATGTATTCATCATCTGTTCATGTTTTACTTAGACGAGATGCTGAAAT
GAGCAGCATTAGAGGTTCTTATCAAAAGAGTCCTGCAGGTTTAAGCATGGGTGGGATATTCTCTCATAT
TCTTCATAATTTTCTTCCATATTCTCGAAACTCCAAAAAGGACAAGAAAGCTGATGGTGATTGGAGGCA
GAAACTAGCAACCAGGGCCAACCAGTTTATGGTGGGTGCTTGTGTTCGATCTACAGAGGCAAGGAAGA
GGGTTTTTGGTGAGATTTGTTGTATCATCAATGAATTTGTTGATTCATGTCATGGCATTAAGCGTCCAGG
AAAAGAAATTCAGGTTTTTGTTGATCTACTAAATGATGTTTTGGCTGCTCGTACACCCGCTGGTTCATCC
ATTTCAGCTGAGGCCTCTACCACTTTTATTGATGCTGGTTTGGTTAAATCATTCACATGTACTCTACAAGT
TTTGGACCTTGACCATGCTGATTCATCTGAAGTTGCTACGGGTATTATTAAAGCTCTTGAGTTGGTAACC
AAGGAGCATGTCCAATTAGTTGATTCTAGTGCAGGGAAGGGTGATAATTCAGCAAAGCCTTCTGTTCTA
AGTCAACCCGGAAGAACAAATAATATTGGTGACATGTCTCAGTCCATGGAGACATCACAAGCCAATCCT
GATTCCCTTCAAGTTGACCGTGTTGGGTCTTATGCAGTTTGCTCCTATGGTGGGTCTGAAGCTGTTACTG
ATGATATGGAACATGATCAAGATCTTGATGGGAGCTTTGCTCCTGCTAATGAGGATGATTACATGCATG
AAAATTCTGAGGATGCAAGAGATCTTGAAAATGGAATGGAAAATGTGGGTCTACAATTTGAAATCCAA
TCTCATGGCCAAGAAAATCTTGATGAGGATGACGATGAGGACGATGATATGTCTGAAGATGAAGGTGA
GGATGTAGATGAAGATGAGGATGATGATGAGGAACACAATGATTTGGAAGAAGTCCATCATTTGCCAC
ATCCTGACACAGATCAAGATGAGCATGAGATTGATGATGAAGATTTTGATGATGAAGTGATGGAGGAA
GAGGATGAGGATGACGAGGAAGATGAAGATGGTGTTATACTGCAACTCGAGGAGGGGATTAATGGA
ATTAATGTTTTTGATCATATTGAGGTTTTTGGCAGAGATAATAGTTTTGCAAATGAAGCTTTTCAAGTGA
TGCCGGTTGAGGTTTTTGGATCCAGACGTCAGGGGAGGACAACATCTATTTATAGTCTTTTGGGAAGAA
CTGGTGATACCGCTGTGCCTTCTCGTCACCCACTCTTGCTTGAACCTTCTTCATTCCCCCCACCTACAGGG
CAATCAGATAGTTCATTGGAGAACAACTCATTGGGTTTGGATAATATATTTCGATCGCTGAGGAGTGGA
CGCCATGGACAGCGTTTGCACTTGTGGACTGATAATAACCAACAAAGTGGTGGGACAAACACTGTTGT
TGTACCCCAAGGCCTTGAGGATTTGCTTGTCACTCAATTAAGGCGACCAATCCCTGAAAAGTCATCCAA
TCAGAACATTGCAGAAGCAGGTTCTCATGGTAAAGTTGGAACGACCCAGGCACAAGATGCAGGGGGT
GCAAGGCCAGAAGTCCCTGTTGAAAGTAATGCTGTTCTGGAAGTTAGTACTATAACTCCCTCGGTTGAT
AACAGTAACAATGCGGGTGTCAGACCAGCTGGGACTGGACCTTCACATACAAATGTTTCAAACACACA
CTCACAGGAAGTTGAGATGCAATTTGAACATGCTGATGGAGCTGTGAGGGATGTTGAAGCTGTCAGCC
AGGAGAGTAGTGGTAGTGGTGCAACTTTTGGTGAAAGCCTTCGGAGCTTGGATGTTGAGATTGGAAGT
GCTGATGGCCATGATGATGGTGGTGAAAGGCAGGTTTCTGCTGATAGGGTGGCAGGTGATTCGCAGG
CAGCACGCACAAGAAGAGCAAATACGCCTTTGAGTCACATTTCTCCTGTGGTTGGAAGAGATGCGTTCC
TTCACAGTGTAACTGAAGTTTCAGAAAATTCAAGCCGTGATGCAGATCAAGATGGTGCAGCAGCAGAG
CAGCAGGTGAACAGTGATGCAGGATCAGGAGCTATTGATCCTGCTTTTCTGGATGCTCTTCCTGAGGA
GCTGCGTGCCGAACTCCTTTCAGCTCAGCAGGGTCAAGTGGCTCAGCCATCAAATGCTGAGTCTCAAAA
CACTGGGGATATTGATCCAGAGTTCCTTGCAGCTCTTCCAGCTGATATTCGAGCAGAAATTCTAGCTCA
GCAGCAAGCACAGAGGCTGCATCAATCTCAGGAGCTGGAAGGCCAACCTGTGGAAATGGATACAGTC
TCAATAATTGCAACTTTTCCATCAGATTTACGAGAAGAGGTTCTGTTGACGTCACCAGATACTATCCTTG
CCAATCTTACACCTGCTCTTGTTGCTGAGGCAAATATGTTGCGGGAGAGGTTTGCACACCGTTACAGTC
GTACCCTCTTTGGTATGTATCCTAGAAGTCGTAGAGGGGAGACTTCAAGACGTGAAGGTATTGGTTCTG
GTCTGGATGGAGCAGGGGGAACCATTTCTTCTCGCCGTTCCAATGGAGTTAAGGTTGTTGAAGCTGAT
GGAGCACCACTAGTTGACACAGAAGCTTTGCATGCTATGATTCGGTTGTTACGCGTAGTGCAGCCACTC
TATAAAGGCCAACTCCAGAGGCTTCTATTAAATCTTTGTGCCCATAGTGAAACAAGAACCTCTCTGGTG
AAAATTCTGATGGACTTGCTAATGCTTGATGTAAAAAGGCCTGTCAGTTATTTTAGTAAAGTTGAGCCA
CCATATAGATTATATGGTTGTCAGAGCAATGTAATGTATTCACGTCCTCAATCTTTTGATGGAGTTCCCC
CATTGCTGTCTCGTAGAATACTTGAAACTCTCACTTATCTTGCTCGCAATCATCTGTATGTGGCAAAAATT
TTGCTTCAGTGTTGGCTACCAAATCCTGCAATAAAAGAACCAGATGATGCACGGGGCAAAGCCGTGAT
GGTTGTTGAAGATGAAGTAAATATAGGTGAAAGTAATGATGGGTACATCGCCATTGCAATGCTATTGG
GTCTCTTGAACCAACCACTTTATTTGAGGAGCATAGCCCACCTTGAGCAGCTGCTAAATTTACTGGATGT
TATCATTGACAGTGCTGGAAACAAGTCATCTGACAAATCCTTGATATCTACTAACCCATCATCAGCTCCA
CAAATTTCTGCCGTGGAAGCCAATGCGAATGCAGATTCTAATATTTTATCTTCTGTGGATGATGCATCTA
AAGTTGATGGTTCCTCCAAACCAACGCCCTCTGGCATAAATGTTGAATGTGAGTCACATGGAGTGTTGA
GTAATCTTTCAAATGCAGAACTCCGGCTCCTGTGCTCACTGCTTGCACAAGAAGGTTTGTCAGATAATG
CATATAATCTTGTTGCCGAGGTAATGAAGAAATTGGTGGCCATTGCTCCAACACATTGTGAGCTTTTTGT
CACTGAGCTGGCAGAAGCAGTTCAAAAGTTGACTTCATCTGCAATGAATGAGTTACGTGTCTTTAGTGA
AGCAATGAAAGCTCTGCTTAGTACCTCTTCTACTGATGGAGCTGCAATTCTGAGAGTTTTGCAAGCCTTG
AGTTCCCTTGTCACCTTACTGACGGAGAAAGAGAATGACAGAGGTACTCCTGCTCTATCTGAGGTTTGG
GAAATCAATTCAGCATTAGAGCCCTTGTGGCATGAGCTTAGCTGTTGCATAAGCAAGATAGAATCCTAC
TCAGAGTCTGCATCTGAGATTTCGACATCTTCTAGTACCTTTGTGTCTAAACCATCTGGTGTAATGCCTC
CACTTCCTGCTGGCTCTCAAAATATCTTACCATACATTGAATCTTTCTTTGTGGTTTGTGAGAAATTGCAT
CCTGCTCAGCCAGGTGATAGTCATGACTCAAGTATCCCTGTTATTTCTGATGTTGAGTATGCCACCACAT
CTGCAACTCCCCAGAAAGCATCTGGAACTGCTGTGAAAGTAGATGAGAAACATATGCCTTTTGTCCGGT
TCTCAGAGAAGCATAGGAAGCTACTAAATGCATTCTTAAGGCAAAACCCTGGTTTGCTTGAGAAATCTT
TCTCACTCATGCTAAAGGTTCCAAGATTTATTGATTTTGATAACAAGCGTGCCCACTTCCGATCAAAAAT
TAAGCATCAGCATGACCATCACCATAGCCCATTGAGAATATCAGTAAGAAGGGCATATGTTCTAGAAG
ATTCTTACAACCAGCTTCGCTTGAGATCAACTCAAGATTTGAAGGGAAGGTTGACTGTTCACTTCCAAG
GGGAGGAGGGTATTGATGCAGGTGGGCTTACAAGGGAATGGTATCAATTATTGTCCAGAGTTATTTTT
GATAAAGGAGCACTGCTTTTTACTACAGTGGGCAATGAATCAACATTTCAGCCTAACCCTAACTCTGTTT
ATCAAACAGAGCATTTATCTTATTTCAAATTTGTTGGTAGAGTGGTTGGCAAAGCATTATTTGATGGTCA
ACTCTTGGATGTTCATTTTACTCGGTCATTCTACAAGCACATTCTTGGAGTCAAAGTTACATATCATGATA
TTGAAGCCATTGATCCTCATTATTTCAGAAATTTGAAATGGATGCTTGAGAATGACATCAGTGATGTTCT
GGATCTTACTTTTAGCATTGATGCAGATGAGGAAAAATTGATCTTATATGAACGAACAGAGGTGACTGA
TTATGAGTTGATTCCCGGGGGACGGAATATCAAAGTTACTGAGGAGAACAAACATCAATATGTTGATTT
GGTTGCCGAGCATCGGCTGACAACTGCCATTCGACCTCAAATAAATTCTTTCTTGGAAGGGTTCAATGA
AATGATTCCCAGGGAGTTGATATCGATATTCAATGACAAAGAGCTGGAATTGTTGATCAGTGGACTTCC
TGATATTGACTTGGATGACTTGAGAGCAAATACAGAATATTCTGGATATAGTGCTGCATCGCCAGTTAT
CCAATGGTTTTGGGAGGTTGTTCAAGGTTTGAGCAAAGAAGACAAAGCTCGACTGTTGCAATTTGTGA
CAGGCACATCCAAGGTGCCTTTGGAAGGCTTTAGCGCTCTTCAAGGAATTTCAGGCTCCCAGAAGTTTC
AGATACACAAAGCATATGGAAGTCCTGATCACTTGCCTTCTGCTCATACTTGCTTCAATCAATTAGATTT
GCCGGAGTATCCATCTAAACACCATTTAGAAGAGAGGTTACTGCTGGCAATTCACGAAGCAAGTGAGG
GTTTTGGATTTGGTTGA
SEQ ID NO: 21 CDS UPL2
>KRH62267 cds: protein_coding
ATGACAAGCGTAAGATCGAGTTGGCCATCAAGGCTGCGCCAACTTCTTTCCAGCGAGGGTTCCATTGGC
CCTTCCGTCAAACTCGACTCTGACCCTTCTCCTAAGATCAAAGCCTTCATTGAGAAGGTCATTCAATGTC
CATTACAAGATATAGCTATACCCCTCTTTGGCTTTCGGTGGGAGTATAATAAGGGGAATTTTCATCACTG
GAGGCCATTGTTTCTTCATTTTGATACATACTTCAAGACATATTTATCATGTCGAAATGACCTGACATTGT
CCGATAATCTAGAAGTTGGCATTCCATTACCAAAACATGCAATTCTACAAATACTACGGGTGATGCAAA
TAATCTTAGAGAACTGTCCAAACAAGAGTTCATTTGATGGCTTAGAGCACTTCAAGCTTTTACTAGCATC
AACAGATCCTGAGATTATTATTGCTACATTAGAAACTCTTGCTGCGCTTGTAAAAATAAATCCTTCTAAG
CTTCATGGAAGTGCAAAAATGGTTGGCTGTGGTTCAGTAAATAGCTATCTCCTGTCCCTAGCACAGGGG
TGGGGAAGCAAGGAGGAGGGCATGGGTTTGTACTCTTGTATTATGGCAAATGAGAAAGCCCAGGATG
AAGCACTGTGTTTGTTTCCTTCTGATGCAGAGAATGGTAGTGACCACTCCAATTACTGCATAGGTTCTAC
TCTTTATTTTGAATTGCGTGGACCCATTGCTCAAAGCAAGGAACAAAGTGTAGATACAGTTTCCTCAAGT
TTGAGAGTTATACACATTCCAGATATGCATTTACACAAAGAAGATGATTTGTCAATGTTGAAGCAATGC
ATTGAGCAGTATAATGTTCCTCCTGAGCTCCGATTTTCATTGCTCACAAGAATTAGATATGCTCGTGCTT
TCCGGTCTGCGAGAATAAGCAGGCTTTATAGCAGGATTTGCCTTCTTGCTTTCACTGTGTTGGTCCAATC
CAGTGATGCTCATGACGAGCTTGTGTCCTTTTTTGCCAACGAACCAGAGTACACAAGCGAATTGATTAG
AGTTGTGCGATCTGAAGAAACAATATCTGGATCTATCAGAACACTTGTAATGCTTGCATTAGGAGCCCA
GTTAGCAGCATACACATCATCTCATGAACGGGCACGGATACTGAGTGGATCTAGTATGAACTTCACTGG
AGGGAACCGCATGATTCTACTGAATGTACTTCAGAGGGCTATTTTGTCATTGAAGAGTTCTAATGATCC
AACTTCCTTTGCTTTTGTTGAGGCACTTCTTCAATTCTATCTGCTGCATGTAGTGTCAACATCATCTTCTG
GGAGTAATATTAGAGGTTCTGGCATGGTACCCACATTCTTGCCTCTGCTGGAGGATTCTGATCTTGCTC
ATATTCATCTTGTTTGTTTAGCAGTGAAAACCCTTCAGAAGCTTATGGATTATAGTAGTTCAGCTGTATC
TTTGTTTAAAGAGTTGGGGGGTGTTGAGCATTTGGCTCAAAGATTACAGATAGAGGTTCATAGGGTCA
TTGGTTTTGCTGGAGAGAATGATAATGTGATGCTCACTGGTGAAAGCTCAAGACATAGTACTCATCAGC
TTTACTCTCAGAAGAGGCTGATAAAAGTGTCCCTTAAGGCCCTTGGTTCTGCAACATATGCTCCTGCAAA
CTCTACCAGATCTCAACACTCCCATGACAGTTCATTACCTGCAACTCTAGTCATGATTTTTCAGAATGTAA
ATAAGTTCGGAGGTGACATTTATTACTCAGCTGTTACTGTTATGAGTGAAATAATCCACAAAGATCCTAC
ATGCTTCTCTTCTTTGCATGAAATGGGTCTTCCAAATGCTTTTTTATCTTCAGTTGCATCTGGAATTCTTCC
TTCATCAAAGGCTCTGACATGCATTCCAAATGGCATTGGGGCCATTTGTCTTAATGCCAAAGGCTTAGA
GGTTGTTCGAGAGACTTCATCACTGCAGTTCCTTTTTAATATCTTTACAAGCAAAAAGTATGTCCTTTCCA
TGAATGAGGCTATTGTTCCGCTAGCAAATTCTGTAGAGGAACTTCTTCGACACGTGTCTCCATTGAGAA
GTACTGGTGTTGACATCATCATTGAAATCATCCATAAGATTGCATCCTTTGGTGATGGTATTGATACAGG
ATCTTCTTCAGGAAAAGCTAATGAGGATAGTGCAATGGAAACCAATTCTGAAGACAAAGGAAATGAAA
ACCATTGTTGCCTCGTGGGCACAGCAGAGTCTGCCGCTGAAGGGATTAATGATGAGCAATTCATTCAGC
TTTGCACTTTTCATTTGATGGTATTGGTTCACCGGACAATGGAAAATTCTGAAACATGTCGGCTATTTGT
AGAAAAATCAGGAATTGAAGCTTTATTGAAGCTGTTATTACGACCTACCATTGCACAATCCTCGGATGG
CATGTCTATTGCTCTGCATAGCACCATGGTATTTAAGGGGTTTGCTCAACATCATTCCGCTCCTTTGGCA
CGTGCCTTTTGTTCCTCTCTTAAAGAGCACTTGAATGAAGCATTAACTGGGTTTGTTGCATCTTCGGGAC
CTTTGTTGCTGGATCCAAAGATGACCACAAATAACATCTTTTCTTCACTTTTCTTGGTTGAGTTTCTTCTCT
TTCTTGCTGCGTCAAAAGACAACCGTTGGGTGACTGCTTTGCTTACAGAATTTGGAAATGGTAGTAAGG
ATGTTCTTGAAAACATTGGACGTGTCCACCGTGAAGTTTTGTGGCAAATTGCTCTTCTTGAAAATACGAA
GCCTGATATTGAGGATGACGTTTCTTGTTCTACTTCTGATTCACAACAGGCAGAAGTGGATGCAAATGA
AACTGCAGAGCAAAGGTACAATTCTATCAGGCAGTTTCTTGATCCATTACTCAGGAGGAGGACTTTAGG
ATGGAGTGTAGAATCACAGTTTTTTGATCTTATTAACCTGTATCGAGATCTGGGTCGTGCCCCTGGTTCC
CAGCACCGATCAAATTCTGTTGGTCCTACAAACAGGCGGTTAGGATCCCCTAATCCGTTGCATCCGTCT
GAGTCTTCAGATGTATTGGGGGATGCTAGTAAGAAAGAATGTGACAAGCAAAGAACATATTATACCTC
TTGTTGTGACATGGCCAGATCACTTTCATTTCACATTATGCATTTGTTCCAAGAGTTAGGAAAAGTAATG
CTGCAACCTTCTCGCCGTCGTGATGATGTTGCAAGTGTAAGTCCTGCTTCAAAATCAGTGGCTTCTACTT
TTGCAAGCATTGCTCTAGATCACATGAATTTTGGGGGCCATGTAGAAGAAGCATCCATATCAACAAAAT
GTCGTTATTTTGGTAAAGTCATTGATTTTGTGGATGGCATTCTAATGGAAAGGCCTGATTCTTGCAATCC
CATTTTACTGAATTGCTTGTATGGGCATGGAGTTATTCAATCTGTATTGACCACATTTGAAGCAACTAGT
CAGTTGTTATTTGCAGTTAATCGGACCCCTGCATCGCCGATGGAAATTGATGATGGAAATGTGAAGCAG
GATGACAAGGAAGATACCGATCATTTGTGGATATATGGTTCTTTAGCCAGTTATGGTAAATTTATGGAC
CATCTAGTAACCTCCTCTTTCATATTATCTTCTTTCACAAAGCCTATACTTGCACAGCCCCTTAGTGGTGA
TACCTCATATCCCCGGGATGCTGAGATATTTGTGAAAGTCCTCCAATCTATGGTGTTGAAGGCTGTGCT
CCCAGTTTGGATGCATCCCCAGTTTGTTGATTGTAGTCATGGATTTATTTCTAATGTTATCTCTATCATCA
GGCATGTTTATTCAGGGGTTGAAGTAAAAAATGTAAATGGCAGCAGCAGTGCTCGTATTACTGGGCCT
CCTCCTAATGAAACAACAATTTCAACCATTGTAGAGATGGGATTTTCCAGGTCGAGAGCAGAAGAAGCT
TTGAGGCATGTTGGATCAAATAGTGTGGAGTTGGCGATGGAGTGGCTGTTTTCCCATCCAGAGGACAC
ACAAGAAGATGACGAACTTGCTCGTGCACTTGCCATGTCCCTTGGGAACTCTGAATCAGACACCAAGG
ATGCTGCTGCAAATGACAGTGTACAACTGCTTGAGGAAGAAATGGTCCATCTTCCTCCTGTTGATGAGT
TGTTATCAACTTGCACTAAACTTCTTCAAAAGGAACCTCTTGCTTTTCCTGTCCGTGACTTGCTCATGATG
ATATGCTCTCAGAATGATGGTCAAAATAGATCTAATGTTCTCACTTTTATTGTTGACCGGATCAAGGAAT
GTGGATTGATTTCTGGTAACGGAAATAATACCATGCTTGCTGCTCTATTTCATGTTCTTGCATTGATTCTT
AATGAGGATGCTGTTGCGCGAGAAGCTGCTTCAAAGAGTGGTTTCATAAAAATTGCCTCAGATCTACTC
TACCAATGGGATTCTAGTCTTGGTAACAGGGAGAAAGAACAGGTTCCAAAATGGGTCACAGCTGCTTT
TCTTGCATTAGACAGGCTGTTGCAAGTGGATCAAAAATTGAATTCTGAAATTGCAGAGCTTTTGAAGAA
GGAAGCTTTGAATGTTCAGCAGACATCAGTTATCATTGATGAGGATAAGCAACACAAATTGCAGTCTGC
GTTGGGACTTTCCACCAAGTATGCAGATATACATGAACAGAAGAGACTTGTTGAGATTGCTTGTAGTTG
CATGAAGAACCAACTTCCCTCAGACACAATGCATGCTATTTTGCTACTATGTTCCAATCTTACAAAGAAT
CACTCTGTTGCTCTTACCTTTTTTGATGCTGGTGGTTTAAGTTTACTTCTTTCTCTGCCAACCGGTAGCCTC
TTTCCTGGGTTTGACAACGTTGCTGCTGGTATTGTCCGTCATGTTATTGAAGATCCACAAACTCTCCAGC
AAGCAATGGAATCTGAGATAAAACACAGTCTTGTAGCTGCGTCTAACCGCCATCCAAATGGGAGGGTC
AATCCACAAAATTTTCTGTTAAGTTTAGCTTCTGTAATTTCCCGGGATCCAATAATATTTATGCAAGCTGC
TCAATCTGCTTGCCAAGTTGAAATGGTGGGTGAAAGACCTTACATTGTCTTGCTGAAAGATCGGGATAA
AGAGAAATCCAAGGATAAGGATAAGTCACTGGAGAAAGATAAAGCACATAATGATGGAAAAATTGGT
TTGGGAAGTACAGCCACAGCAGCTTCAGGGAATGTTCATGGAAAACTTCATGATTCAAACTCAAAGAA
TGCCAAAAGTTACAAAAAGCCTACTCAAAGTTTTGTTAATGTGATAGAACTTCTTCTTGAATCTATATGC
ACATTTGTTGCCCCCCCTTTGAAGGACAATAATGTATCAAATGTTGTCCCTGGCTCCCCAACATCAAGTG
ACATGGACATTGATGTTTCTACAGTTAGGGGGAAAGGAAAAGCAGTTGCCACTGTGCCTGAGGGGAAT
GAAACCAGCAGTGAGGAAGCATCTGCATCACTAGCAAAGATAGTATTTATTTTGAAGCTTCTGATGGA
GATATTGTTGATGTATTCATCGTCTGTTCATGTTCTGCTTCGACGGGATGCTGAAATGAGCAGCTCTAG
GGACATTTATCAAAAGAATCATGGTAGTTTTGGTGCGGGAGTAATATTCTACCATATTCTTCGTAATTTT
CTTCCTTGTTCTCGAAATTCCAAAAAAGACAAGAAAGTTGATGATGATTGGAGGCAGAAACTAGCAACA
AGGGCTAATCAGTTTATGGTAGCTGCTTGTGTTCGTTCTTCAGAGGCAAGGAGGCGGGTTTTTACTGAG
ATTAGCCATATCATTAATGAATTTGTTGATTCATGTAATTGTGTTAAGCCAAAGCCATCAGGCAATGAAA
TTCTGGTTTTTGTTGATCTACTTAATGATGTTTTGGCTGCTCGGACACCTGCTGGCTCAAGCATCTCAGC
AGAGGCCTCTGTCACTTTTATGGATGCTGGTCTACTTAAATCTTTTACCCGTACTCTCCAAGTTTTAGACT
TGGACCATGCTGACTCGTCTAAAGTTGCTACTGGTATTATCAAAGCTCTTGAACTAGTAACCAAGGAGC
ATGTTCACTCAGTTGAACCGAGTGCAGGAAAGGGTGATAATCAAACTAAGCCTTCTGATCCTAGTCAAT
CCGGAAGAACAGATAATATTGGTCACATGTGTCAGTCCATGGAAACAACATCTCAGGCCAATCACGATT
CCCTTCAAGTTGACCATGTTGGGTCTTACAATGTGATTCAGTCTTATGGTGGGTCTGAAGCTGTTATTGG
TGATATGGAACATGATCTTGATGGGGACTTTGCTCCTGCTAATGAAGATGAGTTCATGCATGAAACTGG
TGAGGATGCCAGAGGCCATGGGAATGGAATTGAAAATGTTGGGCTACAATTTGAAATCCAATCCCATG
GACAAGAAAATCTCGATGATGACGATGATGAGGGTGATATGTCTGGAGATGAGGGTGAAGATGTAGA
TGAAGATGACGAAGATGATGAGGAACACAATGATTTGGAAGAAGATGAAGTCCATCACTTGCCACATC
CTGACACTGATCGTGATGATCATGAGATGGATGATGATGATTTTGATGAAGTGATGGAGGGGGAGGA
GGATGAAGATGAGGATGATGAAGATGGTGTTATACTGAGACTTGAGGAGGGCATCAATGGAATTAAT
GTTTTTGACCATATTGAGGTTTTTGGAAGAGACAATAGTTTTCCAAATGAATCCCTTCATGTCATGCCAG
TTGAAGTTTTTGGATCTAGACGTCCAGGGCGGACCACCTCTATTTACAGCCTGTTGGGCAGAAGTGGTG
ATAATGCCGCCCCTTCTTGCCATCCACTTTTAGTTGGTCCTTCTTCCTCATTCCATCTATCTAATGGTCAAT
CAGATAGTATAACAGAGAACTCCACAGGCTTGGATAATATCTTTCGTTCATTGAGGAGCGGACGTCATG
GGCACCGCTTGAACTTGTGGAGTGATAATAGCCAGCAAATCAGTGGGTCAAATACTGGCGCTGTACCA
CAGGGCCTTGAGGAGTTGCTTGTGTCTCAATTGAGGCGACCTACTGCTGAGAAGTCGTCTGATAATAAT
ATAGCAGACGCTGGTCCTCATAATAAAGTTGAGGTCAGCCAGATGCACAGTTCCGGAGGTTCAAAGCT
TGAAATCCCAGTTGAAAGCAATGCAATTCAGGAAGGTGGTAATGTGACTCCTGCATCAATTGATAACAC
TGACATCAATGCTGATATCAGACCTGTAGGAAATGGAACTCTGCAAGCAGATGTATCAAACACTCACTC
TCAGACAGTTGAGATGCAGTTTGAGAATAATGATGCAGCTGTGCGGGATGTTGAAGCTGTGAGCCAGG
AGAGTAGTGGTAGTGGGGCAACTTTTGGTGAAAGCCTTCGGAGCCTAGATGTTGAGATTGGAAGTGCT
GATGGCCATGATGATGGTGGAGAAAGGCAGGTTTCTGCGGATAGGATAGCAGGTGATTCACAGGCTG
CACGCACAAGAAGAGCAACCATGTCTGTTGGTCATTCTTCTCCTGTAGGTGGGAGAGATGCTTCCCTTC
ATAGTGTAACTGAAGTTTCAGAAAATTCAAGCCGAGATGCAGATCAAGATGGTCCAGCAGCTGCGGAG
CAGGTGAACAGTGATGCTGGATCAGGATCAATTGATCCTGCCTTTCTGGAAGCTCTTCCTGAGGAGCTG
CGTGCTGAAGTCCTCTCATCCCAGCAAGGTCACGTGGCTCAACCATCAAATGCTGAGTCTCAAAACAAT
GGGGATATTGATCCAGAATTCCTTGCAGCTCTTCCCCCAGATATTCGAGCAGAAGTTCTAGCTCAGCAG
CAAGCACAAAGACTACATCAAGCTCAGGAGTTGGAAGGGCAACCTGTTGAAATGGACACCGTCTCAAT
AATTGCAACATTTCCTTCTGAATTACGAGAAGAGGTTCTATTAACATCCTCTGATGCTATCCTTGCCAAC
CTTACACCTGCCCTTGTCGCTGAGGCAAATATGTTGCGGGAGAGGTTTGCACATCGATACAGTCGTACC
CTCTTTGGTATGTATCCCAGAAGTCGTAGAGGAGACACTTCTAGGCGTGATGGTATTGGTTCTGGCCTG
GACGGTGCAGGGGGAAGTGTCACTTCACGCAGGTCTGCTGGCGCTAAGGTTATTGAAGCTGATGGAG
CACCTCTACTTGACACCGAAGCTTTGCATGCCATGATTCGGTTATTTCGCGTAGTTCAGCCACTATATAA
AGGTCAATTGCAGAGGCTTCTTTTGAATCTTTGTGCCCATAGTGAAACCCGAATTTCCCTGGTGAATATT
CTGATGGACTTACTAATGCTTGATGTAAGAAAGCCTGCCAATTATTTTAGTGCCGTTGAACCTCCATACA
GACTATATGGTTGTCAGAGCAATGTAATGTATTCACGTCCTCAATCGTTTGATGGAGTTCCCCCGTTACT
CTCTCGGCGAATACTTGAAACTCTCACCTATCTTGCTCGCCATCATCCATTTGTGGCAAAAATTTTGCTTC
AGTTTAGGCTGCATCCTCCTGCATTAAGAGAACCAGATAATGCTGGTGTTGCACGTGGCAAAGCTGTGA
TGGTGGTTGAAGATGAAATAAATGCTGGTTACATATCCATTGCTATGCTTTTGGGTCTCTTGAAGCAAC
CCCTTTATTTGAGGAGCATAGCTCATCTTGAGCAGTTGCTAAATTTACTGGATGTTATCATTGATAGTGC
TGGAAGCATGCCTAGTTCATCTGATAAATCTCAGATATCTACTGAGGCAGTTGTGGGTCCACAAATTTCT
GCAATGGAGGTAGATGCGAATATTGATTCAGCTACATCTTCTGCTCTTGACGCATCTCCTCAAGTCAATG
AATCCTCCAAACCCACACCTCACAGTAATAAGGAATGTCAGGCTCAGCAAGTATTGTGTGATCTGCCGC
AGGCAGAACTTCAGCTCCTTTGCTCATTGCTTGCTCAAGAAGGTTTGTCAGATAATGCATATGGTCTTGT
TGCGGAGGTAATGAAAAAACTAGTGGCCATTGCTCCGATTCACTGTCAGCTTTTTGTCACTCATCTGGC
AGAAGCAGTTCGAAAATTGACTTCATCTGCAATGGATGAGTTACGCACTTTCAGTGAAGCAATGAAAG
CTCTTCTCAGTACAACATCTTCTGATGGCGCTGCAATTTTAAGAGTTTTGCAGGCCTTAAGTTCCCTGGT
AATCTCATTGACCGAGAAAGAGAATGATGGATTAACTCCTGCCCTTTCTGAAGTTTGGGGAATTAATTC
AGCATTAGAGCCCTTGTGGCATGAGCTTAGCTGTTGTATAAGCAAGATAGAAGCCTACTCTGAGTCAGT
ATCTGAGTCTATTACCTCTTCTAGAACATCTGTGTCAAAACCATCCAGTGTCATGCCTCCACTTCCAGCTG
GTTCTCAAAATATCTTACCATACATAGAATCTTTTTTTGTGGTCTGTGAGAAGCTACATCCTGCACAGTC
AGGTGCTAGTAATGACACAAGTGTTCCTGTTATTTCTGATGTGGAAGATGCTAGGACATCTGGTACTCG
GCTGAAAACATCTGGGCCTGCTATGAAGGTAGATGAGAAAAATGCTGCTTTTGCCAAGTTTTCGGAGA
AGCACAGGAAACTATTAAATGCTTTTATCAGGCAAAATCCTGGCTTGCTTGAAAAGTCTCTTTCCCTCAT
GCTGAAGACTCCAAGATTTATTGATTTTGATAACAAGCGTTCCCATTTCCGATCAAAAATTAAACATCAG
CACGACCATCACCACAGCCCATTAAGAATATCAGTAAGAAGAGCGTATGTTCTAGAAGATTCATATAAC
CAGCTTCGCATGAGATCAACTCAAGATTTGAAGGGAAGGTTGACTGTTCATTTCCAAGGGGAAGAAGG
TATCGATGCTGGTGGGCTTACAAGGGAATGGTACCAACTGTTGTCTAGAGTTATTTTTGACAAAGGAGC
GCTACTTTTCACTACAGTAGGCAATGAATCAACATTTCAGCCAAACCCTAACTCTGTTTACCAAACAGAA
CACCTATCTTATTTCAAATTTGTTGGTAGAGTGGTTGGAAAAGCTTTATTTGATGGTCAGCTCTTGGATG
TCCATTTTACTCGGTCATTCTACAAGCACATCCTAGGGGCCAAAGTTACATATCATGATATTGAAGCCAT
TGATCCTGACTATTTCAGAAATTTGAAATGGATGCTTGAGAATGATATCAGTGATGTTCTGGATCTTACT
TTTAGCATTGATGCAGATGAGGAAAAGTTGATTTTGTATGAGCGGACAGAGGTGACTGATTATGAGCT
AATTCCTGGTGGACGGAATACGAAAGTTACGGAGGAGAATAAGCACCAATATGTTGATTTGGTTGCTG
AGCATCGGTTGACCACTGCTATTCGACCTCAAATAAATGCTTTCTTGGAAGGGTTCAATGAATTAATTCC
CAGGGAGTTAATATCTATATTCAATGACAAAGAGCTGGAATTATTGATCAGTGGACTTCCTGATATTGA
TTTGGATGACTTGAGAGCAAATACAGAATATTCTGGATATAGTGGTGCCTCACCAGTTATCCAATGGTT
TTGGGAGGCTGTTCAAGGTTTCAGCAAAGAAGACAAAGCTAGATTGCTGCAGTTTGTGACTGGCACAT
CCAAGGTGCCTTTGGAGGGTTTTAGCGCTCTTCAAGGAATTTCAGGTGCCCAGAGGTTTCAGATACATA
AGGCATATGGGAGTTCTGATCACTTACCTTCTGCTCATACTTGTTTCAATCAATTAGATTTGCCAGAGTA
TCCATCTAAACAACATTTGGAAGAGAGGTTACTGCTTGCCATTCATGAAGCAAATGAGGGATTCGGATT
TGGTTGA
SEQ ID NO: 22 CDS UPL2
>KRH62268 cds: protein_coding
ATGACAAGCGTAAGATCGAGTTGGCCATCAAGGCTGCGCCAACTTCTTTCCAGCGAGGGTTCCATTGGC
CCTTCCGTCAAACTCGACTCTGACCCTTCTCCTAAGATCAAAGCCTTCATTGAGAAGGTCATTCAATGTC
CATTACAAGATATAGCTATACCCCTCTTTGGCTTTCGGTGGGAGTATAATAAGGGGAATTTTCATCACTG
GAGGCCATTGTTTCTTCATTTTGATACATACTTCAAGACATATTTATCATGTCGAAATGACCTGACATTGT
CCGATAATCTAGAAGTTGGCATTCCATTACCAAAACATGCAATTCTACAAATACTACGGGTGATGCAAA
TAATCTTAGAGAACTGTCCAAACAAGAGTTCATTTGATGGCTTAGAGCACTTCAAGCTTTTACTAGCATC
AACAGATCCTGAGATTATTATTGCTACATTAGAAACTCTTGCTGCGCTTGTAAAAATAAATCCTTCTAAG
CTTCATGGAAGTGCAAAAATGGTTGGCTGTGGTTCAGTAAATAGCTATCTCCTGTCCCTAGCACAGGGG
TGGGGAAGCAAGGAGGAGGGCATGGGTTTGTACTCTTGTATTATGGCAAATGAGAAAGCCCAGGATG
AAGCACTGTGTTTGTTTCCTTCTGATGCAGAGAATGGTAGTGACCACTCCAATTACTGCATAGGTTCTAC
TCTTTATTTTGAATTGCGTGGACCCATTGCTCAAAGCAAGGAACAAAGTGTAGATACAGTTTCCTCAAGT
TTGAGAGTTATACACATTCCAGATATGCATTTACACAAAGAAGATGATTTGTCAATGTTGAAGCAATGC
ATTGAGCAGTATAATGTTCCTCCTGAGCTCCGATTTTCATTGCTCACAAGAATTAGATATGCTCGTGCTT
TCCGGTCTGCGAGAATAAGCAGGCTTTATAGCAGGATTTGCCTTCTTGCTTTCACTGTGTTGGTCCAATC
CAGTGATGCTCATGACGAGCTTGTGTCCTTTTTTGCCAACGAACCAGAGTACACAAGCGAATTGATTAG
AGTTGTGCGATCTGAAGAAACAATATCTGGATCTATCAGAACACTTGTAATGCTTGCATTAGGAGCCCA
GTTAGCAGCATACACATCATCTCATGAACGGGCACGGATACTGAGTGGATCTAGTATGAACTTCACTGG
AGGGAACCGCATGATTCTACTGAATGTACTTCAGAGGGCTATTTTGTCATTGAAGAGTTCTAATGATCC
AACTTCCTTTGCTTTTGTTGAGGCACTTCTTCAATTCTATCTGCTGCATGTAGTGTCAACATCATCTTCTG
GGAGTAATATTAGAGGTTCTGGCATGGTACCCACATTCTTGCCTCTGCTGGAGGATTCTGATCTTGCTC
ATATTCATCTTGTTTGTTTAGCAGTGAAAACCCTTCAGAAGCTTATGGATTATAGTAGTTCAGCTGTATC
TTTGTTTAAAGAGTTGGGGGGTGTTGAGCATTTGGCTCAAAGATTACAGATAGAGGTTCATAGGGTCA
TTGGTTTTGCTGGAGAGAATGATAATGTGATGCTCACTGGTGAAAGCTCAAGACATAGTACTCATCAGC
TTTACTCTCAGAAGAGGCTGATAAAAGTGTCCCTTAAGGCCCTTGGTTCTGCAACATATGCTCCTGCAAA
CTCTACCAGATCTCAACACTCCCATGACAGTTCATTACCTGCAACTCTAGTCATGATTTTTCAGAATGTAA
ATAAGTTCGGAGGTGACATTTATTACTCAGCTGTTACTGTTATGAGTGAAATAATCCACAAAGATCCTAC
ATGCTTCTCTTCTTTGCATGAAATGGGTCTTCCAAATGCTTTTTTATCTTCAGTTGCATCTGGAATTCTTCC
TTCATCAAAGGCTCTGACATGCATTCCAAATGGCATTGGGGCCATTTGTCTTAATGCCAAAGGCTTAGA
GGTTGTTCGAGAGACTTCATCACTGCAGTTCCTTTTTAATATCTTTACAAGCAAAAAGTATGTCCTTTCCA
TGAATGAGGCTATTGTTCCGCTAGCAAATTCTGTAGAGGAACTTCTTCGACACGTGTCTCCATTGAGAA
GTACTGGTGTTGACATCATCATTGAAATCATCCATAAGATTGCATCCTTTGGTGATGGTATTGATACAGG
ATCTTCTTCAGGAAAAGCTAATGAGGATAGTGCAATGGAAACCAATTCTGAAGACAAAGGAAATGAAA
ACCATTGTTGCCTCGTGGGCACAGCAGAGTCTGCCGCTGAAGGGATTAATGATGAGCAATTCATTCAGC
TTTGCACTTTTCATTTGATGGTATTGGTTCACCGGACAATGGAAAATTCTGAAACATGTCGGCTATTTGT
AGAAAAATCAGGAATTGAAGCTTTATTGAAGCTGTTATTACGACCTACCATTGCACAATCCTCGGATGG
CATGTCTATTGCTCTGCATAGCACCATGGTATTTAAGGGGTTTGCTCAACATCATTCCGCTCCTTTGGCA
CGTGCCTTTTGTTCCTCTCTTAAAGAGCACTTGAATGAAGCATTAACTGGGTTTGTTGCATCTTCGGGAC
CTTTGTTGCTGGATCCAAAGATGACCACAAATAACATCTTTTCTTCACTTTTCTTGGTTGAGTTTCTTCTCT
TTCTTGCTGCGTCAAAAGACAACCGTTGGGTGACTGCTTTGCTTACAGAATTTGGAAATGGTAGTAAGG
ATGTTCTTGAAAACATTGGACGTGTCCACCGTGAAGTTTTGTGGCAAATTGCTCTTCTTGAAAATACGAA
GCCTGATATTGAGGATGACGTTTCTTGTTCTACTTCTGATTCACAACAGGCAGAAGTGGATGCAAATGA
AACTGCAGAGCAAAGGTACAATTCTATCAGGCAGTTTCTTGATCCATTACTCAGGAGGAGGACTTTAGG
ATGGAGTGTAGAATCACAGTTTTTTGATCTTATTAACCTGTATCGAGATCTGGGTCGTGCCCCTGGTTCC
CAGCACCGATCAAATTCTGTTGGTCCTACAAACAGGCGGTTAGGATCCCCTAATCCGTTGCATCCGTCT
GAGTCTTCAGATGTATTGGGGGATGCTAGTAAGAAAGAATGTGACAAGCAAAGAACATATTATACCTC
TTGTTGTGACATGGCCAGATCACTTTCATTTCACATTATGCATTTGTTCCAAGAGTTAGGAAAAGTAATG
CTGCAACCTTCTCGCCGTCGTGATGATGTTGCAAGTGTAAGTCCTGCTTCAAAATCAGTGGCTTCTACTT
TTGCAAGCATTGCTCTAGATCACATGAATTTTGGGGGCCATGTAGAAGAAGCATCCATATCAACAAAAT
GTCGTTATTTTGGTAAAGTCATTGATTTTGTGGATGGCATTCTAATGGAAAGGCCTGATTCTTGCAATCC
CATTTTACTGAATTGCTTGTATGGGCATGGAGTTATTCAATCTGTATTGACCACATTTGAAGCAACTAGT
CAGTTGTTATTTGCAGTTAATCGGACCCCTGCATCGCCGATGGAAATTGATGATGGAAATGTGAAGCAG
GATGACAAGGAAGATACCGATCATTTGTGGATATATGGTTCTTTAGCCAGTTATGGTAAATTTATGGAC
CATCTAGTAACCTCCTCTTTCATATTATCTTCTTTCACAAAGCCTATACTTGCACAGCCCCTTAGTGGTGA
TACCTCATATCCCCGGGATGCTGAGATATTTGTGAAAGTCCTCCAATCTATGGTGTTGAAGGCTGTGCT
CCCAGTTTGGATGCATCCCCAGTTTGTTGATTGTAGTCATGGATTTATTTCTAATGTTATCTCTATCATCA
GGCATGTTTATTCAGGGGTTGAAGTAAAAAATGTAAATGGCAGCAGCAGTGCTCGTATTACTGGGCCT
CCTCCTAATGAAACAACAATTTCAACCATTGTAGAGATGGGATTTTCCAGGTCGAGAGCAGAAGAAGCT
TTGAGGCATGTTGGATCAAATAGTGTGGAGTTGGCGATGGAGTGGCTGTTTTCCCATCCAGAGGACAC
ACAAGAAGATGACGAACTTGCTCGTGCACTTGCCATGTCCCTTGGGAACTCTGAATCAGACACCAAGG
ATGCTGCTGCAAATGACAGTGTACAACTGCTTGAGGAAGAAATGGTCCATCTTCCTCCTGTTGATGAGT
TGTTATCAACTTGCACTAAACTTCTTCAAAAGGAACCTCTTGCTTTTCCTGTCCGTGACTTGCTCATGATG
ATATGCTCTCAGAATGATGGTCAAAATAGATCTAATGTTCTCACTTTTATTGTTGACCGGATCAAGGAAT
GTGGATTGATTTCTGGTAACGGAAATAATACCATGCTTGCTGCTCTATTTCATGTTCTTGCATTGATTCTT
AATGAGGATGCTGTTGCGCGAGAAGCTGCTTCAAAGAGTGGTTTCATAAAAATTGCCTCAGATCTACTC
TACCAATGGGATTCTAGTCTTGGTAACAGGGAGAAAGAACAGGTTCCAAAATGGGTCACAGCTGCTTT
TCTTGCATTAGACAGGCTGTTGCAAGTGGATCAAAAATTGAATTCTGAAATTGCAGAGCTTTTGAAGAA
GGAAGCTTTGAATGTTCAGCAGACATCAGTTATCATTGATGAGGATAAGCAACACAAATTGCAGTCTGC
GTTGGGACTTTCCACCAAGTATGCAGATATACATGAACAGAAGAGACTTGTTGAGATTGCTTGTAGTTG
CATGAAGAACCAACTTCCCTCAGACACAATGCATGCTATTTTGCTACTATGTTCCAATCTTACAAAGAAT
CACTCTGTTGCTCTTACCTTTTTTGATGCTGGTGGTTTAAGTTTACTTCTTTCTCTGCCAACCGGTAGCCTC
TTTCCTGGGTTTGACAACGTTGCTGCTGGTATTGTCCGTCATGTTATTGAAGATCCACAAACTCTCCAGC
AAGCAATGGAATCTGAGATAAAACACAGTCTTGTAGCTGCGTCTAACCGCCATCCAAATGGGAGGGTC
AATCCACAAAATTTTCTGTTAAGTTTAGCTTCTGTAATTTCCCGGGATCCAATAATATTTATGCAAGCTGC
TCAATCTGCTTGCCAAGTTGAAATGGTGGGTGAAAGACCTTACATTGTCTTGCTGAAAGATCGGGATAA
AGAGAAATCCAAGGATAAGGATAAGTCACTGGAGAAAGATAAAGCACATAATGATGGAAAAATTGGT
TTGGGAAGTACAGCCACAGCAGCTTCAGGGAATGTTCATGGAAAACTTCATGATTCAAACTCAAAGAA
TGCCAAAAGTTACAAAAAGCCTACTCAAAGTTTTGTTAATGTGATAGAACTTCTTCTTGAATCTATATGC
ACATTTGTTGCCCCCCCTTTGAAGGACAATAATGTATCAAATGTTGTCCCTGGCTCCCCAACATCAAGTG
ACATGGACATTGATGTTTCTACAGTTAGGGGGAAAGGAAAAGCAGTTGCCACTGTGCCTGAGGGGAAT
GAAACCAGCAGTGAGGAAGCATCTGCATCACTAGCAAAGATAGTATTTATTTTGAAGCTTCTGATGGA
GATATTGTTGATGTATTCATCGTCTGTTCATGTTCTGCTTCGACGGGATGCTGAAATGAGCAGCTCTAG
GGACATTTATCAAAAGAATCATGGTAGTTTTGGTGCGGGAGTAATATTCTACCATATTCTTCGTAATTTT
CTTCCTTGTTCTCGAAATTCCAAAAAAGACAAGAAAGTTGATGATGATTGGAGGCAGAAACTAGCAACA
AGGGCTAATCAGTTTATGGTAGCTGCTTGTGTTCGTTCTTCAGAGGCAAGGAGGCGGGTTTTTACTGAG
ATTAGCCATATCATTAATGAATTTGTTGATTCATGTAATTGTGTTAAGCCAAAGCCATCAGGCAATGAAA
TTCTGGTTTTTGTTGATCTACTTAATGATGTTTTGGCTGCTCGGACACCTGCTGGCTCAAGCATCTCAGC
AGAGGCCTCTGTCACTTITTATGGATGCTGGTCTACTTAAATCTTTTACCCGTACTCTCCAAGTTTTAGACT
TGGACCATGCTGACTCGTCTAAAGTTGCTACTGGTATTATCAAAGCTCTTGAACTAGTAACCAAGGAGC
ATGTTCACTCAGTTGAACCGAGTGCAGGAAAGGGTGATAATCAAACTAAGCCTTCTGATCCTAGTCAAT
CCGGAAGAACAGATAATATTGGTCACATGTGTCAGTCCATGGAAACAACATCTCAGGCCAATCACGATT
CCCTTCAAGTTGACCATGTTGGGTCTTACAATGTGATTCAGTCTTATGGTGGGTCTGAAGCTGTTATTGG
TGATATGGAACATGATCTTGATGGGGACTTTGCTCCTGCTAATGAAGATGAGTTCATGCATGAAACTGG
TGAGGATGCCAGAGGCCATGGGAATGGAATTGAAAATGTTGGGCTACAATTTGAAATCCAATCCCATG
GACAAGAAAATCTCGATGATGACGATGATGAGGGTGATATGTCTGGAGATGAGGGTGAAGATGTAGA
TGAAGATGACGAAGATGATGAGGAACACAATGATTTGGAAGAAGATGAAGTCCATCACTTGCCACATC
CTGACACTGATCGTGATGATCATGAGATGGATGATGATGATTTTGATGAAGTGATGGAGGGGGAGGA
GGATGAAGATGAGGATGATGAAGATGGTGTTATACTGAGACTTGAGGAGGGCATCAATGGAATTAAT
GTTTTTGACCATATTGAGGTTTTTGGAAGAGACAATAGTTTTCCAAATGAATCCCTTCATGTCATGCCAG
TTGAAGTTTTTGGATCTAGACGTCCAGGGCGGACCACCTCTATTTACAGCCTGTTGGGCAGAAGTGGTG
ATAATGCCGCCCCTTCTTGCCATCCACTTTTAGTTGGTCCTTCTTCCTCATTCCATCTATCTAATGGTCAAT
CAGATAGTATAACAGAGAACTCCACAGGCTTGGATAATATCTTTCGTTCATTGAGGAGCGGACGTCATG
GGCACCGCTTGAACTTGTGGAGTGATAATAGCCAGCAAATCAGTGGGTCAAATACTGGCGCTGTACCA
CAGGGCCTTGAGGAGTTGCTTGTGTCTCAATTGAGGCGACCTACTGCTGAGAAGTCGTCTGATAATAAT
ATAGCAGACGCTGGTCCTCATAATAAAGTTGAGGTCAGCCAGATGCACAGTTCCGGAGGTTCAAAGCT
TGAAATCCCAGTTGAAAGCAATGCAATTCAGGAAGGTGGTAATGTGACTCCTGCATCAATTGATAACAC
TGACATCAATGCTGATATCAGACCTGTAGGAAATGGAACTCTGCAAGCAGATGTATCAAACACTCACTC
TCAGACAGTTGAGATGCAGTTTGAGAATAATGATGCAGCTGTGCGGGATGTTGAAGCTGTGAGCCAGG
AGAGTAGTGGTAGTGGGGCAACTTTTGGTGAAAGCCTTCGGAGCCTAGATGTTGAGATTGGAAGTGCT
GATGGCCATGATGATGGTGGAGAAAGGCAGGTTTCTGCGGATAGGATAGCAGGTGATTCACAGGCTG
CACGCACAAGAAGAGCAACCATGTCTGTTGGTCATTCTTCTCCTGTAGGTGGGAGAGATGCTTCCCTTC
ATAGTGTAACTGAAGTTTCAGAAAATTCAAGCCGAGATGCAGATCAAGATGGTCCAGCAGCTGCGGAG
CAGGTGAACAGTGATGCTGGATCAGGATCAATTGATCCTGCCTTTCTGGAAGCTCTTCCTGAGGAGCTG
CGTGCTGAAGTCCTCTCATCCCAGCAAGGTCACGTGGCTCAACCATCAAATGCTGAGTCTCAAAACAAT
GGGGATATTGATCCAGAATTCCTTGCAGCTCTTCCCCCAGATATTCGAGCAGAAGTTCTAGCTCAGCAG
CAAGCACAAAGACTACATCAAGCTCAGGAGTTGGAAGGGCAACCTGTTGAAATGGACACCGTCTCAAT
AATTGCAACATTTCCTTCTGAATTACGAGAAGAGGTTCTATTAACATCCTCTGATGCTATCCTTGCCAAC
CTTACACCTGCCCTTGTCGCTGAGGCAAATATGTTGCGGGAGAGGTTTGCACATCGATACAGTCGTACC
CTCTTTGGTATGTATCCCAGAAGTCGTAGAGGAGACACTTCTAGGCGTGATGGTATTGGTTCTGGCCTG
GACGGTGCAGGGGGAAGTGTCACTTCACGCAGGTCTGCTGGCGCTAAGGTTATTGAAGCTGATGGAG
CACCTCTACTTGACACCGAAGCTTTGCATGCCATGATTCGGTTATTTCGCGTAGTTCAGCCACTATATAA
AGGTCAATTGCAGAGGCTTCTTTTGAATCTTTGTGCCCATAGTGAAACCCGAATTTCCCTGGTGAATATT
CTGATGGACTTACTAATGCTTGATGTAAGAAAGCCTGCCAATTATTTTAGTGCCGTTGAACCTCCATACA
GACTATATGGTTGTCAGAGCAATGTAATGTATTCACGTCCTCAATCGTTTGATGGAGTTCCCCCGTTACT
CTCTCGGCGAATACTTGAAACTCTCACCTATCTTGCTCGCCATCATCCATTTGTGGCAAAAATTTTGCTTC
AGTTTAGGCTGCATCCTCCTGCATTAAGAGAACCAGATAATGCTGGTGTTGCACGTGGCAAAGCTGTGA
TGGTGGTTGAAGATGAAATAAATGCTGGTTACATATCCATTGCTATGCTTTTGGGTCTCTTGAAGCAAC
CCCTTTATTTGAGGAGCATAGCTCATCTTGAGCAGTTGCTAAATTTACTGGATGTTATCATTGATAGTGC
TGGAAGCATGCCTAGTTCATCTGATAAATCTCAGATATCTACTGAGGCAGTTGTGGGTCCACAAATTTCT
GCAATGGAGGTAGATGCGAATATTGATTCAGCTACATCTTCTGCTCTTGACGCATCTCCTCAAGTCAATG
AATCCTCCAAACCCACACCTCACAGTAATAAGGAATGTCAGGCTCAGCAAGTATTGTGTGATCTGCCGC
AGGCAGAACTTCAGCTCCTTTGCTCATTGCTTGCTCAAGAAGGTTTGTCAGATAATGCATATGGTCTTGT
TGCGGAGGTAATGAAAAAACTAGTGGCCATTGCTCCGATTCACTGTCAGCTTTTTGTCACTCATCTGGC
AGAAGCAGTTCGAAAATTGACTTCATCTGCAATGGATGAGTTACGCACTTTCAGTGAAGCAATGAAAG
CTCTTCTCAGTACAACATCTTCTGATGGCGCTGCAATTTTAAGAGTTTTGCAGGCCTTAAGTTCCCTGGT
AATCTCATTGACCGAGAAAGAGAATGATGGATTAACTCCTGCCCTTTCTGAAGTTTGGGGAATTAATTC
AGCATTAGAGCCCTTGTGGCATGAGCTTAGCTGTTGTATAAGCAAGATAGAAGCCTACTCTGAGTCAGT
ATCTGAGTCTATTACCTCTTCTAGAACATCTGTGTCAAAACCATCCAGTGTCATGCCTCCACTTCCAGCTG
GTTCTCAAAATATCTTACCATACATAGAATCTTTTTTTGTGGTCTGTGAGAAGCTACATCCTGCACAGTC
AGGTGCTAGTAATGACACAAGTGTTCCTGTTATTTCTGATGTGGAAGATGCTAGGACATCTGGTACTCG
GCTGAAAACATCTGGGCCTGCTATGAAGGTAGATGAGAAAAATGCTGCTTTTGCCAAGTTTTCGGAGA
AGCACAGGAAACTATTAAATGCTTTTATCAGGCAAAATCCTGGCTTGCTTGAAAAGTCTCTTTCCCTCAT
GCTGAAGACTCCAAGATTTATTGATTTTGATAACAAGCGTTCCCATTTCCGATCAAAAATTAAACATCAG
CACGACCATCACCACAGCCCATTAAGAATATCAGTAAGAAGAGCGTATGTTCTAGAAGATTCATATAAC
CAGCTTCGCATGAGATCAACTCAAGATTTGAAGGGAAGGTTGACTGTTCATTTCCAAGGGGAAGAAGG
TATCGATGCTGGTGGGCTTACAAGGGAATGGTACCAACTGTTGTCTAGAGTTATTTTTGACAAAGGAGC
GCTACTTTTCACTACAGTAGGCAATGAATCAACATTTCAGCCAAACCCTAACTCTGTTTACCAAACAGAA
CACCTATCTTATTTCAAATTTGTTGGTAGAGTGGTTGGAAAAGCTTTATTTGATGGTCAGCTCTTGGATG
TCCATTTTACTCGGTCATTCTACAAGCACATCCTAGGGGCCAAAGTTACATATCATGATATTGAAGCCAT
TGATCCTGACTATTTCAGAAATTTGAAATGGATGCTTGAGAATGATATCAGTGATGTTCTGGATCTTACT
TTTAGCATTGATGCAGATGAGGAAAAGTTGATTTTGTATGAGCGGACAGAGGTGACTGATTATGAGCT
AATTCCTGGTGGACGGAATACGAAAGTTACGGAGGAGAATAAGCACCAATATGTTGATTTGGTTGCTG
AGCATCGGTTGACCACTGCTATTCGACCTCAAATAAATGCTTTCTTGGAAGGGTTCAATGAATTAATTCC
CAGGGAGTTAATATCTATATTCAATGACAAAGAGCTGGAATTATTGATCAGTGGACTTCCTGATATTGA
TTTGGATGACTTGAGAGCAAATACAGAATATTCTGGATATAGTGGTGCCTCACCAGTTATCCAATGGTT
TTGGGAGGCTGTTCAAGGTTTCAGCAAAGAAGACAAAGCTAGATTGCTGCAGTTTGTGACTGGCACAT
CCAAGGCTTATGATGGAGATAAGACACATTGGGAGCCTTTGCTTAACAAATTTCAAGCCAAGCTCTCAA
AGTGGAATCAGAAAACTTTGTCTATGGGTGGTAGAGTTACCTTGATAAAATCTGTCCTGAGTGCACTCC
CTATATATCTACTATCTTTCTTCAAGATCCCCCAAAGAATAGTGGATAAGTTGGTGACCCTCCAAAGGCA
GTTTCTGTGGGGGGGAACTCAACACCATAACAGAATTCCTTGGGTCAAGTGGGCTGACATCTGCAATCC
GAAGATTGATGGGGGATTGGGAATCAAAGACCTGTCCAATTTCAATGCAGCCTTAAGGGGAAGATGG
ATCTGGGGATTAGCTTCTAATCACAATCAGCTTTGGGCCAGACTTGCAGAGCAGTAG
SEQ ID NO: 23 CDS UPL2 >KRH16871 cds:protein_coding
ATGACAACCCTAAGATCGAGTTGGCCTTCGAGGCTGCGCCAACTTCTGTCGAGCGAGGGCGCCATTGG
TCCTTCCGTCAAGGTGGACACCGAGCCCCCTCCTATGGTCAAAGCCTTCATTGAGAAGATCATCCAGTG
TCCATTACAAGATATTGCCATACCACTTTCTGGCTTTCGGTGGGAGTACAATAAGGGGAATTTTCATCAC
TGGAGACTGTTGTTGCTTCATTTTGATACATACTTCAAGACTTATTTGTCGTGTAGAAATGATCTGACAT
TGTTAGATAATCTAGAAGATGACAGCCCATTACCAAAACATGCAATTCTGCAAATATTGCGAGTGTTGC
AAATAATTTTAGAGAACTGTCCAAACAAGAGTTCCTTTGATGGCTTAGAGCATTTCAAGCTTTTACTAGC
ATCAACAGATCCTGAGATTCTTATTGCTACATTAGAAACTCTTTCTGCACTTGTAAAAATTAATCCCTCTA
AGCTTCATGGAAGTACAAAGATGATTTGCTGTGGTTCGGTGAACAGCTATCTTTTGTCCCTAGCACAAG
GCTGGGGAAGCAAGGAGGAGGGCCTAGGATTGTACTCTTGTGTTATGGCAAATGAGAAAGTCCAAGA
TGAAGCACTGTGCTTATTTCCTTCTGAAGAGATTGGTCATGACCAATCAAATTGCCGCATGGGCACTAC
CCTTTATTTTGAATTGCATGGTCCCAGTGCCCAAAGCAAGGAACATAGTGCAGATGCAGTTTCCCCTGG
CTCAACAGTTATACATATGCCAGATTTGCATCTGCGCAAAGAAGATGATTTGTCCTTGATGAAGCAGTG
CATTGAACAATTTAGCGTTCCTTCTGAGCTCAGATTTTCATTGCTCACTAGAATCAGATATGCTCGTGCC
TTTCGTTCTCCTAGAATATGCAGGCTTTACAGCAGGATTTGCCTACTTTCTTTCATTGTTCTGGTGCAGTC
TGGTGATGCTCAGGAAGAACTCGTCTCCTTTTTTGCCAATGAACCAGAATATACAAATGAATTAATTAG
AATTGTACGTTCAGAGGAAGTTATATCTGGATCTATCAGGACACTTGCAATGCTTGCTTTAGGAGCTCA
ATTAGCCGCATATACATCATCGCATCATCGGGCACGAATACTCAGTGGATCTAGTTTAACTTTTGCTGGT
GGGAACCGCATGATACTCCTAAATGTGCTCCAGAGGGCTATTTTGTCATTGAAGAGTTCTAATGATCCA
TCATCCCTTGCTTTTGTTGAAGCACTTCTTCAGTTCTATCTGCTCCATGTGGTTTCAACCTCAACTTCTGGT
AATAATATTAGAGGTTCTGGCATGGTGCCTACATTCTTGCCGTTGCTGGAGGATTTTGATCCTACACATA
TTCATCTAGTCTGTTTTGCTGTGAAAACACTTCAGAAGCTTATGGATTATAGTAGCTCAGCTGTATCATT
GTTTAAAGAATTGGGGGGCATTGAACTTTTGGCTCAGAGATTACAGAAAGAGGTACACAGAGTCATTG
GTTTGGTTGGAGAAACTGATAACATTATGCTTACTGGTGAAAGCTTGAGATATAGTACTGATCAATTGT
ACTCCCAGAAGAGACTCATAAAGGTCTCCCTTAAGGCGCTTGGTTCTGCAACATACGCGCCTGCAAACT
CTACCAGATCTCAACATTCTCAAGACAGTTCATTACCTGTAACTCTAAGATTGATTTTTCAGAATGTAGA
TAAGTTTGGAGGTGACATTTATTATTCAGCTGTTACTGTTATGAGCGAAATAATCCACAAAGATCCAACC
TGTTTTTCTGCTCTGCATGAAATGGGTCTTCCTGATGCTTTTTTATTGTCAGTTGGATCTGAAATACTTCC
ATCATCAAAGGCTTTGACATGCATTCCAAATGGTCTTGGGGCAATTTGTCTTAATGCCAAAGGGTTAGA
GGCTGTTAGAGAATCTTCATCGCTACGGTTCCTTATTGACATTTTCACTAGCAAGAAGTATATCTTAGCC
ATGAATGAGGCTATTGTTCCTTTGGCAAATGCTGTGGAGGAACTTCTACGTCATGTATCTACATTGAGA
AGCTCCAGTGTTGATATTATCATTGAAATCATCCACAAGATCGCATCTTTTGGGGATGGAAATGGTACT
GGATTTTCTGGAAAAGCTGAGGGCACTGCCATGGAAACAGATTCTGAAAACAAAGAAAAAGAAGGCC
ATTGTTGCATTGTAGGCACATCATATTCAGCCATAGAAGGGATAAGTGATGAGCAGTTTATTCAGCTAT
GTGTCTTTCATTTAATGGTATTGATTCATAGGACTATGGAAAATGCCGAGACATGCCGGTTGTTTGTGG
AAAAATCAGGAATTGAAGCTTTATTGAATTTGTTGTTGCGGCCAACTATTGCACAATCCTCAGATGGCA
TGTCTATTGCTTTACATAGCACGATGGTATTTAAAGGGTTTGCTCAACATCATTCCATTCCTCTGGCACAT
GCCTTCTGTTCTTCTCTTAGAGAGCACTTGAAGAAAGCTTTAGCGGGGCTTGGTGCAGCATCAGAACCT
TTGTTGCTGGATCCAAGGATGACAACTGATGGTGCCATCTTTTCTTCACTTTTCCTGGTTGAGTTCCTTCT
ATTTCTTGCTGCACCAAAAGACAATCGTTGGGTGACTGCCTTGCTTACAGAATTTGGAAATGGAGGTAA
GGATGTTCTTGAAGACATTGGACGTGTACACCGTGAAGTCCTGTGGCAAATTGCTCTACTTGAAAACAG
AAAGCCTGAGATTGAGGAAGATGGTGCTTGTACTTCTGATTTACAACAGGCCGAAGGGGATGCAAGTG
AAACTGAAGAGCAAAGGTTGAATTCTTTCAGGCAGTTTCTTGACCCATTATTGAGAAGAAGAACATCAG
GATGGAGCATTGAATCTCAGTTTTTTAACCTTATAAACCTGTATCGAGATTTGGGCCGTTCCACTGGTTC
TCAACATAGATCAAATTTAGTTGGTCCGAGGTCAAGTTCTAGTAATCAGGTACAGCATTCTGGGTCAGA
TGATAATTCTGGGACTGCTGATAAGAAGGAATCTGACAAGCAGAGACCATATTATACCTCTTGTTGTGA
CATGGTCAGATCACTTTCATTTCACATTACCCATTTGTTCCAAGAGTTGGGAAAAGTAATGTTGCTACCT
TCACGTCGACGTGATGATGTTGTGAATGTAAGTCCTGCTTCAAAATCAGTGGCTTCTACTTTTGCATCCA
TTGCTTTTGATCACATGAATTATGGTGGCCGTTGTGTAAATCTTTCGGGAACAGAAGAATCCATATCAAC
AAAATGTCGATATTTTGGGAAAGTGATTGATTTTATGGATAATGTTCTAATGGAGAGGCCAGATTCATG
CAATCCTATTATGCTGAATTGCTTGTATGGACGTGGAGTTATTGAAACTGTATTAACTACCTTTGAAGCT
ACCAGTCAGCTGCTCTTTACAGTTAATCGGGCCCCTGCCTCGCCCATGGATACTGATGATGCGAATGCA
AAGCAAGATGACAAGGAAGATACAGATAATTCATGGATTTATGGTTCTTTAGCTAGTTATGGGAAATTG
ATGGACCATCTAGTGACCTCCTCTTTTATATTATCATCATTCACAAAGCATTTACTTGCACAGCCCCTTAC
TAATGGTAATACAGCTTTCCCAAGGGATGCTGAGACTTTTGTGAAGGTCCTTCAATCCAGAGTGTTGAA
GACTGTGCTTCCTGTTTGGACTCATCCCCAGTTTGTTGACTGTAGTTATGAATTTATTTCTACAGTTATTT
CTATCATTAGGCATGTCTATACAGGTGTTGAAGTAAAAAATGTGAACGGAAGTGGTGGTGCTCGCATT
ACTGGGCCGCCTCCTAATGAAACAACTATTTCAACCATTGTAGAGATGGGGTTTTCCAGGTCTAGAGCA
GAAGAAGCTTTGAGGCAAGTTGGGTCAAATAGTGTGGAGTTGGCAATGGAGTGGTTGTTCTCTCATCC
AGAGGAGATACAAGAAGATGATGAACTTGCCCGTGCACTTGCCATGTCCCTTGGAAACTCAGAATCAG
ATGCAAAGGATGCAGTTGCTAATGACAATGCCCTGCAGCTTGAAGAAGAGATGGTCCTACTCCCTCCTG
TTGATGAGTTGTTATCTACTTGCACAAAACTTTTGTCGAAGGAACCACTTGCTTTTCCTGTCCGTGACTT
GCTTGTGATGATATGCTCTCATGATGATGGTCACCATAGATCTAATGTGGTCTCATTTATTGTGGAACG
GATCAAAGAATGTGGTTTGGTTCCTAGCAATGGAAATGTTGCCACGCTGGCTGCTCTTTTTCATGTTCTA
GCCTTAATTCTTAATGAGGATGCTGTGGCTAGGGAAGCTGCTTCTACAAGTGGTTTGATCAAAATTGCC
TCAGATCTACTCTACCAGTGGGATTCTAGTCTTGATAGCAGGGAGAAACAGCAGGTACCAAAATGGGT
GACTGCTGCTTTCCTTGCATTAGACAGATTGTTGCAAGTAGATCAAAAATTGAATTCTGAAATCGCAGA
GCAGTTGAAGAAGGAAGCTGTGAATAGCCAGCAGACATCGATTACCATTGATGAAGACAGGCAAAAC
AAGTTGCAGTCTGCATTGGGACTCTCTATGAAGTATGCAGATATACATGAACAGAAGAGACTTGTTGA
GGTTGCTTGTAGTTGTATGAATAATCAACTTCCATCTGACACAATGCATGCTATTCTGCTACTATGTTCC
AATCTTACAAGGAATCATTCTGTAGCTCTTACATTTTTGGATGCTGGTGGTTTGAATCTACTTCTTTCTTT
GCCAACCAGCAGCCTCTTCCCTGGGTTTGACAATGTTGCTGCTAGTATTGTTCGTCATGTTCTTGAAGAT
CCACAAACGCTCCAGCAAGCAATGGAATCTGAGATAAAACATAGTCTTGCAGTGGCATCTAATCGGCAT
CCAAATGGAAGGGTCAATCCTCATAATTTCCTTTTAAATTTAGCTTCTGTTATTTATCGGGATCCAGTAAT
CTTTATGCTAGCTGCTCAATCTGTGTGCCAAGTTGAAATGGTAGGTGAGAGGCCATACATTGTCTTGCT
GAAAGATAGGGATAAAGACAAAGCTAGGGAGAAAGAAAAGGATAAGGATAAAACATTGGAGAAAGA
TAAAGTACAGAACAGTGATGGGAAGGTTGTTTTGGGAAATACAAACACAGCACCTACTGGCAATGGCC
ATGGCAAAATTCAGGATTCAAATACCAAGAGTGCCAAAGGTCACAGAAAACCTAACCAAAGTTTTATTA
ATGTAATAGAGCTTCTTCTTGAATCTATATGCACTTTTGTTCCTCCCTTGAAGGATGACATTGCCTCAAAT
GTTCTTCCTGGAACCCCAGCATCAACTGATATGGACATTGATGTCTCCGTGGTTAAGGGAAAAGGAAAA
GCAGTTGCCACTGTGTCTGACGGCAACGAAACTGGTAGTCAGGTTGCTTCTGCATCACTTGCAAAGATT
GTCTTCATTTTAAAGCTTCTGACAGAGATATTATTGCTGTATTCATCATCTGTTCATGTTCTACTTCGACG
AGATGCTGAAATAAGCTGCATTAGAGGTTCTTATCAAAAGAGTCCTGCAGGTTTAAGCATGGGTTGGA
TATTTTCCCATATTCTTCATAATTTTCTTCCATATTCTCGAAACTCAAAAAAGGACAAGAAAGCTGATGGT
GATTGGAGGCAGAAACTAGCAACCAGGGCCAACCAGTTTATAGTGGGTGCTTGTGTTCGATCTACAGA
GGCAAGGAAGAGGGTTTTTGGTGAGATTAGTTATATCATCAATGAATTTGTTGATTCATGTCATGACAT
TAAGCGTCCAGGAAATGAAATTCAGGTTTTTGTTGATCTACTAAATGATGTTTTGGCTGCTCGTACACCT
GCTGGTTCGTACATTTCAGCTGAGGCCTCTACCACTTTTATAGATGCTGGTTTGGTTAAATCATTCACTT
GCACTCTACAAGTTTTGGACCTTGACCATGCTGGTTCATCTGAAGTTGCTACTGGTATTATTAAAGCTCT
TGAGTTGGTAACCAATGAGCATGTCCATTCAGTTCATTCTAGTGCAGGGAAGGGTGATAATTCAACAAA
ACCTTCTGTTCTAAGTCAACCTGGAAGAACAAATAATATTGGTGAACTGTCTCAGTCCATGGAGACATC
ACAAGCCAATCCTGATTCCCTTCAAGTTGACCATGTTGGGTCTTATGCAGTTCACTCCTATGGTGGGTCT
GAAGCTGTTACTGATGATATGGAACATGATCAAGATCTTGATGGGAGCTTTGTTCCTGCTAATGAGGAT
GATTACATGCATGAAAATTCTGAGGATGCAAGAAATCTTGAAAATGGAATGGAAAATGTGGGTCTACA
ATTTGAAATCCAACCTCATGGCCAAGAAAATCTTGATGAGGATGACGATGAGGATGATGATATGTCTG
GAGATGAAGGTGAGGATGTAGATGAAGATGATGATGATGAGGAGGAACACAATGATTTGGAAGAAG
TCCATCATTTGCCACATCCTGACACAGATCAAGACGAGCATGAGATTGATGATGAAGATTTTGATGATG
AAGTGATGGAGGAAGACGATGAGGATGACGAGGAAGATGAAGATGGTGTTATACTGCGACTTGAGG
AGGGAATTAATGGAATTAATGTTTTTGACCATATTGAGGTTTTTGGCAGAGATAATAGTTTTGCAAATG
AAGCTTTACATGTAATGCCAGTTGAGGTTTTTGGATCCAGACGTCCGGGGAGGACGACATCTATTTATA
GTCTTTTGGGCAGAACTGGTGATGCTGCTGTGCCTTCTCGTCACCCACTCTTGCTTGAACCTTCTTCATTC
CCTCCACCTACAGGGCAATCAGATAGTTCAATGGAGAACAACTCAGTGGGTTTGGATAATATATTTCGA
TCGCTGAGGAGTGGGCGCCATGGACACCGTTTGCACTTGTGGACTGATAATAACCAGCAAAGTGGTGG
GACAAACACTGCTGTTGTACCACAAGGCCTTGAGGAGTTGCTTGTCACTCAATTAAGGCGACCAACCCC
TGAAAAGTCATCCAATCAGAACATAGCAGAAGCAGGTTCTCATGGTAAAATTGGAACAACCCAGGCAC
AAGATGCAGGGGGTGCAAGGCCAGAAGTCCCCGTTGAAAGTAATGCTATTCTGGAAATTAGTACTATA
ACTCCCTCAATTGATAACAGTAACAATGCGGATGTCAGACCAGCAGGGACTGGACCTTCACATACAAAT
GTTTCAAACACCCAATCACGGGCAGTTGAGATGCAATTTGAACATACTGATGGAGCTGTGAGGGATAT
TGAAGCTGTCAGCCAGGAGAGTAGTGGTAGTGGAGCAACTTTCGGTGAAAGCCTTCGGAGCTTGGAA
GTTGAGATTGGAAGTGCTGATGGCCATGATGATGGTGGTGAAAGGCTGGTTTCTGCTGATAGGATGGC
AGGTGATTCACAGGCTGCACGCACAAGAAGAGCAAATACACCTTTGAGTCACTTTTCTCCTGTGGTTGG
AAGAGATGTGTCCCTTCATAGTGTTACTGAAGTTTCAGAAAATTCAAGCCGTGATGCAGATCAACAAGG
TCCTGCAGCAGAGCAGCAGGTGAACAGTGATGCGGGATCAGGAGCTATTGATCCTGCTTTTCTGGATG
CTCTTCCTGAGGAGCTACGTGCTGAAGTCCTTTCAGCTCAGCAGGGTCAAGTGGCTCAGCCATCAAATG
TTGAGTCTCAAAACACTGGGGATATTGACCCAGAGTTCCTAGCAGCTCTTCCAGCTGATATTCGAGCAG
AAGTTCTAGCTCAGCAGCAAGCACAGAGGTTGCATCAGTCTCAGGAGCTGGAAGGTCAACCTGTGGAA
ATGGATACAGTCTCAATAATTGCAACTTTTCCATCAGATTTACGAGAAGAGGTTCTCTTGACATCACCAG
ATACTATCCTTGCCAATCTTACACCTGCTCTTGTTGCTGAGGCAAATATGTTGCGGGAGAGGTTTGCACA
CCGTTATAGTCGTACCCTCTTTGGTATGTATCCAAGAAGTCGTAGAGGGGAGACTTCAAGACGTGAAG
GTATTGGTTCTGGTCTGGATGGAGCAGGAGGAACCATTTCTTCTCGCCGCTCCAGTGGAGTTAAGGTTG
TTGAAGCTGATGGAGCACCTTTAGTTGACACAGAAGCTTTGCATGCTATGATTCGGTTATTTCGTGTAG
TGCAGCCACTCTATAAAGGCCAACTCCAGAGGCTTCTATTAAATCTTTGTGCCCATAGTGAAACAAGAA
CCTCTCTGGTGAAAATTCTCATGGACTTGCTAATGCTTGATGTAAAAAGGCCTGTCAGTTATTTTAGTAA
AGTTGAGCCACCATATAGATTGTATGGTTGTCAGAGCAATGTAATGTATTCACGTCCTCAATCTTTTGAT
GGAGTTCCCCCATTGCTGTCTCGTAGAATACTTGGAATTCTCACTTATCTTGCTCGCAATCATCTGTATGT
GGCAAAATTTTTGCTTCAGTGTAGGCTGTCTCATCCTGCAATAAAAGAACCAGATGATCCACGGGGAAA
AGCTGTGATGGTTGTTGAAGATGAAGTAAATATAAGTGAAAGTAATGATGGGTACATCGCCATTGCAA
TGCTATTGGGTCTGTTGAACCAACCACTTTATTTGAGGAGCATAGCCCACCTTGAGCAGCTGCTAGATTT
ACTGGATGTTATCATTGACAGTGCTGGAAACAAGTCATCTGGCAAATCCTTGATACCTACTAACCCATCA
TCAGCTCCACAAATTTCTGCTGCGGAAGCCGATGCGAATGCAGATTCTAACAATTTACCTTCTGCGGAT
GATGCATCTAAAGTTGATGGTTCCTCCAAACCGACAGTCTCTGGCATTAATGTTGAATGTGAGTTACAT
GGAGTGTTGAGTAATCTTCCAAAAGCAGAACTCCGGCTCCTGTGCTCACTGCTTGCTCAAGAAGGTTTG
TCAGATAATGCGTATAATCTTGTAGCGGAAGTAATGAAGAAATTGGTGGCCATTGCTCCAACACATTGT
GAGCTTTTTGTCACTGAGCTGGCAGAAGCAGTTCAAAAGTTGACTTCATCTGCAATGAATGAGTTACGT
GTCTTTAGTGAAGCAATGAAAGCTCTGCTTAGTACCTCTTCTACTGATGGAGCTGCAATTTTGAGAGTCT
TGCAAGCCTTGAGTTCCCTTGTCACCTTACTGACGGAGAAAGAGAATGATAGAGGTACTCCTGCTCTTT
CTGAGGTTTGGGAAATCAATTCAGCATTAGAACCCTTGTGGCATGAGCTTAGTTGTTGCATAAGCAAGA
TAGAATCCTACTCAGAGTCTGCATCTGAGTTTTCGACATCTTCTAGTACCTTTGTGTCTAAACCGTCTGGT
GTAATGCCTCCACTTCCAGCTGGCTCTCAAAATATCTTACCATACATTGAATCTTTCTTTGTGGTTTGTGA
GAAATTGCATCCTGCTCAGCCAGGTGCTAGTCACGACTCAAGTATTCCTGTTATTTCGGATGTTGAGTAT
GCCACCACATCTGTAACTCCCCAGAAAGCATCTGGAACTGCTGTGAAAGTAGATGAGAAACATATGCCT
TTTGTCCGGTTCTCAGAGAAGCACAGGAAGCTACTAAATGCTTTCATAAGGCAAAACCCTGGTTTGCTT
GAAAAATCTTTCTCACTCATGCTAAAGGTTCCAAGATTTATTGATTTTGATAACAAGCGTGCCCACTTCC
GATCAAAAATTAAGCATCAGCATGACCATCACCATAGTCCCTTGAGAATATCAGTAAGAAGGGCATATG
TTCTAGAAGATTCTTACAACCAGCTTCGCATGAGATCAACTCAAGATTTGAAGGGAAGGTTGACTGTCC
ACTTCCAAGGGGAGGAGGGTATTGATGCAGGTGGGCTTACAAGGGAATGGTATCAATTATTGTCCAGA
GTTATTTTTGATAAAGGAGCACTGCTTTTTACTACAGTGGGCAATGAATCAACATTTCAGCCAAACCCTA
ACTCTGTTTACCAAACAGAACATTTATCTTATTTCAAATTTGTTGGTAGAGTGGTCGGTAAAGCATTATT
TGATGGTCAACTCTTGGATGTTCATTTTACTCGGTCATTCTACAAGCACATTCTTGGAGTCAAAGTTACA
TATCATGATATTGAAGCCATTGATCCTGATTATTTCAAAAATTTGAAATGGATGCTTGAGAATGATATCA
GTGATGTTCTGGATCTTACTTTTAGCATTGACGCAGATGAGGAAAAATTGATCTTATATGAACGAACAG
AGGTGACTGATTATGAGTTGATTCCCGGGGGACGGAATATCAAAGTTACTGAGGAGAACAAGCATCAA
TATGTTGATTTGGTTGCCGAGCATCGGTTGACAACTGCTATTCGACCTCAAATAAATTATTTCTTAGAAG
GGTTCATTGAATTGATTCCCAGGGAGTTGATATCGATATTCAATGACAAAGAGCTGGAATTGTTGATCA
GTGGACTTCCTGATATTGATTTGGATGACTTGAGAGCAAATACAGAATATTCTGGATATAGTGCTGCAT
CGCCAGTTATCCAATGGTTTTGGGAGGTTGTTCAAGGTTTGAGCAAAGAAGACAAGGCTCGACTGTTG
CAATTTGTGACAGGCACATCCAAGGAATTTCAGGCTCCCAGAAGTTTCAGATACACAAAGCATATGGAA
GTCCTGATCACTTGCCTTCTGCTCATACTTGCTTCAATCAATTAG
SEQ ID NO: 24 CDS UPL2 >KRH16870 cds: protein_coding
ATGACAACCCTAAGATCGAGTTGGCCTTCGAGGCTGCGCCAACTTCTGTCGAGCGAGGGCGCCATTGG
TCCTTCCGTCAAGGTGGACACCGAGCCCCCTCCTATGGTCAAAGCCTTCATTGAGAAGATCATCCAGTG
TCCATTACAAGATATTGCCATACCACTTTCTGGCTTTCGGTGGGAGTACAATAAGGGGAATTTTCATCAC
TGGAGACTGTTGTTGCTTCATTTTGATACATACTTCAAGACTTATTTGTCGTGTAGAAATGATCTGACAT
TGTTAGATAATCTAGAAGATGACAGCCCATTACCAAAACATGCAATTCTGCAAATATTGCGAGTGTTGC
AAATAATTTTAGAGAACTGTCCAAACAAGAGTTCCTTTGATGGCTTAGAGCATTTCAAGCTTTTACTAGC
ATCAACAGATCCTGAGATTCTTATTGCTACATTAGAAACTCTTTCTGCACTTGTAAAAATTAATCCCTCTA
AGCTTCATGGAAGTACAAAGATGATTTGCTGTGGTTCGGTGAACAGCTATCTTTTGTCCCTAGCACAAG
GCTGGGGAAGCAAGGAGGAGGGCCTAGGATTGTACTCTTGTGTTATGGCAAATGAGAAAGTCCAAGA
TGAAGCACTGTGCTTATTTCCTTCTGAAGAGATTGGTCATGACCAATCAAATTGCCGCATGGGCACTAC
CCTTTATTTTGAATTGCATGGTCCCAGTGCCCAAAGCAAGGAACATAGTGCAGATGCAGTTTCCCCTGG
CTCAACAGTTATACATATGCCAGATTTGCATCTGCGCAAAGAAGATGATTTGTCCTTGATGAAGCAGTG
CATTGAACAATTTAGCGTTCCTTCTGAGCTCAGATTTTCATTGCTCACTAGAATCAGATATGCTCGTGCC
TTTCGTTCTCCTAGAATATGCAGGCTTTACAGCAGGATTTGCCTACTTTCTTTCATTGTTCTGGTGCAGTC
TGGTGATGCTCAGGAAGAACTCGTCTCCTTTTTTGCCAATGAACCAGAATATACAAATGAATTAATTAG
AATTGTACGTTCAGAGGAAGTTATATCTGGATCTATCAGGACACTTGCAATGCTTGCTTTAGGAGCTCA
ATTAGCCGCATATACATCATCGCATCATCGGGCACGAATACTCAGTGGATCTAGTTTAACTTTTGCTGGT
GGGAACCGCATGATACTCCTAAATGTGCTCCAGAGGGCTATTTTGTCATTGAAGAGTTCTAATGATCCA
TCATCCCTTGCTTTTGTTGAAGCACTTCTTCAGTTCTATCTGCTCCATGTGGTTTCAACCTCAACTTCTGGT
AATAATATTAGAGGTTCTGGCATGGTGCCTACATTCTTGCCGTTGCTGGAGGATTTTGATCCTACACATA
TTCATCTAGTCTGTTTTGCTGTGAAAACACTTCAGAAGCTTATGGATTATAGTAGCTCAGCTGTATCATT
GTTTAAAGAATTGGGGGGCATTGAACTTTTGGCTCAGAGATTACAGAAAGAGGTACACAGAGTCATTG
GTTTGGTTGGAGAAACTGATAACATTATGCTTACTGGTGAAAGCTTGAGATATAGTACTGATCAATTGT
ACTCCCAGAAGAGACTCATAAAGGTCTCCCTTAAGGCGCTTGGTTCTGCAACATACGCGCCTGCAAACT
CTACCAGATCTCAACATTCTCAAGACAGTTCATTACCTGTAACTCTAAGATTGATTTTTCAGAATGTAGA
TAAGTTTGGAGGTGACATTTATTATTCAGCTGTTACTGTTATGAGCGAAATAATCCACAAAGATCCAACC
TGTTTTTCTGCTCTGCATGAAATGGGTCTTCCTGATGCTTTTTTATTGTCAGTTGGATCTGAAATACTTCC
ATCATCAAAGGCTTTGACATGCATTCCAAATGGTCTTGGGGCAATTTGTCTTAATGCCAAAGGGTTAGA
GGCTGTTAGAGAATCTTCATCGCTACGGTTCCTTATTGACATTTTCACTAGCAAGAAGTATATCTTAGCC
ATGAATGAGGCTATTGTTCCTTTGGCAAATGCTGTGGAGGAACTTCTACGTCATGTATCTACATTGAGA
AGCTCCAGTGTTGATATTATCATTGAAATCATCCACAAGATCGCATCTTTTGGGGATGGAAATGGTACT
GGATTTTCTGGAAAAGCTGAGGGCACTGCCATGGAAACAGATTCTGAAAACAAAGAAAAAGAAGGCC
ATTGTTGCATTGTAGGCACATCATATTCAGCCATAGAAGGGATAAGTGATGAGCAGTTTATTCAGCTAT
GTGTCTTTCATTTAATGGTATTGATTCATAGGACTATGGAAAATGCCGAGACATGCCGGTTGTTTGTGG
AAAAATCAGGAATTGAAGCTTTATTGAATTTGTTGTTGCGGCCAACTATTGCACAATCCTCAGATGGCA
TGTCTATTGCTTTACATAGCACGATGGTATTTAAAGGGTTTGCTCAACATCATTCCATTCCTCTGGCACAT
GCCTTCTGTTCTTCTCTTAGAGAGCACTTGAAGAAAGCTTTAGCGGGGCTTGGTGCAGCATCAGAACCT
TTGTTGCTGGATCCAAGGATGACAACTGATGGTGCCATCTTTTCTTCACTTTTCCTGGTTGAGTTCCTTCT
ATTTCTTGCTGCACCAAAAGACAATCGTTGGGTGACTGCCTTGCTTACAGAATTTGGAAATGGAGGTAA
GGATGTTCTTGAAGACATTGGACGTGTACACCGTGAAGTCCTGTGGCAAATTGCTCTACTTGAAAACAG
AAAGCCTGAGATTGAGGAAGATGGTGCTTGTACTTCTGATTTACAACAGGCCGAAGGGGATGCAAGTG
AAACTGAAGAGCAAAGGTTGAATTCTTTCAGGCAGTTTCTTGACCCATTATTGAGAAGAAGAACATCAG
GATGGAGCATTGAATCTCAGTTTTTTAACCTTATAAACCTGTATCGAGATTTGGGCCGTTCCACTGGTTC
TCAACATAGATCAAATTTAGTTGGTCCGAGGTCAAGTTCTAGTAATCAGGTACAGCATTCTGGGTCAGA
TGATAATTCTGGGACTGCTGATAAGAAGGAATCTGACAAGCAGAGACCATATTATACCTCTTGTTGTGA
CATGGTCAGATCACTTTCATTTCACATTACCCATTTGTTCCAAGAGTTGGGAAAAGTAATGTTGCTACCT
TCACGTCGACGTGATGATGTTGTGAATGTAAGTCCTGCTTCAAAATCAGTGGCTTCTACTTTTGCATCCA
TTGCTTTTGATCACATGAATTATGGTGGCCGTTGTGTAAATCTTTCGGGAACAGAAGAATCCATATCAAC
AAAATGTCGATATTTTGGGAAAGTGATTGATTTTATGGATAATGTTCTAATGGAGAGGCCAGATTCATG
CAATCCTATTATGCTGAATTGCTTGTATGGACGTGGAGTTATTGAAACTGTATTAACTACCTTTGAAGCT
ACCAGTCAGCTGCTCTTTACAGTTAATCGGGCCCCTGCCTCGCCCATGGATACTGATGATGCGAATGCA
AAGCAAGATGACAAGGAAGATACAGATAATTCATGGATTTATGGTTCTTTAGCTAGTTATGGGAAATTG
ATGGACCATCTAGTGACCTCCTCTTTTATATTATCATCATTCACAAAGCATTTACTTGCACAGCCCCTTAC
TAATGGTAATACAGCTTTCCCAAGGGATGCTGAGACTTTTGTGAAGGTCCTTCAATCCAGAGTGTTGAA
GACTGTGCTTCCTGTTTGGACTCATCCCCAGTTTGTTGACTGTAGTTATGAATTTATTTCTACAGTTATTT
CTATCATTAGGCATGTCTATACAGGTGTTGAAGTAAAAAATGTGAACGGAAGTGGTGGTGCTCGCATT
ACTGGGCCGCCTCCTAATGAAACAACTATTTCAACCATTGTAGAGATGGGGTTTTCCAGGTCTAGAGCA
GAAGAAGCTTTGAGGCAAGTTGGGTCAAATAGTGTGGAGTTGGCAATGGAGTGGTTGTTCTCTCATCC
AGAGGAGATACAAGAAGATGATGAACTTGCCCGTGCACTTGCCATGTCCCTTGGAAACTCAGAATCAG
ATGCAAAGGATGCAGTTGCTAATGACAATGCCCTGCAGCTTGAAGAAGAGATGGTCCTACTCCCTCCTG
TTGATGAGTTGTTATCTACTTGCACAAAACTTTTGTCGAAGGAACCACTTGCTTTTCCTGTCCGTGACTT
GCTTGTGATGATATGCTCTCATGATGATGGTCACCATAGATCTAATGTGGTCTCATTTATTGTGGAACG
GATCAAAGAATGTGGTTTGGTTCCTAGCAATGGAAATGTTGCCACGCTGGCTGCTCTTTTTCATGTTCTA
GCCTTAATTCTTAATGAGGATGCTGTGGCTAGGGAAGCTGCTTCTACAAGTGGTTTGATCAAAATTGCC
TCAGATCTACTCTACCAGTGGGATTCTAGTCTTGATAGCAGGGAGAAACAGCAGGTACCAAAATGGGT
GACTGCTGCTTTCCTTGCATTAGACAGATTGTTGCAAGTAGATCAAAAATTGAATTCTGAAATCGCAGA
GCAGTTGAAGAAGGAAGCTGTGAATAGCCAGCAGACATCGATTACCATTGATGAAGACAGGCAAAAC
AAGTTGCAGTCTGCATTGGGACTCTCTATGAAGTATGCAGATATACATGAACAGAAGAGACTTGTTGA
GGTTGCTTGTAGTTGTATGAATAATCAACTTCCATCTGACACAATGCATGCTATTCTGCTACTATGTTCC
AATCTTACAAGGAATCATTCTGTAGCTCTTACATTTTTGGATGCTGGTGGTTTGAATCTACTTCTTTCTTT
GCCAACCAGCAGCCTCTTCCCTGGGTTTGACAATGTTGCTGCTAGTATTGTTCGTCATGTTCTTGAAGAT
CCACAAACGCTCCAGCAAGCAATGGAATCTGAGATAAAACATAGTCTTGCAGTGGCATCTAATCGGCAT
CCAAATGGAAGGGTCAATCCTCATAATTTCCTTTTAAATTTAGCTTCTGTTATTTATCGGGATCCAGTAAT
CTTTATGCTAGCTGCTCAATCTGTGTGCCAAGTTGAAATGGTAGGTGAGAGGCCATACATTGTCTTGCT
GAAAGATAGGGATAAAGACAAAGCTAGGGAGAAAGAAAAGGATAAGGATAAAACATTGGAGAAAGA
TAAAGTACAGAACAGTGATGGGAAGGTTGTTTTGGGAAATACAAACACAGCACCTACTGGCAATGGCC
ATGGCAAAATTCAGGATTCAAATACCAAGAGTGCCAAAGGTCACAGAAAACCTAACCAAAGTTTTATTA
ATGTAATAGAGCTTCTTCTTGAATCTATATGCACTTTTGTTCCTCCCTTGAAGGATGACATTGCCTCAAAT
GTTCTTCCTGGAACCCCAGCATCAACTGATATGGACATTGATGTCTCCGTGGTTAAGGGAAAAGGAAAA
GCAGTTGCCACTGTGTCTGACGGCAACGAAACTGGTAGTCAGGTTGCTTCTGCATCACTTGCAAAGATT
GTCTTCATTTTAAAGCTTCTGACAGAGATATTATTGCTGTATTCATCATCTGTTCATGTTCTACTTCGACG
AGATGCTGAAATAAGCTGCATTAGAGGTTCTTATCAAAAGAGTCCTGCAGGTTTAAGCATGGGTTGGA
TATTTTCCCATATTCTTCATAATTTTCTTCCATATTCTCGAAACTCAAAAAAGGACAAGAAAGCTGATGGT
GATTGGAGGCAGAAACTAGCAACCAGGGCCAACCAGTTTATAGTGGGTGCTTGTGTTCGATCTACAGA
GGCAAGGAAGAGGGTTTTTGGTGAGATTAGTTATATCATCAATGAATTTGTTGATTCATGTCATGACAT
TAAGCGTCCAGGAAATGAAATTCAGGTTTTTGTTGATCTACTAAATGATGTTTTGGCTGCTCGTACACCT
GCTGGTTCGTACATTTCAGCTGAGGCCTCTACCACTTTTATAGATGCTGGTTTGGTTAAATCATTCACTT
GCACTCTACAAGTTTTGGACCTTGACCATGCTGGTTCATCTGAAGTTGCTACTGGTATTATTAAAGCTCT
TGAGTTGGTAACCAATGAGCATGTCCATTCAGTTCATTCTAGTGCAGGGAAGGGTGATAATTCAACAAA
ACCTTCTGTTCTAAGTCAACCTGGAAGAACAAATAATATTGGTGAACTGTCTCAGTCCATGGAGACATC
ACAAGCCAATCCTGATTCCCTTCAAGTTGACCATGTTGGGTCTTATGCAGTTCACTCCTATGGTGGGTCT
GAAGCTGTTACTGATGATATGGAACATGATCAAGATCTTGATGGGAGCTTTGTTCCTGCTAATGAGGAT
GATTACATGCATGAAAATTCTGAGGATGCAAGAAATCTTGAAAATGGAATGGAAAATGTGGGTCTACA
ATTTGAAATCCAACCTCATGGCCAAGAAAATCTTGATGAGGATGACGATGAGGATGATGATATGTCTG
GAGATGAAGGTGAGGATGTAGATGAAGATGATGATGATGAGGAGGAACACAATGATTTGGAAGAAG
TCCATCATTTGCCACATCCTGACACAGATCAAGACGAGCATGAGATTGATGATGAAGATTTTGATGATG
AAGTGATGGAGGAAGACGATGAGGATGACGAGGAAGATGAAGATGGTGTTATACTGCGACTTGAGG
AGGGAATTAATGGAATTAATGTTTTTGACCATATTGAGGTTTTTGGCAGAGATAATAGTTTTGCAAATG
AAGCTTTACATGTAATGCCAGTTGAGGTTTTTGGATCCAGACGTCCGGGGAGGACGACATCTATTTATA
GTCTTTTGGGCAGAACTGGTGATGCTGCTGTGCCTTCTCGTCACCCACTCTTGCTTGAACCTTCTTCATTC
CCTCCACCTACAGGGCAATCAGATAGTTCAATGGAGAACAACTCAGTGGGTTTGGATAATATATTTCGA
TCGCTGAGGAGTGGGCGCCATGGACACCGTTTGCACTTGTGGACTGATAATAACCAGCAAAGTGGTGG
GACAAACACTGCTGTTGTACCACAAGGCCTTGAGGAGTTGCTTGTCACTCAATTAAGGCGACCAACCCC
TGAAAAGTCATCCAATCAGAACATAGCAGAAGCAGGTTCTCATGGTAAAATTGGAACAACCCAGGCAC
AAGATGCAGGGGGTGCAAGGCCAGAAGTCCCCGTTGAAAGTAATGCTATTCTGGAAATTAGTACTATA
ACTCCCTCAATTGATAACAGTAACAATGCGGATGTCAGACCAGCAGGGACTGGACCTTCACATACAAAT
GTTTCAAACACCCAATCACGGGCAGTTGAGATGCAATTTGAACATACTGATGGAGCTGTGAGGGATAT
TGAAGCTGTCAGCCAGGAGAGTAGTGGTAGTGGAGCAACTTTCGGTGAAAGCCTTCGGAGCTTGGAA
GTTGAGATTGGAAGTGCTGATGGCCATGATGATGGTGGTGAAAGGCTGGTTTCTGCTGATAGGATGGC
AGGTGATTCACAGGCTGCACGCACAAGAAGAGCAAATACACCTTTGAGTCACTTTTCTCCTGTGGTTGG
AAGAGATGTGTCCCTTCATAGTGTTACTGAAGTTTCAGAAAATTCAAGCCGTGATGCAGATCAACAAGG
TCCTGCAGCAGAGCAGCAGGTGAACAGTGATGCGGGATCAGGAGCTATTGATCCTGCTTTTCTGGATG
CTCTTCCTGAGGAGCTACGTGCTGAAGTCCTTTCAGCTCAGCAGGGTCAAGTGGCTCAGCCATCAAATG
TTGAGTCTCAAAACACTGGGGATATTGACCCAGAGTTCCTAGCAGCTCTTCCAGCTGATATTCGAGCAG
AAGTTCTAGCTCAGCAGCAAGCACAGAGGTTGCATCAGTCTCAGGAGCTGGAAGGTCAACCTGTGGAA
ATGGATACAGTCTCAATAATTGCAACTTTTCCATCAGATTTACGAGAAGAGGTTCTCTTGACATCACCAG
ATACTATCCTTGCCAATCTTACACCTGCTCTTGTTGCTGAGGCAAATATGTTGCGGGAGAGGTTTGCACA
CCGTTATAGTCGTACCCTCTTTGGTATGTATCCAAGAAGTCGTAGAGGGGAGACTTCAAGACGTGAAG
GTATTGGTTCTGGTCTGGATGGAGCAGGAGGAACCATTTCTTCTCGCCGCTCCAGTGGAGTTAAGGTTG
TTGAAGCTGATGGAGCACCTTTAGTTGACACAGAAGCTTTGCATGCTATGATTCGGTTATTTCGTGTAG
TGCAGCCACTCTATAAAGGCCAACTCCAGAGGCTTCTATTAAATCTTTGTGCCCATAGTGAAACAAGAA
CCTCTCTGGTGAAAATTCTCATGGACTTGCTAATGCTTGATGTAAAAAGGCCTGTCAGTTATTTTAGTAA
AGTTGAGCCACCATATAGATTGTATGGTTGTCAGAGCAATGTAATGTATTCACGTCCTCAATCTTTTGAT
GGAGTTCCCCCATTGCTGTCTCGTAGAATACTTGGAATTCTCACTTATCTTGCTCGCAATCATCTGTATGT
GGCAAAATTTTTGCTTCAGTGTAGGCTGTCTCATCCTGCAATAAAAGAACCAGATGATCCACGGGGAAA
AGCTGTGATGGTTGTTGAAGATGAAGTAAATATAAGTGAAAGTAATGATGGGTACATCGCCATTGCAA
TGCTATTGGGTCTGTTGAACCAACCACTTTATTTGAGGAGCATAGCCCACCTTGAGCAGCTGCTAGATTT
ACTGGATGTTATCATTGACAGTGCTGGAAACAAGTCATCTGGCAAATCCTTGATACCTACTAACCCATCA
TCAGCTCCACAAATTTCTGCTGCGGAAGCCGATGCGAATGCAGATTCTAACAATTTACCTTCTGCGGAT
GATGCATCTAAAGTTGATGGTTCCTCCAAACCGACAGTCTCTGGCATTAATGTTGAATGTGAGTTACAT
GGAGTGTTGAGTAATCTTCCAAAAGCAGAACTCCGGCTCCTGTGCTCACTGCTTGCTCAAGAAGGTTTG
TCAGATAATGCGTATAATCTTGTAGCGGAAGTAATGAAGAAATTGGTGGCCATTGCTCCAACACATTGT
GAGCTTTTTGTCACTGAGCTGGCAGAAGCAGTTCAAAAGTTGACTTCATCTGCAATGAATGAGTTACGT
GTCTTTAGTGAAGCAATGAAAGCTCTGCTTAGTACCTCTTCTACTGATGGAGCTGCAATTTTGAGAGTCT
TGCAAGCCTTGAGTTCCCTTGTCACCTTACTGACGGAGAAAGAGAATGATAGAGGTACTCCTGCTCTTT
CTGAGGTTTGGGAAATCAATTCAGCATTAGAACCCTTGTGGCATGAGCTTAGTTGTTGCATAAGCAAGA
TAGAATCCTACTCAGAGTCTGCATCTGAGTTTTCGACATCTTCTAGTACCTTTGTGTCTAAACCGTCTGGT
GTAATGCCTCCACTTCCAGCTGGCTCTCAAAATATCTTACCATACATTGAATCTTTCTTTGTGGTTTGTGA
GAAATTGCATCCTGCTCAGCCAGGTGCTAGTCACGACTCAAGTATTCCTGTTATTTCGGATGTTGAGTAT
GCCACCACATCTGTAACTCCCCAGAAAGCATCTGGAACTGCTGTGAAAGTAGATGAGAAACATATGCCT
TTTGTCCGGTTCTCAGAGAAGCACAGGAAGCTACTAAATGCTTTCATAAGGCAAAACCCTGGTTTGCTT
GAAAAATCTTTCTCACTCATGCTAAAGGTTCCAAGATTTATTGATTTTGATAACAAGCGTGCCCACTTCC
GATCAAAAATTAAGCATCAGCATGACCATCACCATAGTCCCTTGAGAATATCAGTAAGAAGGGCATATG
TTCTAGAAGATTCTTACAACCAGCTTCGCATGAGATCAACTCAAGATTTGAAGGGAAGGTTGACTGTCC
ACTTCCAAGGGGAGGAGGGTATTGATGCAGGTGGGCTTACAAGGGAATGGTATCAATTATTGTCCAGA
GTTATTTTTGATAAAGGAGCACTGCTTTTTACTACAGTGGGCAATGAATCAACATTTCAGCCAAACCCTA
ACTCTGTTTACCAAACAGAACATTTATCTTATTTCAAATTTGTTGGTAGAGTGGTCGGTAAAGCATTATT
TGATGGTCAACTCTTGGATGTTCATTTTACTCGGTCATTCTACAAGCACATTCTTGGAGTCAAAGTTACA
TATCATGATATTGAAGCCATTGATCCTGATTATTTCAAAAATTTGAAATGGATGCTTGAGAATGATATCA
GTGATGTTCTGGATCTTACTTTTAGCATTGACGCAGATGAGGAAAAATTGATCTTATATGAACGAACAG
AGGTGACTGATTATGAGTTGATTCCCGGGGGACGGAATATCAAAGTTACTGAGGAGAACAAGCATCAA
TATGTTGATTTGGTTGCCGAGCATCGGTTGACAACTGCTATTCGACCTCAAATAAATTATTTCTTAGAAG
GGTTCATTGAATTGATTCCCAGGGAGTTGATATCGATATTCAATGACAAAGAGCTGGAATTGTTGATCA
GTGGACTTCCTGATATTGATTTGGATGACTTGAGAGCAAATACAGAATATTCTGGATATAGTGCTGCAT
CGCCAGTTATCCAATGGTTTTGGGAGGTTGTTCAAGGTTTGAGCAAAGAAGACAAGGCTCGACTGTTG
CAATTTGTGACAGGCACATCCAAGGTGCCTTTGGAGGGCTTTAGCGCTCTCCAAGGAATTTCAGGCTCC
CAGAAGTTTCAGATACACAAAGCATATGGAAGTCCTGATCACTTGCCTTCTGCTCATACTTGCTTCAATC
AATTAGATTTGCCGGAGTATCCATCTAAACAACATTTGGAAGAGAGGTTACTGCTGGCAATTCACGAAG
CAAGTGAGGGTTTTGGATTTGGTTGA
SEQ ID NO: 25>KRH16869 cds:protein_coding
ATGACAACCCTAAGATCGAGTTGGCCTTCGAGGCTGCGCCAACTTCTGTCGAGCGAGGGCGCCATTGG
TCCTTCCGTCAAGGTGGACACCGAGCCCCCTCCTATGGTCAAAGCCTTCATTGAGAAGATCATCCAGTG
TCCATTACAAGATATTGCCATACCACTTTCTGGCTTTCGGTGGGAGTACAATAAGGGGAATTTTCATCAC
TGGAGACTGTTGTTGCTTCATTTTGATACATACTTCAAGACTTATTTGTCGTGTAGAAATGATCTGACAT
TGTTAGATAATCTAGAAGATGACAGCCCATTACCAAAACATGCAATTCTGCAAATATTGCGAGTGTTGC
AAATAATTTTAGAGAACTGTCCAAACAAGAGTTCCTTTGATGGCTTAGAGCATTTCAAGCTTTTACTAGC
ATCAACAGATCCTGAGATTCTTATTGCTACATTAGAAACTCTTTCTGCACTTGTAAAAATTAATCCCTCTA
AGCTTCATGGAAGTACAAAGATGATTTGCTGTGGTTCGGTGAACAGCTATCTTTTGTCCCTAGCACAAG
GCTGGGGAAGCAAGGAGGAGGGCCTAGGATTGTACTCTTGTGTTATGGCAAATGAGAAAGTCCAAGA
TGAAGCACTGTGCTTATTTCCTTCTGAAGAGATTGGTCATGACCAATCAAATTGCCGCATGGGCACTAC
CCTTTATTTTGAATTGCATGGTCCCAGTGCCCAAAGCAAGGAACATAGTGCAGATGCAGTTTCCCCTGG
CTCAACAGTTATACATATGCCAGATTTGCATCTGCGCAAAGAAGATGATTTGTCCTTGATGAAGCAGTG
CATTGAACAATTTAGCGTTCCTTCTGAGCTCAGATTTTCATTGCTCACTAGAATCAGATATGCTCGTGCC
TTTCGTTCTCCTAGAATATGCAGGCTTTACAGCAGGATTTGCCTACTTTCTTTCATTGTTCTGGTGCAGTC
TGGTGATGCTCAGGAAGAACTCGTCTCCTTTTTTGCCAATGAACCAGAATATACAAATGAATTAATTAG
AATTGTACGTTCAGAGGAAGTTATATCTGGATCTATCAGGACACTTGCAATGCTTGCTTTAGGAGCTCA
ATTAGCCGCATATACATCATCGCATCATCGGGCACGAATACTCAGTGGATCTAGTTTAACTTTTGCTGGT
GGGAACCGCATGATACTCCTAAATGTGCTCCAGAGGGCTATTTTGTCATTGAAGAGTTCTAATGATCCA
TCATCCCTTGCTTTTGTTGAAGCACTTCTTCAGTTCTATCTGCTCCATGTGGTTTCAACCTCAACTTCTGGT
AATAATATTAGAGGTTCTGGCATGGTGCCTACATTCTTGCCGTTGCTGGAGGATTTTGATCCTACACATA
TTCATCTAGTCTGTTTTGCTGTGAAAACACTTCAGAAGCTTATGGATTATAGTAGCTCAGCTGTATCATT
GTTTAAAGAATTGGGGGGCATTGAACTTTTGGCTCAGAGATTACAGAAAGAGGTACACAGAGTCATTG
GTTTGGTTGGAGAAACTGATAACATTATGCTTACTGGTGAAAGCTTGAGATATAGTACTGATCAATTGT
ACTCCCAGAAGAGACTCATAAAGGTCTCCCTTAAGGCGCTTGGTTCTGCAACATACGCGCCTGCAAACT
CTACCAGATCTCAACATTCTCAAGACAGTTCATTACCTGTAACTCTAAGATTGATTTTTCAGAATGTAGA
TAAGTTTGGAGGTGACATTTATTATTCAGCTGTTACTGTTATGAGCGAAATAATCCACAAAGATCCAACC
TGTTTTTCTGCTCTGCATGAAATGGGTCTTCCTGATGCTTTTTTATTGTCAGTTGGATCTGAAATACTTCC
ATCATCAAAGGCTTTGACATGCATTCCAAATGGTCTTGGGGCAATTTGTCTTAATGCCAAAGGGTTAGA
GGCTGTTAGAGAATCTTCATCGCTACGGTTCCTTATTGACATTTTCACTAGCAAGAAGTATATCTTAGCC
ATGAATGAGGCTATTGTTCCTTTGGCAAATGCTGTGGAGGAACTTCTACGTCATGTATCTACATTGAGA
AGCTCCAGTGTTGATATTATCATTGAAATCATCCACAAGATCGCATCTTTTGGGGATGGAAATGGTACT
GGATTTTCTGGAAAAGCTGAGGGCACTGCCATGGAAACAGATTCTGAAAACAAAGAAAAAGAAGGCC
ATTGTTGCATTGTAGGCACATCATATTCAGCCATAGAAGGGATAAGTGATGAGCAGTTTATTCAGCTAT
GTGTCTTTCATTTAATGGTATTGATTCATAGGACTATGGAAAATGCCGAGACATGCCGGTTGTTTGTGG
AAAAATCAGGAATTGAAGCTTTATTGAATTTGTTGTTGCGGCCAACTATTGCACAATCCTCAGATGGCA
TGTCTATTGCTTTACATAGCACGATGGTATTTAAAGGGTTTGCTCAACATCATTCCATTCCTCTGGCACAT
GCCTTCTGTTCTTCTCTTAGAGAGCACTTGAAGAAAGCTTTAGCGGGGCTTGGTGCAGCATCAGAACCT
TTGTTGCTGGATCCAAGGATGACAACTGATGGTGCCATCTTTTCTTCACTTTTCCTGGTTGAGTTCCTTCT
ATTTCTTGCTGCACCAAAAGACAATCGTTGGGTGACTGCCTTGCTTACAGAATTTGGAAATGGAGGTAA
GGATGTTCTTGAAGACATTGGACGTGTACACCGTGAAGTCCTGTGGCAAATTGCTCTACTTGAAAACAG
AAAGCCTGAGATTGAGGAAGATGGTGCTTGTACTTCTGATTTACAACAGGCCGAAGGGGATGCAAGTG
AAACTGAAGAGCAAAGGTTGAATTCTTTCAGGCAGTTTCTTGACCCATTATTGAGAAGAAGAACATCAG
GATGGAGCATTGAATCTCAGTTTTTTAACCTTATAAACCTGTATCGAGATTTGGGCCGTTCCACTGGTTC
TCAACATAGATCAAATTTAGTTGGTCCGAGGTCAAGTTCTAGTAATCAGGTACAGCATTCTGGGTCAGA
TGATAATTCTGGGACTGCTGATAAGAAGGAATCTGACAAGCAGAGACCATATTATACCTCTTGTTGTGA
CATGGTCAGATCACTTTCATTTCACATTACCCATTTGTTCCAAGAGTTGGGAAAAGTAATGTTGCTACCT
TCACGTCGACGTGATGATGTTGTGAATGTAAGTCCTGCTTCAAAATCAGTGGCTTCTACTTTTGCATCCA
TTGCTTTTGATCACATGAATTATGGTGGCCGTTGTGTAAATCTTTCGGGAACAGAAGAATCCATATCAAC
AAAATGTCGATATTTTGGGAAAGTGATTGATTTTATGGATAATGTTCTAATGGAGAGGCCAGATTCATG
CAATCCTATTATGCTGAATTGCTTGTATGGACGTGGAGTTATTGAAACTGTATTAACTACCTTTGAAGCT
ACCAGTCAGCTGCTCTTTACAGTTAATCGGGCCCCTGCCTCGCCCATGGATACTGATGATGCGAATGCA
AAGCAAGATGACAAGGAAGATACAGATAATTCATGGATTTATGGTTCTTTAGCTAGTTATGGGAAATTG
ATGGACCATCTAGTGACCTCCTCTTTTATATTATCATCATTCACAAAGCATTTACTTGCACAGCCCCTTAC
TAATGGTAATACAGCTTTCCCAAGGGATGCTGAGACTTTTGTGAAGGTCCTTCAATCCAGAGTGTTGAA
GACTGTGCTTCCTGTTTGGACTCATCCCCAGTTTGTTGACTGTAGTTATGAATTTATTTCTACAGTTATTT
CTATCATTAGGCATGTCTATACAGGTGTTGAAGTAAAAAATGTGAACGGAAGTGGTGGTGCTCGCATT
ACTGGGCCGCCTCCTAATGAAACAACTATTTCAACCATTGTAGAGATGGGGTTTTCCAGGTCTAGAGCA
GAAGAAGCTTTGAGGCAAGTTGGGTCAAATAGTGTGGAGTTGGCAATGGAGTGGTTGTTCTCTCATCC
AGAGGAGATACAAGAAGATGATGAACTTGCCCGTGCACTTGCCATGTCCCTTGGAAACTCAGAATCAG
ATGCAAAGGATGCAGTTGCTAATGACAATGCCCTGCAGCTTGAAGAAGAGATGGTCCTACTCCCTCCTG
TTGATGAGTTGTTATCTACTTGCACAAAACTTTTGTCGAAGGAACCACTTGCTTTTCCTGTCCGTGACTT
GCTTGTGATGATATGCTCTCATGATGATGGTCACCATAGATCTAATGTGGTCTCATTTATTGTGGAACG
GATCAAAGAATGTGGTTTGGTTCCTAGCAATGGAAATGTTGCCACGCTGGCTGCTCTTTTTCATGTTCTA
GCCTTAATTCTTAATGAGGATGCTGTGGCTAGGGAAGCTGCTTCTACAAGTGGTTTGATCAAAATTGCC
TCAGATCTACTCTACCAGTGGGATTCTAGTCTTGATAGCAGGGAGAAACAGCAGGTACCAAAATGGGT
GACTGCTGCTTTCCTTGCATTAGACAGATTGTTGCAAGTAGATCAAAAATTGAATTCTGAAATCGCAGA
GCAGTTGAAGAAGGAAGCTGTGAATAGCCAGCAGACATCGATTACCATTGATGAAGACAGGCAAAAC
AAGTTGCAGTCTGCATTGGGACTCTCTATGAAGTATGCAGATATACATGAACAGAAGAGACTTGTTGA
GGTTGCTTGTAGTTGTATGAATAATCAACTTCCATCTGACACAATGCATGCTATTCTGCTACTATGTTCC
AATCTTACAAGGAATCATTCTGTAGCTCTTACATTTTTGGATGCTGGTGGTTTGAATCTACTTCTTTCTTT
GCCAACCAGCAGCCTCTTCCCTGGGTTTGACAATGTTGCTGCTAGTATTGTTCGTCATGTTCTTGAAGAT
CCACAAACGCTCCAGCAAGCAATGGAATCTGAGATAAAACATAGTCTTGCAGTGGCATCTAATCGGCAT
CCAAATGGAAGGGTCAATCCTCATAATTTCCTTTTAAATTTAGCTTCTGTTATTTATCGGGATCCAGTAAT
CTTTATGCTAGCTGCTCAATCTGTGTGCCAAGTTGAAATGGTAGGTGAGAGGCCATACATTGTCTTGCT
GAAAGATAGGGATAAAGACAAAGCTAGGGAGAAAGAAAAGGATAAGGATAAAACATTGGAGAAAGA
TAAAGTACAGAACAGTGATGGGAAGGTTGTTTTGGGAAATACAAACACAGCACCTACTGGCAATGGCC
ATGGCAAAATTCAGGATTCAAATACCAAGAGTGCCAAAGGTCACAGAAAACCTAACCAAAGTTTTATTA
ATGTAATAGAGCTTCTTCTTGAATCTATATGCACTTTTGTTCCTCCCTTGAAGGATGACATTGCCTCAAAT
GTTCTTCCTGGAACCCCAGCATCAACTGATATGGACATTGATGTCTCCGTGGTTAAGGGAAAAGGAAAA
GCAGTTGCCACTGTGTCTGACGGCAACGAAACTGGTAGTCAGGTTGCTTCTGCATCACTTGCAAAGATT
GTCTTCATTTTAAAGCTTCTGACAGAGATATTATTGCTGTATTCATCATCTGTTCATGTTCTACTTCGACG
AGATGCTGAAATAAGCTGCATTAGAGGTTCTTATCAAAAGAGTCCTGCAGGTTTAAGCATGGGTTGGA
TATTTTCCCATATTCTTCATAATTTTCTTCCATATTCTCGAAACTCAAAAAAGGACAAGAAAGCTGATGGT
GATTGGAGGCAGAAACTAGCAACCAGGGCCAACCAGTTTATAGTGGGTGCTTGTGTTCGATCTACAGA
GGCAAGGAAGAGGGTTTTTGGTGAGATTAGTTATATCATCAATGAATTTGTTGATTCATGTCATGACAT
TAAGCGTCCAGGAAATGAAATTCAGGTTTTTGTTGATCTACTAAATGATGTTTTGGCTGCTCGTACACCT
GCTGGTTCGTACATTTCAGCTGAGGCCTCTACCACTTTTATAGATGCTGGTTTGGTTAAATCATTCACTT
GCACTCTACAAGTTTTGGACCTTGACCATGCTGGTTCATCTGAAGTTGCTACTGGTATTATTAAAGCTCT
TGAGTTGGTAACCAATGAGCATGTCCATTCAGTTCATTCTAGTGCAGGGAAGGGTGATAATTCAACAAA
ACCTTCTGTTCTAAGTCAACCTGGAAGAACAAATAATATTGGTGAACTGTCTCAGTCCATGGAGACATC
ACAAGCCAATCCTGATTCCCTTCAAGTTGACCATGTTGGGTCTTATGCAGTTCACTCCTATGGTGGGTCT
GAAGCTGTTACTGATGATATGGAACATGATCAAGATCTTGATGGGAGCTTTGTTCCTGCTAATGAGGAT
GATTACATGCATGAAAATTCTGAGGATGCAAGAAATCTTGAAAATGGAATGGAAAATGTGGGTCTACA
ATTTGAAATCCAACCTCATGGCCAAGAAAATCTTGATGAGGATGACGATGAGGATGATGATATGTCTG
GAGATGAAGGTGAGGATGTAGATGAAGATGATGATGATGAGGAGGAACACAATGATTTGGAAGAAG
TCCATCATTTGCCACATCCTGACACAGATCAAGACGAGCATGAGATTGATGATGAAGATTTTGATGATG
AAGTGATGGAGGAAGACGATGAGGATGACGAGGAAGATGAAGATGGTGTTATACTGCGACTTGAGG
AGGGAATTAATGGAATTAATGTTTTTGACCATATTGAGGTTTTTGGCAGAGATAATAGTTTTGCAAATG
AAGCTTTACATGTAATGCCAGTTGAGGTTTTTGGATCCAGACGTCCGGGGAGGACGACATCTATTTATA
GTCTTTTGGGCAGAACTGGTGATGCTGCTGTGCCTTCTCGTCACCCACTCTTGCTTGAACCTTCTTCATTC
CCTCCACCTACAGGGCAATCAGATAGTTCAATGGAGAACAACTCAGTGGGTTTGGATAATATATTTCGA
TCGCTGAGGAGTGGGCGCCATGGACACCGTTTGCACTTGTGGACTGATAATAACCAGCAAAGTGGTGG
GACAAACACTGCTGTTGTACCACAAGGCCTTGAGGAGTTGCTTGTCACTCAATTAAGGCGACCAACCCC
TGAAAAGTCATCCAATCAGAACATAGCAGAAGCAGGTTCTCATGGTAAAATTGGAACAACCCAGGCAC
AAGATGCAGGGGGTGCAAGGCCAGAAGTCCCCGTTGAAAGTAATGCTATTCTGGAAATTAGTACTATA
ACTCCCTCAATTGATAACAGTAACAATGCGGATGTCAGACCAGCAGGGACTGGACCTTCACATACAAAT
GTTTCAAACACCCAATCACGGGCAGTTGAGATGCAATTTGAACATACTGATGGAGCTGTGAGGGATAT
TGAAGCTGTCAGCCAGGAGAGTAGTGGTAGTGGAGCAACTTTCGGTGAAAGCCTTCGGAGCTTGGAA
GTTGAGATTGGAAGTGCTGATGGCCATGATGATGGTGGTGAAAGGCTGGTTTCTGCTGATAGGATGGC
AGGTGATTCACAGGCTGCACGCACAAGAAGAGCAAATACACCTTTGAGTCACTTTTCTCCTGTGGTTGG
AAGAGATGTGTCCCTTCATAGTGTTACTGAAGTTTCAGAAAATTCAAGCCGTGATGCAGATCAACAAGG
TCCTGCAGCAGAGCAGCAGGTGAACAGTGATGCGGGATCAGGAGCTATTGATCCTGCTTTTCTGGATG
CTCTTCCTGAGGAGCTACGTGCTGAAGTCCTTTCAGCTCAGCAGGGTCAAGTGGCTCAGCCATCAAATG
TTGAGTCTCAAAACACTGGGGATATTGACCCAGAGTTCCTAGCAGCTCTTCCAGCTGATATTCGAGCAG
AAGTTCTAGCTCAGCAGCAAGCACAGAGGTTGCATCAGTCTCAGGAGCTGGAAGGTCAACCTGTGGAA
ATGGATACAGTCTCAATAATTGCAACTTTTCCATCAGATTTACGAGAAGAGGTTCTCTTGACATCACCAG
ATACTATCCTTGCCAATCTTACACCTGCTCTTGTTGCTGAGGCAAATATGTTGCGGGAGAGGTTTGCACA
CCGTTATAGTCGTACCCTCTTTGGTATGTATCCAAGAAGTCGTAGAGGGGAGACTTCAAGACGTGAAG
GTATTGGTTCTGGTCTGGATGGAGCAGGAGGAACCATTTCTTCTCGCCGCTCCAGTGGAGTTAAGGTTG
TTGAAGCTGATGGAGCACCTTTAGTTGACACAGAAGCTTTGCATGCTATGATTCGGTTATTTCGTGTAG
TGCAGCCACTCTATAAAGGCCAACTCCAGAGGCTTCTATTAAATCTTTGTGCCCATAGTGAAACAAGAA
CCTCTCTGGTGAAAATTCTCATGGACTTGCTAATGCTTGATGTAAAAAGGCCTGTCAGTTATTTTAGTAA
AGTTGAGCCACCATATAGATTGTATGGTTGTCAGAGCAATGTAATGTATTCACGTCCTCAATCTTTTGAT
GGAGTTCCCCCATTGCTGTCTCGTAGAATACTTGGAATTCTCACTTATCTTGCTCGCAATCATCTGTATGT
GGCAAAATTTTTGCTTCAGTGTAGGCTGTCTCATCCTGCAATAAAAGAACCAGATGATCCACGGGGAAA
AGCTGTGATGGTTGTTGAAGATGAAGTAAATATAAGTGAAAGTAATGATGGGTACATCGCCATTGCAA
TGCTATTGGGTCTGTTGAACCAACCACTTTATTTGAGGAGCATAGCCCACCTTGAGCAGCTGCTAGATTT
ACTGGATGTTATCATTGACAGTGCTGGAAACAAGTCATCTGGCAAATCCTTGATACCTACTAACCCATCA
TCAGCTCCACAAATTTCTGCTGCGGAAGCCGATGCGAATGCAGATTCTAACAATTTACCTTCTGCGGAT
GATGCATCTAAAGTTGATGGTTCCTCCAAACCGACAGTCTCTGGCATTAATGTTGAATGTGAGTTACAT
GGAGTGTTGAGTAATCTTCCAAAAGCAGAACTCCGGCTCCTGTGCTCACTGCTTGCTCAAGAAGGTTTG
TCAGATAATGCGTATAATCTTGTAGCGGAAGTAATGAAGAAATTGGTGGCCATTGCTCCAACACATTGT
GAGCTTTTTGTCACTGAGCTGGCAGAAGCAGTTCAAAAGTTGACTTCATCTGCAATGAATGAGTTACGT
GTCTTTAGTGAAGCAATGAAAGCTCTGCTTAGTACCTCTTCTACTGATGGAGCTGCAATTTTGAGAGTCT
TGCAAGCCTTGAGTTCCCTTGTCACCTTACTGACGGAGAAAGAGAATGATAGAGGTACTCCTGCTCTTT
CTGAGGTTTGGGAAATCAATTCAGCATTAGAACCCTTGTGGCATGAGCTTAGTTGTTGCATAAGCAAGA
TAGAATCCTACTCAGAGTCTGCATCTGAGTTTTCGACATCTTCTAGTACCTTTGTGTCTAAACCGTCTGGT
GTAATGCCTCCACTTCCAGCTGGCTCTCAAAATATCTTACCATACATTGAATCTTTCTTTGTGGTTTGTGA
GAAATTGCATCCTGCTCAGCCAGGTGCTAGTCACGACTCAAGTATTCCTGTTATTTCGGATGTTGAGTAT
GCCACCACATCTGTAACTCCCCAGAAAGCATCTGGAACTGCTGTGAAAGTAGATGAGAAACATATGCCT
TTTGTCCGGTTCTCAGAGAAGCACAGGAAGCTACTAAATGCTTTCATAAGGCAAAACCCTGGTTTGCTT
GAAAAATCTTTCTCACTCATGCTAAAGGTTCCAAGATTTATTGATTTTGATAACAAGCGTGCCCACTTCC
GATCAAAAATTAAGCATCAGCATGACCATCACCATAGTCCCTTGAGAATATCAGTAAGAAGGGCATATG
TTCTAGAAGATTCTTACAACCAGCTTCGCATGAGATCAACTCAAGATTTGAAGGGAAGGTTGACTGTCC
ACTTCCAAGGGGAGGAGGGTATTGATGCAGGTGGGCTTACAAGGGAATGGTATCAATTATTGTCCAGA
GTTATTTTTGATAAAGGAGCACTGCTTTTTACTACAGTGGGCAATGAATCAACATTTCAGCCAAACCCTA
ACTCTGTTTACCAAACAGAACATTTATCTTATTTCAAATTTGTTGGTAGAGTGGTCGGTAAAGCATTATT
TGATGGTCAACTCTTGGATGTTCATTTTACTCGGTCATTCTACAAGCACATTCTTGGAGTCAAAGTTACA
TATCATGATATTGAAGCCATTGATCCTGATTATTTCAAAAATTTGAAATGGATGCTTGAGAATGATATCA
GTGATGTTCTGGATCTTACTTTTAGCATTGACGCAGATGAGGAAAAATTGATCTTATATGAACGAACAG
AGGTGACTGATTATGAGTTGATTCCCGGGGGACGGAATATCAAAGTTACTGAGGAGAACAAGCATCAA
TATGTTGATTTGGTTGCCGAGCATCGGTTGACAACTGCTATTCGACCTCAAATAAATTATTTCTTAGAAG
GGTTCATTGAATTGATTCCCAGGGAGTTGATATCGATATTCAATGACAAAGAGCTGGAATTGTTGATCA
GTGGACTTCCTGATATTGATTTGGATGACTTGAGAGCAAATACAGAATATTCTGGATATAGTGCTGCAT
CGCCAGTTATCCAATGGTTTTGGGAGGTTGTTCAAGGTTTGAGCAAAGAAGACAAGGCTCGACTGTTG
CAATTTGTGACAGGCACATCCAAGGTGCCTTTGGAGGGCTTTAGCGCTCTCCAAGGAATTTCAGGCTCC
CAGAAGTTTCAGATACACAAAGCATATGGAAGTCCTGATCACTTGCCTTCTGCTCATACTTGCTTCAATC
AATTAGATTTGCCGGAGTATCCATCTAAACAACATTTGGAAGAGAGGTTACTGCTGGCAATTCACGAAG
CAAGTGAGGGTTTTGGATTTGGTTGA
B. napus
SEQ ID NO: 26: Bra038022.1
ATGCCTTCCCAAGTCCAGCCTCCCAAGATCAAATCGTTCATCAATAGCGTCACTGCTGTTC
CCCTCGACCAAATTCAAGAACCCCTTTCCTGTTTTCACTGGGATTTCGACGACAAGGGTGA
CTTCCATCACTGGGTGGATCTCTTCAATCATTTCGACACATATTTTGAGAAGCACATTAAAG
CTAGGAAGGATCTTCATGTTGAGCAACAAGACTCTGAGGACGAATCTACTCCTCCTCTCCC
AAAGGATGCTCTTCTTCAGATTCTCCGTGTTATCCGAGTTGTGTTAGATAACTGCACAAACA
TTCATTTTTTTACTTCTTATGAGCAGCATCTTTCTCTCCTGCTTGCATCTACTGATACAGATG
TCGTTGAAGCCTGTCTGCAGACGTTGGCTTCCTTTTTCAAGAGGCAAAATGATATTTACTTC
ATAAGAGATGCTTCTCTTAATTCAAAACTATTTTCTCTTGCCCAAGGCTGGGGTGGCAAAGA
GGAAGGTCTTGGCTTGACATCATGTGCTACTACAGAAAACACTTGTGATCTGGTTTCTCAC
CTCCTTGGTTCTACTCTTCATTTTGAGTTTTATGCTTCTGGTGAATCATCAACTGAGCTTCC
GGGCGGTTTACAAGTTATCCATCTACCTGATGTCAGCTTGCGTGCAGAGTCTGATCTGGAA
CTTCTCAACAAATTAGTCACTGACCATAACGTTCCTCCCAGTTTAAGGTTCGTGTTGTTGAC
CAGGTTGAGATTTGCAAGGGCGTTTTCATCTTTGTCGACCCGGCTGCAGTACACACGCATT
CGCTTATATGCATTCATTCTTTTGGTTCAAGCTAGTGGCGACACCCAGAAAGTGGTTTCTTT
CTTTAATGGAGAACCCGAGTTTGTAAATGAGTTAGTTACACTGCTGAGCTATGAGACTACTG
TCCCAGAGAAAATAAGGCTACTGTGCCTGCTTTCCTTGGTTGCATTATCGCAAGATCGAAC
TCGGCAGACGACTGTGTTAACTGCAGTCACGTCTCGTGGTTTACTATCTGGCCTTATGCAG
AAAGCTATTGATTCCGTTCTTTGTAATACTTCCAAGTCGTCTCTGGCTTTTGCGGAAGCCTT
GTTATCCCTTGTTACTGTCTTGGTCTCATCATCGTCTGGATGTTCAGCCATGCAAGAAGCTG
GTCTTATTCCAATTCTAGTGCCTCTCATCAAAGATACCGATCCCCAGCACTTGCATTTGGTC
AGTACCGCTGTGCATATATTAGAAGCCTTCATGGATTACAGCAATCCAGCTGCTGCTTTGTT
CAGAGATTTGGGTGGCTTAGATGATACTATCTTTCGGTTGAAACTGGAAGTCTCTCGTACC
GAAGACAATGTCAACGAAAAAGTTTGCGGTTCAGACAGTAATGGGAGGGCTTCACATGTCC
TTGGTGATTCTCTTAATCGGCCTGATACTGAACAGCTTCCCTATTCTGAGGCATTAATTTCG
TATTACAGGAGATTGTTGTTAAAAGCCTTGTTATCTGCAATCTCTCTTGGAACTTATTCTCCT
GGTAATACTAACCTCTATGGTTCCGAGGAGAGCTTGCTGCCTGAATGCTTATGCATTATOTT
CCGGAGAGCAAAATATTTCGGGGGTGGAGTATTCTCGCTTGCTACCACTGTCATGAGTGAT
CTCATTCATAAAGATCCAACTTGTTTTAATACTTTAGACTCGTCTGGTGTAACTTCTGCCTTT
CTTGATGCTATCTCTGATGAGGTCATCTGCTCTGCGCAAGCCATTACATGCATCCCGCAGT
CTCTGGATGCTCTGTGTCTCAACAATAGCGGTCTTCAAGCTGTCAAGGACCGAAATGCACT
AAGGTGTTTTGTTAATATATTTACTTCTTCGTCTTATCTGCGAGCTCTTACTGGTGATACACC
TAGTGCTTTGTCAAGTGGGCTTGATGAACTCCTAAGACACCAATCTTCGTTGCGCACTTAT
GGAGTTGATATGTTCATTGAAATCCTGAACTCCATGTTGATTATTGGATCTGGGATGGAGG
CCTCCACTTCTGTGTCAGCAGATGTGCCTACTGATGCTGCTACCGCTCCTATGGAAATTGA
CGCTGATGAGAAGAGCTTGGCCATTTCGGATGAGGCGGAACCATCTTCTGCTGCTTCTCC
AGCAAATACAGAGTTGTTTCTTCCAGATTGTGTGTGTAATGTTGCTCGTCTCTTTGAAATAG
TTCTTCAGAATGCAGAAGTATGTTCTCTATTTGTTGAGAAGAAAGGAATTGATGTTGTCTTG
CAGCTACTCTCTTTACCCGTTATGCCTCTGTCAACCTCCTTTGGTCAAAACTTTTCTGTCGC
TTTTAAGAACTTCTCCCCTCAGCATTCGGCTAGTCTATCTCGTACAGTGTGCTCCTACTTAC
GAGAACGTCTGAAGGGAACAAATGAGCTTTTGGGTGCCATTAAAGGCACTCAGCTTCTTAA
ACTAGAGTCTGCAGTCCAGATGACGATTTTGAGATCCCTTTTCTGCCTAGAAGGCATGTTG
TCCCTCTCAAACTTTCTGTTGAAAGGAACATCTTCAGTTATCGCGGAATTAAGTGCTGCTGA
TGCTGATGTACTAAAAGAACTTGGTCTAACATACAAGCAAATAATTTGGCAGATGGCTTTGT
CTAGTGAGACCAAGGAAGATGAGAAAAAGAGTGTTGATGGAGGACCTGATAATTCAATTTT
AGCCTCATCTAGTACTGTTGAAAGAGAGAGTGAAGAGGACTCAAGAAATGCTTCAGCAGTT
AGATACACAAACCATGTATCCATTAGAAGAAGTACCTCTCAATCTATTTGGCGTGGTGGTC
GTGATCTGTCTGTTATGCGTTCCATCGAGAGTATGCATGGTCGTACACGACAAGCGATTTC
CCGAACGAGGGGTGGGAGAACTCGTCGACACCTGGAGGCTTTTAATTTTGATTCTGAAATT
CCACCTGATTTACCAGGTACATCATCTTCCCATGAGCTGAAAAAGAAAAGCACTGAAGTCC
TGACTGTTGAAATTTTAGACAAGTTGAATTGTACTCTGCGTCTTTTTTTCACTGCCCTTGTGA
AAGGAGGATTCACCTCTGCGAATCGTCGCAGAATTGATGGAGCACCACTGAGTTCCGCAT
CTAAGAAGACGCTTGGTAATGCCATAGCTAAAGTATTTCTTGAAGCTCTTAACTTCGATGGG
AATGGTGTTACTGCTGAGCATGATATATTTCTGTCCGTAAAGTGCCGATACCTTGGAAAAGT
GGTAGATGACATGGCTTCCCTGACATTTGATACTCGAAGAAGGGTCTGTTTCACAGCTATG
ATTAATAGTTTTTATGTCCATGGAACATTTAAGCAACTTCTCACCACATTTGAAGCGACAAG
CCAGTTGCTTTGGACAGTGCCGTTTTCTGTTACTGCATCTGATACTGAGAATGAGAAGCCA
GGTGAAAGGAACATATGGTCTCGCAAGACGTGGCTGGTGGATACTCTGCAAATCTATTGCC
GAGCACTGGACTATTTTGTTAACTCTACATTTCTGTTATCTCCAGCCTCCACTTCTCAAACG
CAGCTTCTTGTCCAGCAAGAGCAAGCTTCAATTGGTTTGTCGATCGAACTCCATCCTGTAC
CAAGGGAACCTGAAACTTTCGTGCGAAATCTGCAGTCTCAGGTTCTGGATGTCATACTACC
TATATGGAACCACCCTATGTTTCCTGATTGCAATCCTAATTTTGTGGCTTCGGTTACCTCCC
TTGTTACGCATATATACTCTGGTGTTGTGGATGCTACGCAAAATCAAGCCCGGGGTACAAA
CCAAAGAGCCTTGCCTCTACAGCCTGACGAAACCATTGTTGGTATGATTGTTGAAATGGGA
TTTTCAAGGTCAAGGGCAGAATACGCGTTACGAAGAGTTGGAACAAACAGTGTTGAAATAG
CTATTGAGTGGTTGTTTGCCAATCCTGAGCATACTGTGCAGGAAGATGACGAGCTGGCCCA
AGCACTTGCACTATCTCTTGGCAATGCATCCAAAACTCCAAAACCTGTAGATGTCCCTCTG
GAAGAAGCGGATCCAAAAGAACCATCTGTTGATGAAGTTATTACTGCATCGGTGAAGTTAT
TTGAAAGTGATGATTCTATGGCTTTCCCATTGATGGATTTGTTTGTAACACTTTGTAGCCGA
AACAAAGGGGAAGATCGGCCGAAAATTGTGTCGTTTCTTATACAGCAACTGAAGCTAGTAC
AAGTTGATTTCTCCAAGGATACTGGTGCTTTGACTATGCTACCACACATTCTAGCATTAGTT
CTCTCAGAGGATGACAACACACGAGAAATTGCTGCACAGGATGGAATTGTGACCGTAGCA
ATTGATATCTTGACGAATTTCAAGCTTAAGAGTGAATCTGAAAGTCAGATTCTGGCTCCAAA
ATGCATTAGCGCTTTACTTCTTATCTTGAGCATGATGCTGCAGGCTCGGACAAGAATCTCG
TCTGAATTTTTGGAAGGAAATCATGGTGGATCTTTGGAGCCGAGTGATTATCCGCAAGACT
CAGCAGCAGCGTTAAAGAAAGTGTTATCTTCAGATGTTGCTAAAGAGGAGTCGAAACCGGA
TTTGGAATCAGTTTTTGGAAAATCTACAGGCTATCTGACCATGGAAGAGGGTCAAAAAGCT
CTACTAATCGCATGTGGCCTCGTAAAGCAGTGTGTTCCAGAAATGATCATGCAGGCTGTTC
TTCAGTTATGTGCACGTCTAACTAAAACTCATGCTTTAGCTATCCAGTTTCTGGAAAATGGA
GCCTTATCCTCACTTTTTAATCTTCCCAAAAAATGTTTCTTCCCTGGGTATGATACTGTTGCA
TCTGTTATTGTACGTCATCTGGTTGAAGATCCACAGACTCTCCAAATTGCTATGGAATCAGA
AATACGACAGACCTTGAGTGGAAAGAGACATGTAGGTAGGGTATTACCTCAGACATTTCTG
ACAACAATGGCACCTGTAATTTCGAGAGATCCTGTGGTTTTCATGAAAGCCGTGGCTTCTA
CTTGTCAGCTGGAGTCATCAGGAGGGAGGGACTTTGTGATTCCGTCGAAGGAAAAAGAAA
AGCCAAAAGTTTCCAGCAGTGAGCAGGGATTGCCTCTGAATGAACCCCTTCGAATATCCGA
AAATAAGCTTCATGATGGGTCAGGGAAATGTTCGAAAAGCCACAGACGAGTCCCTGCTAAT
TTCATCCAAGTTATCGATCAGCTTATTGATATTGTCTTAAGTTTTCCTAGGGTGAAGAGGCA
GGAAGATGATGAAACCAATTTAATTGCAATGGAAGTTGATGTGCCGGCAACTAAAGTGAAG
GGTAAGTCAAAAGTTGGTGATCCAGAGGAAGCAGAATTTGGATCTGAAGAATTGGCCAGG
GTAACATTTATTTTGAAATTGTTGAGTGATATTGTTATCATGTACTTGCACGGTACCAGTGTC
ATACTGAGGCGGGATACAGAAATATCTCAGCTTCGGGGATCCAATCTACCCGATAATTCAC
CTGGCAATGGAGGGTTAATTTACCATATCATTCACCGATTACTTCCTATATCGCTCAAAAAT
TCTGTTGGATCTGAAGTTTGGAAAGAGAAGTTGTCTGAAAAAGCTTCCTGGTTTCTGGTCG
TTTTTTGCAGCCGTTCCAGTGAGGGACGTAGAAGAATAATCAGTGAGCTTTCGAGTGTTTT
ATCTGTATTGGCTTCCTTGGGAAAGAGTTCTTCTAGTAAAAGTGTTCTGTTACCTGATAAAA
GAGTTCTTGCTTTTGCTGGCCTGGTTTATTCGATATTAACAAAGAATTCATCTTCCAGCAAC
TTACCTGGTTGTGGTTGCTCACCTGACGTTGCAAAGAGCATGATAGATGGGGGAATTATTA
AGTGTCTGACCAGCATTCTTCACGTAATTGACCTCGACCACCCTGATGCTCCAAAGCTTGT
CACTCTTATTCTCAAGTCTCTTGAGACACTGACGAGTGCTGCAAATACTGCTGAGCAGCTA
AAATCAGCAGGGTCAAACGAGACGAAGGGCACAGATTCTAATGAGAGACATGACAGTCGT
GGAACTTCAACTGAGGCTGAAGTTGATGAGTCAAACCGAAACAATAGCAGTCTACAACAAG
TAACTGATGCCGCAGAGAATGGACAGGAGCACCCTCAAATTTCCTCTCAAAGCGAAGGTG
GAAGGGGTTCGAGTCAAACCCAGGCTATGCCTCAAGAGATGAGGATAGAAGGCGAGGAG
ACAATACTGCCTGAACCTATTCAGATGGATTTCATGGGAGAAGAAGATGACCAAATTGAAAT
GAATTTTCATGTTGAAAATAGGGCCGGAGATGATGGAGATGATGCCATGGGAGACGAAGA
GGATGATGATGAGGAAGGATTTGATGACATCGGACCCGAACTGGAGGATGATGAGGATGC
AGATTTAGTGGCAGACGGAGCTCGGAGTGTTATGTCTCTTTCTGGAACTGATGCCGAAGAC
CCTGAAGATACTGGCCTCGGAGATGAATATAATGATGACATGATTGACGAAGATGAGGATG
ATATCCACGAGAATCGTGTAATAGAGGTGCAGTGGAGGGAAGCTCTTGATGGGCTGGATC
ATTTTCAGATTCTTGGGCGATCTGGTGGTGGAAATGAATTTATTGATGACTTTGAAGGAATG
AATATGGGCGATCTGGTTACTCTGCAGAGACCCGGCTTTGATCGTAGACGTCAAGCAGAC
ATAAATTCTTTCCATCGATCTGGTTCCCAAGTACATGGCTTTCAGCATCCGCTCTTCTCGAG
ACCTTTGCGAACTGGCAATACGGCCTCAGTTTCAGCAAGTGCTGGCAGGAATGATATATCA
CAGTTTTACATGTTTGATATGCCGGTTATACCATTTGATCAAGTACCAAGTAATCCTTTCAGT
GATCGCTTAGGAGGTAGTGGGGCACCTCCTCCTTTGACTGATTATTCTGTGGTGGATATGG
ATTCATCAAGAAGAGGGGTTGGTAATAGTCGGTGGACTGATATAGGTCACCCTCAACCAAG
TAGTCAATCTGCGTCGATTGCCCAACTGATAGAAGAACATTTTATTTCCAACCTTCGTGCTT
CTGCGCTAGCAGATAGTGTTGTCGAAAGGGAAACTAATAGCACGGAAGTCCAAGAGCAGC
AGCATCCATCTGTTGGAAGCGAAAGCGTTTTGGGGGATGGTAACGACGGTGGTCAACAAA
GTGAAGCGCATGAAATGTTGAATAATAATGACAATGTTGATAACCCACCTGATGTAACGGC
TGGAATTTTCTCCCAAGCTCGAGCAAATCTAGCTTCCCCTGTACTTCTGCAGCCTCTTCCTA
TGAACAGTACACCAAATGAGATTGACAGAATGGAAGTTGGGGAAGGTGATGGAGTACCTAT
TGAGCAAGCAGATGTCGTAGCTGTGGATCTTGTCTCCACTGCCCAGGGCCAACCTGATAC
GTCCAGTAGTCAAAATGTCTCTGGTATGGGGACGCCAATTCCAGTAGATGATCCCATTTCC
AATTGTCAACCAAGTGGGGATGTACATATGAGTAGTGATGGTGCAGAGGGAAATCAAAGTG
TGGAACCTTCACTATTATCCCGTGATAACAATGAGCTCTCATCCAGGGAAGCTACCCAAGA
TGCGAGTAATGATGAGCAACTTGCTGAAGGTAGCTTGGAGTTGGACGGTAGGGCACCCGA
AGCGAATTCCATCGATCCTACATTTTTAGAGGCGCTCCCTGAAGAATTACGGGCAGAAGTT
CTTGCTTCTCAGCAAGCTCAGTCCGTTCAGCCCCCAACTTATGAACCACCTTCGGTAGAAG
ACATAGATCCTGAATTTTTGGCAGCGCTTCCCCCAGATATCCAAACAGAAGTTCTTGCTCAA
CAAAGGGTACAAAGGATGGCACATCAGTCACAAGGACAGCCAACTGACATGGATAATGCTT
CAATTATTGCTACCCTACCTGCCGATTTACGTGAAGAGGTTCTCTTAACTTCTTCAGAGGCA
GTTTTGGCAGCGTTGCCTTCACCTTTACTTGCAGAAGCGCAGATGCTCAGAGACCGAGCAA
TGAGTCACTATCAGGCTCGTAGCCATAGTAATCGAAGGAATGGTTTGGGTTACAATAGGCT
GACGGGGATGAACAGGAACGTCGGAGTCACTATTGGTCAGAGGGATGTTTCATCTTTTGC
AGATGGCTTGAAAGTAAAAGAGATGGAAGGAGACCGTCTTGTGGATGTCGAGGCCTTGAA
ATCACTAATTAGGCTACTACGACTTGCACAGCCGTTGGGGAAAGGCCTTCTGCATAGGCTT
CTCTTCAAGCTGTGTGCTCACCGTGGTACAAGAGCCAACTTGGTTCAACTTCTGTTGGATT
TGATTAGGCCAGAGATGGAAACATCACCGAGCGAGTTGGCAATAAGTAATCAGCAAAGACT
CTATGGCTGTCAGTCAAATGTTATTTATGGACGATCCCAGCTGTTGAATGGTCTTCCTCCTC
TAGTGTTCCGTCGGGTGCTAGAGGTTCTGACGTATTTGGCTACGAATCATTCGGCTGTTGC
TGACATGTTGTTCTACTTTGATTCGTCACTTGTGTCCCAATTGTCAAAGCCAAAACCCTCTG
TATGTGAAGGCAAGGGTAAGGAGACTGTTACTCATGTGACAGACTCCCGGAATCTGGAGA
TACCTCTCGTTGTCTTCCTAAAGCTGCTTAATCGGCCTCAGCTTTTGCAAAGTACATCCCAT
CTAGCACTGGTCATTGGTTTACTGCAAGAAGTTGTCTACACCGCAGCATCCCGAATTGAGG
GTTGGTCTCCGTTATCAAGTTTATCTGAGAAATCAGAAGAGAAACCGGTTGGTGAAGAAGC
TTCAAGTGAAACACGAAAAGATGCGAAGTCTGAGCAAGTGGATGAAGCTGATAAGCAATCT
GTTGCAAGAGTAAAGAATTGTGCTGATATATATAACATATTCTTGCAGTTGCCACAGTCCGA
TCTCTGCAATCTTTGCCTACTTCTTGGATATGAAGGGTTATCGGATAAAATTTACCTTTTAGC
AGGAAAGGTGATAAAAAAGCTGGCTGCCGTAGATGTGGCTCATCGGAGGTTTTTCGCAAAA
GAACTCTCACAGTTGGCAAGCGGGTTGAGTGCCTCAACTGTCCGCGAGCTGGCAACACTG
AGCAATACAGAGAAGATGAGTCACAGTACAGGTTCCATGGCAGGTGCTTCACTTCTCCGTG
TTCTACAGGTTCTTAGCTCACTAACTTCCACTATTGATGATGGCAATCCTGGAACCGAAAAG
GAAACAGAACAGGAGGAACAAAACATTATGGAGAGACTAAACATGGCATTAGAGCCCCTTT
GGCAGGAACTTAGCCAGTGTATCAGCATGACTGAGGTGCAGCTGGATCATACTTCAGCCA
CAACAACCGTGTCCAGTGTAAACCCCGGTGATCATGCCCTAGGGGTCACTGCTCCGTCCC
CTATTTCTCCGGGAACTCAGAGGTTCCTACCTCTTATTGAGGCTTTCTTTGTTCTGTGTGAG
AAAATTCAAACTCCGTCAATACTACATCAGGATCAGGCGAATGTGACAGCTGGAGAAGTAA
AGGAGTCTGCTCTTAGTTTATCATCTAAGACCAGTGTAGATTCTCAGAAGAAAATTGATGGC
TCCCTTACATTTGCAAAGTTTGCGGAGAAGCATAAGCGACTTTTGAATTCATTTGTTAGGAA
AAACCCAAGTTTACTGGAGAAGTCCCTTTCAATGATGCTCAAGGCACCAAGGCTGATTGAT
TTTGACAACAAGAAAGCTTACTTCAGGTCAAGGATAAAGCACCAGCATGATCAACACATTTC
TGGTCCATTGCGTATCAGTGTCCGCCGAGCTTATATGTTGGAAGATTCATACAACCAGTTA
CGTATGCGCTCCCTACAGGATCTGAGAGGACGTCTGAATGTGCAGTTTCAAGGTGAAGAA
GGTGTTGATGCTGGTGGTCTTACAAGAGAATGGTATCAGTTAGTGTCAAGAGTTATATTTGA
CAAAGGAGCGTTGCTTTTCACTACCGTTGGAAATGATGCCACCTTCCAGCCGAATCCCAAC
TCTGTTTACCAAAATGAGCATCTGTCATACTTCAAATTTGTTGGTCGCATGGTGGCAAAGGC
GTTGTTTGATGGGCAGCTTTTGGATGTTTATTTTACGCGCTCCTTCTATAAACACATACTTG
GTGTGAAGGTAACCTATCATGACATTGAGGCGGTGGATCCTGATTACTACAAGAACTTGAA
GTGGCTGTTAGAGAATGATGTGAGCGACATACTCGACCTCACATTTAGTATGGACGCAGAT
GAGGAAAAACACATTCTATACGAAAAGACTGAGGTGACGGACTATGAGCTTAAACCTAGAG
GAAGAAACATACGGGTAACAGAGGAAACAAAGCATGAATATGTTGACCTTGTGGCCGGAC
ACATACTTACCAATGCTATTCGGCCTCAAATAAACGCCTTCCTGGAAGGCTTTAATGAGTTA
ATACCTCGTGAGCTCGTATCCATTTTTAATGATAAAGAGCTCGAGCTCCTAATCAGCGGATT
GCCTGAGATTGATTTCGATGATCTTAAAGCCAATACCGAGTATACCAGCTACACGGCTGGA
TCCCCTGTGATTCATTGGTTCTGGGAGGTCGTTAAAGCTTTTAGCAAGGAAGACATGGCTA
GATTTCTTCAATTTGTCACCGGAACATCAAAGGTTCCTTTAGAAGGTTTCAAGGCACTGCAA
GGTATTTCTGGACCTCAAAGATTACAAATCCACAAGGCATATGGAGGTCCGGAGCGGCTG
CCATCAGCTCATACATGTTTTAACCAACTAGACCTTCCAGAGTATCCATCTAAGGAACAACT
TGAGGAACGTCTGCTACTTGCCATTCACGAAGCCAGTGAAGGTTTCGGGTTTGCTTGA
CRISPR sequences
Rice
Promoter targets:
SEQ ID NO: 27 (ProTarget 1):
GGCAGTCTTCGTTCTCGTGT
SEQ ID NO: 28 (Pro Target 2):
GGCAGGTCCCGCCTCTAATC
SEQ ID NO: 29 (Pro Target 3):
GTGCCGGGCCGGTTAACAAT
SEQ ID NO 30 (Pro Target 4):
GGCGCGGCGGGTTACCTCTA
SEQ ID NO: 31 (Pro Target 5):
GGAGGGCCCCCGATCGCGGC
SEQ ID NO: 32 (2.6 kb sequence deleted in most indica varieties)
ggggatgcaggaactgcattctttcatttgaagataaaggcgagaagcaggaagcttctcattccaatccttgagcatgat
ggcaggattgccaccacccagcatgacatgcaaagtttggcacgagaatactttgctgcagtgatgtgccctgagtgcag
tgacacgaagttgctgcaatttcaccatattcagatggcaacaactgatctctccagcctcgacagtcctttcactgaagat
gagatttggtcggctatccgtgctttgcctaatgaaaagtcgccagggccggatggttatacaggcttgttttaccaaagatg
ttgggagataattaaacctgaattgatcagcgctcttgctaaattctgtaccggtaacagtcagaacttggagaaactgaat
tcggcaattgtcacgctaataccgaagaaggacagtcctaccctcctcaaggattataggccaattagtttgattcatagttt
ctctaagatagctgcgaagattatggcgcagcggttagcaccgaagctgaatgtcctcattccatcctcccaaactgctttt
atcaagggacgctgcatacacgagaactttgtcttcgtcaaaggattggtacaacaatttcacagacaaaggaaggctat
gatgttgctgaaattagacatctcgaaagctttcgacactgtctcctggggttttcttatgtcgatgttacagttcagaggctttg
gtccactttggagaagatggctctcggcggtttttctcactgcagaaacaagaatattgataaatggtgttctgtctgacaca
atcaagccggcgagggggttgaggcagggtgacccactgtcgccgctgctctttgttctagtaatggatgccttgcaagct
attgtttcccaggcaaggatggcaagactgctctcgcccctcaacgtacgacagaatttgccaccaatttcagtttatgccg
acgatgcggttctgtttttccgccctacagctgaagaagctcgagtcatcaagggtatcctggagttgttcggggctgccac
aagtctcaaaaccaatttctccaaaagcgcaatcactccaatccaatgtgacgagcagcagtatgtgcaagttgaatcca
ttctctcctgccgagtggaaaagttcccaatcacttatcttggactccctctctccactaggaaaccaacgaaggccgagat
ccagccgatccttgataggctggcaaagaaggtagccggttggaagccgaaaatgctgtctattgatgggcgactgtgct
tgatcaagtcggtcctaatggcgctgccggtgcactacatgacagtcctgcagctaccgcgatgggcgattaaggacatc
gagcggaagtgccgtgggtttctttggaaaggacaggaagagatcagcggcgggcattgcctagtctcgtggcgaaag
gtttgctcacccatcgagaaagggggacttggtgtcaaagatcttaatttgttcggtcaagctctccggttgaaatggcttgc
aaaatccttggagcagaaggatagaccctggaccttagcaactttccgtcctggaagcgatgtggaagagatctttcgat
ccgttgctgagcacatcattggtgacggggtgaacacacagttttggacagacaattggacagggaaaggttgcttcgcc
tggaggtggccggtgttgttttcccatgtgagccgtgccaagctgacagtagctgatgccctgattgctaacagatgggttc
gccgattacaaggtgccttgtccaatgaagctctgggtgaattcttccaactttgggatgaagttcacgacgtgtcactgca
gcagatggctaaaacgatcaaatggaagttgactgttgatggtaatttctcagtggcctcggcgtatgatctatttttcatagc
gacagaggactgttcctacggggacacgctgtggcactccagggtgccgtcgcgtgttcgcttcttcatgtggattgcactc
aagggccgctgtctcacggcggacaacctggcaaagagaaactggccgcatgacgccatttgctccctatgccaacac
gagaacgaagactgccattatttgcttgtgtcctgtgattatacggcggcggtttggcgcaagctgagacgttggtgcaaca
ttaacattgcaatccctgcggaagatggcatgccgcttgcagattggtggatcgcgacaagacggcgttttcagaacacg
tataggacggatttcgatagtctgttaatgctaatttgttggcttatctggaaagagcgaaatacaaggatctttcaacacatc
gccaagtcggttgaccggctagcggatgacatcaacgaggaaatcgcaatttggagggcagcagggattttctcccag
gctagcgagtaatcccgattagaggcgggacctgccccattttttccttttctttccgggcttgagtttgcttgagaccggcgc
gacatccttcatgtcgttgtaattaaaactttatttccctcaatcttaataaaattggccggcctacctttggccgtcccggcaa
aaaagaatctagaatatat
Wheat
OsUPL2 has three homologs in Triticum aestivum, TraesCS5A02G121600,
TraesCS5B02G112800 and TraesCS5D02G118000. We choose the following four
target sites that can target all the three wheat UPL2 genes.
Target sequences:
SEQ ID NO: 33 (Target 1): GTGCTTATTCCCAGCAGACANGG
SEQ ID NO: 34 (Target 2): GCCAGACCTGCACCTTCGGANGG
SEQ ID NO: 35 (Target 3): GAGCGAGCTAGGATACTGAGNGG
SEQ ID NO 36 (Target 4): GTCGCTTCTGTGAGTACAGANGG
sgRNA sequences:
SEQ ID NO: 37 (Target 1): GTGCTTATTCCCAGCAGACA
SEQ ID NO: 38 (Target 2): GCCAGACCTGCACCTTCGGA
SEQ ID NO: 39 (Target 3): GAGCGAGCTAGGATACTGAG
SEQ ID NO: 40 (Target 4): GTCGCTTCTGTGAGTACAGA
Maize
OsUPL2 has two homologs in Zea mays, GRMZM2G331368/Zm00001d023795 and
GRMZM2G411536/Zm00001d041105. We choose the following two target sites that can
target both the two corn UPL2 genes.
Target sequences:
SEQ ID NO: 41 GGACTACGGTTAGAGGCTCANGG
SEQ ID NO: 42 GTGCAATCCCTGAGAAGTATNGG
sgRNA sequences:
SEQ ID NO: 43 GGACTACGGTTAGAGGCTCA
SEQ ID NO: 44 GTGCAATCCCTGAGAAGTAT
Millet
OsUPL2 has one homolog in millet, Seita.3G302600. We choose the following two target
sites that can target the millet UPL2 gene
SEQ ID NO: 45 GCCTGCAGCAGATCCTGGCCNGG
SEQ ID NO: 46 GCACTGGCTATGCTAGCGCTNGG
SEQ ID NO: 47 GCCTGCAGCAGATCCTGGCC
SEQ ID NO: 48 GCACTGGCTATGCTAGCGCT
Soybean
Target sites for GLYMA_02G216000
SEQ ID NO: 49 Target site 1: GCGCCAACTTCTGTCCAGCG
SEQ ID NO: 50 Target site 2: CATTGGTCCTTCAGTCAAGG
Target sites for GLYMA_04G096900
SEQ ID NO: 51 Target site 1: CTATCTCCTGTCCCTAGCAC
SEQ ID NO: 52 Target site 2: GAACGGGCACGGATACTGAG
Target sites for GLYMA_14G183000
SEQ ID NO: 53 Target site 1: CGCCAACTTCTGTCGAGCGA
SEQ ID NO: 54 Target site 2: TCGAAGGCCAACTCGATCTT (reverse complement)
B.napus
SEQ ID NO: 55 Target site1: GGGTTCTTGAATTTGGTCGA
SEQ ID NO: 56 Target site2: GCAAGCTGACATCAGGTAGA
SEQ ID NO: 57 Target site3: GGTTCTTGAATTTGGTCGAG
Os UPL2 genomic sequence
SEQ ID NO: 81
Sequence features in order:
Bold: most indica do not have this sequence
atg: start codon
aaag: large 2-4 deletion; frame-shift mutation
g: large2-1: g converts to t, stop code (gaa to taa)

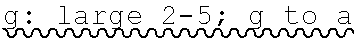

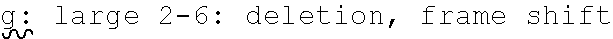

c: large 2-3: deletion, frame shift

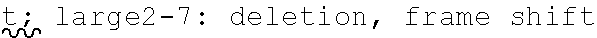

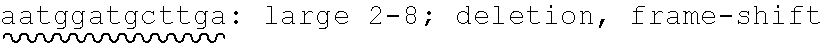

a: large2-9: a to g, leading to two different mutated proteins.
g: large2-2: g to a
taaaagataaaagaaaaagtagagtaattgggccaccaaaactaatgattttcgctactagatcgaagctctagccttttttttttttttgccataagcct
gcttgacatgtatcttttacttgattttagatgatcctcatattcctttatttctaaacttcccaagcaatcaaaagaatagcaaatgttcatctttacac
aaatgaaaactaccattttagcttgattgtgttcttggcccattctaggaagctaaaattatgagaagtagccttttggtagctaaattttgagaatctag
aatatatctgagggaaggggatgcaggaactgcattctttcatttgaagataaaggcgagaagcaggaagcttctcattccaatccttgagcat
gatggcaggattgccaccacccagcatgacatgcaaagtttggcacgagaatactttgctgcagtgatgtgccctgagtgcagtgacac
gaagttgctgcaatttcaccatattcagatggcaacaactgatctctccagcctcgacagtcctttcactgaagatgagatttggtcggcta
tccgtgctttgcctaatgaaaagtcgccagggccggatggttatacaggcttgttttaccaaagatgttgggagataattaaacctgaatt
gatcagcgctcttgctaaattctgtaccggtaacagtcagaacttggagaaactgaattcggcaattgtcacgctaataccgaagaagga
cagtcctaccctcctcaaggattataggccaattagtttgattcatagtttctctaagatagctgcgaagattatggcgcagcggttagcac
cgaagctgaatgtcctcattccatcctcccaaactgcttttatcaagggacgctgcatacacgagaactttgtettcgtcaaaggattggta
caacaatttcacagacaaaggaaggctatgatgttgctgaaattagacatctcgaaagctttcgacactgtctcctggggttttcttatgtc
gatgttacagttcagaggctttggtccactttggagaagatggctctcggcggtttttctcactgcagaaacaagaatattgataaatggtg
ttctgtctgacacaatcaagccggcgagggggttgaggcagggtgacccactgtcgccgctgctctttgttctagtaatggatgccttgca
agctattgtttcccaggcaaggatggcaagactgctctcgcccctcaacgtacgacagaatttgccaccaatttcagtttatgccgacgat
gcggttctgtttttccgccctacagctgaagaagctcgagtcatcaagggtatcctggagttgttcggggctgccacaagtctcaaaacca
atttctccaaaagcgcaatcactccaatccaatgtgacgagcagcagtatgtgcaagttgaatccattctctcctgccgagtggaaaagtt
cccaatcacttatcttggactccctctctccactaggaaaccaacgaaggccgagatccagccgatccttgataggctggcaaagaaggt
agccggttggaagccgaaaatgctgtctattgatgggcgactgtgcttgatcaagtcggtcctaatggcgctgccggtgcactacatgac
agtcctgcagctaccgcgatgggcgattaaggacatcgagcggaagtgccgtgggtttctttggaaaggacaggaagagatcagcggc
gggcattgcctagtctcgtggcgaaaggtttgctcacccatcgagaaagggggacttggtgtcaaagatcttaatttgttcggtcaagctc
tccggttgaaatggcttgcaaaatccttggagcagaaggatagaccctggaccttagcaactttccgtcctggaagcgatgtggaagag
atctttcgatccgttgctgagcacatcattggtgacggggtgaacacacagttttggacagacaattggacagggaaaggttgcttcgcct
ggaggtggccggtgttgttttcccatgtgagccgtgccaagctgacagtagctgatgccctgattgctaacagatgggttcgccgattaca
aggtgccttgtccaatgaagctctgggtgaattcttccaactttgggatgaagttcacgacgtgtcactgcagcagatggctaaaacgatc
aaatggaagttgactgttgatggtaatttctcagtggcctcggcgtatgatctatttttcatagcgacagaggactgttcctacggggacac
gctgtggcactccagggtgccgtcgcgtgttcgcttcttcatgtggattgcactcaagggccgctgtctcacggcggacaacctggcaaag
agaaactggccgcatgacgccatttgctccctatgccaacacgagaacgaagactgccattatttgcttgtgtcctgtgattatacggcgg
cggtttggcgcaagctgagacgttggtgcaacattaacattgcaatccctgcggaagatggcatgccgcttgcagattggtggatcgcga
caagacggcgttttcagaacacgtataggacggatttcgatagtctgttaatgctaatttgttggcttatctggaaagagcgaaatacaag
gatctttcaacacategccaagtcggttgaccggctagcggatgacatcaacgaggaaatcgcaatttggagggcagcagggattttctc
ccaggctagcgagtaatcccgattagaggcgggacctgccccattttttccttttctttccgggcttgagtttgcttgagaccggcgcgaca
tccttcatgtcgttgtaattaaaactttatttccctcaatcttaataaaattggccggcctacctttggccgtcccggcaaaaaagaatctag
aatatatagctacatattctcaaaatcgaatctggactgttttggagagtagccgctagaaacttcctagaacaaaacccttatatttgttctttaagtc
acatcatacttgctgatgaaatcactatccattagttactccatccgtcccaaaaatacttaatctaggagaagatgtgactccttctgatacaataaatt
tggataaagagctatcagatttgttaggatcacacatttatttgtaggttaagttttttttaacggaagtagtacgcataaaggattggcttacccaattgt
taaccggcccggcactggaacagaaaggtcttgaacccaaacgggacgccgagaaggcccttccctgacgaaagcaaagggcttaattagct
agcaagaaacccaaaccgaccegagcccgtcacgegegegeccgtgacctacegtgcgctgegcegcctcctecetcccacctcccttcaca
aaagcagcgacccctcctccctccccaagtttcctccccacacegcaacccttctctctctctctctctcccctctegacttctctectetccgccgcc
tccgagtcccgccgcgccgegegcccgtcttccccggeggccgatgtgtctgcctegteggcacgaaaccctagaggtaacccgccgcgccg
ctccccgccgcttcccgccgegategggggccctcccccctagggttttcgggggacttttgagggtggatgatttgggggtgtggggggctttg
ggggcggtctaacctgtttgtggtttctggtgcaggtgcggtgcagttgaggggtcccgatcggagatggcggcggcggcggccatggcggcg
caccgggccagcttcccgctccggctgcagcagatcctgtccgggagccgcgccgtgtcgccgtcgatcaaggtggagtccgagccggtgag
tccctegegccgttcccctgtttcctcgccctagggttttgatcgtcggggttgaggggttgtagatgcgaagttgagatggtatgtaggatcgaatc
ctccctaggtgcttcctctagggttttgatcggctgcctgtgttgatgtggcgtgctgttggggtgaggtagttaggccgtaaggagtttgctccgttt
atgatcggtgttgagcatggggaccagtggtgtggtgtgcagggtagttgttactgctttaggccatctcaaatttgggtttccttggtcaggggtag
aagagacaccggtttgaagtttctggttatcttgcttgtgctgttattgtactatattgtagtagggatacatgctcgtgttattctgttaccttgtttaagca
tgtctatgcccctcaatgcttagttgccgctgcagccgtaatcttttaggcttagccgcttaggtatccccattacatttgtattatcttgttattactacgg
tgtcccattggacatttattagttcagactttcttgcacttgtaattccttctgcaaaacatacgagtcaatacagaatgccacatctagcaaattactatgtta
tcattgatgcttaggtgcccatgatcagtacttatggacttgtactggccattttataatgttattttttcattctgttattgctatagctttttaatccttttt
tacgtatttttatttctgtgcacaactgcacttatgttgaccaatcctgtatcatgttttggataatggcttactacataaatatatgacgttggatagtagcc
tcaagattgatgcattgatttagttcacttgatattacagctcaagagttgagacatgtggttttgtggatgatatacatttggtctttacagttattttaatta
catctaagtttgttttgcacaatactggttgaatgtctcaacattttgcctgatctgtgggtctaaatatataacaatgagaatttaattggaagatatattt
ataattcacagattcattcatgagctttgaattttaatctcttaacaataatagtatttgcttttgagattttaaggaagatagacgagtgttaggaaccttg
caccaggtagaatatgaagtggatttggatgctttatctctgatgacttgtttggtaactacttagacaaactattggtctgttcaccttttcaaggagat
atggcatacagctaactatgttgattggattatcaatgtacgggttgtgttgcatttagtagacaatcatattctttgaataaataagtgcattatatttaaa
gataaagtggttacatgttgcataagggcaatcatttgtaccattttagtatgcttgctgttctctgaaagtttattctgtttcttgcaacaccagccaacat
gattgatcaatcattttttttgtgtttgcagccagcaaaagttaaagcatttattgatcgtgtaatcagtattccactacatgacattgctataccattgtca
ggcttccgttgggagttcaataaggtgacttctttattagagaagctcttacatgactttttcacatccttactttcctgtagtttcttacttcaaatatgtgg
ccacagggaaatttccaccattggaagcctctttttatgcattttgatacatatttcaagacacaaatttcttcgaggaaggatcttcttttatctgatgata
tggctgagggtgatcctttgcctaaaaataccatcctgcagattttgagagtaatgcagattgttttggaaaattgccagaacaaaacatcgtttgctggtct
tgaggtaatctggttttaatgttctttaacttgattttccaaatattagatgcaagagcccatgcttatttatatgttttaatttgctatctccttctatcag
cattttaggcttctgctggcatcatcagatcctgagatagttgtggctgctttagagacacttgctgcattggttaaaataaatccttcgaagttgcatat
gaacggaaagctcataaattgtggagctataaacagtcatcttctatcattggcacaaggatggggtagcaaggaggaaggtttgggcttatattct
tgtgttgtggcaaatgaaagaaaccagcaggagggtttgtgcttattcccagcagacatggagaacaaatacgatggcacgcagcaccgtctcg
gttcaactcttcattttgaatataatttggcacctgcccaagatcctgaccaatccagtgacaaggctaagccatctaatctgtgtgtgatacatatccc
agacttgcaccttcagaaggaggatgacttgagcatattgaagcaatgtgttgataagtttaatgtgccttcagagcacagattttccttgtttacaag
gataagatatgcccatgcctttaattcgccacggacatgtaggctatatagccgcataagtcttcttgctttcattgttcttgtgcaatccagegatgcc
catgatgaactcacatctttctttacaaatgagccagagtacataaatgagttaatcagacttgtccgatcagaggaatttgttcctggacccatacga
gcgctggctatgcttgcactgggagcacagttagcagcgtatgcatcatctcatgaacgagctcggatacttagtggctcaagtatcatatctgctg
gtggaaaccgcatggtcttgctcagtgttttgcaaaaagctatatcatcactcagtagccctaatgatacatcatctccattaattgttgatgcccttctg
cagttttttctgctccatgtgctatcttcttcgagttctgggaccactgttagaggttcagggatggttcccccgctcttgccccttttgcaagataatgat
ccttcacacatgcatcttgtctgtctggcagtgaaaactcttcaaaagttgatggagtacagcagccctgctgtttctctatttaaagatttgggtggtgt
agaacttttgtctcagaggttgcacgtggaggtgcagcgtgttattggtgttgacagtcataattcaatggttacaagtgatgcattgaaatcagaag
aggatcatctctactctcagaagcgattgattaaggcgctgctaaaggcattggggtctgctacatattctcctgcaaatcctgctcgttcacaaagct
caaatgataattctttgcccatctcgctttcccttatatttcagaatgttgacaagtttggtggtgacatttatttctcagcagttactgttatgagtgagata
attcacaaggatccaacatgctttccttctttgaaggaacttggtcttccagatgcttttctatcgtcagtgagtgctggggtaataccatcttgtaaagc
tctcatctgtgtgcctaatggtctgggtgcaatatgccttaataaccaaggacttgaggctgtcagggaaacttcagctctgogttttcttgttgacaca
ttcaccagcaggaagtacttgataccaatgaatgaaggtgttgtcctattagctaatgcagtggaagagcttctacgtcacgtgcagtccctaagaa
gcactggggttgacatcattattgaaataattaataaactttcttcacctcgtgaagataagagcaatgaaccagcggccagttctgatgaaagaaca
gaaatggaaactgacgcggaaggacgtgatttggtaagtgctatggattccagtgaggatggcactaatgatgaacagttttctcatttgagcatttt
ccatgtgatggtattggttcatcggacaatggagaactccgaaacctgccggttatttgtggagaaaggaggcctgcaagcacttttgacactcctg
ttgcgacctagcattacccaatcatctggaggaatgccgattgctttgcatagcaccatggtattcaagggctttactcagcatcactctactccactt
gcacgtgcattttgctcttccttaaaggagcatttaaagaatgccttgcaggaacttgatacagttgcaagctctggtgaagtggcaaagttagaaaa
aggagcaattccatctctttttgttgttgagttcttactcttccttgcggcatccaaagataatcgctggatgaatgctctactctcagaatttggagatag
cagtagggatgtcctggaagatattggacgagtacaccgagaagtgctttggcaaatttcactttttgaagaaaagaaagttgagcctgaaacaag
ttctcctttagcaaatgactcccagcaagacgcagctgtgggggatgttgatgatagcagatacacatcctttaggcaatatcttgatcctcttttgag
gcgaaggggctctgggtggaatattgaatcacaggtgtctgacctcattaatatctaccgtgatattggccgtgcagctggtgactctcagaggtat
cctagtgcagggttgccctcaagttcttctcaagaccagcctcccagttcatctgatgcaagtgctagcacaaaatcagaagaggacaagaaaag
atctgagcattcttcctgctgtgacatgatgaggtcactgtcttaccatatcaatcatcttttcatggagcttgggaaagcaatgcttcttacatctegtc
gggagaacagccctgtgaatttatctgcatctattgtatctgttgctagcaatattgcttctattgtgttggagcacctcaattttgaggggcacacaatc
agttctgaaagagagactactgtttccacaaaatgccgataccttgggaaggtggttgagttcattgatggtatattgttggacaggccggaatcgtg
caacccaatcatgctgaattcattttattgccgtggtgttattcaggctattttaaccacatttgaagctaccagtgagttgctcttttctatgaacaggctt
ccgtcatcgcctatggagacagacagtaaaagtgttaaggaagacagggagacagattcgtcatggatatatggtccactctccagctatggtgc
aattctggaccatctagtaacatcatogtttattctttcttcctcaacaagacaattacttgagcagcctatttttagtggaaatatcaggtttccccaaga
tgcagagaagttcatgaagctgcttcagtcaagagttctgaagactgttcttcccatctggacccatcctcagtttccagaatgtaatgttgagttaatt
agttcagtcacatctatcatgaggcatgtttactctggggttgaagtgaaaaacactgctatcaacactggtgctcgtttggctggtccaccccctgat
gagaatgcaatttctctgattgtagagatgggcttttctcgcgccagagctgaggaagcactcaggcaagttggaacgaacagtgttgaaattgca
actgattggttattctcacacccagaggaaccacaagaggatgacgaacttgctcgagctcttgcaatgtctttaggcaattctgatacgtctgcaca
agaggaagatggcaaatcgaatgatcttgaacttgaagaagaaactgttcagctgcctcccatagatgaagtattgtcttcatgtcttaggttgcttca
gacaaaggaatcattagctttccctgttcgggacatgcttttgactatgagctcacagaatgatggtcaaaaccgagtaaaggttcttacgtatttgatt
gatcacctgaaaaattgtctgatgtcatctgatcctttaaagagcactgcattatcagctctttttcatgtccttgctttgattctccatggagatactgctg
ctcgggaagttgcttcaaaggctggtcttgtcaaggttgctttgaacctgctgtgcagctgggagttggagccgaggcaaggcgagataagtgat
gttccaaattgggttccttcatgctttctttctattgataggatgctccagttggacccaaagttgccagatgttactgaactcgatgtccttaaaaagga
taattcaaatacacaaacatcagtggtgattgatgatagcaagaaaaaggactcagaagcttcatcgagcacagggttattggacttggaggacca
gaagcaacttttgaagatttgctgtaaatgcattcagaagcagttgccttctgctaccatgcatgctattcttcagttatgtgccacgttgactaaacttc
atgctgctgctatttgttttcttgagtctggtggtctgcatgcattgctaagtttgcccacaagtagcttgttttctggattcaacagtgtggcttctacaat
cattcgtcatattttggaagatccccacactcttcagcaagcaatggaattagagatacgccacagtcttgtcaccgctgcaaatcgtcatgcaaatc
caagggttacaccgcgcaattttgtccagaacttggcgtttgttgtatatagagacccagtgatatttatgaaagctgcccaagctgtgtgccagatt
gagatggttggtgatagaccatatgttgttctgttgaaggatcgtgaaaaagaaaagaacaaggaaaaagagaaggacaagcctgctgataagg
ataaaacatcaggtgcagccacaaagatgacatcaggggacatggctttaggatctcctgtaagttctcaagggaagcagactgatctgaataca
aagaatgtgaaatctaatcgcaaaccaccacaaagctttgtcactgttattgagtatctgctagatctggttatgtccttcattccacctcctagagcag
aagatcgacctgatggtgaatctagtactgcatcatctacagacatggatattgacagctcagcaaaaggcaaaggtaaagctgttgctgtcacac
ctgaagagtccaagcatgcaattcaagaggctactgcatctctcgctaaaagtgcatttgttctgaagctgctaacagatgttcttctgacttatgcat
catctattcaagttgttcttcgacatgatgctgatttgagcaatgcacgtggtcctaaccggattggtattagcagtggtggggttttcagtcatatactg
cagcatttccttccgcattctacaaagcaaaagaaagagaggaaagctgatggagattggaggtacaaattggcaacaagggctaatcaattcttg
gtggcttcatctattcggtctgcagaaggtagaaaaaggatcttttctgaaatctgcagcatatttgttgacttcacagactcccctgctggttgcaaac
ccccaatattaaggatgaatgcatatgttgatttgcttaatgatattctgtcagcccgttcgccaactggttcctccttgtcagcagaatctgcagttact
tttgttgaagttggtcttgttcagtatttatcaaaaacactgcaagttatagatttggatcatcctgattcagcaaagattgtaactgctattgttaaggcc
cttgaggttgtcacaaaggaacatgttcattcggcagatttgaatgccaaaggggagaactcatcaaaggttgtgtctgaccagagcaatctagac
ccgtcttcaaatagattccaagctcttgacacaactcaacccactgagatggttactgatcatagggaagctttcaatgctgttcaaacttcacaaagt
tcagattcagtggctgatgagatggaccatgaccgtgatctggatggaggatttgctcgtgatggtgaagatgactttatgcacgagattgctgaag
atggaactccaaatgagtccacaatggaaatcagatttgaaattccacgaaatagagaggatgatatggctgatgatgacgaggacagtgatgag
gacatgtcagccgatgatggtgaggaggttgatgaagatgaagacgaggatgaggatgaagagaacaacaacctggaggaggatgatgccca
tcaaatgtctcatcctgacacagatcaggaggaccgtgagatggatgaagaggagtttgacgaggatctgctagaagaagatgatgatgaggat

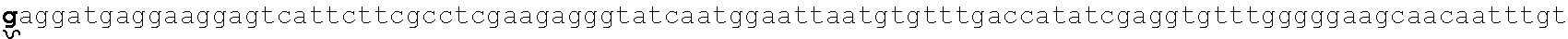

ctggggatacactgcgtgtaatgccgttggacatttttggaacaagacggcaaggtcgtagtacatctatatataaccttcttgggagagcaggcga
tcatggtgtttttgaccacccgctcttggaggagccttcttcggtgctacaccttccacagcaaagacaacaaggtatgccttctttccttccctgttcatg
ttgattctgttccatgtaatcatccattggcaaactagtaagcaactgtctgattatttttttttgactttctaatatgttactgatatacctagatggtacc
aattctggcatacatcactaattcaaattaccgtttgtttcagaaaatttagttgagatggccttctctgatcggaatcatgataatagttcttcccgcttg
gatgcaattttccggagcctgcgaagtggccggagtggacaccgttttaatatgtggctagatgacagtccccaacgcactggatcagctgctcct
gcagtacctgaaggcattgaggagctgctggtctctcagttgagacgacccacccctgaacaacctgatgagcagagtacacctgctggtggcg
ctgaagaaaatgaccaatctaatcagcaacatttgcatcaatcagaaactgaggcaggaggagatgcaccaacagaacaaaatgaaaacaatga
taatgcagttactccggcagcaaggtctgagttagatggttctgaaagtgctgatcctgcacctcccagcaatgcacttcaaagagaagtgtctggt
gcaagtgagcatgccacggagatgcaatatgaacgtagtgatgctgtagtacgtgatgtggaagcagtcagccaggcaagcagtggtagcggt
gctactttaggggaaagccttagaagtttagaggtggagataggaagtgttgaagggcatgatgatggtgatcgccacggagcttcagacaggct

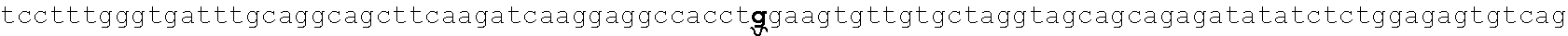

cgaggttcctcaaaatcaaaatcaagaatctgatcagaatgctgatgaaggggatcaggagcctaacagagctgctgacactgactcaattgatc
ctacatttttggaggctcttccagaggatttacgggctgaagttctttcttcacgtcaaaatcaagtgacccagacttctaatgaacaacctcagaatg
atggggatattgatcctgaattccttgctgcacttcctcctgatatacgtgaagaagttctagctcaacaacgtgogcaaaggttgcagcagtcacag
gaattagaaggacaaccagttgaaatggatgctgtttcaattatcgcaacattcccttcagaaattcgggaggaggtatatagtttgttctgtaccagt
cccatttttcatttctttgtcataatgtgatcttatggttgagttattttgcaggtgcttttaacatctccagatacattactggctacacttacgcctgcacta
gttgctgaagcaaacatgttaagggagagatttgctcatcggtatcacagtggctccctttttggcatgaactccaggggcaggagaggtgagtcc
tctcgacgtggtgacataattggttcaggtcttgatagaaatgctggtgattcttctcgacaaccaactagcaagccaattgaaacggaaggatctcc
tcttgttgacaaggatgctcttaaagctcttattaggctactccgggttgttcaggtaatataccattaacttctgtgtgttcaactgtgtaaagttctctgg
aaaaaaaatcttctactaactttacccattgtttacagcctctatacaaaggtcaattgcagaggcttctcttgaacctttgtgctcatagggaaagcag
aaagtccttggttcaaattctagtggacatgcttatgcttgatctgcagggctcttctaagaaatcaattgatgcaactgagccaccatttaggctatat
gggtgccatgcaaatattacgtactcacgccctcaatcgacagatggtaacctaactacccttgtttctgtgtttttaattagctgaatggtgctcttggtatc
taggttaacatttgcctgttgagaattatagttgatattgattgattttctttattgtggttaataggcgtgcctccattagtttctcgtcgtgttcttgaaa
ctttgacatacttggcaagaaatcatccaaatgtggctaaactcttgctatttcttgagttcccttgccccccaacttgccatgctgaaacatctgatca
gaggcgtggcaaggctgttcttatggaaggtgacagtgaacagaacgcttatgcacttgtcctacttttaaccttgttgaatcagccactttatatgag
gagcgtagctcatcttgaacaggttaacattctttcttgttttttattttctgttgtggctctttattaaaatttccagtcatatttttatcctaaccattggaa
cttgtgtagctactaaaccttctcgaagttgttatgctcaatgccgagaatgaaattacacaagctaagctggaagcagcatctgaaaaaccatctggac
ctgagaatgcaacgcaagatgcccaagagggtgcgaatgctgctggatcatctggatcgaagtccaatgctgaggatagcagcaaactccctcc
tgttgatggtgaaagtagcctgcaaaaagttctgcagagtcttccccaagcagagcttcgactgctatgttcactgcttgcacatgatgggtataaactttc
ccaattttggtgaattgcttataattcatttttttctcctatttaattctattactttcatagtgtaagcacattgaggaaatcataaatgcagctattgcaaca
ttacttctcttctctttagtacttgtgcatatggtgggtttcaacttacattgcagatttgattaagtttgattattctctggttatgttgtttaggtcttcat
caaatagatgataagaaactaggctgctagttgcatcagcattttcttgtccttggtttcgttctttgtgatctgtgtttccttttagaaacatagatggcagag
ctgtaactttttcatatatttgtttctgctattatttctgttacgactaataaaagaaatgcttgtgtttgtctttcaggttgtcagacaatgegtatctcctg

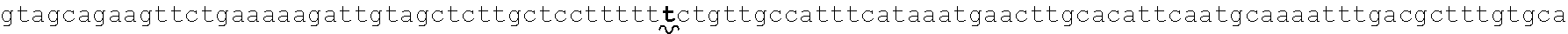

atgaaggagcttcacttgtatgaggattctgaaaaggctcttcttagcacatcatcagccaatggcactgcaattcttagagttgtgcaggctttgagt
tctcttgtcaccactctgcaagagaaaaaggatccagatcatcctgctgaaaaagatcattctgatgcattgtcccagatttctgaaattaacactgca
ttggatgcattatggttggagctgagtaattgcataagcaaaatagagagctcttcagaatacgcatcgaatctaagtcctgcttctgcaaatgcagc
cacattaacaacaggtgtagcacctccattgcctgccggaactcagaacatattaccgtacatagaatcatttttcgtgacatgtgagaagttacgcc
ctgggcaacctgatgctattcaagaagcttcaacatctgacatggaggatgcatcaacttctagtggtgggcagaaatcatctggaagccatgcaa
atcttgatgagaagcacaatgcgtttgttaaattctcagagaaacacagaagattgttgaacgcatttatccgccaaaaccctgggctattggagaa
gtcattctctctgatgttgaaaatccctcgcttgattgaatttgacaacaagcgtgcatatttccggtctaaaattaagcatcagcatgatcatcatcata
gccctgttagaatttctgtgcgccgggcatatattttggaggattcatataaccagcttaggatgcgttcaccacaggatttgaagggtagactgact
gttcatttccaaggtgaagaaggcattgatgctggtggactaacaagggaatggtatcagctgctatcacgagtgatttttgataagggtgcccttct
attcacaactgttggaaatgacttgacatttcaaccaaaccctaactcggtgtatcagactgaacacctctcatatttcaaatttgttgggcgagtggtgagt
gatattgctccttgtttttcactttcagctttgtgcaattgttgttggttctaaaagttgtccctccaggttggtaaagctctatttgatggccaacttttg

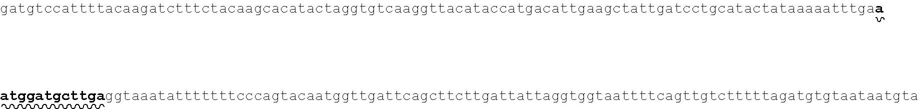

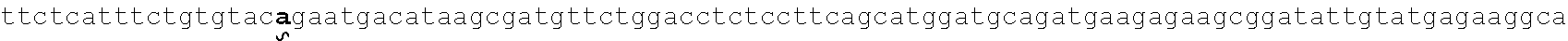

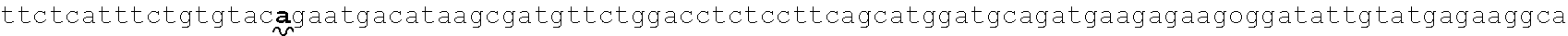

gaggtataagcctatctctgtgtttgtctgtcttttcgctgttgcttgtctttgcttgaaacttagtcctgaacccatctatgcaggtgactgattatgagtt
gattcctggaggccgaaacatcaaggtcaccgaggagaacaagcatgaatatgtgaaccgggttgcagaacatcgtttaaccactgctattaggc
ctcaaatcacctcttttatggagggatttaatgagctcattcctgaggagctgatatcaatctttaatgacaaagaacttgaactgctaatcagtggactccca
gacattgactgtgagtatcacccatgatttaggactgtttaattatctgtttttttatcttacagttaattaacttgttttgtatctctcgctttcagtgga
cgatctaaaagcaaatacagaatattctgggtacagcatagcttctccagtcattcagtggttctgggagattgtccaagggttcagcaaggaggac
aaagcccggttccttcagtttgttactggcacctcaaaggtactttgctgatgatgccttgtgaagtattttttatttagaagcgttagcccacatgattct
atcttacttggtgattccccgcgctttgctacgggaattactaaacatttctcaatatatttttttcaacaatcaagctagaagtgaaagggaaaaaataa
taatgaaacattagggtttatccatacttatcatgaaacaaacaaatggtatttgcgctttgatgtgaaacatattgataagtatatgtttaatattataaaa
atgatagatttgtaaaaatattgtgcaaataatgtgaggggtgatgattggatatgcttgcatgttgatttgtagaattaaataaattgtagataatgttga
agttaaatatgtaattattgctcgtgggtgatgatgtggcatagttgcatgttgaacgacttagtgggctataactttatagtaagataggattagtctgg
tgttctccctcaaagctcactaatttttttaccccgcccatgtgataagctgagttattcagcatgatttgagtattggctggcacattcaattctttaatga
taatcttttgcgtattatatttggtttcttattcttttgtgttaaactactgtaggtacctctggaaggtttcagtgcactccaaggaatatctggaccacaac
gattccagatacacaaggcctacggaagcaccaaccatctgccttcagcacatacttggtaatccatcttcactgcactcacttttgacactagaaaa
aaaatcatttggcacaattaagggttcaaaatcctgctggtggttaccctttttgtccagctataatgttgttttattttatttacttgagatcagtcctgaca
actctactgcccttgctcactgcagctttaaccaactagaccttcctgagtacacatcgaaagagcagctccaggagagattgctactggctattcat
gaggcgaatgaaggtttcggatttggttaatcagtcactcctgcacctgtgtgcaagaaatttcagggagtaatgtacagataccgtcggagttgca
ataggcgaggggaatgtgcgcggactcttacataacctgctactagattcatttgttgcctgcatcaaccatcggcgttggtccctgaagactgatg
agatttgttgacaaagtaccggcctgcccacgatgctttataggactggttgctgcgaaggatgtgcagggaggtgtagcaggaagtgctagaagacag
caactatttggtgttcataatattttttttctttcccttttgggtcttttttggccattgccccgttaatagatttcaccttctctatacattggacctgtat
ggaatttttttccttttttattcaagtttgtttttggggggcatagaccggtggaatgcaacattaagtagaatgcaagttttccatcgctattgcatattca
atgcacattgactaaaagtgtctggagccacgggctgcggctataaattttactcctaaggtgatgtgtgttcgtcggtgacttgttcgtacggttatgt
gtgtctgttttgagatgtgaaacttggcttggaccctaaatttggcataatagtgccgtaccaccagttcaccactatttgttaggccaacaccatctaa
tattcgatttccgctacatagtacgctacattcagtaattaagatcaaatccttccgctacataataacataatcaagcctogtagggctccatactgca
ccgttttttgtaaattttttttgctgcttccatgatttgtcacgtgggtgttcagtgctatagctcctcgagtagccggttccccggttcctttggcagatgt
gttttacttttttttttctacttgtttgatatacctgtcagcatgcagcatgctatctcagtcttttccattgctaacgtgttagcacgcatgtttgctttgtc
ttcttactttgcacctategccggcaggatgcacatgtccctgcaccgcctgatgcacagtcttctccccttttgaaattttcaaaaaagtcctctgatttgtg
tat

### REFERENCES

Cermak, T et al. Efficient design and assembly of custom TALEN and other TAL effector-based constructs for DNA targeting. Nucleic acid Res. 39 (2011).
Kunkel T A. 1985. Rapid and efficient dite-specifc mutagenesis without phenotypic selection. PNAS. 82 (2): 488-92.
Kunkel T A, Roberts J D, Zakour R A. 1987. Rapid and efficient dite-specifc mutagenesis without phenotypic selection. Methods Enzmol. 154. 367-82.
Henikoff S, Till B J, Comai L. 2004. TILLING. Traditional mutagenesis meets functional genomics. Plant Physiol. 135 (2): 630-6.
Comai L, Young K, Till B J, Reynolds S H, Greene E A, Codoma C A, Enns L C, Johnson J E, Burtner C, Odden A R, Heinkoff. 2004. Efficient discovery of DNA polymorphisms in natural populations by Ecotilling. Plant J. 37 (5): 778-86.
Clough S J, Bent A F. 1998. Floral dip: a simplified method for Agrobacterium-mediated transformation of Arabidopsis thaliana. Plant J. 16 (6): 735-43.
Ma X, Zhang Q, Zhu Q, Liu W, Chen Y, Qiu R, Wang B, Yang Z, Li H, Lin Y, Xie Y, Shen R, Chen S, Wang Z, Chen Y, Guo J, Chen L, Zhao X, Dong Z, Liu Y. 2015. A Robust CRISPR/Cas9 System for Convenient, High-Efficiency Multiplex Genome Editing in Monocot and Dicot Plants. 8 (8): 1274-84.
Abe, A., Kosugi, S., Yoshida, K., Natsume, S., Takagi, H., Kanzaki, H., Matsumura, H., Yoshida, K., Mitsuoka, C., Tamiru, M., Innan, H., Cano, L., Kamoun, S., and Terauchi, R. (2012). Genome sequencing reveals agronomically important loci in rice using MutMap. Nat Biotechnol 30, 174-178.
Ashikari, M., Sakakibara, H., Lin, S., Yamamoto, T., Takashi, T., Nishimura, A., Angeles, E. R., Qian, Q., Kitano, H., and Matsuoka, M. (2005). Cytokinin oxidase regulates rice grain production. Science 309, 741-745.
Bates, P. W., and Vierstra, R. D. (1999). UPL1 and 2, two 405 kDa ubiquitin-protein ligases from Arabidopsis thaliana related to the HECT-domain protein family. Plant J 20, 183-195.
Callis, J. (2014). The ubiquitination machinery of the ubiquitin system. Arabidopsis Book 12, e0174.
Chae, E., Tan, Q. K., Hill, T. A., and Irish, V. F. (2008). An Arabidopsis F-box protein acts as a transcriptional co-factor to regulate floral development. Development 135, 1235-1245.
Cui, X., Jin, P., Cui, X., Gu, L., Lu, Z., Xue, Y., Wei, L., Qi, J., Song, X., Luo, M., An, G., and Cao, X. (2013). Control of transposon activity by a histone H3K4 demethylase in rice. Proc Natl Acad Sci USA 110, 1953-1958.
Downes, B. P., Stupar, R. M., Gingerich, D. J., and Vierstra, R. D. (2003). The HECT ubiquitin-protein ligase (UPL) family in Arabidopsis: UPL3 has a specific role in trichome development. Plant J 35, 729-742.
Duan, P., Rao, Y., Zeng, D., Yang, Y., Xu, R., Zhang, B., Dong, G., Qian, Q., and Li, Y. (2014). SMALL GRAIN 1, which encodes a mitogen-activated protein kinase kinase 4, influences grain size in rice. Plant J 77, 547-557.
Fang, N., Xu, R., Huang, L., Zhang, B., Duan, P., Li, N., Luo, Y., and Li, Y. (2016). SMALL GRAIN 11 Controls Grain Size, Grain Number and Grain Yield in Rice. Rice (N Y) 9, 64.
Guo, T., Chen, K., Dong, N. Q., Shi, C. L., Ye, W. W., Gao, J. P., Shan, J. X., and Lin, H. X. (2018). GRAIN SIZE AND NUMBER1 Negatively Regulates the OsMKKK10-OsMKK4-OsMPK6 Cascade to Coordinate the Trade-off between Grain Number per panicle and Grain Size in Rice. Plant Cell 30, 871-888.
Herr, J. M., Jr. (1982). An analysis of methods for permanently mounting ovules cleared in four-and-a-half type clearing fluids. Stain Technol 57, 161-169.
Hershko, A., and Ciechanover, A. (1998). THE UBIQUITIN SYSTEM. Annu. Rev. Biochem. 67, 425-479.
Huang, K., Wang, D., Duan, P., Zhang, B., Xu, R., Li, N., and Li, Y. (2017). WIDE AND THICK GRAIN 1, which encodes an otubain-like protease with deubiquitination activity, influences grain size and shape in rice. Plant J 91, 849-860.
Huang, X., Qian, Q., Liu, Z., Sun, H., He, S., Luo, D., Xia, G., Chu, C., Li, J., and Fu, X. (2009). Natural variation at the DEP1 locus enhances grain yield in rice. Nat Genet 41, 494-497.
Huo, X., Wu, S., Zhu, Z., Liu, F., Fu, Y., Cai, H., Sun, X., Gu, P., Xie, D., Tan, L., and Sun, C. (2017). NOG1 increases grain production in rice. Nat Commun 8, 1497.
Ikeda-Kawakatsu, K., Maekawa, M., Izawa, T., Itoh, J., and Nagato, Y. (2012). ABERRANT PANICLE ORGANIZATION 2/RFL, the rice ortholog of Arabidopsis LEAFY, suppresses the transition from panicle meristem to floral meristem through interaction with APO1. Plant J 69, 168-180.
Ikeda-Kawakatsu, K., Yasuno, N., Oikawa, T., Iida, S., Nagato, Y., Maekawa, M., and Kyozuka, J. (2009). Expression level of ABERRANT PANICLE ORGANIZATION1 determines rice panicle form through control of cell proliferation in the meristem. Plant Physiol 150, 736-747.
Ikeda, K., Nagasawa, N., and Nagato, Y. (2005). ABERRANT PANICLE ORGANIZATION 1 temporally regulates meristem identity in rice. Dev Biol 282, 349-360.
Ikeda, K., Ito, M., Nagasawa, N., Kyozuka, J., and Nagato, Y. (2007). Rice ABERRANT PANICLE ORGANIZATION 1, encoding an F-box protein, regulates meristem fate. Plant J 51, 1030-1040.
Itoh, J., Nonomura, K., Ikeda, K., Yamaki, S., Inukai, Y., Yamagishi, H., Kitano, H., and Nagato, Y. (2005). Rice plant development: from zygote to spikelet. Plant Cell Physiol 46, 23-47.
Jiao, Y., Wang, Y., Xue, D., Wang, J., Yan, M., Liu, G., Dong, G., Zeng, D., Lu, Z., Zhu, X., Qian, Q., and Li, J. (2010). Regulation of OsSPL14 by OsmiR156 defines ideal plant architecture in rice. Nat Genet 42, 541-544.
Komatsu, K., Maekawa, M., Ujiie, S., Satake, Y., Furutani, I., Okamoto, H., Shimamoto, K., and Kyozuka, J. (2003). LAX and SPA: major regulators of shoot branching in rice. Proc Natl Acad Sci USA 100, 11765-11770.
Kurakawa, T., Ueda, N., Maekawa, M., Kobayashi, K., Kojima, M., Nagato, Y., Sakakibara, H., and Kyozuka, J. (2007). Direct control of shoot meristem activity by a cytokinin-activating enzyme. Nature 445, 652-655.
Kyozuka, J., Konishi, S., Nemoto, K., Izawa, T., and Shimamoto, K. (1998). Down-regulation of RFL, the FLO/LFY homolog of rice, accompanied with panicle branch initiation. Proc Natl Acad Sci USA 95, 1979-1982.
Lee, Z. H., Hirakawa, T., Yamaguchi, N., and Ito, T. (2019). The Roles of Plant Hormones and Their Interactions with Regulatory Genes in Determining Meristem Activity. Int J Mol Sci 20.
Li, N., and Li, Y. (2016). Signaling pathways of seed size control in plants. Curr Opin Plant Biol 33, 23-32.
Li, N., Liu, Z., Wang, Z., Ru, L., Gonzalez, N., Baekelandt, A., Pauwels, L., Goossens, A., Xu, R., Zhu, Z., Inze, D., and Li, Y. (2018). STERILE APETALA modulates the stability of a repressor protein complex to control organ size in Arabidopsis thaliana. PLOS Genet 14, e1007218.
Li, S., Zhao, B., Yuan, D., Duan, M., Qian, Q., Tang, L., Wang, B., Liu, X., Zhang, J., Wang, J., Sun, J., Liu, Z., Feng, Y., Yuan, L., and Li, C. (2013). Rice zinc finger protein DST enhances grain production through controlling Gn1a/OsCKX2 expression. Proc Natl Acad Sci USA 110, 3167-3172.
Liu, X., Zhou, S., Wang, W., Ye, Y., Zhao, Y., Xu, Q., Zhou, C., Tan, F., Cheng, S., and Zhou, D. X. (2015). Regulation of histone methylation and reprogramming of gene expression in the rice panicle meristem. Plant Cell 27, 1428-1444.
Miao, Y., and Zentgraf, U. (2010). A HECT E3 ubiquitin ligase negatively regulates Arabidopsis leaf senescence through degradation of the transcription factor WRKY53. Plant J 63, 179-188.
Miller, C., Wells, R., Mckenzie, N., Trick, M., Ball, J., Fatihi, A., Dubreucq, B., Chardot, T., Lepiniec, L., and Bevan, M. W. (2019). Variation in Expression of the HECT E3 Ligase UPL3 Modulates LEC2 Levels, Seed Size, and Crop Yields in Brassica napus. Plant Cell 31, 2370-2385.
Miura, K., Ikeda, M., Matsubara, A., Song, X. J., Ito, M., Asano, K., Matsuoka, M., Kitano, H., and Ashikari, M. (2010). OsSPL14 promotes panicle branching and higher grain productivity in rice. Nat Genet 42, 545-549.
Ookawa, T., Hobo, T., Yano, M., Murata, K., Ando, T., Miura, H., Asano, K., Ochiai, Y., Ikeda, M., Nishitani, R., Ebitani, T., Ozaki, H., Angeles, E. R., Hirasawa, T., and Matsuoka, M. (2010). New approach for rice improvement using a pleiotropic QTL gene for lodging resistance and yield. Nat Commun 1, 132.
Patra, B., Pattanaik, S., and Yuan, L. (2013). Ubiquitin protein ligase 3 mediates the proteasomal degradation of GLABROUS 3 and ENHANCER OF GLABROUS 3, regulators of trichome development and flavonoid biosynthesis in Arabidopsis. Plant J 74, 435-447.
Rao, N. N., Prasad, K., Kumar, P. R., and Vijayraghavan, U. (2008). Distinct regulatory role for RFL, the rice LFY homolog, in determining flowering time and plant architecture. Proc Natl Acad Sci USA 105, 3646-3651.
Sakamoto, T., and Matsuoka, M. (2008). Identifying and exploiting grain yield genes in rice. Curr Opin Plant Biol 11, 209-214.
Smalle, J., and Vierstra, R. D. (2004). The ubiquitin 26S proteasome proteolytic pathway. Annu Rev Plant Biol 55, 555-590.
Souer, E., Rebocho, A. B., Bliek, M., Kusters, E., de Bruin, R. A., and Koes, R. (2008). Patterning of panicles and flowers by the F-Box protein DOUBLE TOP and the LEAFY homolog ABERRANT LEAF AND FLOWER of petunia. Plant Cell 20, 2033-2048.
Tsuda, K., Ito, Y., Sato, Y., and Kurata, N. (2011). Positive autoregulation of a KNOX gene is essential for shoot apical meristem maintenance in rice. Plant Cell 23, 4368-4381.
Tsuda, K., Kurata, N., Ohyanagi, H., and Hake, S. (2014). Genome-wide study of KNOX regulatory network reveals brassinosteroid catabolic genes important for shoot meristem function in rice. Plant Cell 26, 3488-3500.
Vierstra, R. D. (2009). The ubiquitin-26S proteasome system at the nexus of plant biology. Nat Rev Mol Cell Biol 10, 385-397.
Wang, B., Smith, S. M., and Li, J. (2018). Genetic Regulation of Shoot Architecture. Annu Rev Plant Biol 69, 437-468.
Wang, J., Wang, R., Wang, Y., Zhang, L., Zhang, L., Xu, Y., and Yao, S. (2017). Short and Solid Culm/RFL/APO2 for culm development in rice. Plant J 91, 85-96.
Wang, X., Lu, G., Li, L., Yi, J., Yan, K., Wang, Y., Zhu, B., Kuang, J., Lin, M., Zhang, S., and Shao, G. (2014). HUWE1 interacts with BRCA1 and promotes its degradation in the ubiquitin-proteasome pathway. Biochem Biophys Res Commun 444, 290-295.
Wang, Z., Li, N., Jiang, S., Gonzalez, N., Huang, X., Wang, Y., Inze, D., and Li, Y. (2016). SCF (SAP) controls organ size by targeting PPD proteins for degradation in Arabidopsis thaliana. Nat Commun 7, 11192.
Werner, T., Motyka, V., Strnad, M., and Schmulling, T. (2001). Regulation of plant growth by cytokinin. Proc Natl Acad Sci USA 98, 10487-10492.
Wu, Y., Wang, Y., Mi, X., Shan, J., Li, X., Xu, J., and Lin, H. (2016). The QTL GNP1 Encodes GA20ox1, Which Increases Grain Number and Yield by Increasing Cytokinin Activity in Rice panicle Meristems. PLOS Genet 12, e1006386.
Xia, T., Li, N., Dumenil, J., Li, J., Kamenski, A., Bevan, M. W., Gao, F., and Li, Y. (2013). The ubiquitin receptor DA1 interacts with the E3 ubiquitin ligase DA2 to regulate seed and organ size in Arabidopsis. Plant Cell 25, 3347-3359.
Xu, R., Yu, H., Wang, J., Duan, P., Zhang, B., Li, J., Li, Y., Xu, J., Lyu, J., Li, N., Chai, T., and Li, Y. (2018a). A mitogen-activated protein kinase phosphatase influences grain size and weight in rice. Plant J.
Xu, R., Duan, P., Yu, H., Zhou, Z., Zhang, B., Wang, R., Li, J., Zhang, G., Zhuang, S., Lyu, J., Li, N., Chai, T., Tian, Z., Yao, S., and Li, Y. (2018b). Control of Grain Size and Weight by the OsMKKK10-OsMKK4-OsMAPK6 Signaling Pathway in Rice. Mol Plant 11, 860-873.
Yau, R., and Rape, M. (2016). The increasing complexity of the ubiquitin code. Nat Cell Biol 18, 579-586.
Yoshida, A., Sasao, M., Yasuno, N., Takagi, K., Daimon, Y., Chen, R., Yamazaki, R., Tokunaga, H., Kitaguchi, Y., Sato, Y., Nagamura, Y., Ushijima, T., Kumamaru, T., lida, S., Maekawa, M., and Kyozuka, J. (2013). TAWAWA1, a regulator of rice panicle architecture, functions through the suppression of meristem phase transition. Proc Natl Acad Sci USA 110, 767-772.
Zhao, L., Tan, L., Zhu, Z., Xiao, L., Xie, D., and Sun, C. (2015). PAY1 improves plant architecture and enhances grain yield in rice. Plant J 83, 528-536.
Zheng, N., and Shabek, N. (2017). Ubiquitin Ligases: Structure, Function, and Regulation. Annu Rev Biochem 86, 129-157.
Zuo, J., and Li, J. (2014). Molecular genetic dissection of quantitative trait loci regulating rice grain size. Annu Rev Genet 48, 99-118.

1. A genetically altered plant, plant part or plant cell comprising at least one mutation in at least one UPL2 gene and/or UPL2 promoter.

2. The plant of claim 1, wherein the mutation is a loss of function or partial loss of function mutation.

3. The plant of claim 1, wherein the plant is heterozygous for the mutation.

4. The plant of claim 1, wherein the UPL2 gene encodes a E3 ubiquitin ligase comprising a HECT domain, and wherein the mutation results in a non-functional HECT domain, wherein preferably the mutation results in the deletion or partial deletion of the HECT domain.

5. The plant of claim 1, wherein the E3 ligase comprises a Glu/Asp-rich domain, and wherein the mutation is in the Glu/Asp-rich domain.

6. The plant of claim 1, wherein the UPL2 gene encodes a polypeptide as defined in SEQ ID NO: 2 or a functional variant or homologue thereof, and wherein the UPL2 promoter comprises or consists of SEQ ID NO: 3 or a functional variant or homologue thereof.

7. The plant of claim 1, wherein the plant is a crop plant, and is preferably selected from rice, wheat, maize, soybean, sorghum, oilseed rape and other vegetable brassicas, barley and millet.

8. (canceled)

9. A seed obtained or obtainable from the plant of claim 1.

10. A method of increasing yield in a plant, the method comprising reducing or abolishing the expression of a UPL2 nucleic acid and/or reducing the activity of a UPL2 polypeptide in said plant.

11. The method of claim 10, wherein the method comprises reducing the E3 ligase activity of the UPL2 polypeptide.

12. The method of claim 10, wherein the method comprises introducing at least one mutation into at least one UPL2 gene and/or UPL2 promoter.

13. (canceled)

14. The method of claim 12, wherein the mutation is a loss of function or partial loss of function mutation.

15. The method of claim 12, wherein the UPL2 gene encodes a E3 ubiquitin ligase comprising a HECT domain, and wherein the mutation results in a non-functional HECT domain, wherein preferably the mutation results in the deletion or partial deletion of the HECT domain.

16. The method of claim 10, wherein the method increases at least one of inflorescence size, grain number per plant, grain width and thousand grain weight.

17. The method of claim 10, wherein the method comprises using RNAi interference to reduce or abolish the expression of a UPL2 nucleic acid.

18. The method of claim 10, wherein the UPL2 gene encodes a polypeptide as defined in SEQ ID NO: 2 or a functional variant or homologue thereof, and wherein the UPL2 promoter comprises or consists of SEQ ID NO: 3 or a functional variant or homologue thereof.

19. The method of claim 10, wherein the plant is a crop plant, and is preferably selected from rice, wheat, maize, soybean, sorghum, oilseed rape and other vegetable brassicas, barley and millet.

20. (canceled)

21. A plant, plant part, part cell or seed obtained by the method of claim 10.

22. A method for identifying and/or selecting a plant that will have an increased yield phenotype, the method comprising detecting in the plant or plant germplasm at least one polymorphism, wherein the polymorphism is a mutation in the UPL2 gene or promoter and selecting said plant, and wherein the mutation is a loss or partial loss of function mutation.

23. (canceled)

24. The method of claim 9, wherein the method further comprises expressing a nucleic acid construct comprising a nucleic acid sequence encoding a sgRNA, wherein the sgRNA comprises a sequence selected from SEQ ID NO: 27, 28, 29, 30, 31, 33, 34, 35, 36, 41, 42, 45, 46, 49, 50, 51, 52, 53, 54, 65, 66, 67, 68, 70, 71, 72, 73 or 74 or a variant thereof.

25. (canceled)
